(19) United States
(12) Patent Application Publication
(10) Pub. No.: US 2009/0110662 A1
(43) Pub. Date: Apr. 30, 2009

(54) MODIFICATION OF BIOLOGICAL TARGETING GROUPS FOR THE TREATMENT OF CANCER

(75) Inventors: Kurt Breitenkamp, Amherst, MA (US); Jonathan Rios-Doria, Land O Lakes, FL (US); Rebecca Breitenkamp, Amherst, MA (US); Kevin N. Sill, Tampa, FL (US); Habib Skaff, Tampa, FL (US)

Correspondence Address: CHOATE, HALL & STEWART LLP, TWO INTERNATIONAL PLACE, BOSTON, MA, 02110, US

(73) Assignee: Intezyne Technologies, Inc., Tampa, FL (US)

(21) Appl. No.: 12/113,101

(22) Filed: Apr. 30, 2008

Related U.S. Application Data
(60) Provisional application No. 60/915,070, filed on Apr. 30, 2007.

(51) Int. Cl. A61K 38/21; C07K 2/00; C07D 409/14; C07D 487/22; C07D 405/12; C07D 209/18; C07K 7/08; C07D 487/04; A61K 38/00; A61K 35/76; A61K 31/7115; A61K 39/395

(52) U.S. Cl. 424/85.7; 530/300; 530/328; 530/327; 530/330; 549/60; 540/145; 546/196; 548/506; 530/326; 544/350; 514/2; 424/93.6; 514/44; 424/141.1

(57) ABSTRACT

The present invention relates to the field of polymer chemistry and more particularly to click-functionalized targeting compounds and methods for using the same.

### CROSS-REFERENCE TO RELATED APPLICATIONS

The present application claim priority to U.S. provisional patent application Ser. No. 60/915,070, filed Apr. 30, 2007, the entirety of which is hereby incorporated herein by reference.

### FIELD OF THE INVENTION

The present invention relates to the field of polymer chemistry and more particularly to encapsulated contrast agents and uses thereof.

### BACKGROUND OF THE INVENTION

The development of new therapeutic agents has dramatically improved the quality of life and survival rate of patients suffering from a variety of disorders. However, drug delivery innovations are needed to improve the success rate of these treatments. Specifically, delivery systems are still needed which effectively minimize premature excretion and/or metabolism of therapeutic agents and deliver these agents specifically to diseased cells thereby reducing their toxicity to healthy cells.

Rationally-designed, nanoscopic drug carriers, or “nanovectors,” offer a promising approach to achieving these goals due to their inherent ability to overcome many biological barriers. Moreover, their multi-functionality permits the incorporation of cell-targeting groups, diagnostic agents, and a multitude of drugs in a single delivery system. Polymer micelles, formed by the molecular assembly of functional, amphiphilic block copolymers, represent one notable type of multifunctional nanovector.

Polymer micelles are particularly attractive due to their ability to deliver large payloads of a variety of drugs (e.g. small molecule, proteins, and DNA/RNA therapeutics), their improved in vivo stability as compared to other colloidal carriers (e.g. liposomes), and their nanoscopic size which allows for passive accumulation in diseased tissues, such as solid tumors, by the enhanced permeation and retention (EPR) effect. Using appropriate surface functionality, polymer micelles are further decorated with cell-targeting groups and permeation enhancers that can actively target diseased cells and aid in cellular entry, resulting in improved cell-specific delivery.

The ability to target the nanoparticles is of importance in allowing for specific imaging of unhealthy cells, e.g. tumors. In order to accomplish this several groups have shown that over expressed receptors can be used as targeting groups. Examples of these targeting groups include Folate, Her-2 peptide, etc. Typically, conjugation reactions are carried out using the primary amine functionality on proteins (e.g. lysine or protein end-group). Because most proteins contain a multitude of lysines and arginines, such conjugation occurs uncontrollably at multiple sites on the protein. This is particularly problematic when lysines or arginines are located around the active site of an enzyme or other biomolecule. Moreover, the attachment of targeting units directly to the nanoparticle surface through ligand attachment include the fact that this bonding is not permanent. The ligands have the tendency to debond from the nanoparticle surface, especially as the nanoparticles are diluted. Thus, it would be advantageous to provide targeting groups that are readily conjugated to a nanoparticle, or other biologically relevant material, in a manner that is sufficiently stable for targeted delivery.

### DETAILED DESCRIPTION OF CERTAIN EMBODIMENTS OF THE INVENTION

### 1. General Description

According to one embodiment, the present invention provides a “click-functionalized” targeting group. As used herein, the term “click-functionalized” means that the targeting group comprises a functionality suitable for click chemistry. Click chemistry is a popular method of bioconjugation due to its high reactivity and selectivity, even in biological media. See Kolb, H. C.; Finn, M. G.; Sharpless, K. B. Angew. Chem. Int. Ed. 2001, 40, 2004-2021; and Wang, Q.; Chan, T. R.; Hilgraf, R.; Fokin, V. V.; Sharpless, K. B.; Finn, M. G. J. Am. Chem. Soc. 2003, 125, 3192-3193. In addition, currently available recombinant techniques permit the introduction of azides and alkyne-bearing non-canonical amino acids into proteins, cells, viruses, bacteria, and other biological entities that consist of or display proteins. See Link, A. J.; Vink, M. K. S.; Tirrell, D. A. J. Am. Chem. Soc. 2004, 126, 10598-10602; Deiters, A.; Cropp, T. A.; Mukherji, M.; Chin, J. W.; Anderson, C.; Schultz, P. G. J. Am. Chem. Soc. 2003, 125, 11782-11783.

In one embodiment, the “click-functionalized” moiety is an acetylene or an acetylene derivative which is capable of undergoing [3+2]cycloaddition reactions with complementary azide-bearing molecules and biomolecules. In another embodiment, the “click-functionalized” functionality is an azide or an azide derivative which is capable of undergoing [3+2]cycloaddition reactions with complementary alkyne-bearing molecules and biomolecules (i.e. click chemistry).

In another embodiment, the [3+2]cycloaddition reaction of azide or acetylene-bearing nanovectors and complimentary azide or acetylene-bearing biomolecules are transition metal catalyzed. Copper-containing molecules which catalyze the “click” reaction include, but are not limited to, copper wire, copper bromide (CuBr), copper chloride (CuCl), copper sulfate (CuSO₄), copper sulfate pentahydrate (CuSO₄.5H₂O), copper acetate (Cu₂(AcO₄), copper iodide (CuI), [Cu(MeCN)₄](OTf), [Cu(MeCN)₄](PF₆), colloidal copper sources, and immobilized copper sources. Reducing agents as well as organic and inorganic metal-binding ligands can be used in conjunction with metal catalysts and include, but are not limited to, sodium ascorbate, tris(triazolyl)amine ligands, tris(carboxyethyl)phosphine (TCEP), sulfonated bathophenanthroline ligands, and benzimidazole-based ligands.

### 2. Definitions

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As used herein, the term “contrast agent” (also known as “contrast media” and “radiocontrast agents”) refers to a compound used to improve the visibility of internal bodily structures during MRI, PET, ultrasound, X-ray, or fluorescence imaging. Such agents include semiconductor materials, such as CdSe, CdS, CdTe, PdSe, CdSe/CdS, CdSe/ZnS, CdS/ZnS, and CdTe/ZnS. Contrast agents also include magnetic materials such as: Fe, Fe₂O₃, Fe₃O₄, MnFe₂O₄, CoFe₂O₄, NiFe₂O₄, Co, Ni, FePt, CoPt, CoO, Fe₃Pt, Fe₂Pt, CO₃Pt, CO₂Pt, and FeOOH.

The term “targeting group”, as used herein refers to any molecule, macromolecule, or biomacromolecule which selectively binds to receptors that are expressed or over-expressed on specific cell types. Such molecules can be attached to the functionalized end-group of a PEG or drug carrier for cell specific delivery of proteins, viruses, DNA plasmids, oligonucleotides (e.g. siRNA, miRNA, antisense therapeutics, aptamers, etc.), drugs, dyes, and primary or secondary labels which are bound to the opposite PEG end-group or encapsulated within a drug carrier. Such targeting groups include, but or not limited to monoclonal and polyclonal antibodies (e.g. IgG, IgA, IgM, IgD, IgE antibodies), sugars (e.g. mannose, mannose-6-phosphate, galactose), proteins (e.g. transferrin), oligopeptides (e.g. cyclic and acylic RGD-containing oligopeptides), oligonucleotides (e.g. aptamers), and vitamins (e.g. folate).

The term “oligopeptide”, as used herein refers to any peptide of 2-65 amino acid residues in length. In some embodiments, oligopeptides comprise amino acids with natural amino acid side-chain groups. In some embodiments, oligopeptides comprise amino acids with unnatural amino acid side-chain groups. In certain embodiments, oligopeptides are 2-50 amino acid residues in length. In certain embodiments, oligopeptides are 2-40 amino acid residues in length. In some embodiments, oligopeptides are cyclized variations of the linear sequences.

The term “permeation enhancer”, as used herein refers to any molecule, macromolecule, or biomacromolecule which aids in or promotes the permeation of cellular membranes and/or the membranes of intracellular compartments (e.g. endosome, lysosome, etc.) Such molecules can be attached to the functionalized end-group of a PEG or drug carrier to aid in the intracellular and/or cytoplasmic delivery of proteins, viruses, DNA plasmids, oligonucleotides (e.g. siRNA, miRNA, antisense therapeutics, aptamers, etc.), drugs, dyes, and primary or secondary labels which are bound to the opposite PEG end-group or encapsulated within a drug carrier. Such permeation enhancers include, but are not limited to, oligopeptides containing protein transduction domains such as the HIV-1Tat peptide sequence (GRKKRRQRRR), oligoarginine (RRRRRRRRR), or other arginine-rich oligopeptides or macromolecules. Oligopeptides which undergo conformational changes in varying pH environments such oligohistidine (HHHHH) also promote cell entry and endosomal escape.

As used herein, the term “sequential polymerization”, and variations thereof, refers to the method wherein, after a first monomer (e.g. NCA, lactam, or imide) is incorporated into the polymer, thus forming an amino acid “block”, a second monomer (e.g. NCA, lactam, or imide) is added to the reaction to form a second amino acid block, which process may be continued in a similar fashion to introduce additional amino acid blocks into the resulting multi-block copolymers.

As used herein, the term “multiblock copolymer” refers to a polymer comprising one synthetic polymer portion and two or more poly(amino acid) portions. Such multi-block copolymers include those having the format W—X′—X″, wherein W is a synthetic polymer portion and X and X′ are poly(amino acid) chains or “amino acid blocks”. In certain embodiments, the multiblock copolymers of the present invention are triblock copolymers. As described herein, one or more of the amino acid blocks may be “mixed blocks”, meaning that these blocks can contain a mixture of amino acid monomers thereby creating multiblock copolymers of the present invention. In some embodiments, the multiblock copolymers of the present invention comprise a mixed amino acid block and are tetrablock copolymers.

As used herein, the term “triblock copolymer” refers to a polymer comprising one synthetic polymer portion and two poly(amino acid) portions.

As used herein, the term “tetrablock copolymer” refers to a polymer comprising one synthetic polymer portion and either two poly(amino acid) portions, wherein 1 poly(amino acid) portion is a mixed block or a polymer comprising one synthetic polymer portion and three poly(amino acid) portions.

As used herein, the term “inner core” as it applies to a micelle of the present invention refers to the center of the micelle formed by the second (i.e., terminal) poly(amino acid) block. In accordance with the present invention, the inner core is not crosslinked. By way of illustration, in a triblock polymer of the format W—X′—X″, as described above, the inner core corresponds to the X″ block. It is contemplated that the X″ block can be a mixed block.

As used herein, the term “outer core” as it applies to a micelle of the present invention refers to the layer formed by the first poly(amino acid) block. The outer core lies between the inner core and the hydrophilic shell. In accordance with the present invention, the outer core is either crosslinkable or is cross-linked. By way of illustration, in a triblock polymer of the format W—X′—X″, as described above, the outer core corresponds to the X′ block. It is contemplated that the X′ block can be a mixed block.

As used herein, the terms “drug-loaded” and “encapsulated”, and derivatives thereof, are used interchangeably. In accordance with the present invention, a “drug-loaded” micelle refers to a micelle having a drug, or therapeutic agent, situated within the core of the micelle. This is also referred to as a drug, or therapeutic agent, being “encapsulated” within the micelle.

As used herein, the term “polymeric hydrophilic block” refers to a polymer that is not a poly(amino acid) and is hydrophilic in nature. Such hydrophilic polymers are well known in the art and include polyethylene oxide (also referred to as PEO, polyethylene glycol, or PEG), and derivatives thereof, poly(N-vinyl-2-pyrolidone), and derivatives thereof, poly(N-isopropylacrylamide), and derivatives thereof, poly(hydroxyethyl acrylate), and derivatives thereof, poly(hydroxylethyl methacrylate), and derivatives thereof, and polymers of N-(2-hydroxypropoyl)methacrylamide (HMPA) and derivatives thereof.

As used herein, the term “poly(amino acid)” or “amino acid block” refers to a covalently linked amino acid chain wherein each monomer is an amino acid unit. Such amino acid units include natural and unnatural amino acids. In certain embodiments, each amino acid unit is in the L-configuration. Such poly(amino acids) include those having suitably protected functional groups. For example, amino acid monomers may have hydroxyl or amino moieties which are optionally protected by a suitable hydroxyl protecting group or a suitable amine protecting group, as appropriate. Such suitable hydroxyl protecting groups and suitable amine protecting groups are described in more detail herein, infra. As used herein, an amino acid block comprises one or more monomers or a set of two or more monomers. In certain embodiments, an amino acid block comprises one or more monomers such that the overall block is hydrophilic. In other embodiments, an amino acid block comprises one or more monomers such that the overall block is hydrophobic. In still other embodiments, amino acid blocks of the present invention include random amino acid blocks (i.e. blocks comprising a mixture of amino acid residues).

As used herein, the phrase “natural amino acid side-chain group” refers to the side-chain group of any of the 20 amino acids naturally occurring in proteins. Such natural amino acids include the nonpolar, or hydrophobic amino acids, glycine, alanine, valine, leucine isoleucine, methionine, phenylalanine, tryptophan, and proline. Cysteine is sometimes classified as nonpolar or hydrophobic and other times as polar. Natural amino acids also include polar, or hydrophilic amino acids, such as tyrosine, serine, threonine, aspartic acid (also known as aspartate, when charged), glutamic acid (also known as glutamate, when charged), asparagine, and glutamine. Certain polar, or hydrophilic, amino acids have charged side-chains, depending on environmental pH. Such charged amino acids include lysine, arginine, and histidine. One of ordinary skill in the art would recognize that protection of a polar or hydrophilic amino acid side-chain can render that amino acid nonpolar. For example, a suitably protected tyrosine hydroxyl group can render that tyroine nonpolar and hydrophobic by virtue of protecting the hydroxyl group.

As used herein, the phrase “unnatural amino acid side-chain group” refers to amino acids not included in the list of 20 amino acids naturally occurring in proteins, as described above. Such amino acids include the D-isomer of any of the 20 naturally occurring amino acids. Unnatural amino acids also include homoserine, ornithine, and thyroxine. Other unnatural amino acids side-chains are well know to one of ordinary skill in the art and include unnatural aliphatic side chains. Other unnatural amino acids include modified amino acids, including those that are N-alkylated, cyclized, phosphorylated, acetylated, amidated, azidylated, labelled, and the like.

As used herein, the phrase “living polymer chain-end” refers to the terminus resulting from a polymerization reaction which maintains the ability to react further with additional monomer or with a polymerization terminator.

As used herein, the term “termination” refers to attaching a terminal group to a polymer chain-end by the reaction of a living polymer with an appropriate compound.

Alternatively, the term “termination” may refer to attaching a terminal group to an amine or hydroxyl end, or derivative thereof, of the polymer chain.

As used herein, the term “polymerization terminator” is used interchangeably with the term “polymerization terminating agent” and refers to a compound that reacts with a living polymer chain-end to afford a polymer with a terminal group. Alternatively, the term “polymerization terminator” may refer to a compound that reacts with an amine or hydroxyl end, or derivative thereof, of the polymer chain, to afford a polymer with a terminal group.

As used herein, the term “polymerization initiator” refers to a compound, which reacts with, or whose anion or free base form reacts with, the desired monomer in a manner which results in polymerization of that monomer. In certain embodiments, the polymerization initiator is the compound that reacts with an alkylene oxide to afford a polyalkylene oxide block. In other embodiments, the polymerization initiator is the amine salt described herein.

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-20 carbon atoms. In some embodiments, aliphatic groups contain 1-10 carbon atoms. In other embodiments, aliphatic groups contain 1-8 carbon atoms. In still other embodiments, aliphatic groups contain 1-6 carbon atoms, and in yet other embodiments aliphatic groups contain 1-4 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon. This includes any oxidized form of nitrogen, sulfur, phosphorus, or silicon; the quaternized form of any basic nitrogen, or; a substitutable nitrogen of a heterocyclic ring including ═N— as in 3,4-dihydro-2H-pyrrolyl, —NH— as in pyrrolidinyl, or ═N(R^{†})— as in N-substituted pyrrolidinyl.

The term “unsaturated”, as used herein, means that a moiety has one or more units of unsaturation.

The term “aryl” used alone or as part of a larger moiety as in “aralkyl”, “aralkoxy”, or “aryloxyalkyl”, refers to monocyclic, bicyclic, and tricyclic ring systems having a total of five to fourteen ring members, wherein at least one ring in the system is aromatic and wherein each ring in the system contains three to seven ring members. The term “aryl” may be used interchangeably with the term “aryl ring”.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O—(CH₂)₀₋₄C(O)O R^{o}; —(CH₂)₀₋₄CH(O R^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —NO₂; —CN; —N₃; —(CH₂)₀₋₄N(R^{o})₂; —(CH₂)₀₋₄N(R^{o})C(O) R^{o}; —N(R^{o})C(S) R^{o}; —(CH₂)₀₋₄N(R^{o})C(O)N(R^{o}₂) ; —N(R^{o})C(S)N(R^{o}₂) ; —(CH₂)₀₋₄N(R^{o})C(O)OR^{o}; —N(R^{o})N(R^{o})C(O)R^{o}; —N(R^{o})N(R^{o})C(O)N(R^{o}₂) ; —N(R^{o})N(R^{o})C(O)OR^{o}; —(CH₂)₀₋₄C(O) R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O) R^{o}; —OC(O)(CH₂)₀₋₄SR^{o}₃, SC(S)SR^{o}; —(CH₂)₀₋₄SC(O) R^{o}; —(CH₂)₀₋₄C(O)N(R^{o}₂) ; —C(S)N(R^{o}₂) ; —C(S)SR^{o}; —SC(S)SR^{o}, —(CH₂)₀₋₄OC(O)N(R^{o}₂) ; —C(O)N(OR^{o})R^{o}; —C(O)C(O)R^{o}; —C(O)CH₂C(O) R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂N(R^{o}₂) ; —(CH₂)₀₋₄S(O) R^{o}; —N(R^{o})S(O)₂N(R^{o}₂; —N(R^{o})S(O)₂R^{o}; —N(OR^{o})R^{o}; —C(NH)N(R^{o}₂; —P(O)₂R^{o}; —P(O)(R^{o}₂; —OP(O)(R^{o}₂; —OP(O)(OR^{o})₂; SiR₁₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{o}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{o}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. A suitable tetravalent substituent that is bound to vicinal substitutable methylene carbons of an “optionally substituted” group is the dicobalt hexacarbonyl cluster represented by

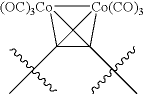

when depicted with the methylenes which bear it.

Suitable substituents on the aliphatic group of R* include halogen, —R^{•}, -(halo R^{•}), —OH, —OR^{•}, —O(halo R^{•}), —CN, —C(O)OH, —C(O)O R^{•}, —NH₂, —NHR^{•}, —N(R^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(halo R^{•}), —OH, —OR^{•}, —O(halo R^{•}), —CN, —C(O)OH, —C(O)O R^{•}, —NH₂, —NHR^{•}, —N(R^{}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Protected hydroxyl groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, 2,2,5,5-tetramethyl-[1,2,5]azadisilolidine and the like, and azide.

Protected aldehydes are well known in the art and include those described in detail in Greene (1999). Suitable protected aldehydes further include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl)acetal, 1,3-dioxanes, 1,3-dioxolanes, semicarbazones, and derivatives thereof.

Protected carboxylic acids are well known in the art and include those described in detail in Greene (1999). Suitable protected carboxylic acids further include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

A “crown ether moiety” is the radical of a crown ether. A crown ether is a monocyclic polyether comprised of repeating units of —CH₂CH₂O—. Examples of crown ethers include 12-crown-4,15-crown-5, and 18-crown-6.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C— or ¹⁴C-enriched carbon are within the scope of this invention. Such compounds are useful, for example, as in neutron scattering experiments, as analytical tools or probes in biological assays.

As used herein, the term “detectable moiety” is used interchangeably with the term “label” and relates to any moiety capable of being detected (e.g., primary labels and secondary labels). A “detectable moiety” or “label” is the radical of a detectable compound.

“Primary” labels include radioisotope-containing moieties (e.g., moieties that contain ³²P, ³³P, ³⁵S, or ¹⁴C), mass-tags, and fluorescent labels, and are signal-generating reporter groups which can be detected without further modifications.

Other primary labels include those useful for positron emission tomography including molecules containing radioisotopes (e.g. ¹⁸F) or ligands with bound radioactive metals (e.g. ⁶²Cu). In other embodiments, primary labels are contrast agents for magnetic resonance imaging such as gadolinium, gadolinium chelates, or iron oxide (e.g Fe₃O₄ and Fe₂O₃) particles. Similarly, semiconducting nanoparticles (e.g. cadmium selenide, cadmium sulfide, cadmium telluride) are useful as fluorescent labels. Other metal nanoparticles (e.g colloidal gold) also serve as primary labels.

“Secondary” labels include moieties such as biotin, or protein antigens, that require the presence of a second compound to produce a detectable signal. For example, in the case of a biotin label, the second compound may include streptavidin-enzyme conjugates. In the case of an antigen label, the second compound may include an antibody-enzyme conjugate. Additionally, certain fluorescent groups can act as secondary labels by transferring energy to another compound or group in a process of nonradiative fluorescent resonance energy transfer (FRET), causing the second compound or group to then generate the signal that is detected.

Unless otherwise indicated, radioisotope-containing moieties are optionally substituted hydrocarbon groups that contain at least one radioisotope. Unless otherwise indicated, radioisotope-containing moieties contain from 1-40 carbon atoms and one radioisotope. In certain embodiments, radioisotope-containing moieties contain from 1-20 carbon atoms and one radioisotope.

The terms “fluorescent label”, “fluorescent group”, “fluorescent compound”, “fluorescent dye”, and “fluorophore”, as used herein, refer to compounds or moieties that absorb light energy at a defined excitation wavelength and emit light energy at a different wavelength. Examples of fluorescent compounds include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The term “substrate”, as used herein refers to any material or macromolecular complex to which a functionalized end-group of a block copolymer can be attached. Examples of commonly used substrates include, but are not limited to, glass surfaces, silica surfaces, plastic surfaces, metal surfaces, surfaces containing a metallic or chemical coating, membranes (eg., nylon, polysulfone, silica), micro-beads (eg., latex, polystyrene, or other polymer), porous polymer matrices (eg., polyacrylamide gel, polysaccharide, polymethacrylate), macromolecular complexes (eg., protein, polysaccharide).

### 3. Description of Exemplary Embodiments

A. Click-Functionalized Targeting Groups

As described above, the present invention provides targeting groups that are functionalized in a manner suitable for click chemistry. In certain embodiments, the present invention provides a click-functionalized Her-2 binding peptide. Her-2 is a clinically validated receptor target and is over-expressed in 20-30% of breast cancers (Stern D. F., Breast Cancer Res. 2000, 2(3), 176, Fantin V. R., et. al., Cancer Res. 2005, 65(15), 6891). Her-2 over-expression leads to constitutive activation of cell signaling pathways that result in increased cell growth and survival. Her-2-binding peptides have been developed which retain much of the potency of full-length antibodies such as trastuzamab (i.e. Herceptin) (Fantin V. R. et. al., Cancer Res. 2005, 65(15), 6891, Park B. W., et. al., Nat. Biotechnol. 2000, 18(2), 194, Karasseva, N., et. al., J. Protein Chem. 2002, 21(4), 287).

In certain embodiments, the present invention provides a compound of formula I-a, I-b, or I-c:

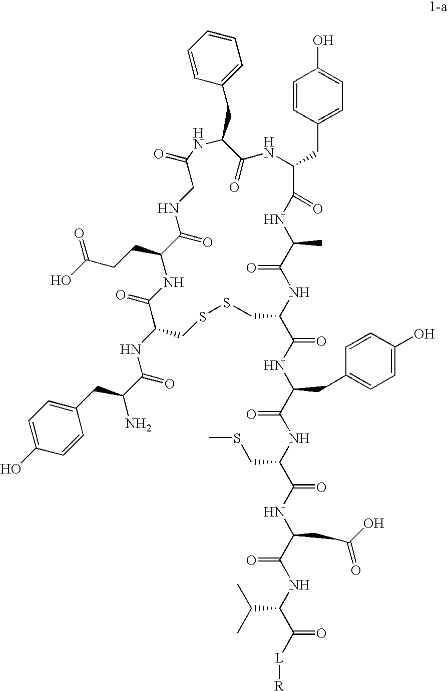

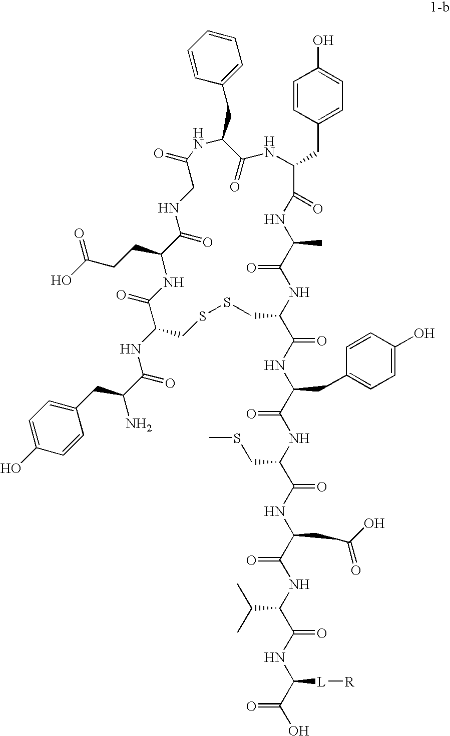

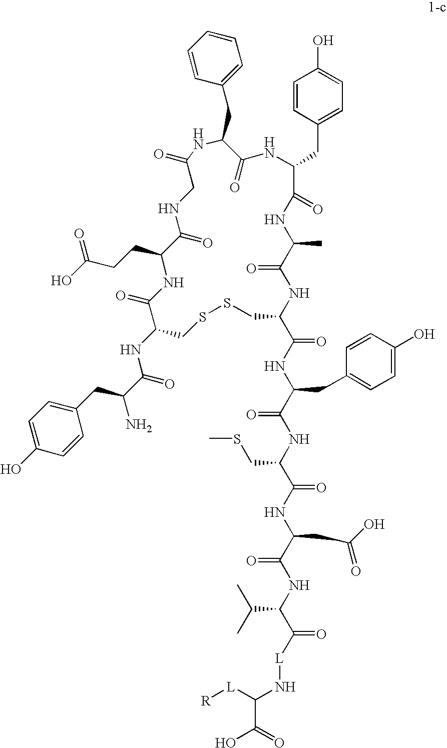

or a salt thereof, wherein each L is independently a valence bond or a bivalent, saturated or unsaturated, straight or branched C₁₋₁₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
      each R is independently alkyne or azide.

Exemplary click-functionalized Her-2 binding peptides are set forth below.

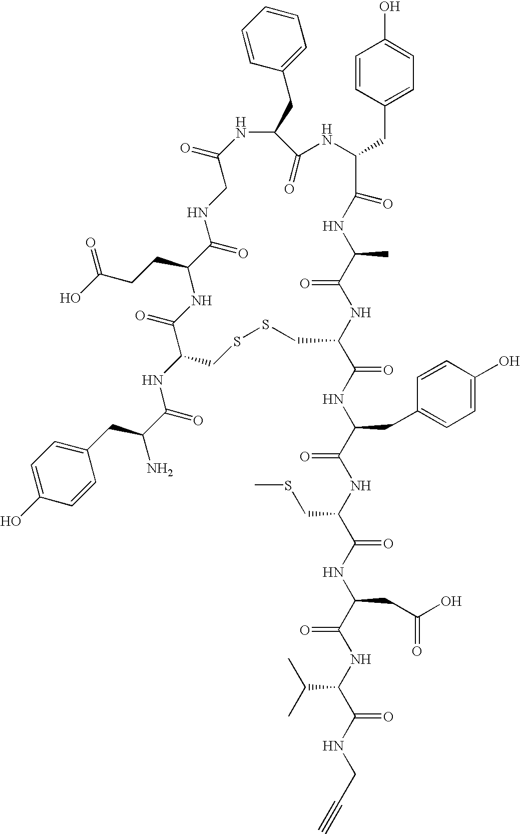

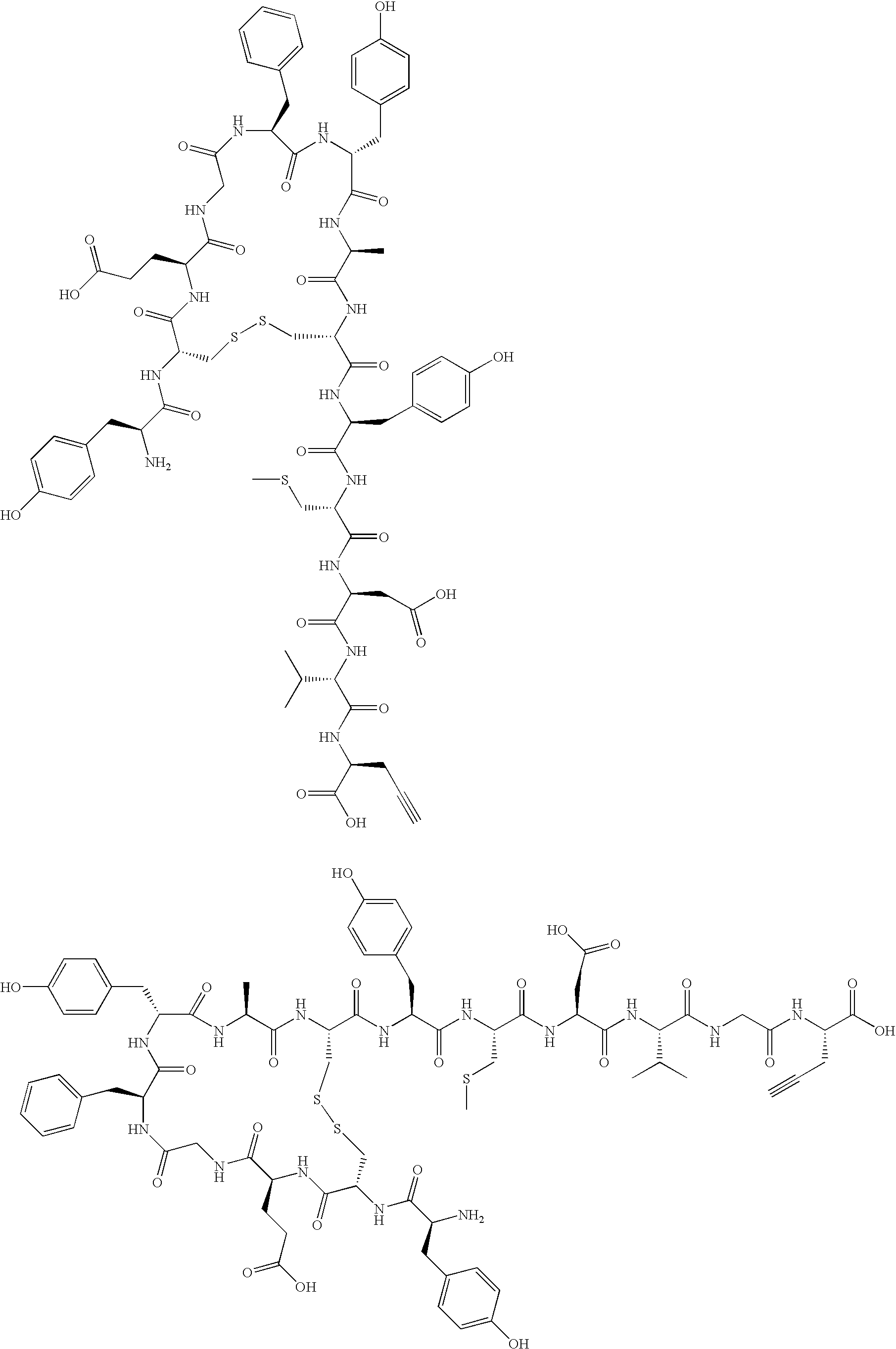

In certain embodiments, a click-functionalized Her-2 binding peptide, in accordance with the present invention, is conjugated to a polymer. In certain embodiments, the polymer is PEG or a functionalized PEG. In other embodiments, a click-functionalized Her-2 binding peptide, in accordance with the present invention, is conjugated to a polymer micelle for tumor-specific targeting of cancer. In still other embodiments, a click-functionalized Her-2 binding peptide, in accordance with the present invention, is conjugated to micelle having a chemotherapeutic agent encapsulated therein.

In certain embodiments, the present invention provides a click-functionalized uPAR antagonist. The urokinase-type plasminogen activator receptor (uPAR) is a transmembrane receptor that plays a key role in cell motility and invasion (Mazar A. P., Anticancer Drugs 2001, 12(5), 387). uPAR is an attractive target in cancer therapy as it over-expressed in many types of cancer and expression is usually indicative of a poor patient prognosis (Foekens, J. A., et. al. Cancer Res. 2000, 60(3), 636). Indeed, many antagonists toward uPAR, or uPAR itself, have been developed and have been shown to suppress tumor growth and metastasis both in vitro and in vivo (Reuning, U. et. al., Curr. Pharm. Des. 2003, 9(19), 1529, Romer, J., et. al. Curr. Pharm. Des. 2004, 10(19), 2359).

In certain embodiments, the present invention provides a compound of formulae II-a, II-b, II-c, II-d, II-e, II-f, II-g, II-h, II-i, II-j, II-k, II-l, II-m, II-n, and II-o, below:

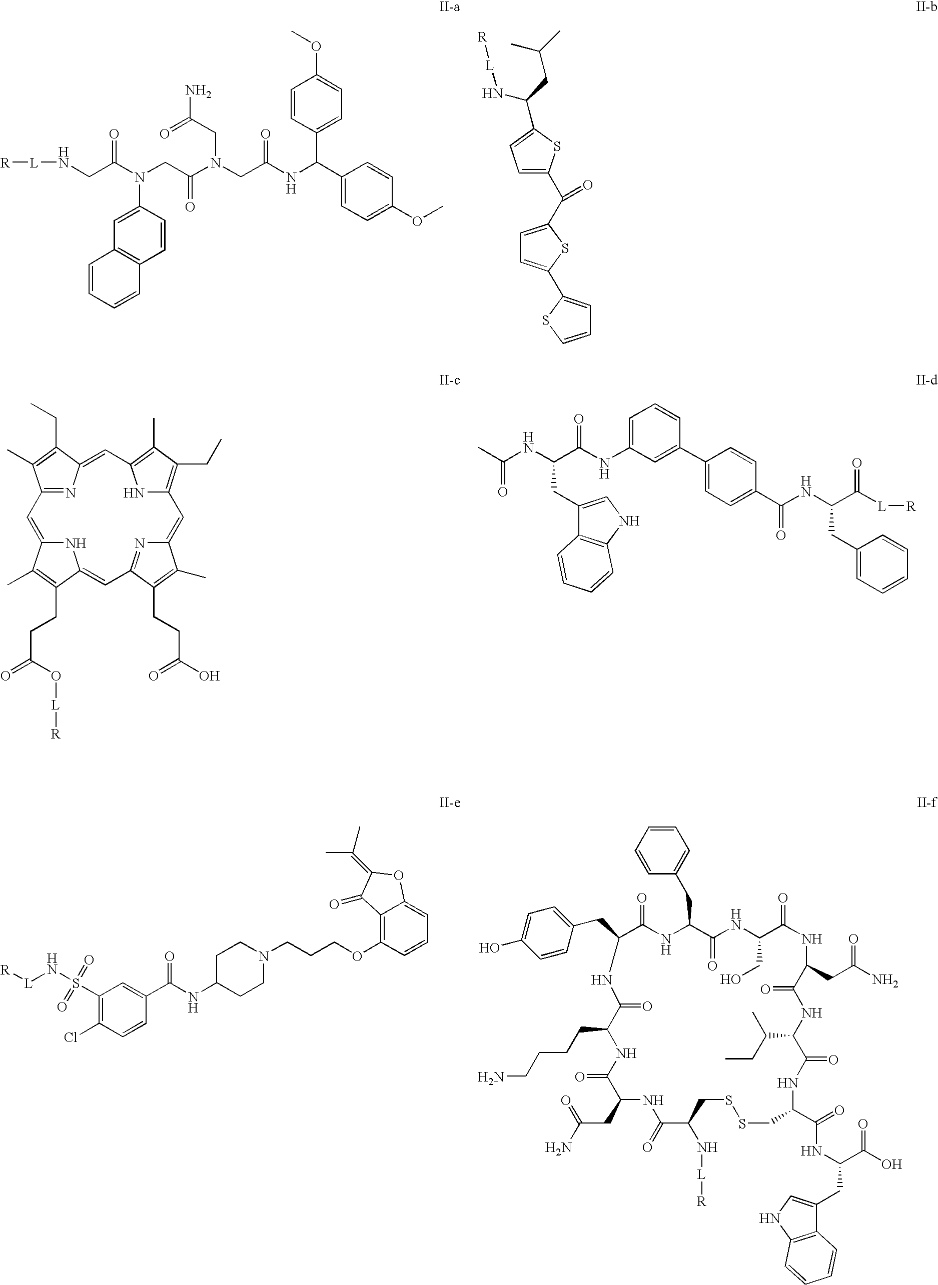

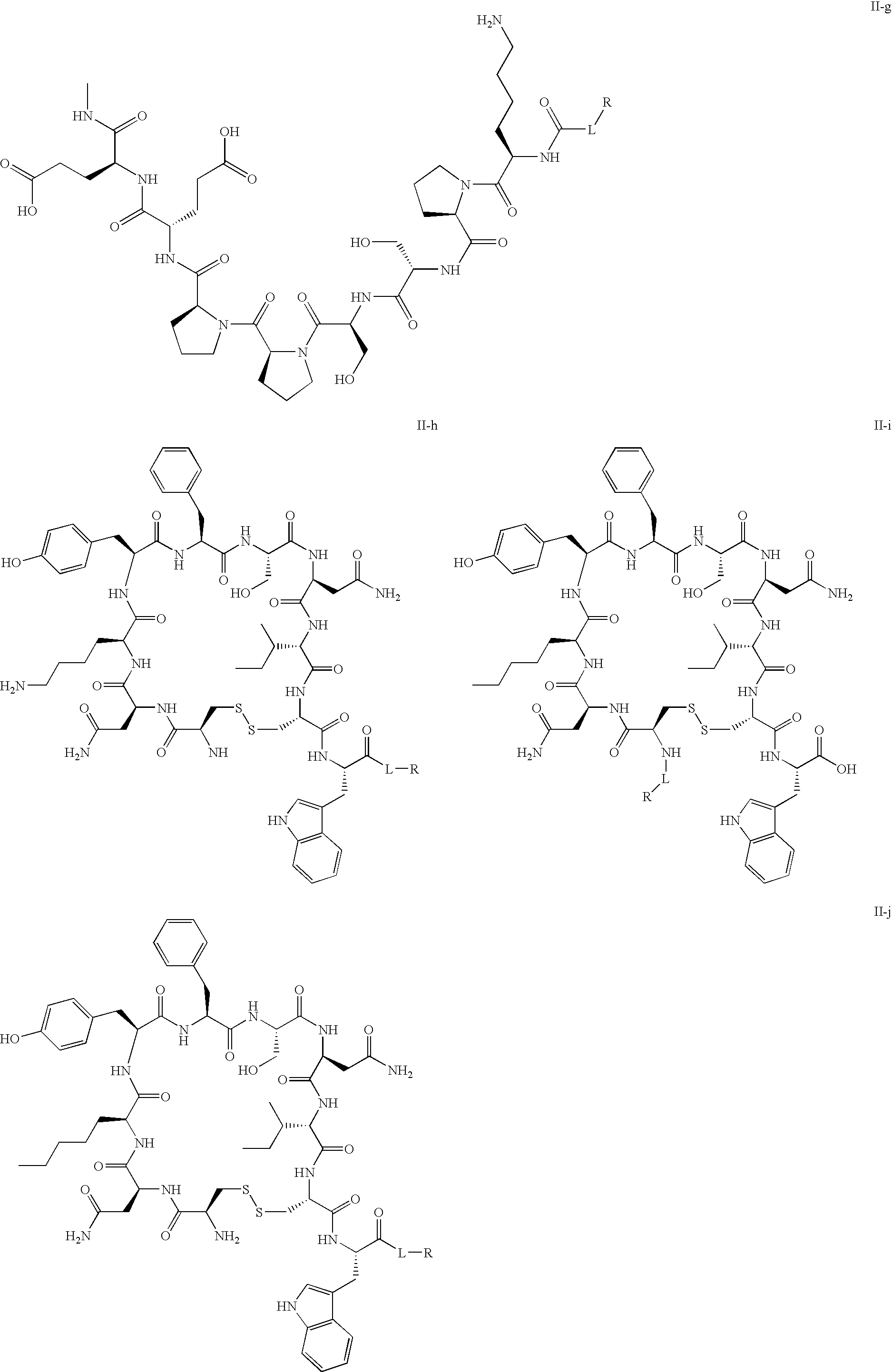

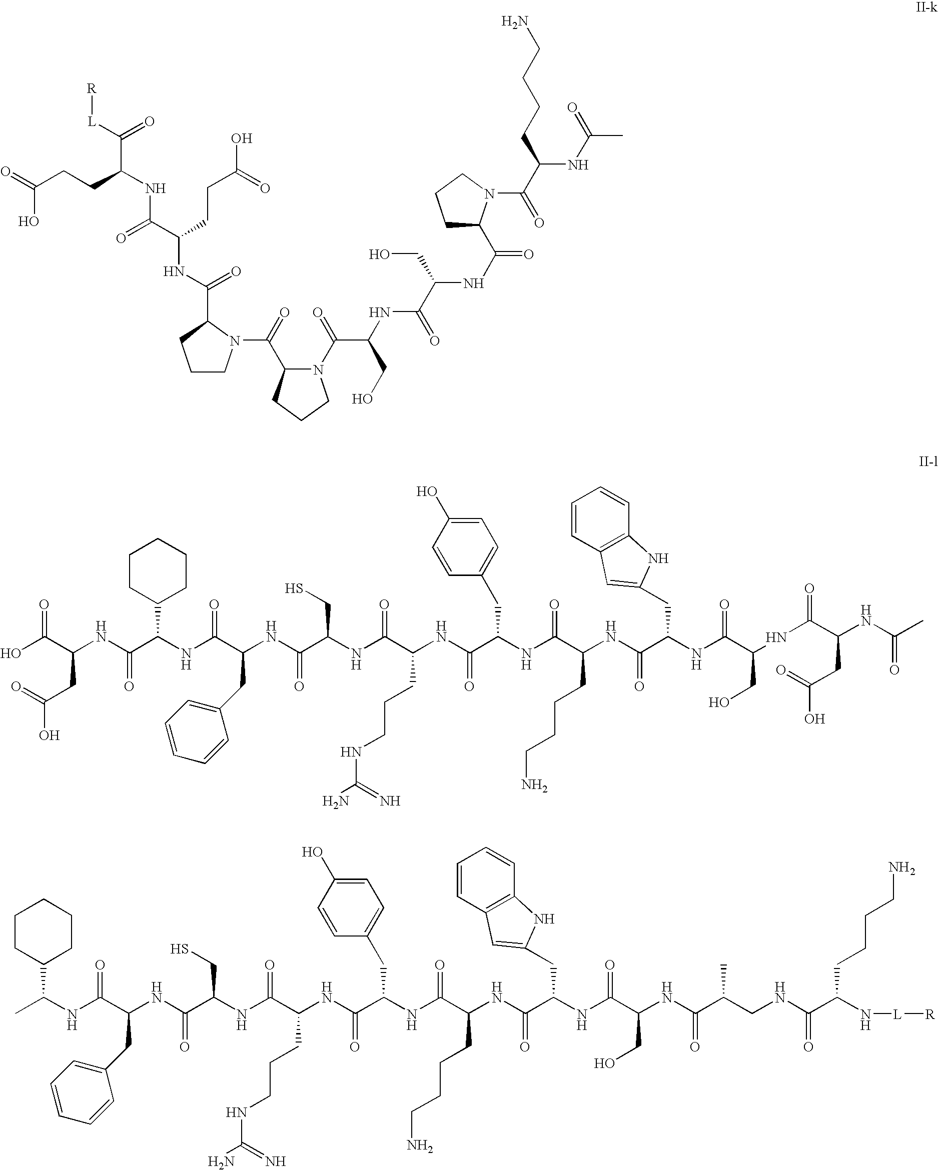

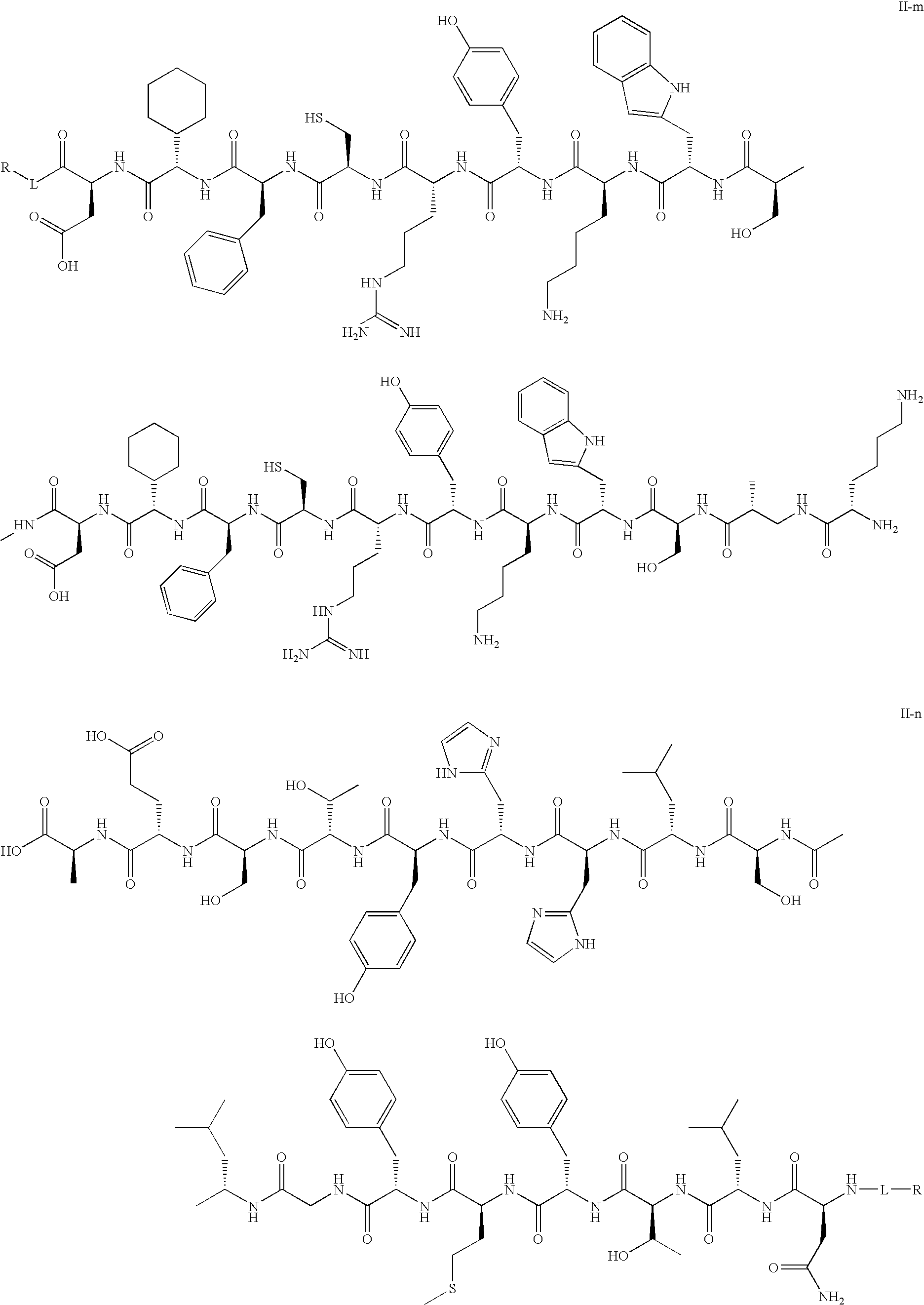

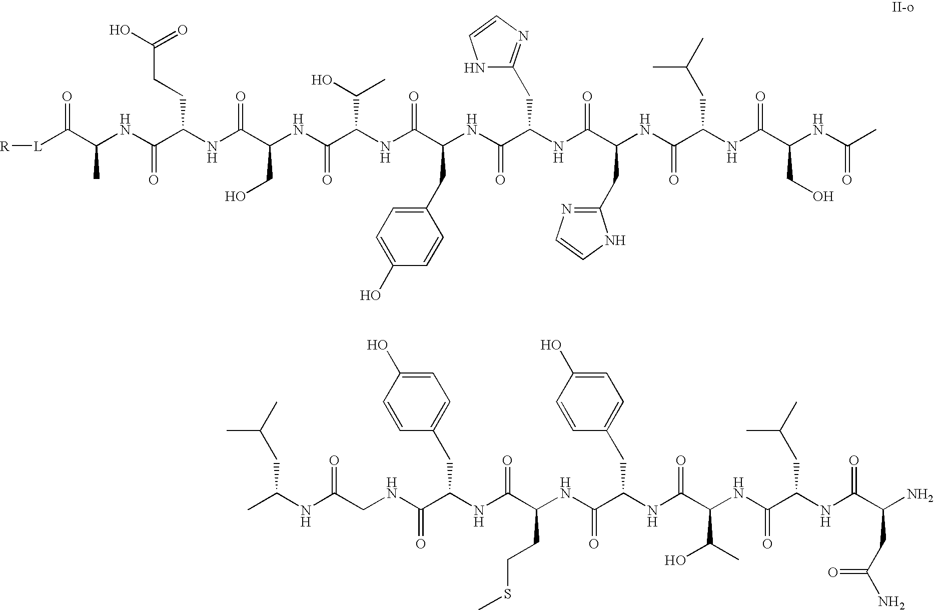

or a salt thereof, wherein each L is independently a valence bond or a bivalent, saturated or unsaturated, straight or branched C₁₋₁₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
      each R is independently alkyne or azide.

One of ordinary skill in the art will recognize that a uPAR antagonist can be click-functionalized at an amine-terminus or at a carboxylate-terminus.

In certain embodiments, a click-functionalized uPAR antagonist, in accordance with the present invention, is conjugated to a polymer. In certain embodiments, the polymer is PEG or a functionalized PEG. In other embodiments, a click-functionalized uPAR antagonist, in accordance with the present invention, is conjugated to a polymer micelle for tumor-specific targeting of cancer. In still other embodiments, a click-functionalized uPAR antagonist, in accordance with the present invention, is conjugated to micelle having a chemotherapeutic agent encapsulated therein.

In certain embodiments, the present invention provides a click-functionalized CXCR4 antagonist. CXCR4 is a chemokine receptor that was identified as a co-receptor for HIV entry (De Clercq, E., Nat. Rev. Drug Discov. 2003, 2(7), 581). CXCR4 has also been found to be over-expressed in a majority of breast cancers as described by Muller and colleagues (Muller, A., et. al., Nature 2001, 410(6824), 50). A number of small molecular antagonists have also been developed towards CXCR4 (De Clercq, E., Nat. Rev. Drug Discov. 2003, 2(7), 581, Gerlach, L. O., et. al., J. Biol. Chem. 2001, 276(17), 14153, Tamamura, H., et. al., Org. Biomol. Chem. 2003, 1(21), 3656, Tamamura, H., et. al., Mini Rev. Med. Chem. 2006, 6(9), 989, Tamamura, H., et. al., Org. Biomol. Chem. 2006, 4(12), 2354). Other inhibitors of CXCR4, such as short interfering RNA, have also shown remarkable anti-cancer activity in vivo, verifying CXCR4 as a pre-clinical target for cancer therapy (Lapteva, N., et. al., Cancer Gene Ther. 2005, 12(1), 84, Liang, Z., et. al., Cancer Res. 2004, 64(12), 4302, Liang, Z. et. al., Cancer Res. 2005, 65(3), 967, Smith, M. C., et. al., Cancer Res. 2004, 64(23), 8604).

In certain embodiments, the present invention provides a click-functionalized folate targeting group. The folate receptor is over-expressed in many epithelial cancers, such as ovarian, colorectal, and breast cancer (Ross, J. F., et. al., Cancer 1994, 73(9), 2432, Jhaveri, M. S., et. al., Mol. Cancer. Ther. 2004, 3(12), 1505). In addition to being highly overexpressed in cancer cells, little or no expression is found in normal cells (Elnakat, H., et. al., Adv. Drug Deliv. Rev. 2004, 56(8), 1067, Weitman, S. D., et. al., Cancer Res. 1992, 52(12), 3396). The non-toxic and non-immunogenic properties of folate make it an excellent ligand for cancer cell targeting.

In certain embodiments, the present invention provides a a click-functionalized compound of formula III:

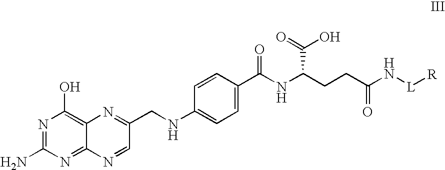

or a salt thereof, wherein each L is independently a valence bond or a bivalent, saturated or unsaturated, straight or branched C₁₋₁₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
      each R is independently alkyne or azide.

In certain embodiments, the present invention provides a compound of formula III wherein L is other than —(CH₂CH₂CH₂)— when R is N₃.

In certain embodiments, a click-functionalized folic acid in accordance with the present invention, is conjugated to a polymer. In certain embodiments, the polymer is PEG or a functionalized PEG. In other embodiments, a click-functionalized folic acid, in accordance with the present invention, is conjugated to a polymer micelle for tumor-specific targeting of cancer. In still other embodiments, a click-functionalized folic acid, in accordance with the present invention, is conjugated to micelle having a chemotherapeutic agent encapsulated therein.

In certain embodiments, the present invention provides a click-functionalized GRP78 peptide antagonist. GRP78 (glucose-regulated protein) is a heat shock protein that functions to regulate protein folding and vesicle trafficking (Kim, Y., et. al., Biochemistry 2006, 45(31), 9434). Although expressed in the endoplasmic reticulum in normal cells, it is over-expressed on the surface of many cancer cells (Kim, Y., et. al., Biochemistry 2006, 45(31), 9434, Arap, M. A., et. al., Cancer Cell 2004, 6(3), 275, Liu, Y., et. al., Mol. Pharm. 2007). Two groups have independently designed peptides that target GRP78 in vitro and in vivo (Arap, M. A., et. al., Cancer Cell 2004, 6(3), 275, Liu, Y., et. al., Mol. Pharm. 2007).

In certain embodiments, the present invention provides a click-functionalized GRP78 targeting group of formulae IV-a through IV-f:

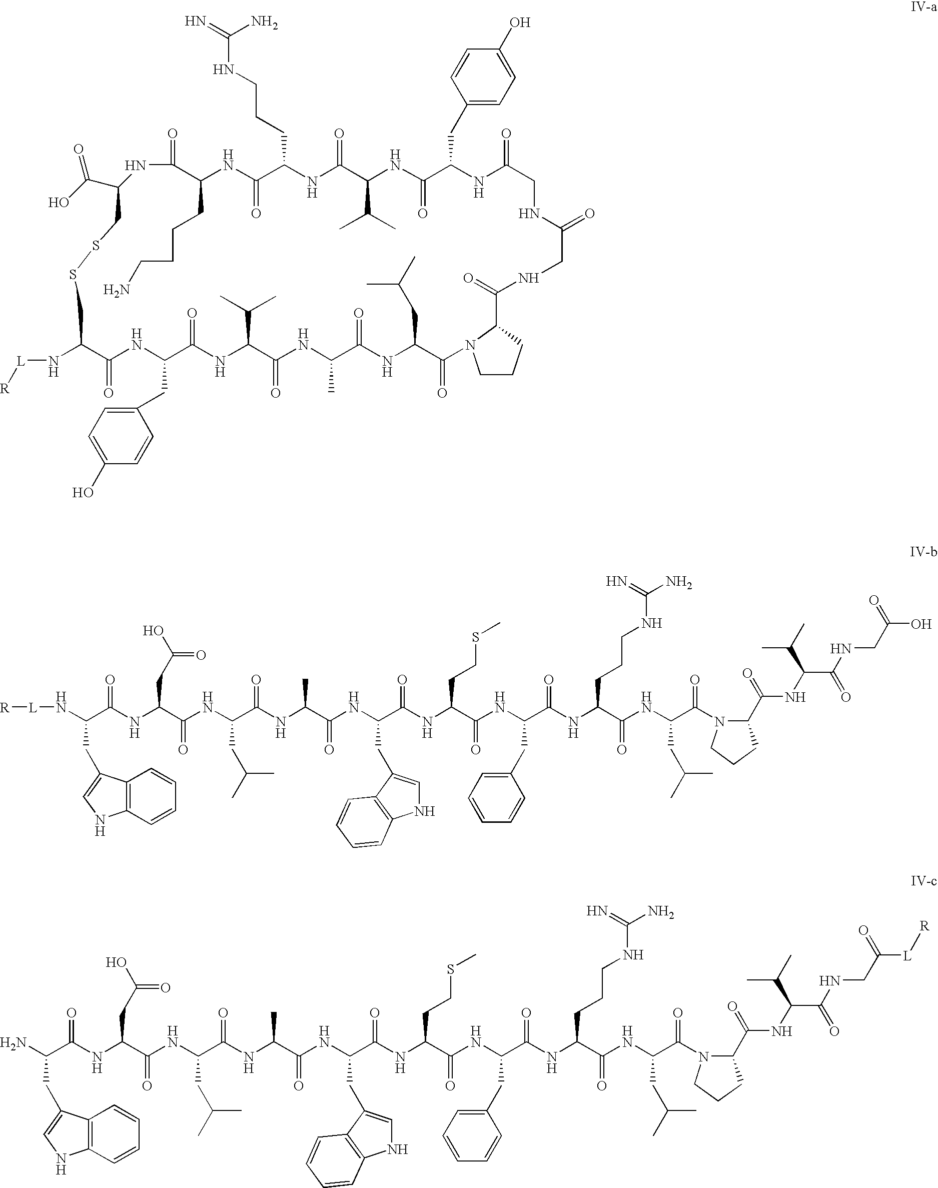

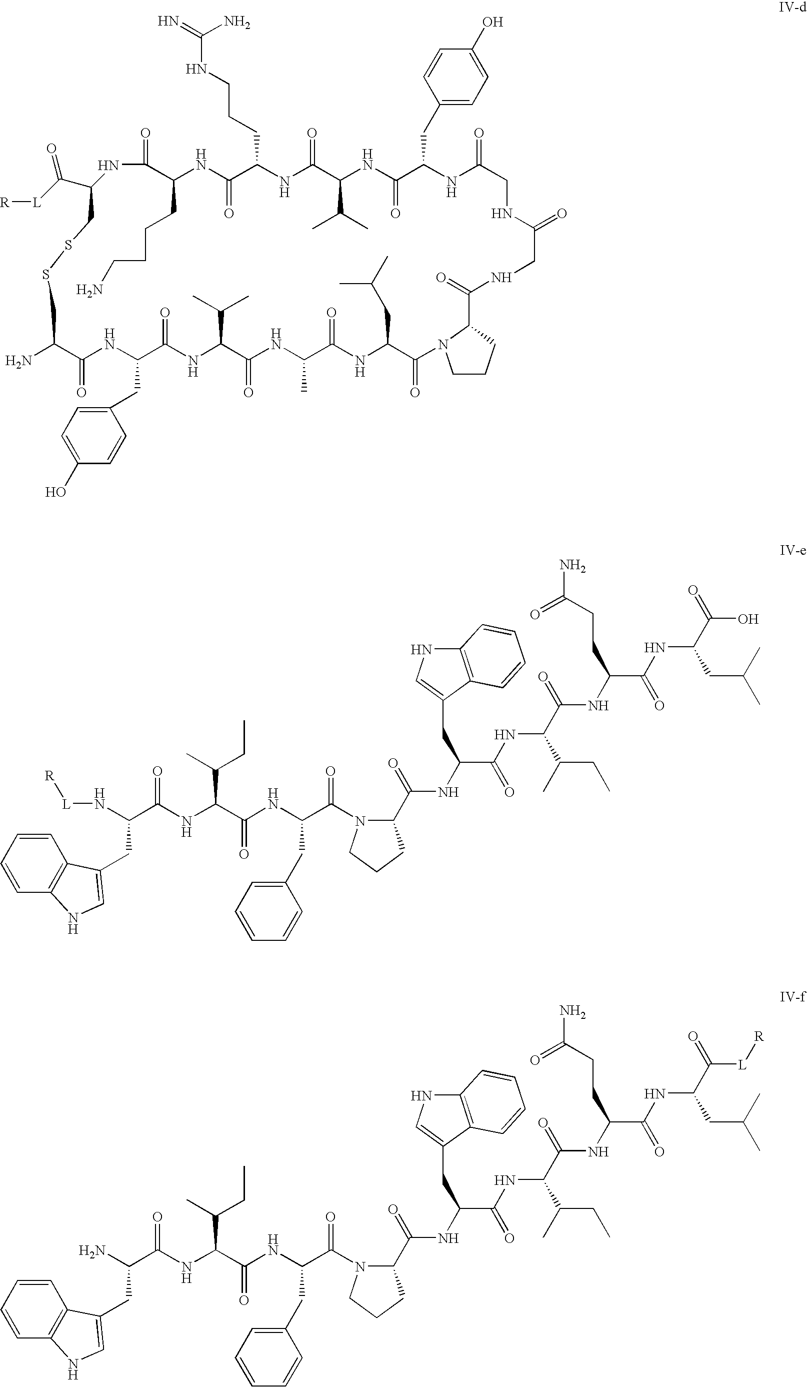

or a salt thereof, wherein each L is independently a valence bond or a bivalent, saturated or unsaturated, straight or branched C₁₋₁₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
      each R is independently alkyne or azide.

In certain embodiments, a click-functionalized GRP78 peptide antagonist in accordance with the present invention, is conjugated to a polymer. In certain embodiments, the polymer is PEG or a functionalized PEG. In other embodiments, a click-functionalized GRP78 peptide antagonist, in accordance with the present invention, is conjugated to a polymer micelle for tumor-specific targeting of cancer. In still other embodiments, a click-functionalized GRP78 peptide antagonist, in accordance with the present invention, is conjugated to micelle having a chemotherapeutic agent encapsulated therein.

Exemplary click-functionalized GRP78 peptide antagonists are set forth below.

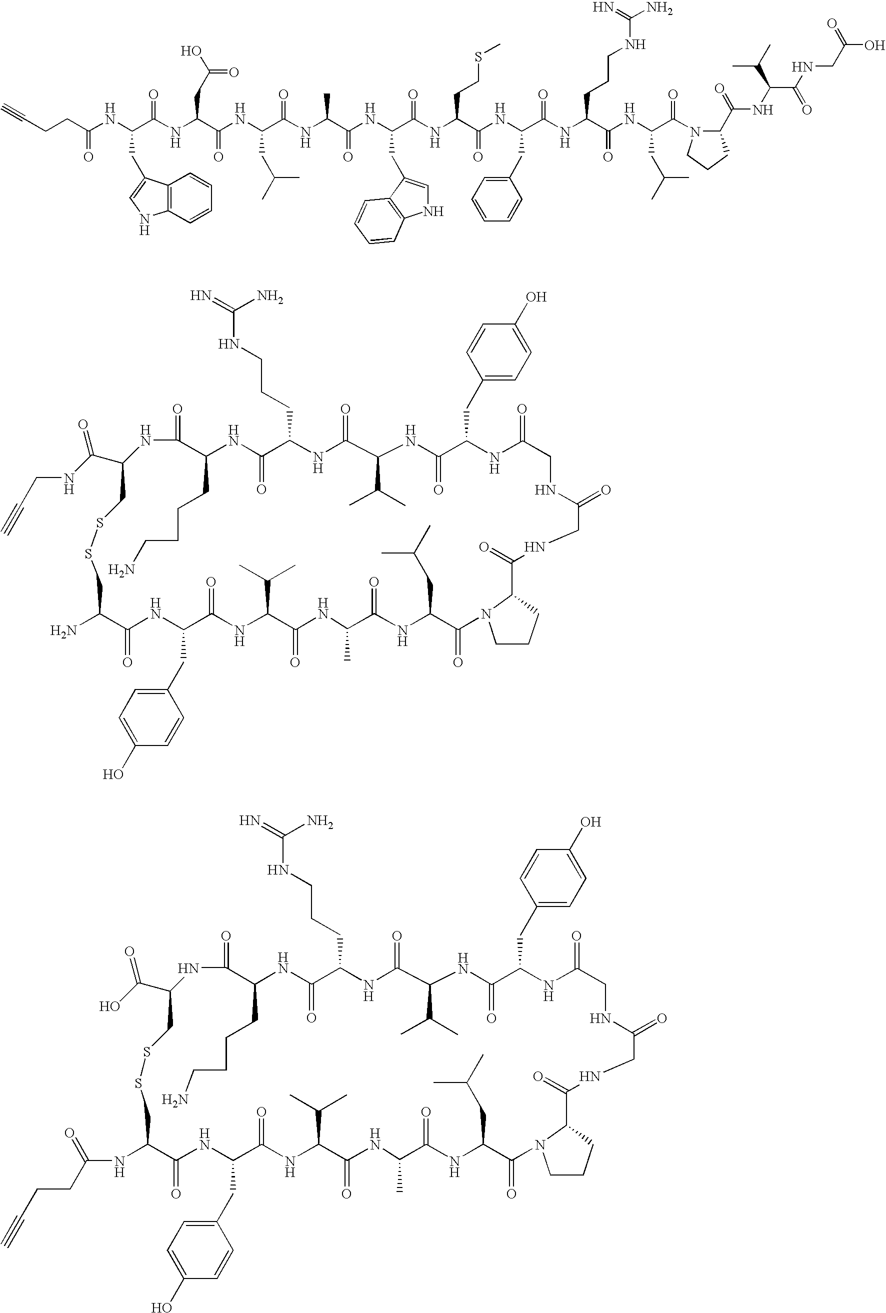

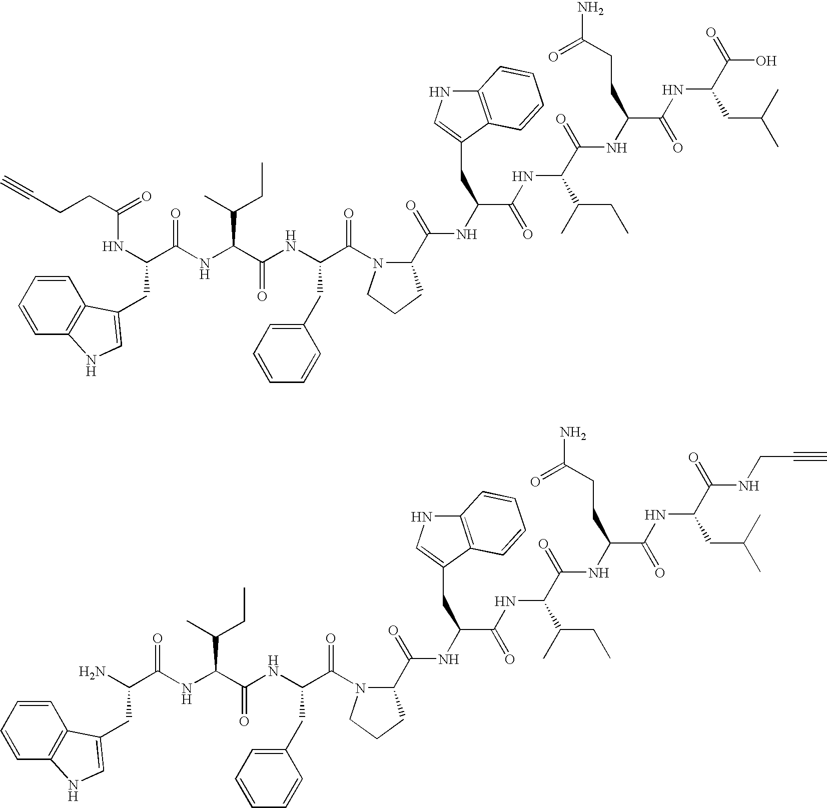

In some embodiments, the present invention provides a click-functionalized integrin binding peptide. In other embodiments, the present invention provides a click-functionalized RGD peptide. Integrins are transmembrane receptors that function in binding to the extracellular matrix. Attachment of cells to substrata via intergrins induces cell signaling pathways that are essential for cell-survival; therefore, disruption of integrin-mediated attachment is a logical intervention for cancer therapy (Hehlgans, S., et. al., Biochim. Biophys. Acta 2007, 1775(1), 163). Small linear and cyclic peptides based on the peptide motif RGD have shown excellent integrin binding (Ruoslahti, E., et. al., Science 1987, 238(4826), 491). In one embodiment, linear and cyclic RGD peptides are conjugated to polymer micelles for tumor-specific targeting of cancer.

In certain embodiments, the present invention provides a compound of formulae V-a, V-b, V-c, V-d, V-e, and V-f:

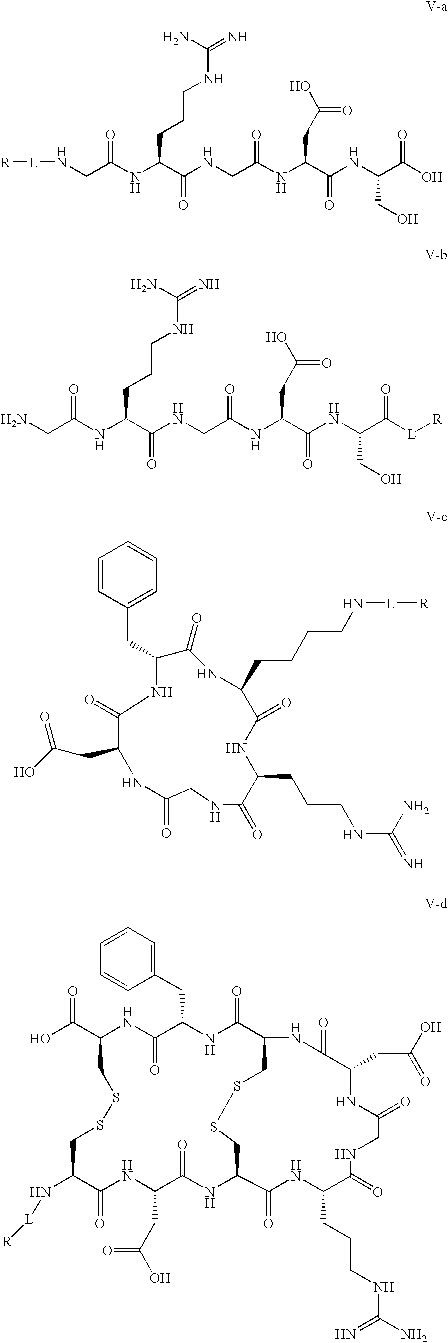

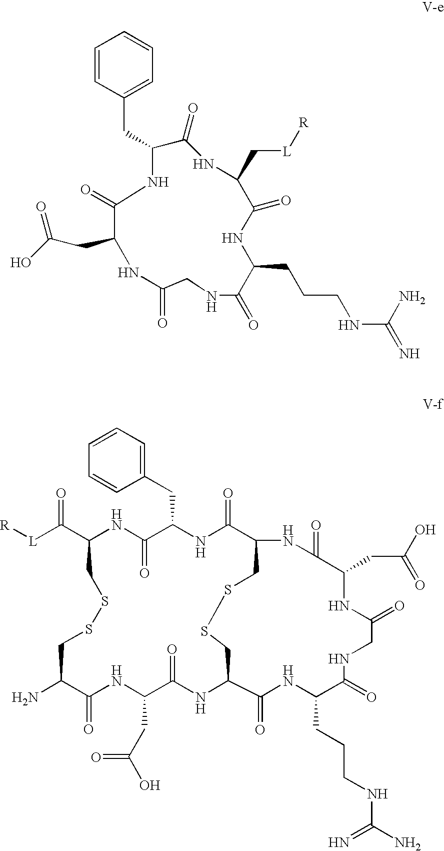

or a salt thereof, wherein each L is independently a valence bond or a bivalent, saturated or unsaturated, straight or branched C₁₋₁₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
      each R is independently alkyne or azide.

In certain embodiments, a click-functionalized RGD peptide in accordance with the present invention, is conjugated to a polymer. In certain embodiments, the polymer is PEG or a functionalized PEG. In other embodiments, a click-functionalized RGD peptide, in accordance with the present invention, is conjugated to a polymer micelle for tumor-specific targeting of cancer. In still other embodiments, a click-functionalized RGD peptide, in accordance with the present invention, is conjugated to micelle having a chemotherapeutic agent encapsulated therein.

Exemplary compounds of formulae V-a, V-b, V-c, V-d, V-e, and V-f are set forth below.

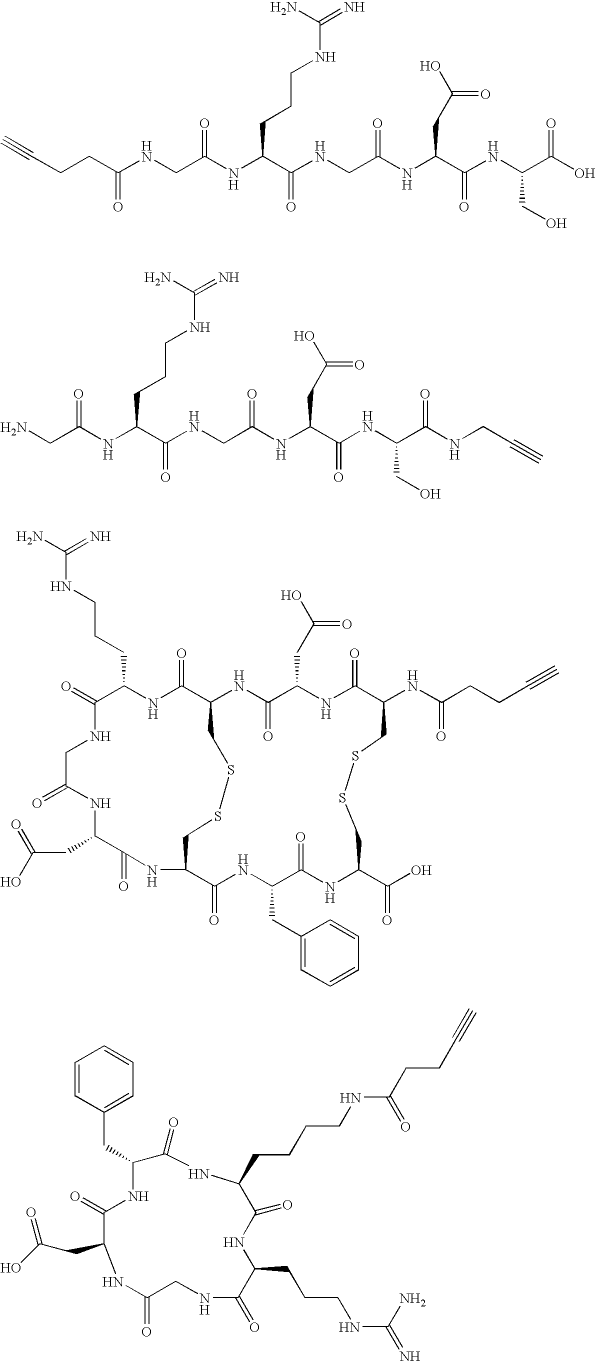

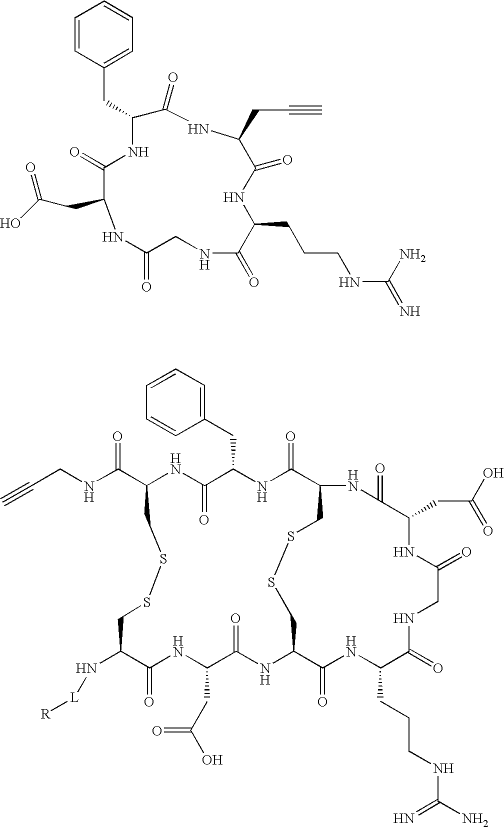

In some embodiments, the present invention provides a click-functionalized luteinizing hormone-releasing hormone (LHRH) antagonist peptides. The luteinizing hormone-releasing hormone receptor (LHRHR) was found to be overexpressed in a number of cancer types, including breast, ovarian and prostate cancer cells (Dharap, S. S., et. al., Proc. Natl. Acad. Sci. U.S.A. 2005, 102(36), 12962). LHRH antagonist peptides have been synthesized are are effective in cancer-cell targeting (Dharap, S. S., et. al., Proc. Natl. Acad. Sci. U.S.A. 2005, 102(36), 12962). In one embodiment, peptide antagonists toward LHRHR are conjugated to polymer micelles for tumor-specific targeting of cancer.

In certain embodiments, the present invention provides a compound of formulae VI-a, VI-b, VI-c, VI-d, and VI-e:

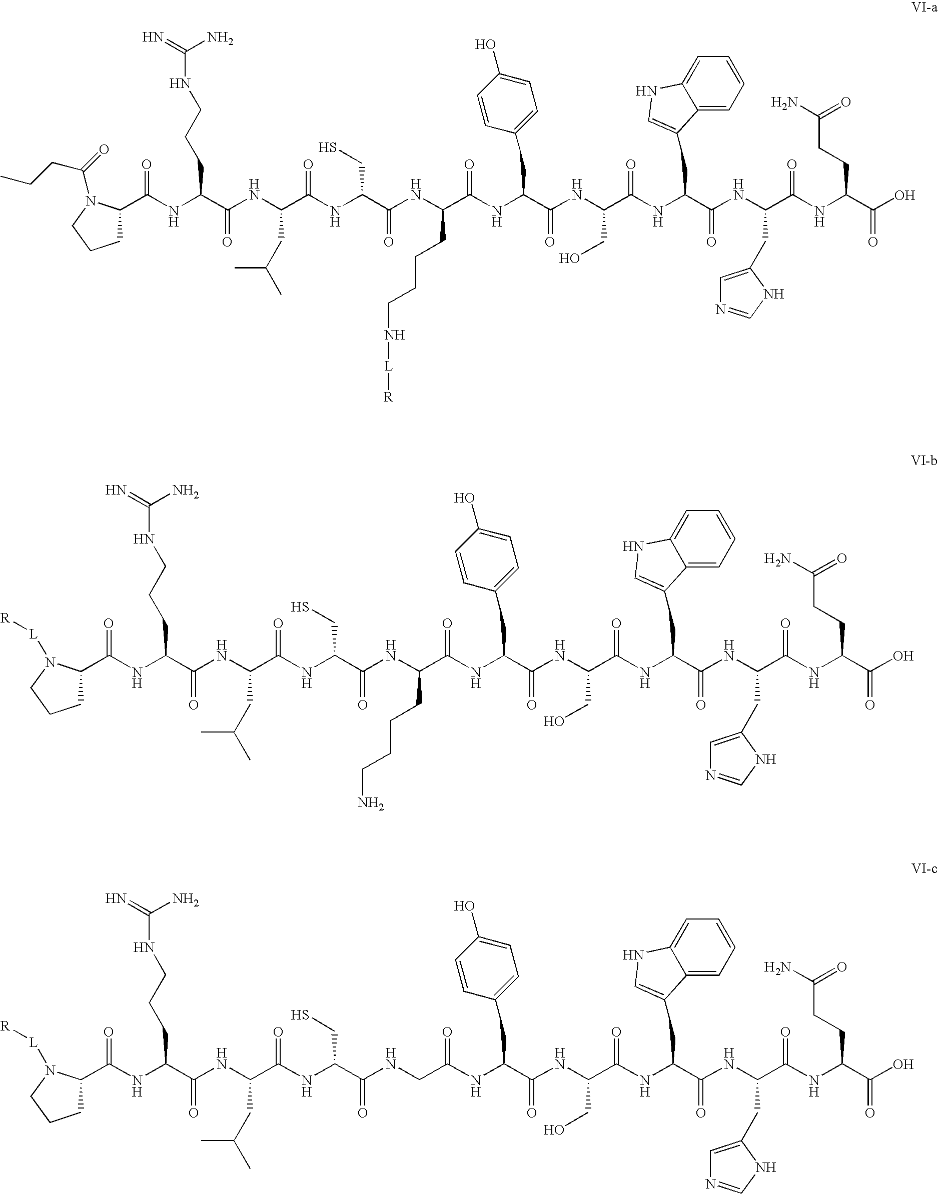

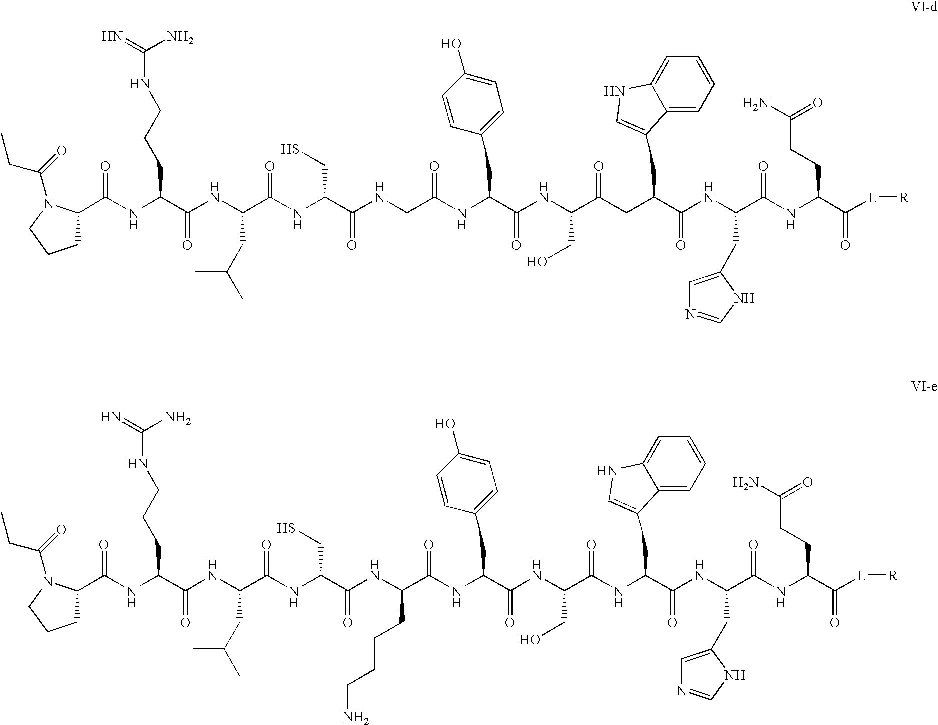

or a salt thereof, wherein each L is independently a valence bond or a bivalent, saturated or unsaturated, straight or branched C₁₋₁₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
      each R is independently alkyne or azide.

In certain embodiments, a click-functionalized LHRH antagonist peptide in accordance with the present invention, is conjugated to a polymer. In certain embodiments, the polymer is PEG or a functionalized PEG. In other embodiments, a click-functionalized LHRH antagonist peptide, in accordance with the present invention, is conjugated to a polymer micelle for tumor-specific targeting of cancer. In still other embodiments, a click-functionalized LHRH antagonist peptide, in accordance with the present invention, is conjugated to micelle having a chemotherapeutic agent encapsulated therein.

Exemplary compounds of formulae VI-a, VI-b, VI-c, VI-d, and VI-e are set forth below.

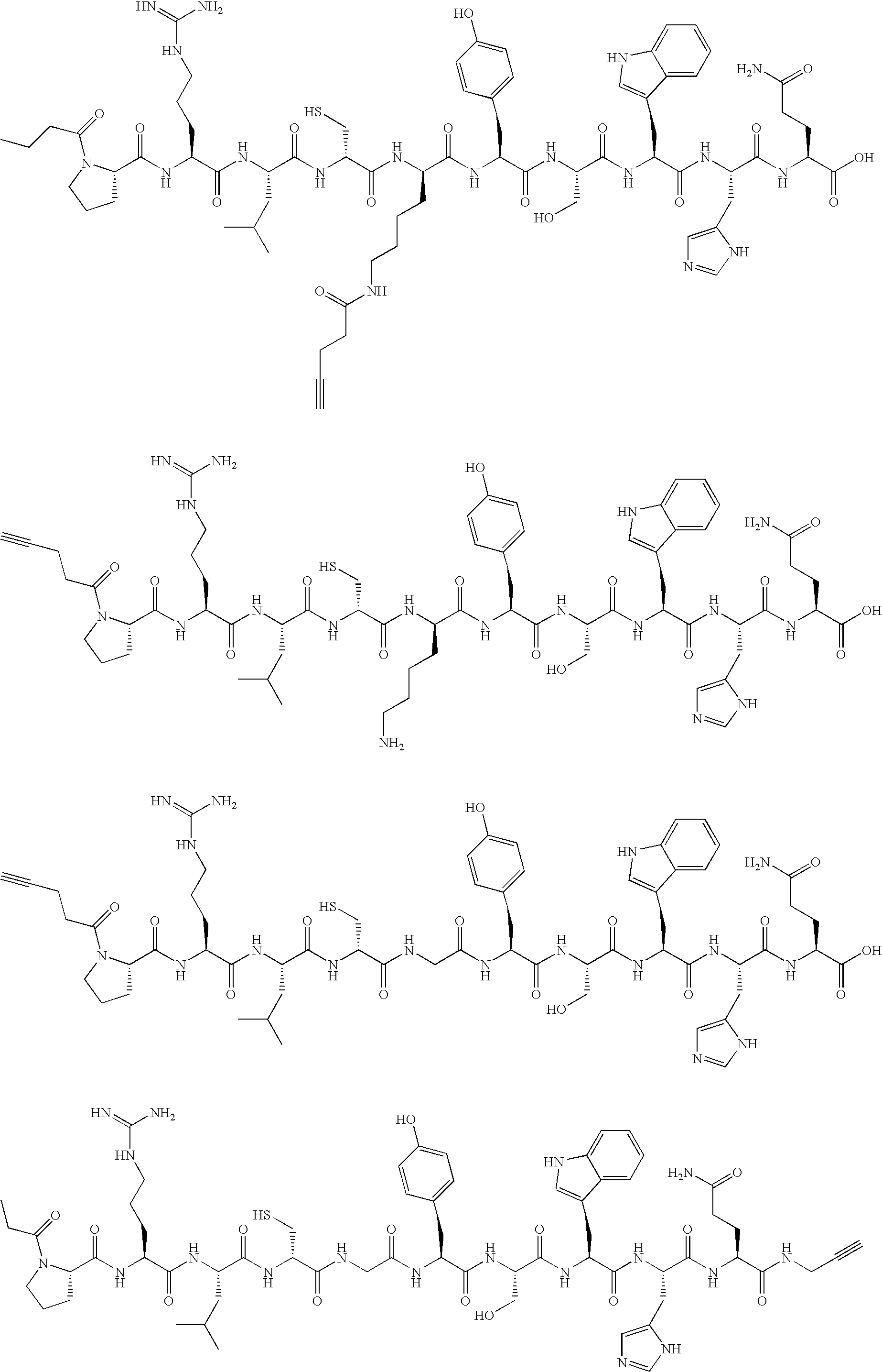

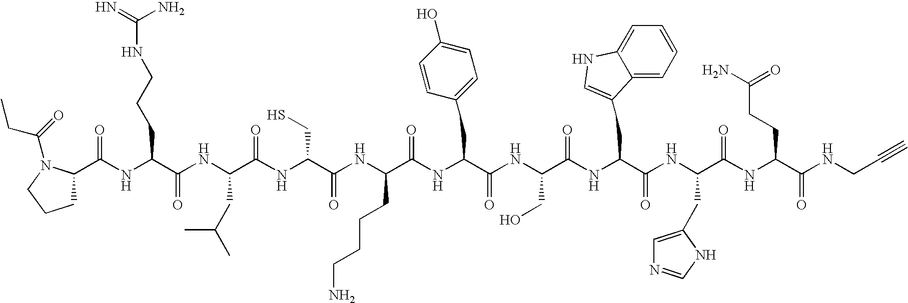

In some embodiments, the present invention provides a click-functionalized aminopeptidase targeting peptide. Aminopeptidase N (CD13) is a tumor specific receptor that is predominantly expressed in blood vessels surrounding solid tumors. A three amino acid peptide (NGR) was identified to be a cell-binding motif that bound to the receptor aminopeptidase N (Arap, W., et. al., Science 1998, 279(5349), 377, Pasqualini, R., et. al., Cancer Res. 2000, 60(3), 722). The NGR peptide, along with other peptides that target the closely related aminopeptidase A (Marchio, S., et. al., Cancer Cell 2004, 5(2), 151) are targeting group for cancer cells.

In certain embodiments, the present invention provides a compound of formulae VII-a, VII-b, VII-c, and VII-d:

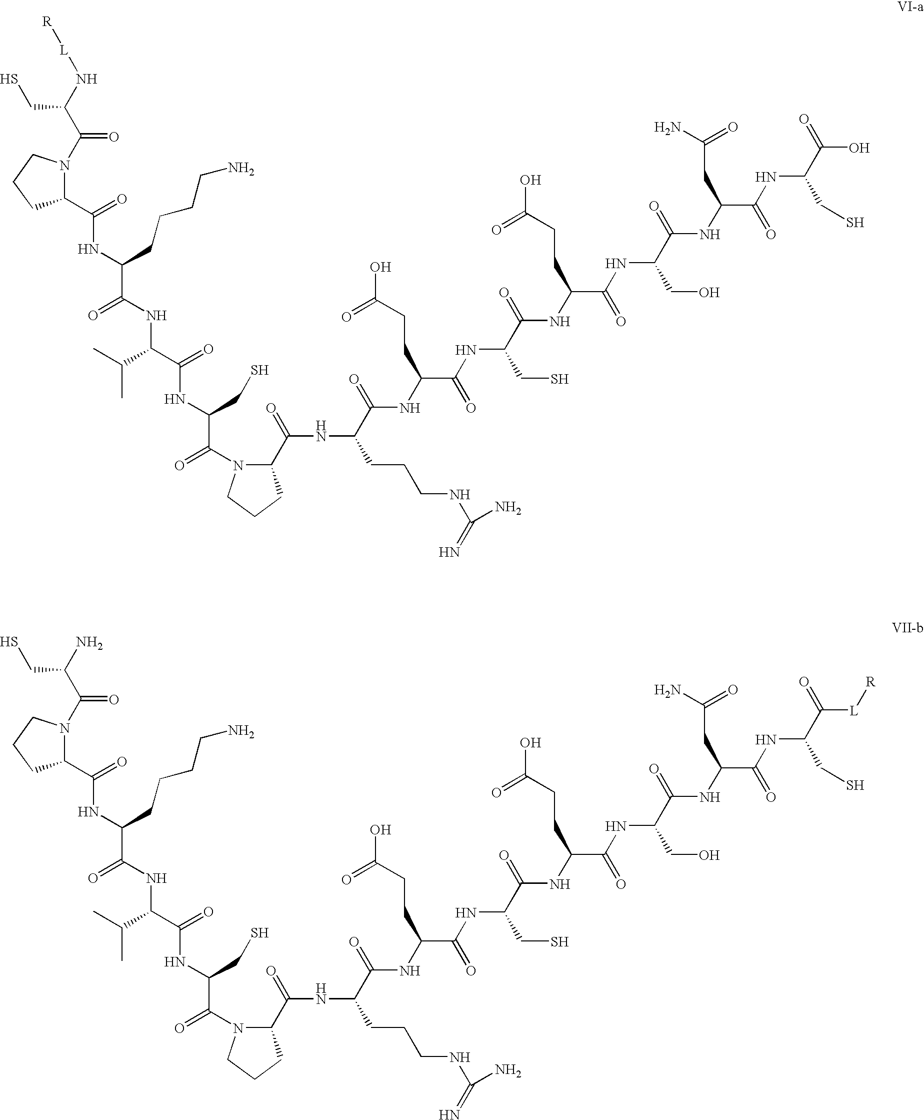

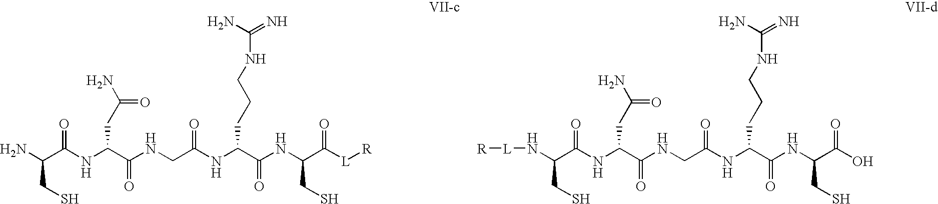

or a salt thereof, wherein each L is independently a valence bond or a bivalent, saturated or unsaturated, straight or branched C₁₋₁₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
      each R is independently alkyne or azide.

In certain embodiments, a click-functionalized aminopeptidase targeting peptide in accordance with the present invention, is conjugated to a polymer. In certain embodiments, the polymer is PEG or a functionalized PEG. In other embodiments, a click-functionalized aminopeptidase targeting peptide, in accordance with the present invention, is conjugated to a polymer micelle for tumor-specific targeting of cancer. In still other embodiments, a click-functionalized peptides targeting Aminopeptidase N and A, in accordance with the present invention, is conjugated to micelle having a chemotherapeutic agent encapsulated therein.

Exemplary compounds of formulae VII-a, VII-b, VII-c, and VII-d are set forth below.

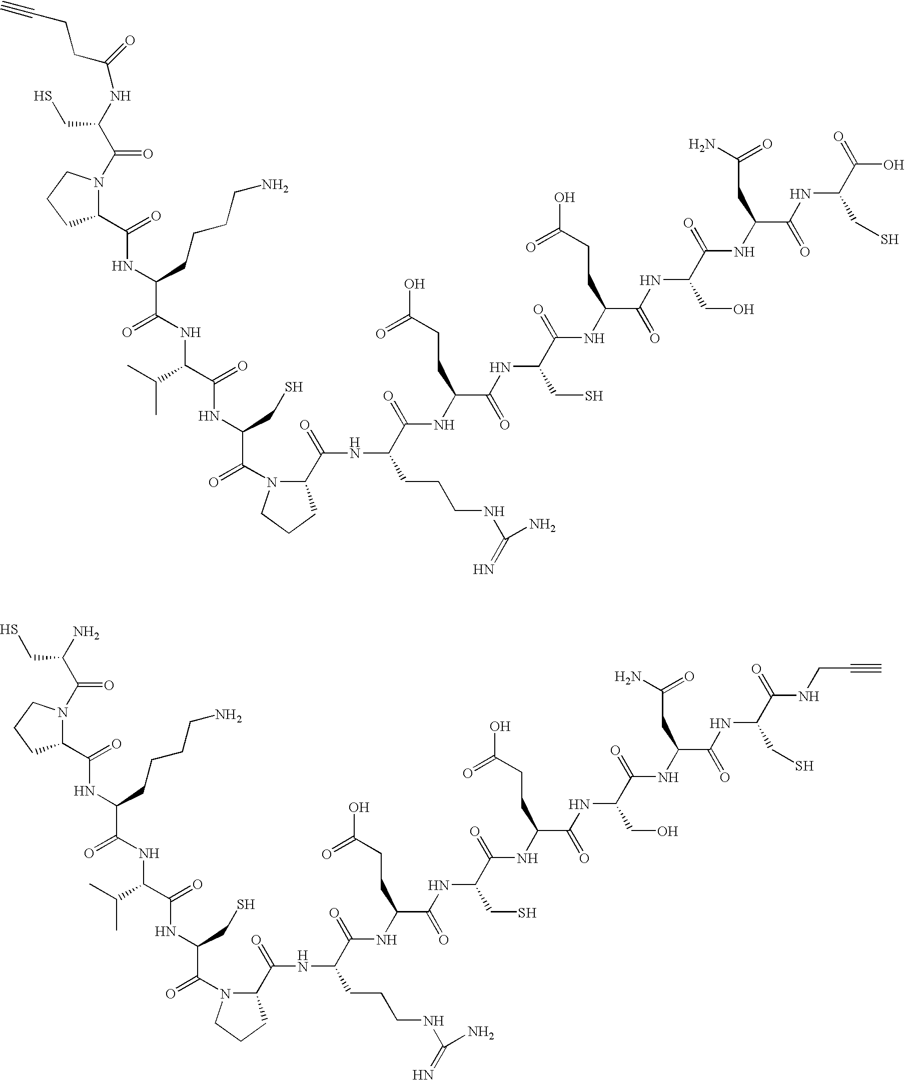

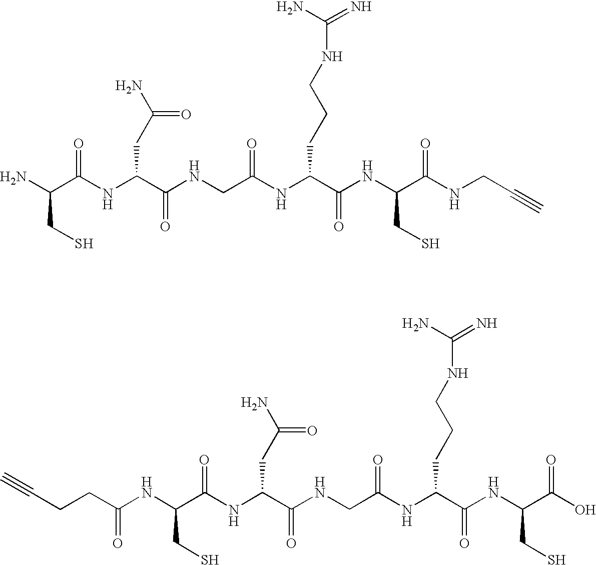

In some embodiments, the present invention provides a click-functionalized cell permeating peptide. Cell permeating peptides based on transduction domains such as those derived from the HIV-1 Tat protein are promising candidates to improve the intracellular delivery of therapeutic macromolecules and drug delivery systems. HIV-1 Tat, and other protein transduction domains, efficiently cross the plasma membranes of cells in an energy dependent fashion, demonstrate effective endosomal escape, and localize in the cell nucleus. (Lindgren, M., et. al., Trends Pharmacol. Sci. 2000, 21, 99, Jeang, K. T., et. al., J. Biol. Chem. 1999, 274, 28837, Green, M., et. al., Cell 1988, 55, 1179). The domain responsible for the cellular uptake of HIV-1 Tat consists of the highly basic region, amino acid residues 49-57 (RKKRRQRRR) (Pepinsky, R. B., et. al., DNA Cell Biol. 1994, 13, 1011, Vive's, E., et. al., J. Biol. Chem. 1997, 272, 16010, Fawell, S., et. al., Proc. Natl. Acad. Sci. U.S.A. 1994, 91, 664). While the detailed mechanism for the cellular uptake of HIV-1 Tat remains speculative, the attachment of the HIV TAT PTD and other cationic PTDs (e.g. oligoarginine and penetratin) has been shown to dramatically increase the permeability of drug delivery systems to cells in vitro. (Torchilin, V. P., et. al., Proc. Natl. Acad. Sci. U.S.A. 2001, 98, 8786, Snyder, E. L., et. al., Pharm. Res. 2004, 21, 389, Letoha, T., et. al. J. Mol. Recognit. 2003, 16(5), 272). In one embodiment, cell permeating peptides are conjugated to polymer micelles to improve uptake into cancer cells.

In certain embodiments, the present invention provides a compound of formulae VIII-a, VIII-b, VIII-c, VIII-d, VIII-e, and VIII-f:

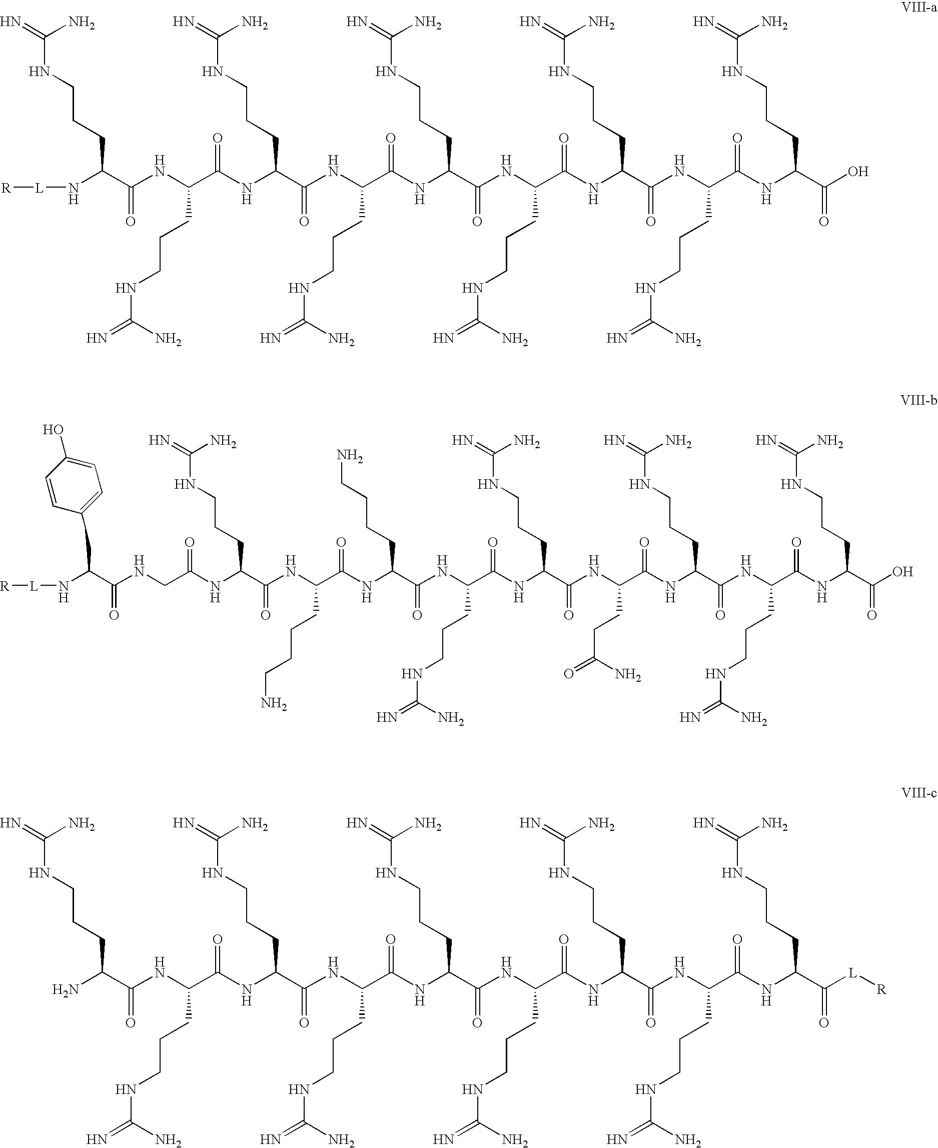

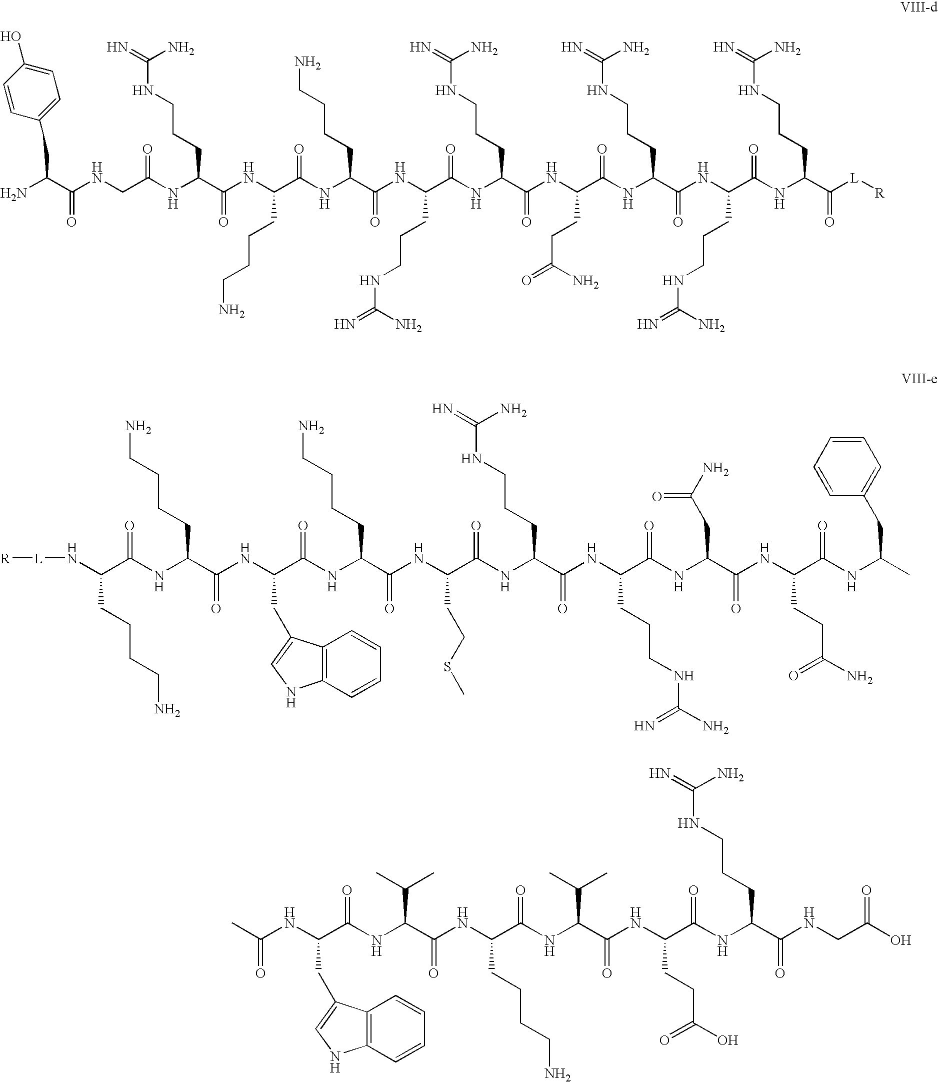

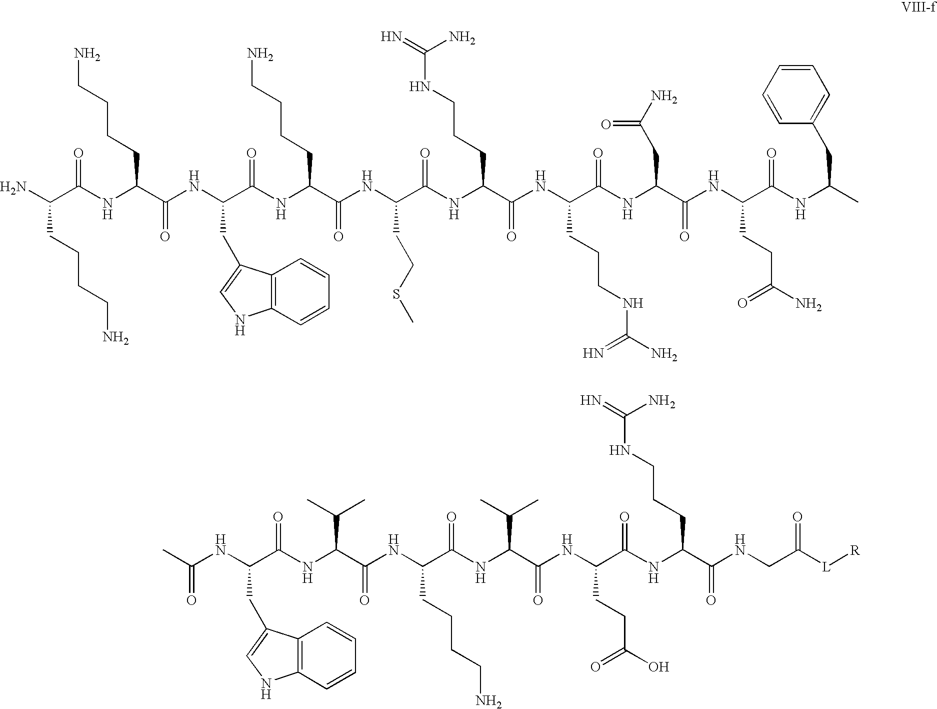

or a salt thereof, wherein each L is independently a valence bond or a bivalent, saturated or unsaturated, straight or branched C₁₋₁₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
      each R is independently alkyne or azide.

In certain embodiments, a click-functionalized cell permeating peptide, in accordance with the present invention, is conjugated to a polymer. In certain embodiments, the polymer is PEG or a functionalized PEG. In other embodiments, a click-functionalized cell permeating peptide, in accordance with the present invention, is conjugated to a polymer micelle for tumor-specific targeting of cancer. In still other embodiments, a click-functionalized cell permeating peptide, in accordance with the present invention, is conjugated to micelle having a chemotherapeutic agent encapsulated therein.

Exemplary compounds of formulae VIII-a, VIII-b, VIII-c, VIII-d, VIII-e, and VIII-f are set forth below.

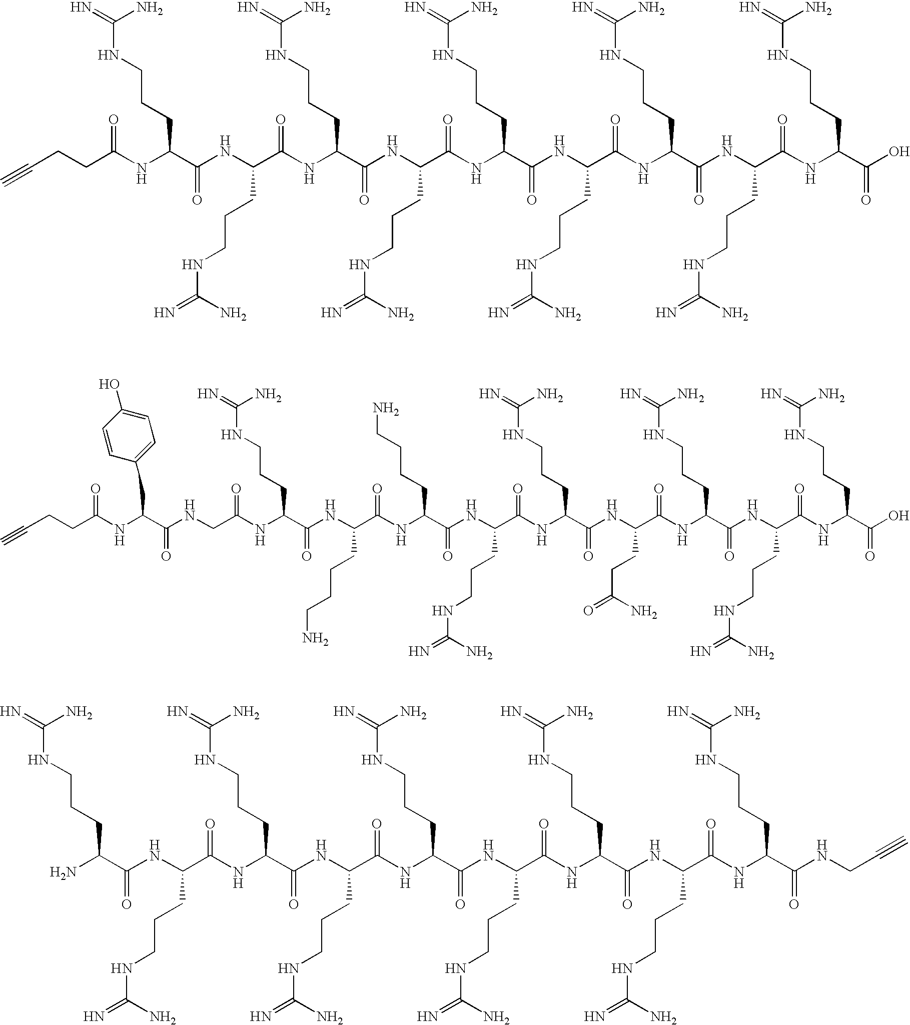

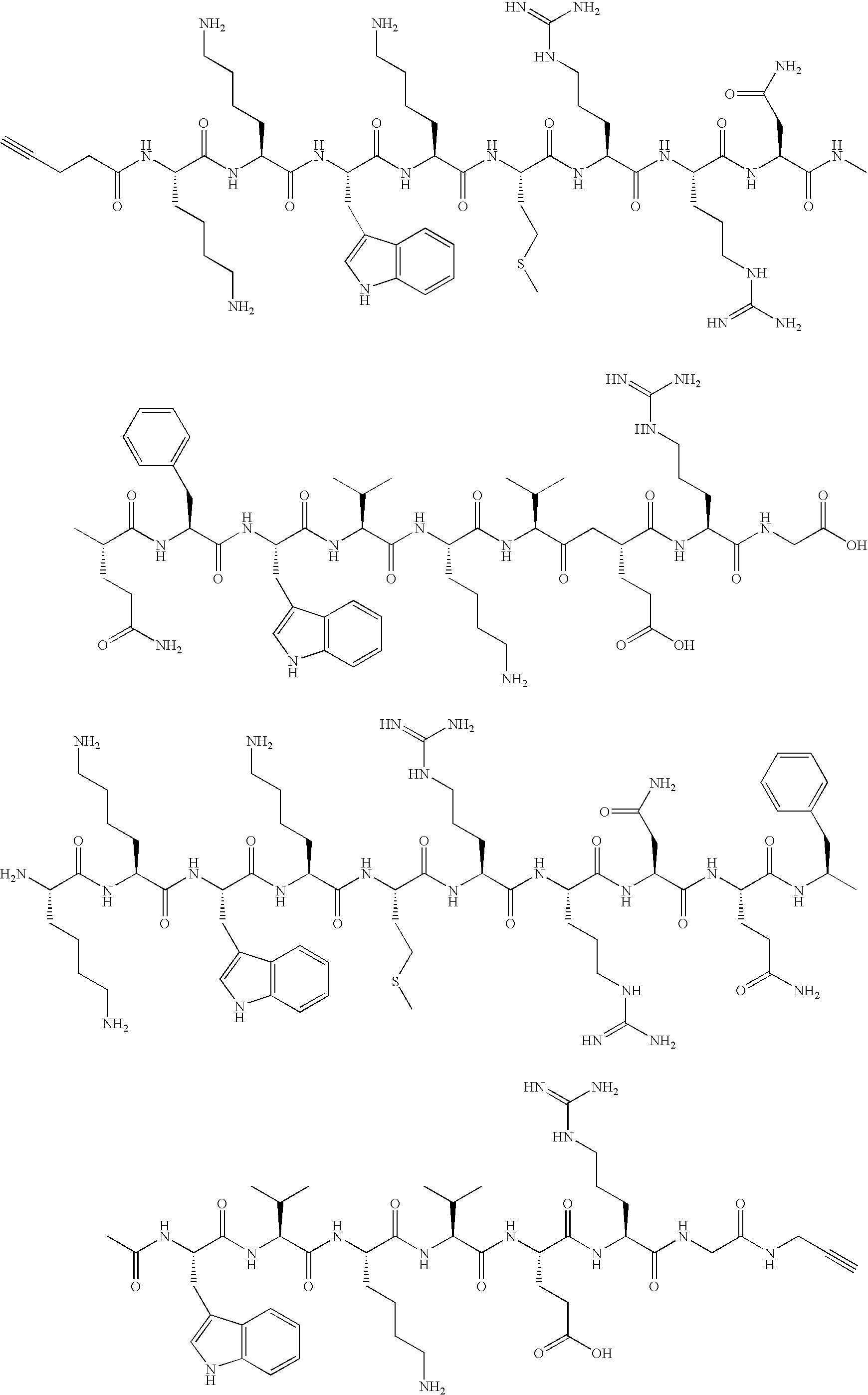

As described herein, the present invention provides targeting groups functionalized for click chemistry. In some embodiments, said functionalization comprises an azide or alkyne moiety. As described above, targeting groups include synthetic peptides having an ability to selectively bind to receptors that are over-expressed on specific cell types. Exemplary targeting groups suitable for derivitization as click-functionalized targeting groups in accordance with the present invention include those set forth in Tables 1-31, below. It will be appreciated that the peptide sequences shown in Tables 1-31, are presented N-terminus to C-terminus, left to right. In a case where a sequence runs over to multiple lines in a row, the each line is a continuation of the sequence on the line above it, left to right. In some embodiments, the peptide sequences listed in Tables 1-31 are cyclized variations of the linear sequences.

TABLE 1
Brain Homing Peptides

| | |
|---|---|
| SEQ ID NO: 1 | CLSSRLDAC |
| SEQ ID NO: 2 | CNSRLQLRC |
| SEQ ID NO: 3 | CKDWGRIC |
| SEQ ID NO: 4 | CTRITESC |
| SEQ ID NO: 5 | CRTGTLFC |
| SEQ ID NO: 6 | CRHWFDVVC |
| SEQ ID NO: 7 | CGNPSYRC |
| SEQ ID NO: 8 | YPCGGEAVAGVS |
| | SVRTMCSE |
| SEQ ID NO: 9 | CNSRLHLRCCENWWG |
| | DVC |
| SEQ ID NO: 10 | CVLRGGC |
| SEQ ID NO: 11 | CLDWGRIC |
| SEQ ID NO: 12 | CETLPAC |
| SEQ ID NO: 13 | CGRSLDAC |
| SEQ ID NO: 14 | CANAQSHC |
| SEQ ID NO: 15 | WRCVLREGPAGGCAW |
| | FNRHRL |
| SEQ ID NO: 16 | LNCDYQGTNPATSVSV |
| | PCTV |
| SEQ ID NO: 17 | WRCVLREGPAGGGAW |
| | FNRHRL |

TABLE 2
Kidney Homing Peptides

| | |
|---|---|
| SEQ ID NO: 18 | CLPVASC |
| SEQ ID NO: 19 | CKGRSSAC |
| SEQ ID NO: 20 | CLGRSSVC |
| SEQ ID NO: 21 | CMGRWRLC |
| SEQ ID NO: 22 | CVAWLNC |
| SEQ ID NO: 23 | CLMGVHC |
| SEQ ID NO: 24 | CFVGHDLC |
| SEQ ID NO: 25 | CKLMGEC |
| SEQ ID NO: 26 | CGAREMC |
| SEQ ID NO: 27 | CWARAQGC |
| SEQ ID NO: 28 | CTSPGGSC |
| SEQ ID NO: 29 | CVGECGGC |
| SEQ ID NO: 30 | CRRFQDC |
| SEQ ID NO: 31 | CKLLSGVC |
| SEQ ID NO: 32 | CRCLNVC |

TABLE 3
Heart Homing Peptides

| | |
|---|---|
| SEQ ID NO: 33 | GGGVFWQ |
| SEQ ID NO: 34 | HGRVRPH |
| SEQ ID NO: 35 | VVLVTSS |
| SEQ ID NO: 36 | CRPPR |
| SEQ ID NO: 37 | CLHRGNSC |
| SEQ ID NO: 38 | CRSWNKADNRSC |
| SEQ ID NO: 39 | WLSEAGPVVTVRALRG |
| | TGSW |

TABLE 4
Gut Homing Peptides

| | |
|---|---|
| SEQ ID NO: 40 | YSGKWGW |
| SEQ ID NO: 41 | VRRGSPQ |
| SEQ ID NO: 42 | MRRDEQR |
| SEQ ID NO: 43 | WELVARS |
| SEQ ID NO: 44 | YAGFFLV |
| SEQ ID NO: 45 | LRAVGRA |
| SEQ ID NO: 46 | GISAVLS |
| SEQ ID NO: 47 | LSPPYMW |
| SEQ ID NO: 48 | GCRCWA |
| SEQ ID NO: 49 | CVESTVA |
| SEQ ID NO: 50 | GAVLPGE |
| SEQ ID NO: 51 | RGDRPPY |
| SEQ ID NO: 52 | RVRGPER |
| SEQ ID NO: 53 | GVSASDW |
| SEQ ID NO: 54 | RSGARSS |
| SEQ ID NO: 55 | LCTAMTE |
| SEQ ID NO: 56 | SKVWLLL |
| SEQ ID NO: 57 | LVSEQLR |
| SEQ ID NO: 58 | SRLSGGT |
| SEQ ID NO: 59 | SRRQPLS |
| SEQ ID NO: 60 | QVRRVPE |
| SEQ ID NO: 61 | MVQSVG |
| SEQ ID NO: 62 | MSPQLAT |
| SEQ ID NO: 63 | WIEEAER |
| SEQ ID NO: 64 | GGRGSWE |
| SEQ ID NO: 65 | FRVRGSP |

TABLE 5
Integrin Homing Peptides

| | |
|---|---|
| SEQ ID NO: 66 | SLIDIP |
| SEQ ID NO: 67 | NGRAHA |
| SEQ ID NO: 68 | VVLVTSS |
| SEQ ID NO: 69 | CRGDC |
| SEQ ID NO: 70 | KRGD |
| SEQ ID NO: 71 | RCDVVV |
| SEQ ID NO: 72 | GACRGDCLGA |
| SEQ ID NO: 73 | HRWMPHVFAVR |
| | QGAS |
| SEQ ID NO: 74 | CRGDCA |
| SEQ ID NO: 75 | RGDL |
| SEQ ID NO: 76 | TIRSVD |
| SEQ ID NO: 77 | DGRAHA |
| SEQ ID NO: 78 | CRGDCL |
| SEQ ID NO: 79 | RRGD |
| SEQ ID NO: 80 | FGRIPSPLAYTYSFR |
| SEQ ID NO: 81 | VSWFSRHRYSPFAVS |

TABLE 6
RGD-Binding Determinants

| | |
|---|---|
| SEQ ID NO: 82 | CSFGRGDIRNC |
| SEQ ID NO: 83 | CSFGKGDNRIC |
| SEQ ID NO: 84 | CSFGRVDDRNC |
| SEQ ID NO: 85 | CSFGRSVDRNC |
| SEQ ID NO: 86 | CSFGRWDARNC |
| SEQ ID NO: 87 | CSFGRDDGRNC |
| SEQ ID NO: 88 | CSFGRTDQRIC |
| SEQ ID NO: 89 | CSFGRNDSRNC |
| SEQ ID NO: 90 | CSFGRADRRNC |
| SEQ ID NO: 91 | CSFGKRDMRNC |
| SEQ ID NO: 92 | CSFGRQDVRNC |

TABLE 7
Angiogenic Tumor Endothelium Homing Peptides

| | |
|---|---|
| SEQ ID NO: 93 | CDCRGDCFC |
| SEQ ID NO: 94 | CNGRCVSGCAGRC |

TABLE 8
Ovary Homing Peptides

| | |
|---|---|
| SEQ ID NO: 95 | GVRTSIW |
| SEQ ID NO: 96 | KLVNSSW |
| SEQ ID NO: 97 | EVRSRLS |
| SEQ ID NO: 98 | RPVGMRK |
| SEQ ID NO: 99 | LCERVWR |
| SEQ ID NO: 100 | RYGLVAR |
| SEQ ID NO: 101 | FGSQAFV |
| SEQ ID NO: 102 | AVKDYFR |
| SEQ ID NO: 103 | FFAAVRS |
| SEQ ID NO: 104 | WLERPEY |
| SEQ ID NO: 105 | GGDVMWR |
| SEQ ID NO: 106 | VRARLMS |
| SEQ ID NO: 107 | RVRLVNL |
| SEQ ID NO: 108 | TLRESGP |

TABLE 9
Uterus Homing Peptides

| | |
|---|---|
| SEQ ID NO: 109 | GLSGGRS |
| SEQ ID NO: 110 | SWCEPGWCR |

TABLE 10
Sperm Homing Peptides

| | |
|---|---|
| SEQ ID NO: 111 | XLWLLXXG |

TABLE 11
Microglia Homing Peptides

| | |
|---|---|
| SEQ ID NO: 112 | SFTYWTN |

TABLE 12
Synovium Homing Peptides

| | |
|---|---|
| SEQ ID NO: 113 | CKSTHDRLC |

TABLE 13
Urothelium Homing Peptides

| | |
|---|---|
| SEQ ID NO: 114 | I/LGSGL |

TABLE 14
Prostate Homing Peptides

| | |
|---|---|
| SEQ ID NO: 115 | EVQSAKW |
| SEQ ID NO: 116 | GRLSVQV |
| SEQ ID NO: 117 | FAVRVVG |
| SEQ ID NO: 118 | GFYRMLG |
| SEQ ID NO: 119 | GSRSLGA |
| SEQ ID NO: 120 | GDELLA |
| SEQ ID NO: 121 | GSEPMFR |
| SEQ ID NO: 122 | WHQPL |
| SEQ ID NO: 123 | RGRWLAL |
| SEQ ID NO: 124 | LWLSGNW |
| SEQ ID NO: 125 | WTFLERL |
| SEQ ID NO: 126 | REVKES |
| SEQ ID NO: 127 | GEWLGEC |
| SEQ ID NO: 128 | PNPLMPL |
| SEQ ID NO: 129 | DPRATPGS |
| SEQ ID NO: 130 | CXFXXXYXYLMC |
| SEQ ID NO: 131 | CVXYCXXXXCW |
| | XC |
| SEQ ID NO: 132 | SLWYLGA |
| SEQ ID NO: 133 | KRVYVLG |
| SEQ ID NO: 134 | WKPASLS |
| SEQ ID NO: 135 | LVRPLEG |
| SEQ ID NO: 136 | EGRPMVY |
| SEQ ID NO: 137 | RVWQGDV |
| SEQ ID NO: 138 | FYWLYGS |
| SEQ ID NO: 139 | VSFLEYR |
| SEQ ID NO: 140 | SMSIARL |
| SEQ ID NO: 141 | QVEEFPC |
| SEQ ID NO: 142 | GPMLSVM |
| SEQ ID NO: 143 | VLPGGQW |
| SEQ ID NO: 144 | RTPAAVM |
| SEQ ID NO: 145 | YVGGWEL |
| SEQ ID NO: 146 | CVFXXXYXXC |
| SEQ ID NO: 147 | CVXYCXXXXCYVC |

TABLE 15
Lung Homing Peptides

| | |
|---|---|
| SEQ ID NO: 148 | CGFECVRQCPER |
| | C |
| SEQ ID NO: 149 | CIKGNVNC |
| SEQ ID NO: 150 | CLYIDRRC |
| SEQ ID NO: 151 | CSKLMMTC |
| SEQ ID NO: 152 | CNSDVDLC |
| SEQ ID NO: 153 | CEKKLLYC |
| SEQ ID NO: 154 | CVDSQSMKGLVC |
| SEQ ID NO: 155 | CRPAQRDAGTSC |
| SEQ ID NO: 156 | GGEVASNERIQC |
| SEQ ID NO: 157 | CTLRDRNC |
| SEQ ID NO: 158 | CRHESSSC |
| SEQ ID NO: 159 | CYSLGADC |
| SEQ ID NO: 160 | CTFRNASC |
| SEQ ID NO: 161 | CRTHGYQGC |
| SEQ ID NO: 162 | CKTNHMESC |
| SEQ ID NO: 163 | CKDSAMTIC |
| SEQ ID NO: 164 | CMSWDAVSC |
| SEQ ID NO: 165 | CMSPQRSDC |
| SEQ ID NO: 166 | CPQDIRRNC |
| SEQ ID NO: 167 | CQTRNFAQC |
| SEQ ID NO: 168 | CQDLNIMQC |
| SEQ ID NO: 169 | CGYIDPNRISQC |
| SEQ ID NO: 170 | CRLRSYGTLSLC |
| SEQ ID NO: 171 | TRRTNNPLT |
| SEQ ID NO: 172 | CTVNEAYKTRMC |
| SEQ ID NO: 173 | CAGTCATGCNGV |
| | C |
| SEQ ID NO: 174 | CPKARPAPQYKC |
| SEQ ID NO: 175 | CQETRTEGRKKC |
| SEQ ID NO: 176 | CHEGYLTC |
| SEQ ID NO: 177 | CIGEVEVC |
| SEQ ID NO: 178 | CLRPYLNC |
| SEQ ID NO: 179 | CMELSKQG |
| SEQ ID NO: 180 | CGNETLRC |
| SEQ ID NO: 181 | CMGSEYWC |
| SEQ ID NO: 182 | CAHQHIQC |
| SEQ ID NO: 183 | CAQNMLCC |
| SEQ ID NO: 184 | CADYDLALGLMC |
| SEQ ID NO: 185 | CSSHQGGFQHGC |
| SEQ ID NO: 186 | CRPWHNQAHTEC |
| SEQ ID NO: 187 | CSEAASRMIGVC |
| SEQ ID NO: 188 | CWDADQIEGIKC |
| SEQ ID NO: 189 | CRLQTMGQGQSC |
| SEQ ID NO: 190 | CGGRDRGTYGPC |
| SEQ ID NO: 191 | CNSKSSAELEKC |
| SEQ ID NO: 192 | CRGKPLANFEDC |
| SEQ ID NO: 193 | CRDRGDRMKSLC |
| SEQ ID NO: 194 | CSFGTHDTEPHC |
| SEQ ID NO: 195 | CWEEHPSIKWWC |
| SEQ ID NO: 196 | CIFREANVC |
| SEQ ID NO: 197 | CTRSTNTGC |
| SEQ ID NO: 198 | CLVGCEVGCSPA |
| | C |
| SEQ ID NO: 199 | CDTSCENNCQGP |
| | C |
| SEQ ID NO: 200 | CRGDCGIGCRRL |
| | C |
| SEQ ID NO: 201 | CSEGCGPVCWPE |
| | C |
| SEQ ID NO: 202 | RNVPPIFNDVYY |
| | WIAF |
| SEQ ID NO: 203 | VSQTMRQTAVPL |
| | LWFWTGSL |
| SEQ ID NO: 204 | RGDLATLRQLAQ |
| | EDGVVGVR |
| SEQ ID NO: 205 | CGFELETC |
| SEQ ID NO: 206 | CVGNLSMC |
| SEQ ID NO: 207 | CKGQRDFC |
| SEQ ID NO: 208 | CNMGLTRC |
| SEQ ID NO: 209 | CGTFGARC |
| SEQ ID NO: 210 | CSAHSQEMNVNC |
| SEQ ID NO: 211 | CGFECVRQCPERC |
| SEQ ID NO: 212 | CRSGCVEGCGGRC |
| SEQ ID NO: 213 | CGGECGWECEVSC |
| SEQ ID NO: 214 | CKWLCLLLCAVAC |
| SEQ ID NO: 215 | CGAACGVGCGGRC |
| SEQ ID NO: 216 | CGASCALGCRAYC |
| SEQ ID NO: 217 | CSRQCRGACGQPC |
| SEQ ID NO: 218 | CAGGGAVRCGGTC |
| SEQ ID NO: 219 | CGRPCVGECRMGC |
| SEQ ID NO: 220 | CVLNFKNQARDC |
| SEQ ID NO: 221 | CEGHSMRGYGLC |
| SEQ ID NO: 222 | CDNTCTYGVDDC |
| SEQ ID NO: 223 | CGAACGVGCRGRC |
| SEQ ID NO: 224 | CLVGCRLSCGGEC |
| SEQ ID NO: 225 | CYWWCDGVCALQC |
| SEQ ID NO: 226 | CRISAHPC |
| SEQ ID NO: 227 | CSYPKILC |
| SEQ ID NO: 228 | CSEPSGTC |
| SEQ ID NO: 229 | CTLSNRFC |
| SEQ ID NO: 230 | CLFSDENC |
| SEQ ID NO: 231 | CWRGDRKIC |
| SEQ ID NO: 232 | CCFTNFDCYLGC |
| SEQ ID NO: 233 | CYEEKSQSC |
| SEQ ID NO: 234 | CGGACGGVCTGGC |
| SEQ ID NO: 235 | CLHSPRSKC |
| SEQ ID NO: 236 | CLYTKEQRC |
| SEQ ID NO: 237 | CTGHLSTDC |
| SEQ ID NO: 238 | CIARCGGACGRHC |
| SEQ ID NO: 239 | CGVGCPGLCGGAC |
| SEQ ID NO: 240 | CLAKENVVC |
| SEQ ID NO: 241 | CSGSCRRGCGIDC |
| SEQ ID NO: 242 | CKGQGDWC |
| SEQ ID NO: 243 | CPRTCGAACASPC |
| SEQ ID NO: 244 | CERVVGSSC |
| SEQ ID NO: 245 | CKWSRLHSC |
| SEQ ID NO: 246 | QPFMQCLCIYDASC |
| SEQ ID NO: 247 | VFRVRPWYQSTSQS |
| SEQ ID NO: 248 | MTVCNASQRQAHAQA |
| | TAVSL |

TABLE 16
Skin Homing Peptides

| | |
|---|---|
| SEQ ID NO: 249 | CVGACDLKCTGG |
| | C |
| SEQ ID NO: 250 | CSTLCGLRCMG |
| SEQ ID NO: 251 | CSSGCSKINCLEM |
| | C |
| SEQ ID NO: 252 | CQGGCGVSCPIFC |
| SEQ ID NO: 253 | CGFGCSGSCQMQ |
| | C |
| SEQ ID NO: 254 | CTMGCTAGCAFA |
| | C |
| SEQ ID NO: 255 | CNQGCSGSCDVM |
| | C |
| SEQ ID NO: 256 | CVEGCSSGCGPG |
| | C |
| SEQ ID NO: 257 | CYADCEGTCGMV |
| | C |
| SEQ ID NO: 258 | CWNICPGGCRAL |
| | C |
| SEQ ID NO: 259 | CMPRCGVNCKW |
| | AC |
| SEQ ID NO: 260 | CGGGCQWGCAG |
| | EC |
| SEQ ID NO: 261 | CPSNCVALCTSGC |
| SEQ ID NO: 262 | CGKRK |
| SEQ ID NO: 263 | TSPLNIHNGQKL |
| SEQ ID NO: 264 | CRVVCADGCRLTC |
| SEQ ID NO: 265 | CFTFCEYHCQLTC |
| SEQ ID NO: 266 | CGRPCRGGCAASC |
| SEQ ID NO: 267 | CSTLCGLRCMGTC |
| SEQ ID NO: 268 | GPGCEEECQPAC |
| SEQ ID NO: 269 | CKGTCVLGCSEEC |
| SEQ ID NO: 270 | CVALCREACGEGC |
| SEQ ID NO: 271 | CAVRCDGSCVPEC |
| SEQ ID NO: 272 | CRVVCADGCRFIC |
| SEQ ID NO: 273 | CEGKCGLTCECTC |
| SEQ ID NO: 274 | CASGCSESCYVGC |
| SEQ ID NO: 275 | CSVRCKSVCIGLC |
| SEQ ID NO: 276 | CSRPRRSEC |
| SEQ ID NO: 277 | CDTRL |

TABLE 17
Retina Homing Peptides

| | |
|---|---|
| SEQ ID NO: 278 | CRRIWYAVC |
| SEQ ID NO: 279 | CSCFRDVCC |
| SEQ ID NO: 280 | CTDNRVGS |
| SEQ ID NO: 281 | CTSDISWWDYKC |
| SEQ ID NO: 282 | CVGDCIGSCWMF |
| | C |
| SEQ ID NO: 283 | CVSGHLNC |
| SEQ ID NO: 284 | CYTGETWTC |
| SEQ ID NO: 285 | CDCRGDCFC |
| SEQ ID NO: 286 | CERSQSKGVHHC |
| SEQ ID NO: 287 | CFWHNRAC |
| SEQ ID NO: 288 | CGEFKVGC |
| SEQ ID NO: 289 | CGPGYQAQCSLR |
| | C |
| SEQ ID NO: 290 | CHMGCVSPCAYV |
| | C |
| SEQ ID NO: 291 | CISRPYFC |
| SEQ ID NO: 292 | CKERPSNGLSAC |
| SEQ ID NO: 293 | CKSGCGVACRHM |
| | C |
| SEQ ID NO: 294 | CMDSQSSC |
| SEQ ID NO: 295 | CNIPVTTPIFGC |
| SEQ ID NO: 296 | CNRKNSNEQRAC |
| SEQ ID NO: 297 | CQIRPIDKC |
| SEQ ID NO: 298 | CGRFDTAPQRGC |
| SEQ ID NO: 299 | CLLNYTYC |
| SEQ ID NO: 300 | CMSLGNNC |
| SEQ ID NO: 301 | CQASASDHC |
| SEQ ID NO: 302 | CQRVNSVENASC |
| SEQ ID NO: 303 | CRRHMERC |
| SEQ ID NO: 304 | CTHLVTLC |
| SEQ ID NO: 305 | CVTSNLRVC |
| SEQ ID NO: 306 | CSAYTTSPC |
| SEQ ID NO: 307 | CTDKSWPC |
| SEQ ID NO: 308 | CTIADFPC |
| SEQ ID NO: 309 | CTVDNELC |
| SEQ ID NO: 310 | CVKFTYDC |
| SEQ ID NO: 311 | CYGESQQMC |
| SEQ ID NO: 312 | CAVSIPRC |
| SEQ ID NO: 313 | CGDVCPSECPGWC |
| SEQ ID NO: 314 | CGLDCLGDCSGAC |
| SEQ ID NO: 315 | CGSHCGQLCKSLC |
| SEQ ID NO: 316 | CILSYDNPC |
| SEQ ID NO: 317 | CKERLEYTRGVC |
| SEQ ID NO: 318 | CKPFRTEC |
| SEQ ID NO: 319 | CLKPGGQEC |
| SEQ ID NO: 320 | CMNILSGC |
| SEQ ID NO: 321 | CNQRTNRESGNC |
| SEQ ID NO: 322 | CNRMEMPC |
| SEQ ID NO: 323 | CAIDIGGAC |
| SEQ ID NO: 324 | CKRANRLSC |
| SEQ ID NO: 325 | CLNGLVSMC |
| SEQ ID NO: 326 | CNRNRMTPC |
| SEQ ID NO: 327 | CQLINSSPC |
| SEQ ID NO: 328 | CRKEHYPC |
| SEQ ID NO: 329 | CSGRPFKYC |
| SEQ ID NO: 330 | CTSSPAYNC |
| SEQ ID NO: 331 | CWDSGSHIC |
| SEQ ID NO: 332 | CERSHGRLC |
| SEQ ID NO: 333 | CINCLSQC |
| SEQ ID NO: 334 | CNSRSENC |
| SEQ ID NO: 335 | CSHHDTNC |
| SEQ ID NO: 336 | CYAGSPLC |
| SEQ ID NO: 337 | CQWSMNVC |
| SEQ ID NO: 338 | CRDVVSVIC |
| SEQ ID NO: 339 | CGNLLTRRC |
| SEQ ID NO: 340 | CLRHDFYVC |
| SEQ ID NO: 341 | CRYKGPSC |
| SEQ ID NO: 342 | CSRWYTTC |
| SEQ ID NO: 343 | CQTTSWNC |
| SEQ ID NO: 344 | CRARIRAEDISC |
| SEQ ID NO: 345 | CRREYSAC |
| SEQ ID NO: 346 | CDSLCGGACAARC |
| SEQ ID NO: 347 | CFKSTLLC |

TABLE 18

| Pancreas Homing Peptides | |
|---|---|
| SEQ ID NO: 348 | EICQLGSCT |
| SEQ ID NO: 349 | RKCLRPDCG |
| SEQ ID NO: 350 | LACFVTGCL |
| SEQ ID NO: 351 | DMCWLIGCG |
| SEQ ID NO: 352 | QRCPRSFCL |
| SEQ ID NO: 353 | RECTNEICY |
| SEQ ID NO: 354 | SCVFCDWLS |
| SEQ ID NO: 355 | QNCPVTRCV |
| SEQ ID NO: 356 | CDNREMSC |
| SEQ ID NO: 357 | CGEYGREC |
| SEQ ID NO: 358 | CKKRLLNVC |
| SEQ ID NO: 359 | CMTGRVTC |
| SEQ ID NO: 360 | CPDLLVAC |
| SEQ ID NO: 361 | CSKAYDLAC |
| SEQ ID NO: 362 | CTLKHTAMC |
| SEQ ID NO: 363 | CTTEIDYC |
| SEQ ID NO: 364 | CRGRRST |
| SEQ ID NO: 365 | BCDDDGQRLGNQ |
| | WAVGHLM |
| SEQ ID NO: 366 | CHVLWSTRC |
| SEQ ID NO: 367 | GAWEAVRDRIAE |
| | WGSWGIPS |
| SEQ ID NO: 368 | KAA |
| SEQ ID NO: 369 | WRCEGFNCQ |
| SEQ ID NO: 370 | SWCEPGWCR |
| SEQ ID NO: 371 | GLCNGATCM |
| SEQ ID NO: 372 | SGCRTMVCV |
| SEQ ID NO: 373 | LSCAPVICG |
| SEQ ID NO: 374 | NECLMISCR |
| SEQ ID NO: 375 | WACEELSCF |
| SEQ ID NO: 376 | CATLTNDEC |
| SEQ ID NO: 377 | CFMDHSNC |
| SEQ ID NO: 378 | CHMKRDRTC |
| SEQ ID NO: 379 | CLDYHPKC |
| SEQ ID NO: 380 | CNKIVRRC |
| SEQ ID NO: 381 | CSDTQSIGC |
| SEQ ID NO: 382 | CSKKGPSYC |
| SEQ ID NO: 383 | CTQHIANC |
| SEQ ID NO: 384 | CVGRSGELC |
| SEQ ID NO: 385 | CKAAKNK |
| SEQ ID NO: 386 | CVSNPRWKC |
| SEQ ID NO: 387 | LSGTPERSGQAVKVKL |
| | KAIP |
| SEQ ID NO: 388 | RSR |
| SEQ ID NO: 389 | RGR |

TABLE 19
Liver Homing Peptides

| | |
|---|---|
| SEQ ID NO: 390 | ARRGWTL |
| SEQ ID NO: 391 | QLTGGCL |
| SEQ ID NO: 392 | KAYFRWR |
| SEQ ID NO: 393 | VGSFIYS |
| SEQ ID NO: 394 | LSTVLWF |
| SEQ ID NO: 395 | GRSSLAC |
| SEQ ID NO: 396 | CGGAGAR |
| SEQ ID NO: 397 | DFLRCRV |
| SEQ ID NO: 398 | RALYDAL |
| SEQ ID NO: 399 | GMAVSSW |
| SEQ ID NO: 400 | WQSVVRV |
| SEQ ID NO: 401 | CGNGHSC |
| SEQ ID NO: 402 | SLRPDNG |
| SEQ ID NO: 403 | TACHQHVRMVRP |
| SEQ ID NO: 404 | SRRFVGG |
| SEQ ID NO: 405 | ALERRSL |
| SEQ ID NO: 406 | RWLAWTV |
| SEQ ID NO: 407 | LSLLGIA |
| SEQ ID NO: 408 | SLAMRDS |
| SEQ ID NO: 409 | SELLGDA |
| SEQ ID NO: 410 | WRQNMPL |
| SEQ ID NO: 411 | QAGLRCH |
| SEQ ID NO: 412 | WVSVLGF |
| SEQ ID NO: 413 | SWFFLVA |
| SEQ ID NO: 414 | VKSVCRT |
| SEQ ID NO: 415 | AEMEGRD |
| SEQ ID NO: 416 | PAMGLIR |

TABLE 20
Lymph Node Homing Peptides

| | |
|---|---|
| SEQ ID NO: 417 | WGCKLRFCS |
| SEQ ID NO: 418 | GICATVKCS |
| SEQ ID NO: 419 | TTCMSQLCL |
| SEQ ID NO: 420 | GCVRRLLCN |
| SEQ ID NO: 421 | KYCTPVECL |
| SEQ ID NO: 422 | MCPQRNCL |
| SEQ ID NO: 423 | AGCSVTVCG |
| SEQ ID NO: 424 | GSCSMFPCS |
| SEQ ID NO: 425 | SECAYRACS |
| SEQ ID NO: 426 | SLCGSDGCR |
| SEQ ID NO: 427 | MRCQFSGCT |
| SEQ ID NO: 428 | STCGNWTCR |
| SEQ ID NO: 429 | CSCTGQLCR |
| SEQ ID NO: 430 | GLCQIDECR |
| SEQ ID NO: 431 | DRCLDIWCL |
| SEQ ID NO: 432 | PLCMATRCA |
| SEQ ID NO: 433 | NPCLRAACI |
| SEQ ID NO: 434 | LECVANLCT |
| SEQ ID NO: 435 | EPCTWNACL |
| SEQ ID NO: 436 | LYCLDASCL |
| SEQ ID NO: 437 | LVCQGSPCL |
| SEQ ID NO: 438 | DXCXDIWCL |
| SEQ ID NO: 439 | KTCVGVRV |
| SEQ ID NO: 440 | LTCWDWSCR |
| SEQ ID NO: 441 | KTCAGSSCI |
| SEQ ID NO: 442 | NPCFGLLV |
| SEQ ID NO: 443 | RTCTPSRCM |
| SEQ ID NO: 444 | QYCWSKGCR |
| SEQ ID NO: 445 | VTCSSEWCL |
| SEQ ID NO: 446 | STCISVHCS |
| SEQ ID NO: 447 | IACDGYLCG |
| SEQ ID NO: 448 | XGCYQKRCT |
| SEQ ID NO: 449 | IRCWGGRCS |
| SEQ ID NO: 450 | AGCVQSQCY |
| SEQ ID NO: 451 | KACFGADCX |
| SEQ ID NO: 452 | SACWLSNCA |
| SEQ ID NO: 453 | GLCQEHRCW |
| SEQ ID NO: 454 | EDCREWGCR |
| SEQ ID NO: 455 | CGNKRTRGC |
| SEQ ID NO: 456 | CLSDGKRKC |
| SEQ ID NO: 457 | CREAGRKAC |
| SEQ ID NO: 458 | MECIKYSCL |
| SEQ ID NO: 459 | PRCQLWACT |
| SEQ ID NO: 460 | SHCPMASLC |
| SEQ ID NO: 461 | TSCRLFSCA |
| SEQ ID NO: 462 | RGCNGSRCS |
| SEQ ID NO: 463 | PECEGYSCI |
| SEQ ID NO: 464 | IPCYWESCR |
| SEQ ID NO: 465 | QDCVKRPCV |
| SEQ ID NO: 466 | WSCARPLCG |
| SEQ ID NO: 467 | RLCPSSPCT |
| SEQ ID NO: 468 | RYCYPDGCL |
| SEQ ID NO: 469 | LPCTGASCP |
| SEQ ID NO: 470 | LECRRWRCD |
| SEQ ID NO: 471 | TACKVAACH |
| SEQ ID NO: 472 | XXXQGSPCL |
| SEQ ID NO: 473 | RDCSHRSCE |
| SEQ ID NO: 474 | PTCAYGWCA |
| SEQ ID NO: 475 | RKCGEEVCT |
| SEQ ID NO: 476 | LVCPGTACV |
| SEQ ID NO: 477 | ERCPMAKCY |
| SEQ ID NO: 478 | QQCQDPYCL |
| SEQ ID NO: 479 | QPCRSMVCA |
| SEQ ID NO: 480 | WSCHEFNCR |
| SEQ ID NO: 481 | SLCRLSTCS |
| SEQ ID NO: 482 | VICTGRQCG |
| SEQ ID NO: 483 | SLCTAFNCH |
| SEQ ID NO: 484 | QSCLWRICI |
| SEQ ID NO: 485 | LGCFPSWCG |
| SEQ ID NO: 486 | RLCSWGGCA |
| SEQ ID NO: 487 | EVCLVLSCQ |
| SEQ ID NO: 488 | RDCVKNLCR |
| SEQ ID NO: 489 | LGCFXSWCG |
| SEQ ID NO: 490 | IPCSLLGCA |
| SEQ ID NO: 491 | PRCWERVCS |
| SEQ ID NO: 492 | TLCPLVACE |
| SEQ ID NO: 493 | SECYTGSCP |
| SEQ ID NO: 494 | VECGFSAVF |
| SEQ ID NO: 495 | HWCRLLACR |
| SEQ ID NO: 496 | CAGRRSAYC |
| SEQ ID NO: 497 | CNRRTKAGC |

TABLE 21
Adrenal Gland Homing Peptides

| | |
|---|---|
| SEQ ID NO: 498 | WGCKLRFCS |
| SEQ ID NO: 499 | GICATVKCS |
| SEQ ID NO: 500 | TTCMSQLCL |
| SEQ ID NO: 501 | GCVRRLLCN |
| SEQ ID NO: 502 | KYCTPVECL |
| SEQ ID NO: 503 | MCPQRNCL |
| SEQ ID NO: 504 | AGCSVTVCG |
| SEQ ID NO: 505 | GSCSMFPCS |
| SEQ ID NO: 506 | SECAYRACS |
| SEQ ID NO: 507 | SLCGSDGCR |
| SEQ ID NO: 508 | MRCQFSGCT |
| SEQ ID NO: 509 | STCGNWTCR |
| SEQ ID NO: 510 | CSCTGQLCR |
| SEQ ID NO: 511 | GLCQIDECR |
| SEQ ID NO: 512 | DRCLDIWCL |
| SEQ ID NO: 513 | PLCMATRCA |
| SEQ ID NO: 514 | NPCLRAACI |
| SEQ ID NO: 515 | LECVANLCT |
| SEQ ID NO: 516 | EPCTWNACL |
| SEQ ID NO: 517 | LYCLDASCL |
| SEQ ID NO: 518 | LVCQGSPCL |
| SEQ ID NO: 519 | DXCXDIWCL |
| SEQ ID NO: 520 | KTCVGVRV |
| SEQ ID NO: 521 | LTCWDWSCR |
| SEQ ID NO: 522 | KTCAGSSCI |
| SEQ ID NO: 523 | NPCFGLLV |
| SEQ ID NO: 524 | RTCTPSRCM |
| SEQ ID NO: 525 | QYCWSKGCR |
| SEQ ID NO: 526 | VTCSSEWCL |
| SEQ ID NO: 527 | STCISVHCS |
| SEQ ID NO: 528 | IACDGYLCG |
| SEQ ID NO: 529 | XGCYQKRCT |
| SEQ ID NO: 530 | IRCWGGRCS |
| SEQ ID NO: 531 | AGCVQSQCY |
| SEQ ID NO: 532 | KACGGADCX |
| SEQ ID NO: 533 | SACWLSNCA |
| SEQ ID NO: 534 | GLCQEHRCW |
| SEQ ID NO: 535 | EDCREWGCR |
| SEQ ID NO: 536 | LMLPRAD |
| SEQ ID NO: 537 | MECIKYSCL |
| SEQ ID NO: 538 | PRCQLWACT |
| SEQ ID NO: 539 | SHCPMASLC |
| SEQ ID NO: 540 | TSCRLFSCA |
| SEQ ID NO: 541 | RGCNGSRCS |
| SEQ ID NO: 542 | PECEGVSCI |
| SEQ ID NO: 543 | IPCYWESCR |
| SEQ ID NO: 544 | QDCVKRPCV |
| SEQ ID NO: 545 | WSCARPLCG |
| SEQ ID NO: 546 | RLCPSSPCT |
| SEQ ID NO: 547 | RYCYPDGCL |
| SEQ ID NO: 548 | LPCTGASCP |
| SEQ ID NO: 549 | LECRRWRCD |
| SEQ ID NO: 550 | TACKVAACH |
| SEQ ID NO: 551 | XXXQGSPCL |
| SEQ ID NO: 552 | RDCSHRSCE |
| SEQ ID NO: 553 | PTCAYGWCA |
| SEQ ID NO: 554 | RKCGEEVCT |
| SEQ ID NO: 555 | LVCPGTACV |
| SEQ ID NO: 556 | ERCPMAKCY |
| SEQ ID NO: 557 | QQCQDPYCL |
| SEQ ID NO: 558 | QPCRSMVCA |
| SEQ ID NO: 559 | WSCHEFNCR |
| SEQ ID NO: 560 | SLCRLSTCS |
| SEQ ID NO: 561 | VICTGRQCG |
| SEQ ID NO: 562 | SLCTAFNCH |
| SEQ ID NO: 537 | QSCLWRICI |
| SEQ ID NO: 538 | LGCFPSWCG |
| SEQ ID NO: 539 | RLCSWGGCA |
| SEQ ID NO: 540 | EVCLVLSCQ |
| SEQ ID NO: 541 | RDCVKNLCR |
| SEQ ID NO: 542 | LGCFXSWCG |
| SEQ ID NO: 543 | IPCSLLGCA |
| SEQ ID NO: 544 | PRCWERVCS |
| SEQ ID NO: 545 | TLCPLVACE |
| SEQ ID NO: 546 | SECYTGSCP |
| SEQ ID NO: 547 | VECGFSAVF |
| SEQ ID NO: 548 | HWCRLLACR |

TABLE 22
Thyroid Homing Peptides

| | |
|---|---|
| SEQ ID NO: 549 | SRESPHP |
| SEQ ID NO: 550 | HTFEPGV |

TABLE 23
Bladder Homing Peptides

| | |
|---|---|
| SEQ ID NO: 551 | CSNRDARRC |
| SEQ ID NO: 552 | CXNXDXR(X)/(R)C |

TABLE 24
Breast Homing Peptides

| | |
|---|---|
| SEQ ID NO: 553 | PRP |
| SEQ ID NO: 554 | SSSPL |
| SEQ ID NO: 555 | SPW |
| SEQ ID NO: 556 | PHSK |
| SEQ ID NO: 557 | LSAN |
| SEQ ID NO: 558 | KHST |
| SEQ ID NO: 559 | TLLS |
| SEQ ID NO: 560 | SSTA |
| SEQ ID NO: 561 | TSAH |
| SEQ ID NO: 562 | CPGPEGAGC |

TABLE 25
Neuroblastoma Homing Peptides

| | |
|---|---|
| SEQ ID NO: 563 | VPWMEPAYQRFL |
| SEQ ID NO: 564 | HLQLQPWYPQIS |

TABLE 26
Lymphoma Homing Peptides

| | |
|---|---|
| SEQ ID NO: 565 | LVRSTGQFV |
| SEQ ID NO: 566 | ALRPSGEWL |
| SEQ ID NO: 567 | QILASGRWL |
| SEQ ID NO: 568 | DNNRPANSM |
| SEQ ID NO: 569 | PLSGDKSST |
| SEQ ID NO: 570 | RMWPSSTVNLSA |
| | GRR |
| SEQ ID NO: 571 | GRVPSMFGGHFF |
| | FSR |
| SEQ ID NO: 572 | LVSPSGSWT |
| SEQ ID NO: 573 | AIMASGQWL |
| SEQ ID NO: 574 | RRPSHAMAR |
| SEQ ID NO: 575 | LQDRLRFAT |
| SEQ ID NO: 576 | IELLQAR |
| SEQ ID NO: 577 | PNLDFSPTCSFRFGC |

TABLE 27
Muscle Homing Peptides

| | |
|---|---|
| SEQ ID NO: 578 | TARGEHKEEELI |
| SEQ ID NO: 579 | TGGETSGIKKAPY |
| | ASTTRNR |
| SEQ ID NO: 580 | SHHGVAGVDLGGGAD |
| | FKSIA |
| SEQ ID NO: 581 | ASSLNIA |

TABLE 28
Wound Vasculature Homing Peptides

| | |
|---|---|
| SEQ ID NO: 582 | CGLIIQKNEC |
| SEQ ID NO: 583 | CNAGESSKNC |

TABLE 29
Adipose Tissue Homing Peptides

| | |
|---|---|
| SEQ ID NO: 584 | CKGGRAKDC |

TABLE 30
Virus-binding Peptides

| | |
|---|---|
| SEQ ID NO: 585 | RRKKAAVALLPA |
| | VLLALLAP |
| SEQ ID NO: 586 | TDVILMCFSIDSPDSLEN |
| | I |

TABLE 31
Fusogenic Peptides

| | |
|---|---|
| SEQ ID NO: 587 | KALA |
| SEQ ID NO: 588 | RQIKIWFQNRRMKWKK |

Additional exemplary targeting groups suitable for derivitization as click-functionalized targeting groups in accordance with the present invention include those set forth in Tables 32-38, below. Exemplary peptides that have been shown to be useful for targeting tumors in general in vivo are listed in Table 32. In some cases, the peptide sequences listed in Tables 32-38 are cyclized variations of the linear sequences.

TABLE 32
Tumor Homing Peptides

| | |
|---|---|
| SEQ ID NO: 589 | CGRECPRLCQSSC |
| SEQ ID NO: 590 | SKVLYYNWE |
| SEQ ID NO: 591 | CPTCNGRCVR |
| SEQ ID NO: 592 | CAVCNGRCGF |
| SEQ ID NO: 593 | CVQCNGRCAL |
| SEQ ID NO: 594 | CEGVNGRRLR |
| SEQ ID NO: 595 | KMGPKVW |
| SEQ ID NO: 596 | CWSGVDC |
| SEQ ID NO: 597 | CVMVRDGDC |
| SEQ ID NO: 598 | CPEHRSLVC |
| SEQ ID NO: 599 | CAQLLQVSC |
| SEQ ID NO: 600 | CTAMRNTDC |
| SEQ ID NO: 601 | CYLVNVDC |
| SEQ ID NO: 602 | QWCSRRWCT |
| SEQ ID NO: 603 | AGCINGLCG |
| SEQ ID NO: 604 | LDCLSELCS |
| SEQ ID NO: 605 | RWCREKSCW |
| SEQ ID NO: 606 | CEQCNGRCGQ |
| SEQ ID NO: 607 | CSCCNGRCGD |
| SEQ ID NO: 608 | CASNNGRVVL |
| SEQ ID NO: 609 | CEVCNGRCAL |
| SEQ ID NO: 610 | SPGSWTW |
| SEQ ID NO: 611 | SKSSGVS |
| SEQ ID NO: 612 | CQLAAVC |
| SEQ ID NO: 613 | CYVELHC |
| SEQ ID NO: 614 | CKALSQAC |
| SEQ ID NO: 615 | CGTRVDHC |
| SEQ ID NO: 616 | ISCAVDACL |
| SEQ ID NO: 617 | NRCRGVSCT |
| SEQ ID NO: 618 | CGEACGGQCALP |
| | C |
| SEQ ID NO: 619 | CERACRNLCREG |
| | C |
| SEQ ID NO: 620 | CRNCNGRCEG |
| SEQ ID NO: 621 | CWGCNGRCRM |
| SEQ ID NO: 622 | CGRCNGRCLL |
| SEQ ID NO: 623 | CGSLVRC |
| SEQ ID NO: 624 | NPRWFWD |
| SEQ ID NO: 625 | IVADYQR |
| SEQ ID NO: 626 | CGVGSSC |
| SEQ ID NO: 627 | CWRKYC |
| SEQ ID NO: 628 | CTDYVRC |
| SEQ ID NO: 629 | VTCRSLMCQ |
| SEQ ID NO: 630 | RHCFSQWCS |
| SEQ ID NO. 631 | NACESAICG |
| SEQ ID NO: 632 | KGCGTRQCW |
| SEQ ID NO: 633 | IYCPGQECE |
| SEQ ID NO: 634 | CNKTDGDEGVTC |
| SEQ ID NO: 635 | CVTCNGRCRV |
| SEQ ID NO: 636 | CKSCNGRCLA |
| SEQ ID NO: 637 | CSKCNGRCGH |
| SEQ ID NO: 638 | HHTRFVS |
| SEQ ID NO: 639 | IKARASP |
| SEQ ID NO: 640 | VVDRFPD |
| SEQ ID NO: 641 | CGLSDSC |
| SEQ ID NO: 642 | CYSYFLAC |
| SEQ ID NO: 643 | VPCRFKQCW |
| SEQ ID NO: 644 | CYLGVSNC |
| SEQ ID NO: 645 | RSCIKHQCP |
| SEQ ID NO: 646 | FGCVMASCR |
| SEQ ID NO: 647 | PSCAYMCIT |
| SEQ ID NO: 648 | CKVCNGRCCG |
| SEQ ID NO: 649 | CTECNGRCQL |
| SEQ ID NO: 650 | CVPCNGRCHE |
| SEQ ID NO: 651 | CVWCNGRCGL |
| SEQ ID NO: 652 | SKGLRHR |
| SEQ ID NO: 653 | SGWCYRC |
| SEQ ID NO: 654 | LSMFTRP |
| SEQ ID NO: 655 | CGEGHPC |
| SEQ ID NO: 656 | CPRGSRC |
| SEQ ID NO: 657 | TDCTPSRCT |
| SEQ ID NO: 658 | CISLDRSC |
| SEQ ID NO: 659 | EACEMAGCL |
| SEQ ID NO: 660 | EPCEGKKCL |
| SEQ ID NO: 661 | KRCSSSLCA |
| SEQ ID NO: 662 | EDCTSRFCS |
| SEQ ID NO: 663 | CPLCNGRCAL |
| SEQ ID NO: 664 | CETCNGRCAL |
| SEQ ID NO: 665 | CRTCNGRCQV |
| SEQ ID NO: 666 | CGECNGRCVE |
| SEQ ID NO: 667 | WRVLAAF |
| SEQ ID NO: 668 | LWAEMTG |
| SEQ ID NO: 669 | IMYPGWL |
| SEQ ID NO: 670 | CELSLISKC |
| SEQ ID NO: 671 | CDDSWKC |
| SEQ ID NO: 672 | CMEMGVKC |
| SEQ ID NO: 673 | LVCLPPSCE |
| SEQ ID NO: 674 | GICKDLWCQ |
| SEQ ID NO: 675 | DTCRALRCN |
| SEQ ID NO: 676 | YRCIARECE |
| SEQ ID NO: 677 | RKCEVPGCQ |
| SEQ ID NO: 678 | CEMCNGRCMG |
| SEQ ID NO: 679 | CRTCNGRCLE |
| SEQ ID NO: 680 | CQSCNGRCVR |
| SEQ ID NO: 681 | CIRCNGRCSV |
| SEQ ID NO: 682 | VASVSVA |
| SEQ ID NO: 683 | ALVGLMR |
| SEQ ID NO: 684 | GLPVKWS |
| SEQ ID NO: 685 | CYTADPC |
| SEQ ID NO: 686 | CRLGIAC |
| SEQ ID NO: 687 | SWCQFEKCL |
| SEQ ID NO: 688 | CAMVSMED |
| SEQ ID NO: 689 | PRCESQLCP |
| SEQ ID NO: 690 | ADCRQKPCL |
| SEQ ID NO: 691 | ICLLAHCA |
| SEQ ID NO: 692 | LECVVDSCR |
| SEQ ID NO: 693 | IWSGYGVYW |
| SEQ ID NO: 694 | CPRGCLAVCVSQ |
| | C |
| SEQ ID NO: 695 | QACPMLLCM |
| SEQ ID NO: 696 | EICVDGLCV |
| SEQ ID NO: 697 | CGVCNGRCGL |
| SEQ ID NO: 698 | CRDLNGRKVM |
| SEQ ID NO: 699 | CRCCNGRCSP |
| SEQ ID NO: 700 | CLSCNGRCPS |
| SEQ ID NO: 701 | IFSGSRE |
| SEQ ID NO: 702 | DTLRLRI |
| SEQ ID NO: 703 | CVRIRPC |
| SEQ ID NO: 704 | CLVVHEAAC |
| SEQ ID NO: 705 | CYPADPC |
| SEQ ID NO: 706 | CRESLKNC |
| SEQ ID NO: 707 | CIRSAVSC |
| SEQ ID NO: 708 | MFCRMRSCD |
| SEQ ID NO: 709 | RSCAEPWCY |
| SEQ ID NO: 710 | AGCRVESC |
| SEQ ID NO: 711 | FRCLERVCT |
| SEQ ID NO: 712 | WESLYFPRE |
| SEQ ID NO: 713 | RLCRIVVIRVCR |
| SEQ ID NO: 714 | HTCLVALCA |
| SEQ ID NO: 715 | RPCGDQACE |
| SEQ ID NO: 716 | CVLCNGRCWS |
| SEQ ID NO: 717 | CPLCNGRCAR |
| SEQ ID NO: 718 | CWLCNGRCGR |
| SEQ ID NO: 719 | GRSQMQI |
| SEQ ID NO: 720 | GRWYKWA |
| SEQ ID NO: 721 | VWRTGHL |
| SEQ ID NO: 722 | CVSGPRC |
| SEQ ID NO: 723 | CFWPNRC |
| SEQ ID NO: 724 | CGETMRC |
| SEQ ID NO: 725 | CNNVGSYC |
| SEQ ID NO: 726 | FYCPGVGCR |
| SEQ ID NO: 727 | APCGLLACI |
| SEQ ID NO: 728 | GRCVDGGCT |
| SEQ ID NO: 729 | RLCSLYGCV |
| SEQ ID NO: 730 | CNGRCVSGCAGRC |
| SEQ ID NO: 731 | CGLMCQGACFDVC |
| SEQ ID NO: 732 | YVPLPNVPQPGRRPFPT |
| | FPGQGPFNPKIKWPQG |
| | Y |
| SEQ ID NO: 733 | VFIDILDKVENAIHNAA |
| | QVGIGFAKPFEKHLINP |
| | K |
| SEQ ID NO: 734 | GNNRPVYIPQPRPPHPRI |
| SEQ ID NO: 735 | GNNRPVYIPQPRPPHPR |
| | L |
| SEQ ID NO: 736 | GNNRPIYIPQPRPPHPRL |
| SEQ ID NO: 737 | RFRPPIRRPPIRPPFYPPF |
| | RPPIRPPIFPPIRPPFRPPL |
| | RFP |
| SEQ ID NO: 738 | RRIRPRPPRLPRPRPRPL |
| | PFPRPGPRPIPRPLPFPRP |
| | GPRPIPRLPLPFFRPGPR |
| | PIPRP |
| SEQ ID NO: 739 | PRPIPRPLPFFRPGPRPIP |
| | R |
| SEQ ID NO: 740 | WNPFKELERAGQRVRD |
| | AVISAAPAVATVGQAA |
| | LARG |
| SEQ ID NO: 741 | WNPFKELERAGQRVRD |
| | AIISAGPAVATVGQAAA |
| | IA |
| SEQ ID NO: 742 | WNPFKELERAGQRVRD |
| | AIISAAPAVATVGQAAA |
| | IARG |
| SEQ ID NO: 743 | WNPFKELERAGQRVRD |
| | AVISAAPAVATVGQAA |
| | AIARGG |
| SEQ ID NO: 744 | GIGALSAKGALKGLAK |
| | GLAZHFAN |
| SEQ ID NO. 745 | GIGASILSAGKSALKGL |
| | AKGLAEHFAN |
| SEQ ID NO: 746 | GIGSAILSAGKSALKGL |
| | AKGLAEHFAN |
| SEQ ID NO: 747 | IKITTMLAKILGKVLAH |
| | V |
| SEQ ID NO: 748 | SKITDILAKLGKVLAIIV |
| SEQ ID NO: 749 | RPDFCLEPPYTGPCKAR |
| | II |
| SEQ ID NO: 750 | RYFYNAKAGLCQTFVY |
| | G |
| SEQ ID NO: 751 | GCRAKRINNFKSAEDC |
| | MRTCGGA |
| SEQ ID NO: 752 | FLPLLAGLAANFLPKIF |
| | CKITRKC |
| SEQ ID NO: 753 | GIMDTLKNLAKTAGKG |
| | ALQSLLNKASCKLSGQ |
| | C |
| SEQ ID NO: 754 | KWKLFKKIEKVGQNIR |
| | DGIIKAGPAVAVVGQA |
| | TQIAK |
| SEQ ID NO: 755 | KWKVFKIKIEKMGRNI |
| | RNGIVKAGPAIAVLGEA |
| | KAL |
| SEQ ID NO: 756 | GWILKKLGKRIERIGQH |
| | TRDATIQGLGIAQQAA |
| | NVAATARG |
| SEQ ID NO: 757 | WNPFKELEKVGQRVRD |
| | AVISAGPAVATVAAQA |
| | TALAK |
| SEQ ID NO: 758 | SWLSKTAKKLENSAKK |
| | RISEGIAIAIQGGPR |
| SEQ ID NO: 759 | ZFTNVSCTTSKECWSV |
| | CQRLHNTSRGKCMNK |
| | KCRCYS |
| SEQ ID NO: 760 | FLPLILRKIVTAL |
| SEQ ID NO: 761 | LRDLVCYCRSRGCKGR |
| | ERMNGTCRKGHLLYTL |
| | CCR |
| SEQ ID NO: 762 | LRDLVCYCRTRGCKRR |
| | ERMNGTCRKGHLMYT |
| | LCCR |
| SEQ ID NO: 763 | VVCACRRALCLPRERR |
| | AGFCRIRGRIHTPLCCR |
| | R |
| SEQ ID NO: 764 | VVCACRRALCLPLERR |
| | AGFCRIRGRIHPLCCRR |
| SEQ ID NO: 765 | RRCICTTRTCRFPYRRL |
| | GTCIFQNRVYTFCC |
| SEQ ID NO: 766 | RRCICTTRTCRFPYRRL |
| | GTCLFQNRVYTFCC |
| SEQ ID NO: 767 | ACYCRIPACIAGERRYG |
| | TCIYQGRLWAFCC |
| SEQ ID NO: 768 | CYCRIPACIAGERRYGT |
| | CIYQGRLWAFCC |
| SEQ ID NO: 769 | VVCACRRALCLPRERR |
| | AGFCRIRGRIHPLCCRR |
| SEQ ID NO: 770 | VVCACRRALCLPLERR |
| | AGFCRIRGRIHPLCCRR |
| SEQ ID NO: 771 | VTCYCRRTRCGFRERLS |
| | GACGYRGRIYRLCCR |
| SEQ ID NO: 772 | VTCYCRSTRCGFRERLS |
| | GACGYRGRIYRLCCR |
| SEQ ID NO: 773 | DFASCHTNGGICLPNRC |
| | PGHMIQIGICFRPRVKC |
| | CRSW |
| SEQ ID NO: 774 | VRNHVTCRINRGFCVPI |
| | RCPGRTRQIGTCFGPRI |
| | KCCRSW |
| SEQ ID NO: 775 | NPVSCVRNKGICVPIRC |
| | PGSMKQIGTCVGRAVK |
| | CCRKK |
| SEQ ID NO: 776 | ATCDLLSGTGINHSACA |
| | AHCLLRGNRGGYCNG |
| | KAVCVCRN |
| SEQ ID NO: 777 | GFGCPLDQMQCHRHCQ |
| | TITGRSGGYCSGPLKLT |
| | CTCYR |
| SEQ ID NO: 778 | GFGCPLNQGACHRHCR |
| | SIRRRGGYCAGFFKQTC |
| | TCYRN |
| SEQ ID NO: 779 | ALWKTMLKKLGTMAL |
| | HAGKAALGAADTISQT |
| | Q |
| SEQ ID NO: 780 | GKPRPYSPRPTSHPRPIR |
| | V |
| SEQ ID NO: 781 | GIFSKIGRKKIKNLLISG |
| | LKNVGKEVGMDVVRT |
| | GIDIAGCKIKGEC |
| SEQ ID NO: 782 | ILPWKWPWWPWRR |
| SEQ ID NO: 783 | FKCRRWQWRMKKLGA |
| | PSITCVRRAP |
| SEQ ID NO: 784 | ITSISLCTPGCKTGALM |
| | GCNMKTATCHCSIHVS |
| | K |
| SEQ ID NO: 785 | TAGPAIRASVKQCQKT |
| | LKATRLFTVSCKGKNG |
| | CK |
| SEQ ID NO: 786 | MSKFDDFDLDVVKVSK |
| | QDSKITPQWKSESLCTP |
| | GCVTGALQTCFLQTLT |
| | CNCKISK |
| SEQ ID NO: 787 | KYYGNGVHCTKSGCSV |
| | N |
| SEQ ID NO: 788 | WGEAFSAGVHRLANG |
| | GNGFW |
| SEQ ID NO: 789 | GIGKFLHSAGKFGKAF |
| | VGEIMKS |
| SEQ ID NO: 790 | GIGKFLHSAKKFGKAF |
| | VGEIMNS |
| SEQ ID NO: 791 | GMASKAGAIAGKIAKV |
| | ALKAL |
| SEQ ID NO: 792 | GVLSNVIGYLKKLGTG |
| | ALNAVLKG |
| SEQ ID NO: 793 | GWASKIGQTLGKIAKV |
| | GLKELIQPK |
| SEQ ID NO: 794 | INLKALAALAKKIL |
| SEQ ID NO: 795 | GIGAVLKVLTTGLPALI |
| | SWIKRKRQQ |
| SEQ ID NO: 796 | ATCDLLSGTGINHSACA |
| | AHCLLRGNRGGYCNG |
| | KGVCVCRN |
| SEQ ID NO: 797 | ATCDLLSGTGINHSACA |
| | AHCLLRGNRGGYCNRK |
| | GVCVRN |
| SEQ ID NO: 798 | RRWCFRVCYRGFCYRK |
| | CR |
| SEQ ID NO: 799 | RRWCFRVCYKGFCYRK |
| | CR |
| SEQ ID NO: 800 | RGGRLCYCRRRFCVCV |
| | GR |
| SEQ ID NO: 801 | RGGGLCYCRRRFCVCV |
| | GR |
| SEQ ID NO: 802 | VTCDLLSFKGQVNDSA |
| | CAANCLSLGKAGGHCE |
| | KGVCICRKTSFKDLWD |
| | KYF |
| SEQ ID NO: 803 | GWLKKIGKKIERVGQH |
| | TRDATIQGLGIAQQAA |
| | NVAATAR |
| SEQ ID NO: 804 | SDEKASPDKHHRFSLSR |
| | YAKLANRLANPKLLET |
| | FLSKWIGDRGNRSV |
| SEQ ID NO: 805 | KWCFRVCYRGICYRRC |
| | R |
| SEQ ID NO: 806 | RWCFRVCYRGICYRKC |
| | R |
| SEQ ID NO: 807 | KSCCKDTLARNCYNTC |
| | RFAGGSRPVCAGACRC |
| | KIIGPKCPSDYPK |
| SEQ ID NO: 808 | GGKPDLRPCIIPPCHYIP |
| | RPKPR |
| SEQ ID NO: 809 | VKDGYIVDDVNCTYFC |
| | GRNAYCNEECTKLKGE |
| | SGYCQWASPYGNACY |
| | CKLPDHVRTKGPGRCH |
| SEQ ID NO: 810 | KDEPQRRSARLSAKPAP |
| | PKPEPKPKKAPAKK |
| SEQ ID NO: 811 | AESGDDYCVLVFTDSA |
| | WTKICDWSHFRN |

Additional exemplary targeting groups suitable for derivitization as click-functionalized targeting groups in accordance with the present invention include those set forth in Tables 33-38, below. Exemplary peptides that have been shown to be potentially useful for targeting specific receptors on tumors cells or specific tumor types are listed in Tables 33-38. In some cases, the peptide sequences listed in Tables 33-38 are cyclized variations of the linear sequences.

TABLE 33

Prostate Specific Membrane Antigen (PSMA) Homing
Peptides

| | |
|---|---|
| SEQ ID NO: 812 | WQPDTAHHWAT |
| | L |
| SEQ ID NO: 813 | CTITSKRTC |
| SEQ ID NO: 814 | CQKHHNYLC |
| SEQ ID NO: 815 | CTLVPHTRC |

Lupold S and Rodriguez R Mol Cancer Ther 2004; 3(5): 597-603
Aggarwal S, Cancer Res 2006, 66(18) 9171

TABLE 34
Aminopeptidase N Homing Peptides

| | |
|---|---|
| SEQ ID NO: 816 | CNGRCVSGCAGR |
| | C |
| SEQ ID NO: 817 | CVCNGRMEC |

TABLE 35
HER-2 Homing Peptides

| | |
|---|---|
| SEQ ID NO: 818 | KCCYSL |

Karasseva N J Protein Chem 2002; 21(4): 287-96

TABLE 36
Colon Cancer Homing Peptides

| | |
|---|---|
| SEQ ID NO: 819 | VHLGYAT |
| SEQ ID NO: 820 | CPIEDRPMC |

TABLE 37
VEGFR1 Homing Peptides

| | |
|---|---|
| SEQ ID NO: 821 | NGYEIEWYSWVT |
| | HGMY |
| SEQ ID NO: 822 | RRKRRR |
| SEQ ID NO: 823 | ATWLPPR |
| SEQ ID NO: 824 | ASSSYPLIHWRPWAR |

TABLE 38
CXCR4 Homing Peptides

| | |
|---|---|
| SEQ ID NO: 825 | KGVSLSYR-K- |
| | RYSLSVGK |

Kim S., Clin. Exp. Met 2008 25, 201

One of ordinary skill in the art will recognize that the peptide sequences in Tables 1-38 can be click-functionalized at an amine-terminus or at a carboxylate-terminus.

As described above, Tables 1-38 represent lists of synthetic homing peptides, i.e., peptides that home to specific tissues, both normal and cancer. Such peptides are described in, e.g., U.S. Pat. Nos. 6,576,239, 6,306,365, 6,303,573, 6,296,832, 6,232,287, 6,180,084, 6,174,687, 6,068,829, 5,622,699, U.S. Patent Application Publication Nos. 2001/0046498, 2002/0041898, 2003/0008819, 2003/0077826, PCT application PCT/GB02/04017(WO 03/020751), and by Aina, O. et al., Mol Pharm 2007, 4(5), 631.

Those skilled in the art will recognize methods for identifying and characterizing tissue-homing peptides. For example, see Arap, W., et al., Science 1998, 279(5349), 377, Pasqualini R. and Ruoslahti, E., Nature 1996, 380(6572), 364, Rajotte, D. et al., J. Clin Invest 1998, 102(2), 430, Laakkonen, P., et al., Nat. Med. 2002, 8(7), 751, Essler, M. and Ruoslahti E. Proc Natl Acad Sci USA 2002, 99(4), 2252, Joyce J., et al., Cancer Cell 2003, 4(5), 393, Montet X., et al., Bioconjug Chem 2006, 17(4), 905, and Hoffman J. et al., Cancer Cell 2003, 4(5), 383.

In certain embodiments, a click-functionalized targeting group, in accordance with the present invention, is conjugated to a polymer. In certain embodiments, the polymer is PEG or a functionalized PEG. In other embodiments, a click-functionalized targeting group, in accordance with the present invention, is conjugated to a polymer micelle for targeting of tissues to which the targeting group homes. In still other embodiments, a click-functionalized targeting group, in accordance with the present invention, is conjugated to a micelle having a chemotherapeutic agent encapsulated therein.

As described above, the present invention provides targeting groups that are functionalized in a manner suitable for click chemistry. In certain embodiments, the targeting group is an oligopeptide. In some embodiments, a click functionalized moiety is introduced to an oligopeptide by reaction of a click-functionalized carboxylic acid with the N-terminus of an oligopeptide. Such click-functionalized carboxylic acids include, but are not limited to:

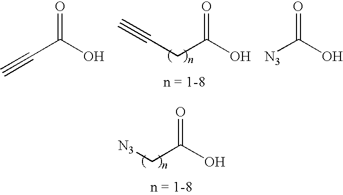

One of ordinary skill in the art will recognize that such carboxylic acids can be introduced to the oligopeptide while on the solid-phase resin or after the peptide has been cleaved from the resin. Such coupling methods include, but are not limited to: aminium/phosphonium-based coupling reagents (e.g. HATU, HBTU, HCTU, TBTU, BOP, PyBOP, PyAOP or HATU/HOBt, HBTU/HOBt, TBTU/HOBt, HCTU/HOBt combinations), carbodiimide-based reagents (e.g. diisopropylcarbodiimide (DIC), dicyclohexylcarbodiimide (DCC), 1-ethyl-3-(3-dimethylaminopropyl)-carbodiimide (EDC), or DIC/HOBt, DCC/HOBt, EDC/HOBt combinations), reaction with symmetrical anhydrides of click-functionalized carboxylic acids (prepared through reaction with carbodiimide reagents), reaction with activated esters (e.g. N-hydroxysuccinimide (NHS), pentafluorophenyl (OPfp)) of click-functionalized carboxylic acids, reaction of acid chloride or acid fluoride derivatives of click-functionalized carboxylic acids, and the like.

In another embodiment, a click functionalized moiety is introduced to an oligopeptide by reaction of a click-functionalized carboxylic acid with primary or secondary amines present on the oligopeptide side-chain. Common amine-functionalized amino acids include natural amino acids such as lysine, arginine, and histidine.

In one embodiment, a click functionalized moiety is introduced to an oligopeptide by reaction of a click-functionalized amine with the C-terminus of an oligopeptide. Such click-functionalized amines include, but are not limited to:

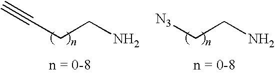

One of ordinary skill in the art will recognize that such amines can be introduced to the C-terminus of an oligopeptide after the peptide has been cleaved from the resin. Such coupling methods include, but are not limited to: aminium/phosphonium-based coupling reagents (e.g. HATU, HBTU, HCTU, TBTU, BOP, PyBOP, PyAOP or HATU/HOBt, HBTU/HOBt, TBTU/HOBt, HCTU/HOBt combinations), carbodiimide-based reagents (e.g. diisopropylcarbodiimide (DIC), dicyclohexylcarbodiimide (DCC), 1-ethyl-3-(3-dimethylaminopropyl)-carbodiimide (EDC), or DIC/HOBt, DCC/HOBt, EDC/HOBt combinations), reaction with activated esters (e.g. N-hydroxysuccinimide (NHS), pentafluorophenyl (OPfp)) of oligopeptides, reaction of acid chloride or acid fluoride derivatives of oligopeptides, and the like.

In another embodiment, a click functionalized moiety is introduced to an oligopeptide by reaction of a click-functionalized amines with carboxylic acids present on the oligopeptide side-chain. Common carboxylic acid-functionalized amino acids include natural amino acids such as aspartic acid and glutamic acid.

In yet another embodiment, a click-ready moiety is introduced through incorporation of a click-functionalized amino acid into the oligopeptide backbone. Such click-functionalized amino acids include, but are not limited to:

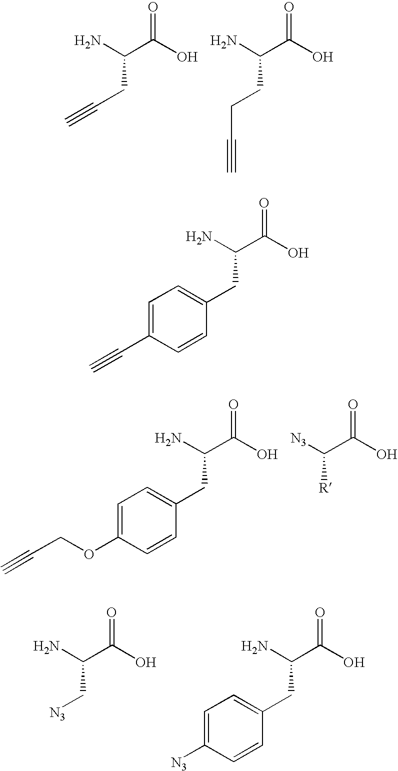

wherein R′ is a natural or unnatural amino acid side-chain group. It will be appreciated that, while L amino acids are depicted above, D amino acids or racemic mixtures may also be used.

In some embodiments, amino acids which are suitably protected for solid-phase chemistry are introduced. Such protected amino acids include, but are not limited to:

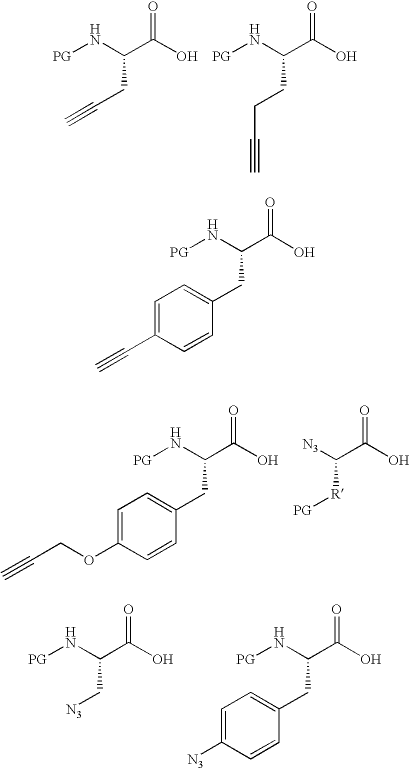

wherein R′ is a natural or unnatural amino acid side-chain group, and PG is a suitable protecting group. It will be appreciated that, while L amino acids are depicted above, D amino acids or racemic mixtures may also be used. Suitable protecting groups are known in the art and include those described above and by Greene (supra). In some embodiments, PG is an acid (e.g. Boc) or base (e.g. Fmoc) labile protecting group. One of ordinary skill in the art will recognize that such amino acids can be introduced to the N-terminus of an oligopeptide during chain extension on a solid-phase resin. Such coupling methods include, but are not limited to: aminium/phosphonium-based coupling reagents (e.g. HATU, HBTU, HCTU, TBTU, BOP, PyBOP, PyAOP or HATU/HOBt, HBTU/HOBt, TBTU/HOBt, HCTU/HOBt combinations), carbodiimide-based reagents (e.g. diisopropylcarbodiimide (DIC), dicyclohexylcarbodiimide (DCC), 1-ethyl-3-(3-dimethylaminopropyl)-carbodiimide (EDC), or DIC/HOBt, DCC/HOBt, EDC/HOBt combinations), preparation of symmetrical anhydrides of click-functionalized amino acids (prepared through reaction with carbodiimide reagents), reaction with activated esters (e.g. N-hydroxysuccinimide (NHS), pentafluorophenyl (OPfp)) of click-functionalized amino acids, reaction of acid chloride or acid fluoride derivatives of click-functionalized amino acids, and the like.

B. Bifunctional PEG's

As described herein, provided targeting groups may be conjugated to a suitably functionalized PEG. Such functionalized PEG's are described in detail in U.S. Patent Application Publication Numbers 2006/0240092, 2006/0172914, 2006/0142506, and 2008/0035243, and Published PCT Applications WO07/127,473, WO07/127,440, and WO06/86325, the entirety of each of which is hereby incorporated herein by reference.

In certain embodiments, the present invention provides a method for conjugating a provided click-functionalized targeting group with a compound of formula A:

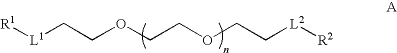

or a salt thereof, wherein:
n is 10-2500;
R¹ and R² are each independently hydrogen, halogen, NO₂, CN, N₃, —N═C═O, —C(R)═NN(R)₂, —P(O)(OR)₂, —P(O)(X)₂, a 9-30 membered crown ether, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, provided that one of R¹ and R² is a moiety suitable for click chemistry;
each X is independently halogen;
each R is independently hydrogen or an optionally substituted selected from aliphatic or a 3-8 membered, saturated, partially unsaturated, or aryl ring having 0-4 heteroatoms independently selected from nitrogen, oxygen, or sulfur; and
L¹ and L² are each independently a valence bond or a bivalent, saturated or unsaturated, straight or branched C₁₋₁₂ hydrocarbon chain, wherein 0-6 methylene units of L¹ and L² are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur,
      comprising the steps of:
   (a) providing a compound of formula A,
   (b) providing a click-functionalized targeting compound, and
   (c) conjugating the compound of formula A to the targeting compound via click chemistry.

In some embodiments, the preceding steps (a) through (c) provide a compound of formula A-1, A-2, A-3, or A-4:

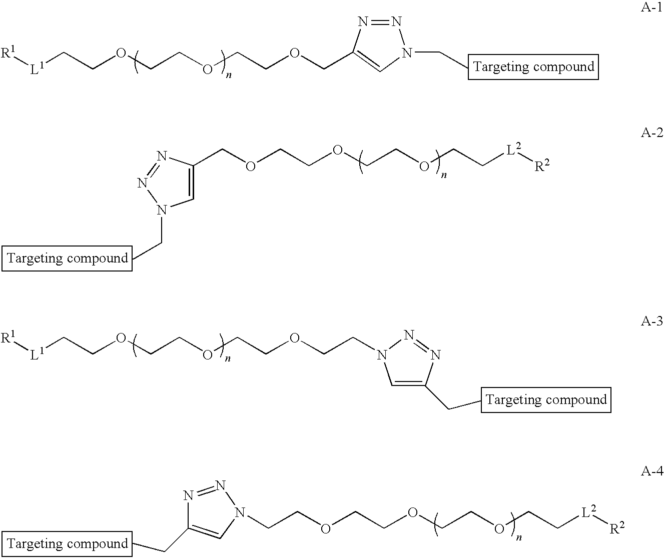

wherein the targeting compound is selected from those described herein and each n is 10-2500. In certain embodiments, each n is independently about 225. In other embodiments, n is about 270. In other embodiments, n is about 350. In other embodiments, n is about 10 to about 40. In other embodiments, n is about 40 to about 60. In other embodiments, n is about 60 to about 90. In still other embodiments, n is about 90 to about 150. In other embodiments, n is about 150 to about 200. In still other embodiments, n is about 200 to about 250. In other embodiments, n is about 300 to about 375. In other embodiments, n is about 400 to about 500. In still other embodiments, n is about 650 to about 750. In certain embodiments, n is selected from 50±10. In other embodiments, n is selected from 80±10, 115±10, 180±10, 225±10, 275±10, 315±10, or 340±10.

In certain embodiments, the present invention provides a click functionalized targeting group, wherein said click functionalized targeting group is other than:

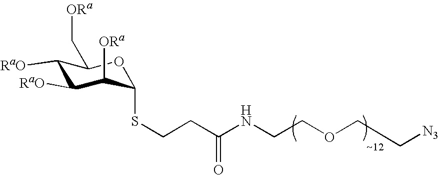

wherein each R^{a} is independently hydrogen or acetyl.

Table 39 sets forth exemplary compounds of the present invention having the formula:

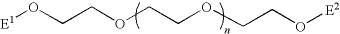

wherein n=10-2500.

TABLE 39

| Compound # | E¹ | E² |
|---|---|---|
| 1 | 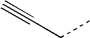 | 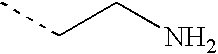 |
| 2 | 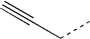 | 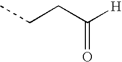 |
| 3 | 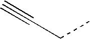 | 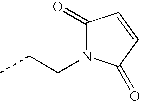 |
| 4 | 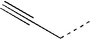 | 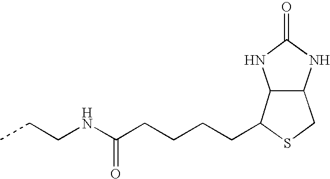 |
| 5 | 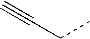 | 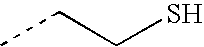 |
| 6 | 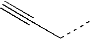 | 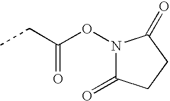 |
| 7 | 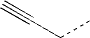 | 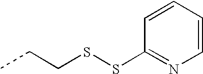 |
| 8 | 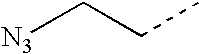 | 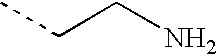 |
| 9 | 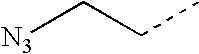 | 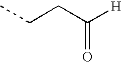 |
| 10 | 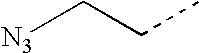 | 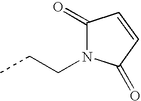 |
| 11 | 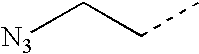 | 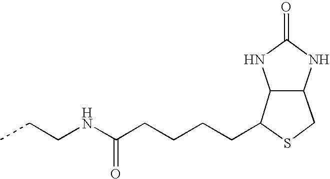 |
| 12 | 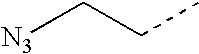 | 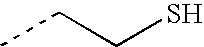 |
| 13 | 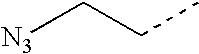 | 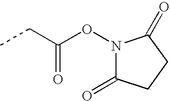 |
| 14 | 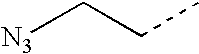 | 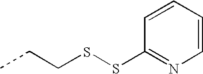 |

C. Multiblock Copolymers

As described herein, provided targeting groups may be conjugated to a polymer micelle. Such polymer micelles are described in detail in U.S. Patent Application Publication Number 2006/0240092, the entirety of which is hereby incorporated herein by reference.

In certain embodiments, the present invention provides a method for conjugating an inventive click-functionalized targeting group with a compound of formula B:

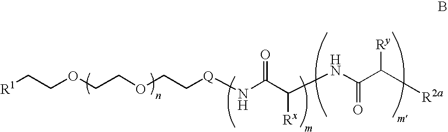

wherein:
   n is 10-2500;
   m is 0 to 1000;
   m′ is 1 to 1000;
   R^{x} is a natural or unnatural amino acid side-chain group that is capable of crosslinking;
   R^{y} is a hydrophobic or ionic, natural or unnatural amino acid side-chain group;
   R¹ is -Z(CH₂CH₂Y)ₚ(CH₂)ₜR³, wherein:
      Z is —O—, —S—, —C≡C—, or —CH₂—;
      each Y is independently —O— or —S—;
      p is 0-10;
      t is 0-10; and
      R³ is —N₃ or alkyne;
   Q is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
      -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
   R^{2a} is a mono-protected amine, a di-protected amine, —N(R⁴)₂, —NR⁴C(O)R⁴, —NR⁴C(O)N(R⁴)₂, —NR⁴C(O)OR⁴, or —NR⁴SO₂R⁴, provided that one of R¹ and R^{2a} is a moiety suitable for click chemistry; and
   each R⁴ is independently an optionally substituted group selected from hydrogen, aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or:
      two R⁴ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur,
         comprising the steps of:
         (a) providing a compound of formula B,
         (b) providing a click-functionalized targeting compound, and
         (c) conjugating the compound of formula B to the targeting compound via click chemistry.

In certain embodiments, a compound of formula B is a triblock copolymer comprising a polymeric hydrophilic block, a poly(amino acid) block, and a mixed random copolymer block. In some embodiments, a compound of formula B further comprises a crosslinked or crosslinkable block, wherein R^{x} is a natural or unnatural amino acid side-chain group that is capable of crosslinking (e.g., aspartate, histidine). In some embodiments, a compound of formula B comprises triblock copolymers comprising a polymeric hydrophilic block, a crosslinked or crosslinkable poly(amino acid) block, and an mixed random copolymer block. In some embodiments, m is 0, and a compound of formula B comprises diblock copolymers comprising a hydrophilic block and a mixed random copolymer block. Methods making and using said copolymers and micelles thereof are described in U.S. Patent Application Publication Numbers 2006/0142506, 2006/0172914, and 2006/0240092.

In certain embodiments, the preceeding steps (a) through (c) provide a compound of formula B-1 or B-2:

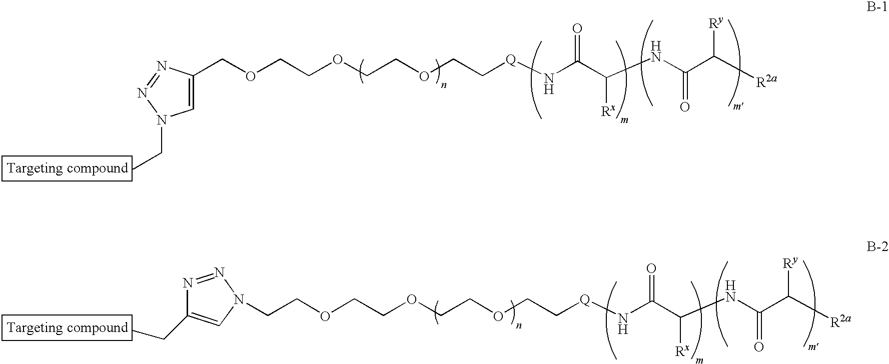

wherein the targeting compound is selected from those described herein.

Table 40 sets forth exemplary compounds of the present invention having the formula:

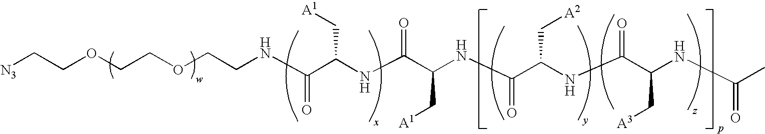

Wherein w=150-400, x=3-30, y=1-50, z=1-50 and p=sum of y and z.

TABLE 40

| Compound # | A¹ | A² | A³ |
|---|---|---|---|
| 15 | 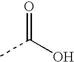 | 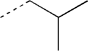 | 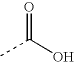 |
| 16 | 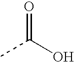 | 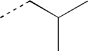 | 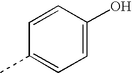 |
| 17 | 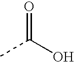 | 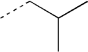 | 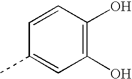 |
| 18 | 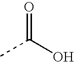 | 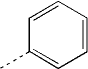 | 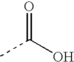 |
| 19 | 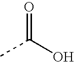 | 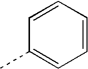 | 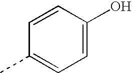 |
| 20 | 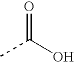 | 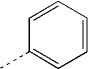 | 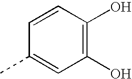 |
| 21 | 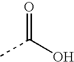 | 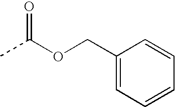 | 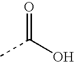 |
| 22 | 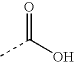 | 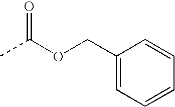 | 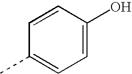 |
| 23 | 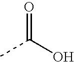 | 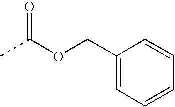 | 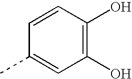 |
| 24 | 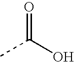 | 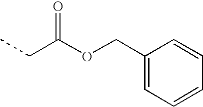 | 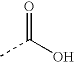 |
| 25 | 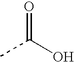 | 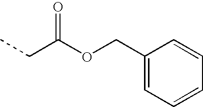 | 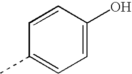 |
| 26 | 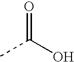 | 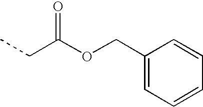 | 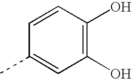 |
| 27 | 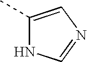 | 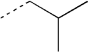 | 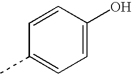 |
| 28 | 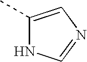 | 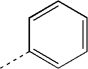 | 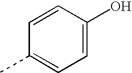 |
| 29 | 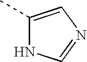 | 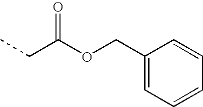 | 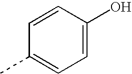 |
| 30 | 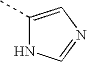 | 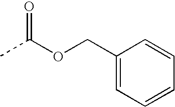 | 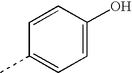 |
| 31 | 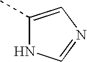 | 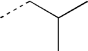 | 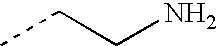 |
| 32 | 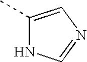 | 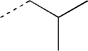 | 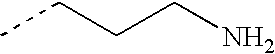 |
| 33 | 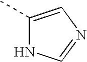 | 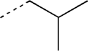 | 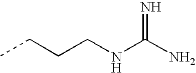 |

Table 41 sets forth exemplary compounds of the present invention having the formula:

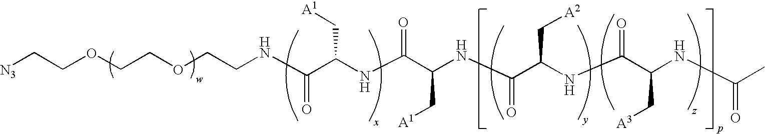

wherein w=150-400, x=3-30, y=1-50, z=1-50 and p=sum of y and z.

TABLE 41

| Compound # | A¹ | A² | A³ |
|---|---|---|---|
| 34 | 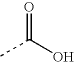 | 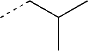 | 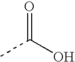 |
| 35 | 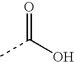 | 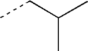 | 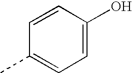 |
| 36 | 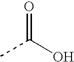 | 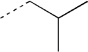 | 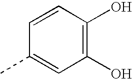 |
| 37 | 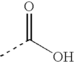 | 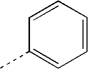 | 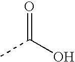 |
| 38 | 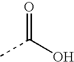 | 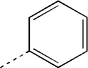 | 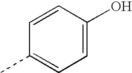 |
| 39 | 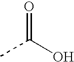 | 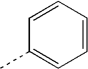 | 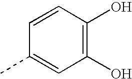 |
| 40 | 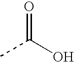 | 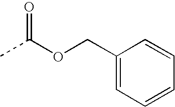 | 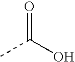 |
| 41 | 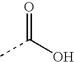 | 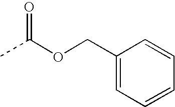 | 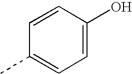 |
| 42 | 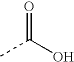 | 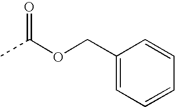 | 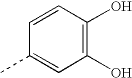 |
| 43 | 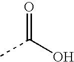 | 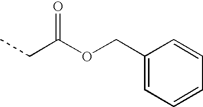 | 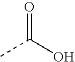 |
| 44 | 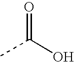 | 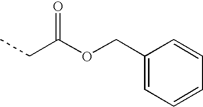 | 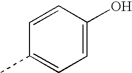 |
| 45 | 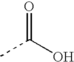 | 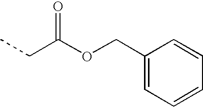 | 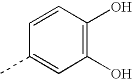 |
| 46 | 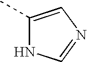 | 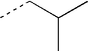 | 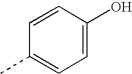 |
| 47 | 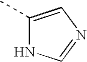 | 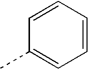 | 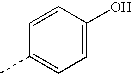 |
| 48 | 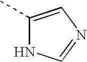 | 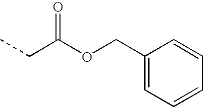 | 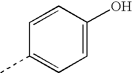 |
| 49 | 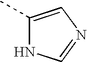 | 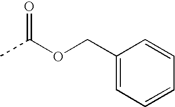 | 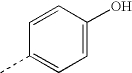 |
| 50 | 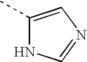 | 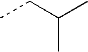 | 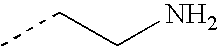 |
| 51 | 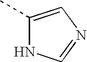 | 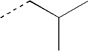 | 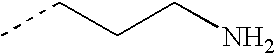 |
| 52 | 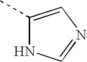 | 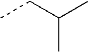 | 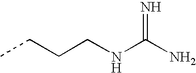 |

Table 42 sets forth exemplary compounds of the present invention having the formula:

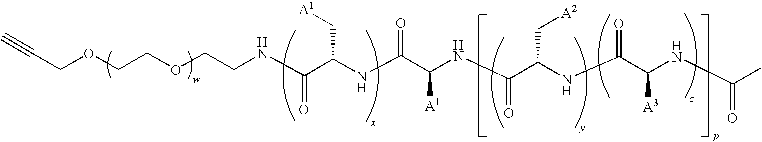

wherein w=150-400, x=3-30, y=1-50, z=1-50 and p=sum of y and z.

TABLE 42

| Compound # | A¹ | A² | A³ |
|---|---|---|---|
| 53 | 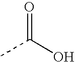 | 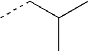 | 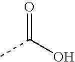 |
| 54 | 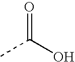 | 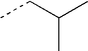 | 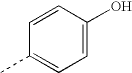 |
| 55 | 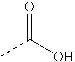 | 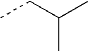 | 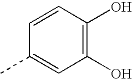 |
| 56 | 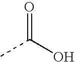 | 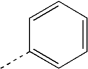 | 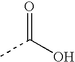 |
| 57 | 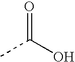 | 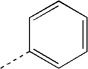 | 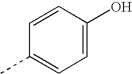 |
| 58 | 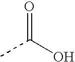 | 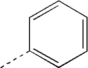 | 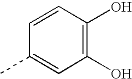 |
| 59 | 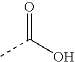 | 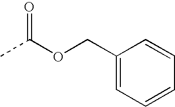 | 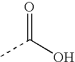 |
| 60 | 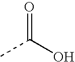 | 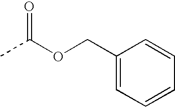 | 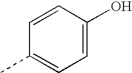 |
| 61 | 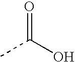 | 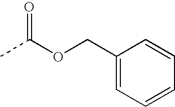 | 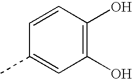 |
| 62 | 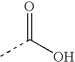 | 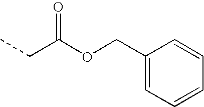 | 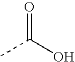 |
| 63 | 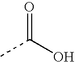 | 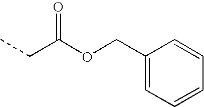 | 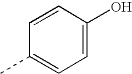 |
| 64 | 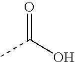 | 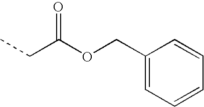 | 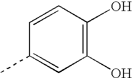 |
| 65 | 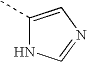 | 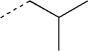 | 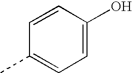 |
| 66 | 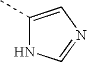 | 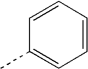 | 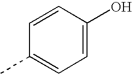 |
| 67 | 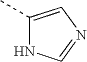 | 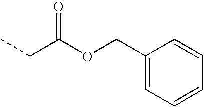 | 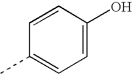 |
| 68 | 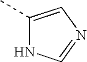 | 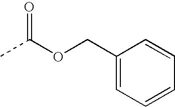 | 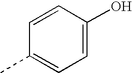 |
| 69 | 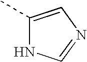 | 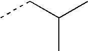 | 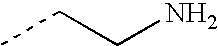 |
| 70 | 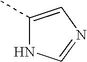 | 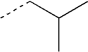 | 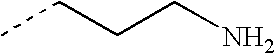 |
| 71 | 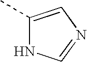 | 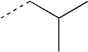 | 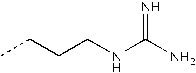 |

Table 43 sets forth exemplary compounds of the present invention having the formula:

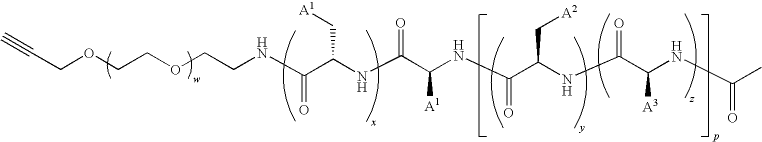

wherein w=150-400, x=3-30, y=1-50, z=1-50 and p=sum of y and z.

TABLE 43

| Compound # | A¹ | A² | A³ |
|---|---|---|---|
| 72 | 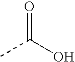 | 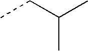 | 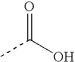 |
| 73 | 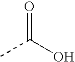 | 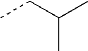 | 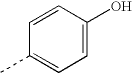 |
| 74 | 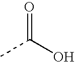 | 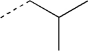 | 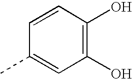 |
| 75 | 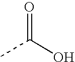 | 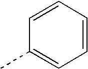 | 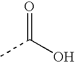 |
| 76 | 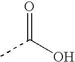 | 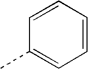 | 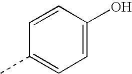 |
| 77 | 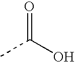 | 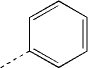 | 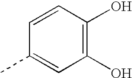 |
| 78 | 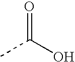 | 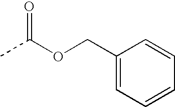 | 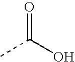 |
| 79 | 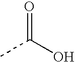 | 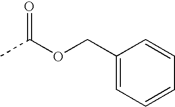 | 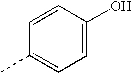 |
| 80 | 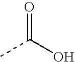 | 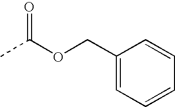 | 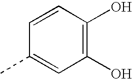 |
| 81 | 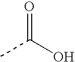 | 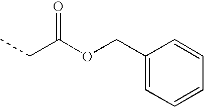 | 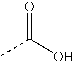 |
| 82 | 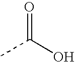 | 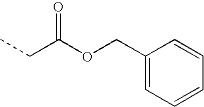 | 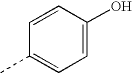 |
| 83 | 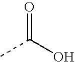 | 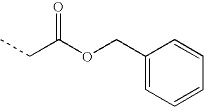 | 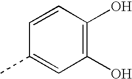 |
| 84 | 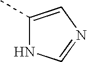 | 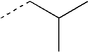 | 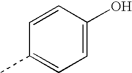 |
| 85 | 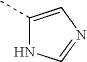 | 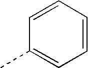 | 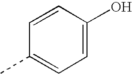 |
| 86 | 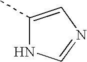 | 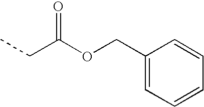 | 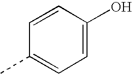 |
| 87 | 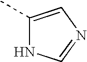 | 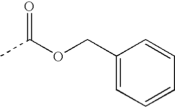 | 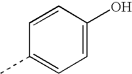 |
| 88 | 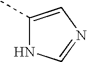 | 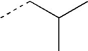 | 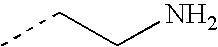 |
| 89 | 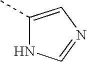 | 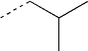 | 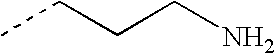 |
| 90 | 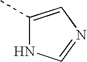 | 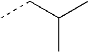 | 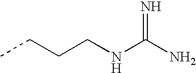 |

Table 44 sets forth exemplary compounds of the present invention having the formula:

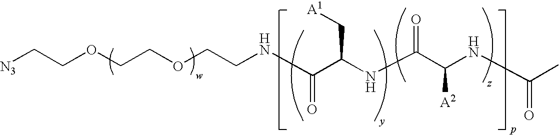

wherein w=150-400, y=1-50, z=1-50, and p is the sum of y and z.

TABLE 44

| Compound # | A¹ | A² |
|---|---|---|
| 91 | 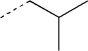 | 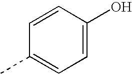 |
| 92 | 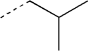 | 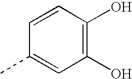 |
| 93 | 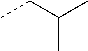 | 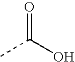 |
| 94 | 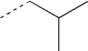 | 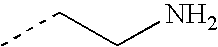 |
| 95 | 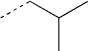 | 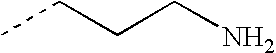 |
| 96 | 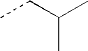 | 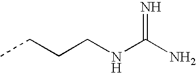 |
| 97 | 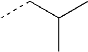 | 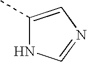 |
| 98 | 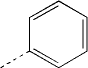 | 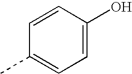 |
| 99 | 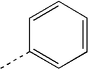 | 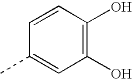 |
| 100 | 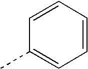 | 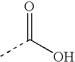 |
| 101 | 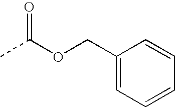 | 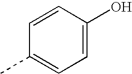 |
| 102 | 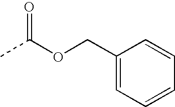 | 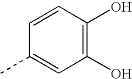 |
| 103 | 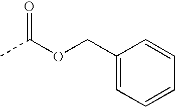 | 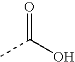 |
| 104 | 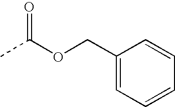 | 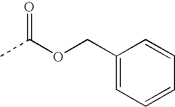 |
| 105 | 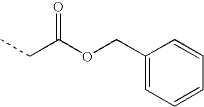 | 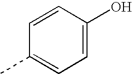 |
| 106 | 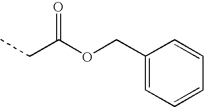 | 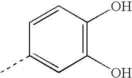 |
| 107 | 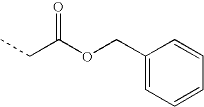 | 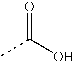 |
| 108 | 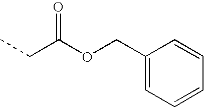 | 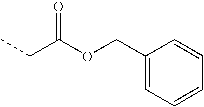 |

Table 45 sets forth exemplary compounds of the present invention having the formula:

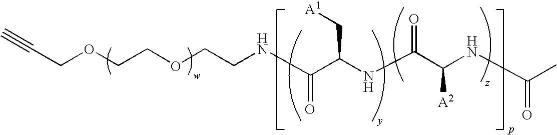

wherein w=150-400, y=1-50, z=1-50, and p is the sum of y and z.

TABLE 45

| Compound # | A¹ | A² |
|---|---|---|
| 109 | 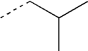 | 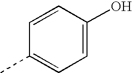 |
| 110 | 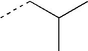 | 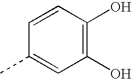 |
| 111 | 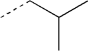 | 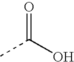 |
| 112 | 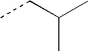 | 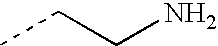 |
| 113 | 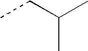 | 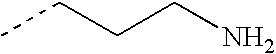 |
| 114 | 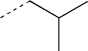 | 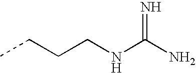 |
| 115 | 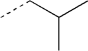 | 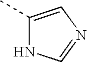 |
| 116 | 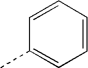 | 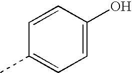 |
| 117 | 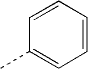 | 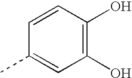 |
| 118 | 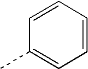 | 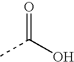 |
| 119 | 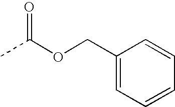 | 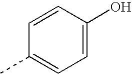 |
| 120 | 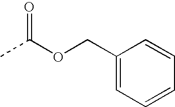 | 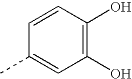 |
| 121 | 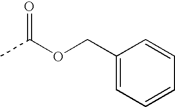 | 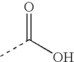 |
| 122 | 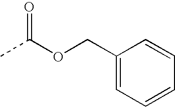 | 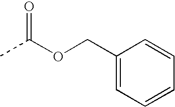 |
| 123 | 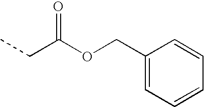 | 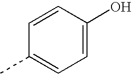 |
| 124 | 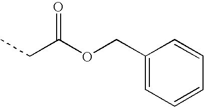 | 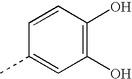 |
| 125 | 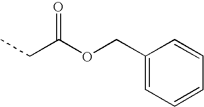 | 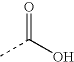 |
| 126 | 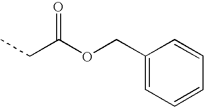 | 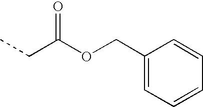 |

### General Methods for Providing Compounds of the Present Invention

Bifunctional PEG's are prepared according to U.S. Patent Application Publication Numbers 2006/0240092, 2006/0172914, 2006/0142506, and 2008/0035243, and Published PCT Applications WO07/127,473, WO07/127,440, and WO06/86325, the entirety of each of which is hereby incorporated by reference. Multiblock copolymers of the present invention are prepared by methods known to one of ordinary skill in the art and those described in detail in U.S. patent application Ser. No. 11/325,020 filed Jan. 4, 2006, the entirety of which is hereby incorporated herein by reference. Generally, such multiblock copolymers are prepared by sequentially polymerizing one or more cyclic amino acid monomers onto a hydrophilic polymer having a terminal amine salt wherein said polymerization is initiated by said amine salt. In certain embodiments, said polymerization occurs by ring-opening polymerization of the cyclic amino acid monomers. In other embodiments, the cyclic amino acid monomer is an amino acid NCA, lactam, or imide.

### 5. Uses, Methods, and Compositions

Compositions

According to another embodiment, the invention provides a composition comprising a polymer or polymer micelle conjugated to a targeting group described herein or a pharmaceutically acceptable derivative thereof and a pharmaceutically acceptable carrier, adjuvant, or vehicle. In certain embodiments, such compositions are formulated for administration to a patient in need of such composition. In other embodiments, the composition of this invention is formulated for oral administration to a patient. In some embodiments, compositions of the present invention are formulated for parenteral administration.

In certain embodiments, a micelle conjugated to a provided targeting group is drug loaded. Such drug-loaded micelles of the present invention are useful for treating any disease wherein the targeting of said micelle to the diseased tissue or cell is beneficial for the delivery of said drug. In certain embodiments, drug-loaded micelles of the present invention are useful for treating cancer. Accordingly, another aspect of the present invention provides a method for treating cancer in a patient comprising administering to a patient a multiblock copolymer which comprises a polymeric hydrophilic block, optionally a crosslinkable or crosslinked poly(amino acid block), and a hydrophobic D,L-mixed poly(amino acid block), characterized in that said micelle has a drug-loaded inner core, optionally a crosslinkable or crosslinked outer core, and a hydrophilic shell, wherein said micelle encapsulates a chemotherapeutic agent.

According to another embodiment, the present invention relates to a method of treating a cancer selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon-rectum, large intestine, rectum, brain and central nervous system, and leukemia, comprising administering a micelle in accordance with the present invention wherein said micelle encapsulates a chemotherapeutic agent suitable for treating said cancer.

P-glycoprotein (Pgp, also called multidrug resistance protein) is found in the plasma membrane of higher eukaryotes where it is responsible for ATP hydrolysis-driven export of hydrophobic molecules. In animals, Pgp plays an important role in excretion of and protection from environmental toxins; when expressed in the plasma membrane of cancer cells, it can lead to failure of chemotherapy by preventing the hydrophobic chemotherapeutic drugs from reaching their targets inside cells. Indeed, Pgp is known to transport hydrophobic chemotherapeutic drugs out of tumor cells. According to one aspect, the present invention provides a method for delivering a hydrophobic chemotherapeutic drug to a cancer cell while preventing, or lessening, Pgp excretion of that chemotherapeutic drug, comprising administering a drug-loaded micelle comprising a multiblock polymer of the present invention loaded with a hydrophobic chemotherapeutic drug. Such hydrophobic chemotherapeutic drugs are well known in the art and include those described herein.

In certain embodiments, the present invention provides a micelle, as described herein, loaded with an antiproliferative or chemotherapeutic agent selected from any one or more of Abarelix, aldesleukin, Aldesleukin, Alemtuzumab, Alitretinoin, Allopurinol, Altretamine, Amifostine, Anastrozole, Arsenic trioxide, Asparaginase, Azacitidine, BCG Live, Bevacuzimab, Avastin, Fluorouracil, Bexarotene, Bleomycin, Bortezomib, Busulfan, Calusterone, Capecitabine, Camptothecin, Carboplatin, Carmustine, Celecoxib, Cetuximab, Chlorambucil, Cisplatin, Cladribine, Clofarabine, Cyclophosphamide, Cytarabine, Dactinomycin, Darbepoetin alfa, Daunorubicin, Denileukin, Dexrazoxane, Docetaxel, Doxorubicin (neutral), Doxorubicin hydrochloride, Dromostanolone Propionate, Epirubicin, Epoetin alfa, Erlotinib, Estramustine, Etoposide Phosphate, Etoposide, Exemestane, Filgrastim, floxuridine fludarabine, Fulvestrant, Gefitinib, Gemcitabine, Gemtuzumab, Goserelin Acetate, Histrelin Acetate, Hydroxyurea, Ibritumomab, Idarubicin, Ifosfamide, Imatinib Mesylate, Interferon Alfa-2a, Interferon Alfa-2b, Irinotecan, Lenalidomide, Letrozole, Leucovorin, Leuprolide Acetate, Levamisole, Lomustine, Megestrol Acetate, Melphalan, Mercaptopurine, 6-MP, Mesna, Methotrexate, Methoxsalen, Mitomycin C, Mitotane, Mitoxantrone, Nandrolone, Nelarabine, Nofetumomab, Oprelvekin, Oxaliplatin, Paclitaxel, Palifermin, Pamidronate, Pegademase, Pegaspargase, Pegfilgrastim, Pemetrexed Disodium, Pentostatin, Pipobroman, Plicamycin, Porfimer Sodium, Procarbazine, Quinacrine, Rasburicase, Rituximab, Sargramostim, Sorafenib, Streptozocin, Sunitinib Maleate, Talc, Tamoxifen, Temozolomide, Teniposide, VM-26, Testolactone, Thioguanine, 6-TG, Thiotepa, Topotecan, Toremifene, Tositumomab, Trastuzumab, Tretinoin, ATRA, Uracil Mustard, Valrubicin, Vinblastine, Vincristine, Vinorelbine, Zoledronate, or Zoledronic acid.

Targeting the delivery of potent, cytotoxic agents specifically to cancer cells using responsive nanovectors would have a clear impact on the well-being of the many thousands of people who rely on traditional small molecule therapeutics for the treatment of cancer. In certain embodiments, the present invention provides micelle-encapsulated forms of the common chemotherapy drugs, doxorubicin (adriamycin), a topoisomerase II inhibitor, camptothecin (CPT), a topoisomerase I inhibitor, or paclitaxel (Taxol), an inhibitor of microtubule assembly.

According to one aspect, the present invention provides a micelle, as described herein, loaded with a hydrophobic drug selected from any one or more of a Exemestance (aromasin), Camptosar (irinotecan), Ellence (epirubicin), Femara (Letrozole), Gleevac (imatinib mesylate), Lentaron (formestane), Cytadren/Orimeten (aminoglutethimide), Temodar, Proscar (finasteride), Viadur (leuprolide), Nexavar (Sorafenib), Kytril (Granisetron), Taxotere (Docetaxel), Taxol (paclitaxel), Kytril (Granisetron), Vesanoid (tretinoin) (retin A), XELODA (Capecitabine), Arimidex (Anastrozole), Casodex/Cosudex (Bicalutamide), Faslodex (Fulvestrant), Iressa (Gefitinib), Nolvadex, Istubal, Valodex (tamoxifen citrate), Tomudex (Raltitrexed), Zoladex (goserelin acetate), Leustatin (Cladribine), Velcade (bortezomib), Mylotarg (gemtuzumab ozogamicin), Alimta (pemetrexed), Gemzar (gemcitabine hydrochloride), Rituxan (rituximab), Revlimid (lenalidomide), Thalomid (thalidomide), Alkeran (melphalan), and derivatives thereof.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N+(C₁₋₄ alkyl)₄ salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In certain embodiments, pharmaceutically acceptable compositions of the present invention are enterically coated.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, the pharmaceutically acceptable compositions of this invention are formulated for oral administration.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the drug can be administered to a patient receiving these compositions.

It will be appreciated that dosages typically employed for the encapsulated drug are contemplated by the present invention. In certain embodiments, a patient is administered a drug-loaded micelle of the present invention wherein the dosage of the drug is equivalent to what is typically administered for that drug. In other embodiments, a patient is administered a drug-loaded micelle of the present invention wherein the dosage of the drug is lower than is typically administered for that drug.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

In order that the invention described herein may be more fully understood, the following examples are set forth. It will be understood that these examples are for illustrative purposes only and are not to be construed as limiting this invention in any manner.

### EXEMPLIFICATION

### General Synthesis of Click-Functionalized Saccharides

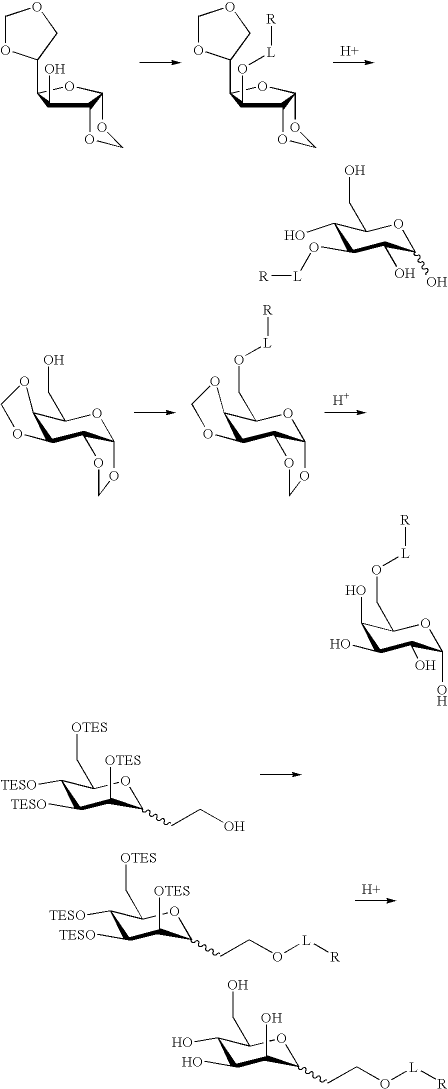

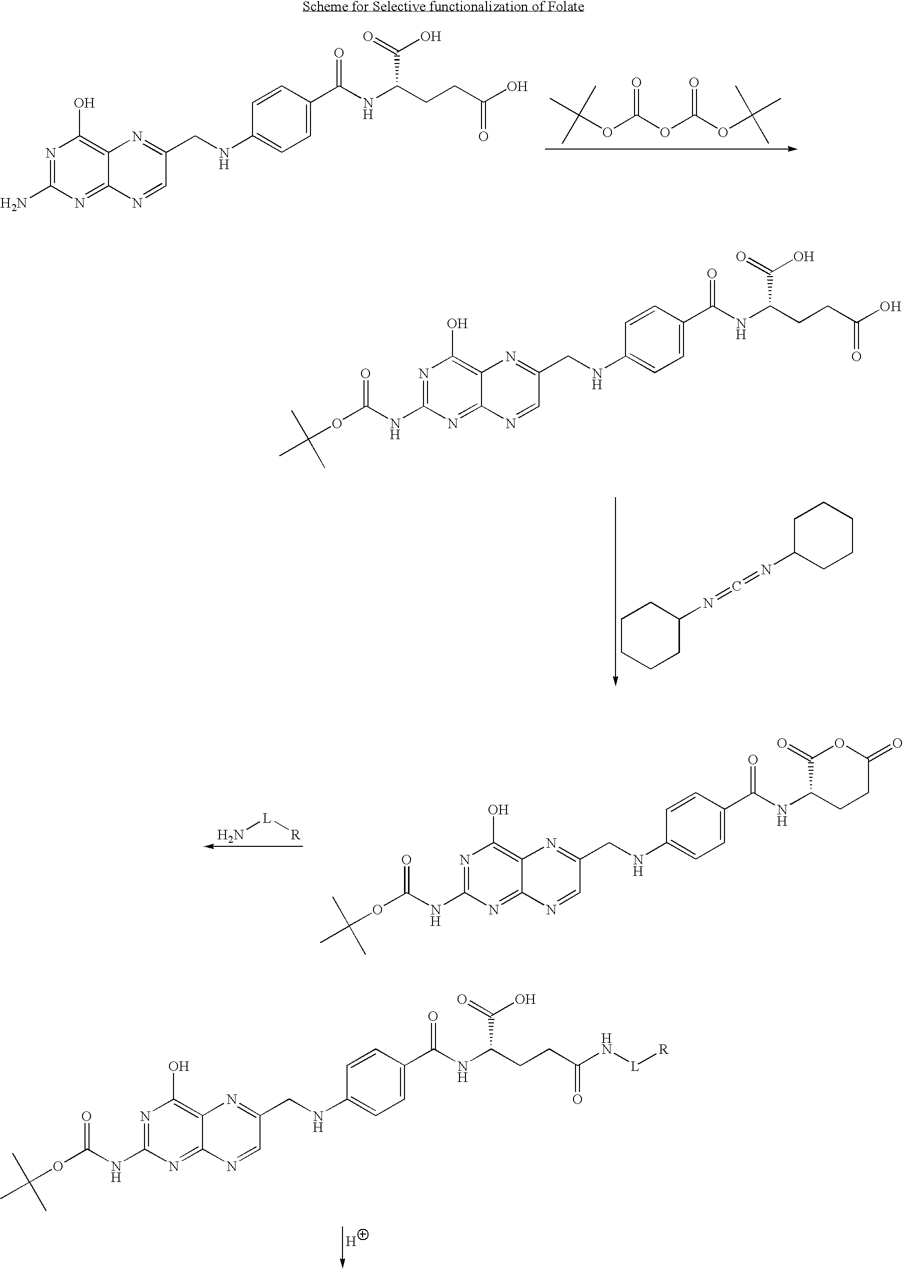

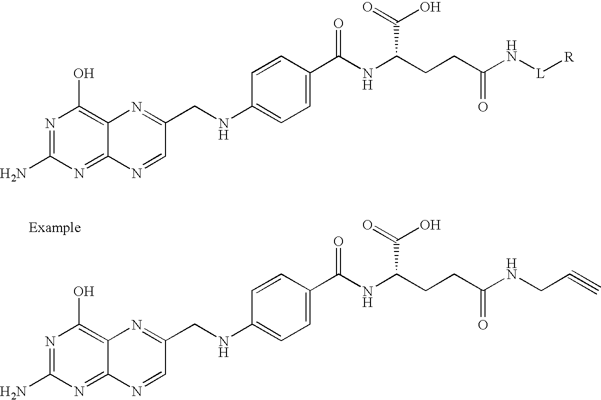

### Example 1

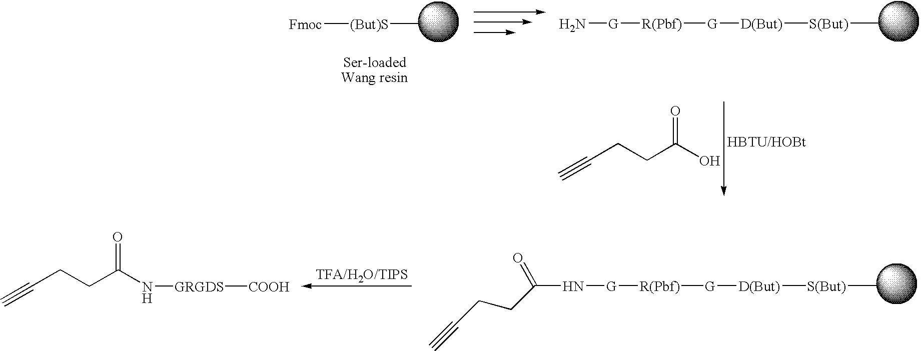

Synthesis of Acetylene-terminated GRGDS peptide—The oligopeptide sequence GRGDS was synthesized according to standard Fmoc solid phase peptide synthesis using a batch wise process and the peptide coupling agent HBTU. Fmoc-Ser(But)-loaded Wang resin (3.2 g with loading density of 0.6 mmol/g) was weighed into an oven-dried glass-fritted reaction tube and swollen with 30 mL dry CH₂Cl₂ for 5-10 minutes. The Fmoc group at the N-terminus was cleaved by the addition of a 25/75 solution of piperidine/DMF (30 mL), followed by agitation with nitrogen for three minutes. The resin was filtered, and fresh piperidine/DMF (30 mL) was added. After agitating for 20 minutes, the resin was filtered and washed with DMF six times.

A solution of Fmoc-Asp(OBut)-OH (3.85 g, 9.35 mmol), HBTU (3.48 g, 9.17 mmol), and HOBt (1.26 g, 9.35 mmol) in 20 mL of anhydrous DMF was prepared. After the solution became homogeneous, DIPEA (3.28 mL, 18.70 mmol) was added, and the resulting mixture was added immediately to the resin. The resin was then agitated for one hour, filtered, and washed with DMF (three times). A 25/75 solution of piperidine/DMF (30 mL) was added, and the resin agitated for three minutes. After filtration, piperidine/DMF was again added to the resin followed by agitation for 20 minutes. The resin was then washed with DMF (six times). The above amino acid addition procedure was repeated for Fmoc-Gly-OH, Fmoc-Arg(Pbf)-OH, and a second unit of Fmoc-Gly-OH.

Following the addition of the second Gly unit, a solution of 4-pentynoic acid (0.90 g, 9.0 mmol), HBTU (3.4 g, 8.8 mmol), and HOBt (1.4 g, 9.0 mmol) was prepared in 15 mL of dry DMF. After the solution became homogeneous, DIPEA (3.2 mL, 18.0 mmol) was added, and the resulting mixture was added immediately to the resin. The resin was then agitated for one hour, filtered, and washed with DMF (six times). After filtration, the resin was washed with DMF (six times) followed by CH₂Cl₂ (four times) to remove any residual DMF. The oligopeptide was then cleaved by agitating the resin with 95/2.5/2.5 TFA/H₂O/TIPS (30 ml) for three hours. The filtrated was collected in a clean flask, and the resin was washed with fresh cleavage solution and DCM several times. The solution was concentrated on a rotary evaporator and dissolved in minimal MeOH. The oligopeptide was precipitated from diethyl ether and isolated by filtration.

### Example 2

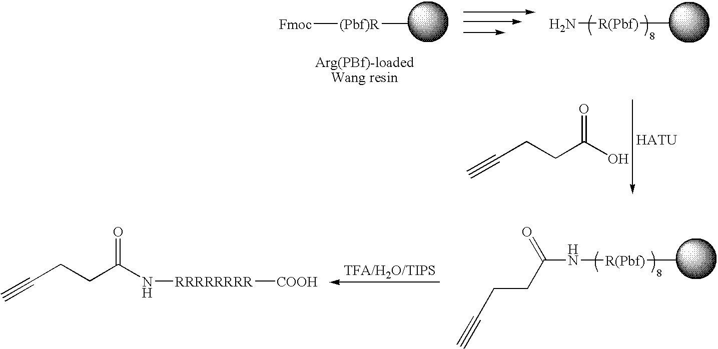

Synthesis of Acetylene-terminated RRRRRRRR peptide—The oligopeptide sequence RRRRRRRR was synthesized according to standard Fmoc solid phase peptide synthesis using a batch wise process and the peptide coupling agent HBTU. Fmoc-Arg(Pbf)-loaded Wang resin (3.0 g with loading density of 0.6 mmol/g) was weighed into an oven-dried glass-fritted reaction tube and swollen with 30 mL dry CH₂Cl₂ for 5-10 minutes. The Fmoc group at the N-terminus was cleaved by the addition of a 25/75 solution of piperidine/DMF (30 mL), followed by agitation with nitrogen for three minutes. The resin was filtered, and fresh piperidine/DMF (30 mL) was added. After agitating for 20 minutes, the resin was filtered and washed with DMF six times.

A solution of Fmoc-Arg(Pbf)-OH (5.8 g, 9.0 mmol) and HATU (3.3 g, 8.7 mmol), in 20 mL of anhydrous DMF was prepared. After the solution became homogeneous, DIPEA (3.2 mL, 18.0 mmol) was added, and the resulting mixture was added immediately to the resin. The resin was then agitated for thirty minutes, filtered, and washed with DMF (three times). A 25/75 solution of piperidine/DMF (30 mL) was added, and the resin agitated for three minutes. After filtration, piperidine/DMF was again added to the resin followed by agitation for 20 minutes. The resin was then washed with DMF (six times). The above amino acid addition procedure was repeated for the remaining six couplings of Fmoc-Arg(Pbf)-OH.

Following the addition of the eighth Arg unit, a solution of 4-pentynoic acid (0.90 g, 9.0 mmol) and HATU (3.3 g, 8.7 mmol) was prepared in 15 mL of dry DMF. After the solution became homogeneous, DIPEA (3.2 mL, 18.0 mmol) was added, and the resulting mixture was added immediately to the resin. The resin was then agitated for thirty minutes, filtered, and washed with DMF (six times). After filtration, the resin was washed with DMF (six times) followed by CH₂Cl₂ (four times) to remove any residual DMF. The oligopeptide was then cleaved by agitating the resin with 95/2.5/2.5 TFA/H₂O/TIPS (30 ml) for three hours. The filtrated was collected in a clean flask, and the resin was washed with fresh cleavage solution and DCM several times. The solution was concentrated on a rotary evaporator and dissolved in minimal MeOH. The oligopeptide was precipitated from diethyl ether and isolated by filtration to give 1.6 g of an off-white powder.

### Example 3

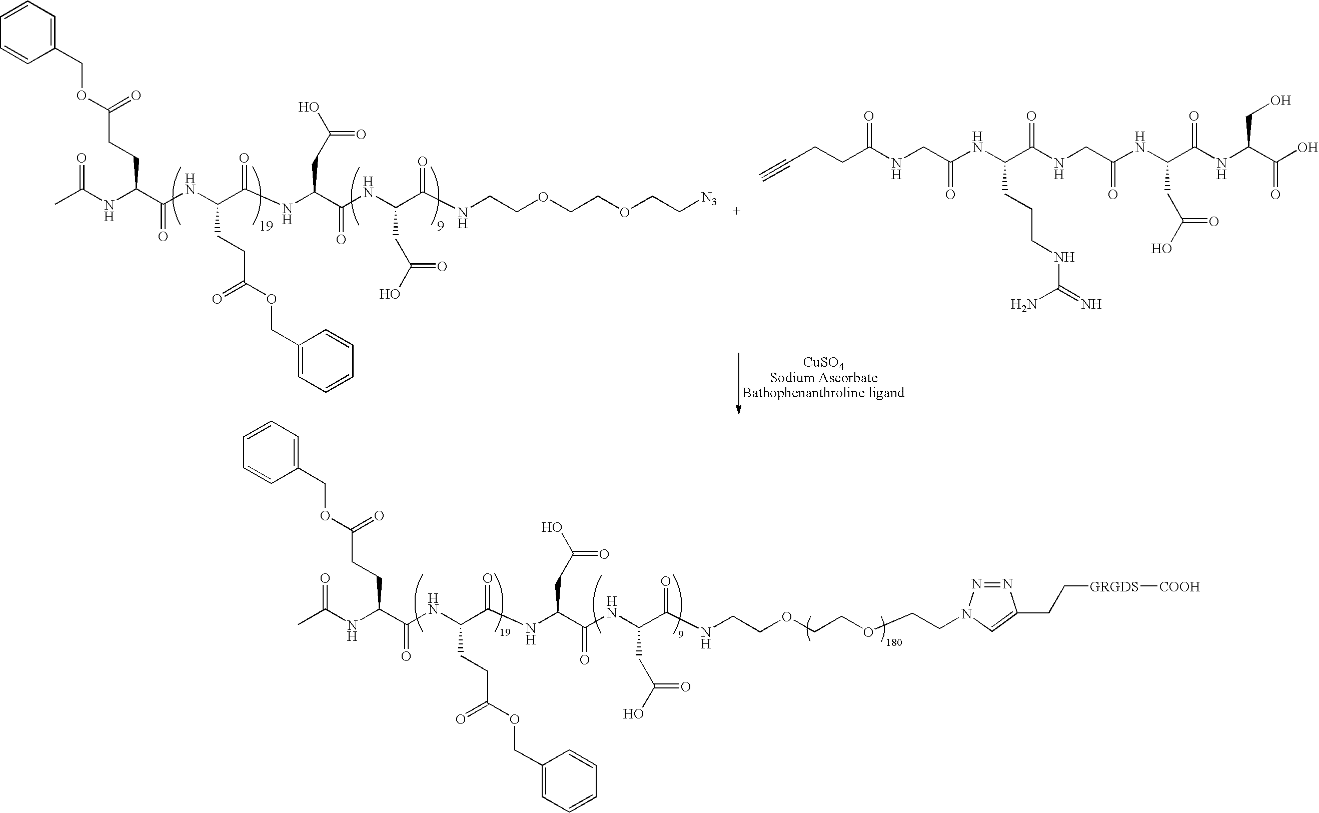

Conjugation of GRGDS to N₃—PEG8K-b-Poly(Asp₁₀)-b-Poly(Glu(Bzl)₂₀) via “Click” chemistry —N₃—PEG8K-b-Poly(Asp₁₀)-b-Poly(Glu(Bzl)₂₀) (96.0 mg), alkyne-GRGDS (2.4 mg), CuSO₄ (70 μL of a 10 mM stock solution in degassed, deionized water), sodium ascorbate (93 μL of a 150 mM stock solution in degassed, deionized water), and bathophenanthrolinedisulfonic acid (70 μL of a 30 mM stock solution in degassed, deionized water) and 0.5 mL of degassed, deionized water were combined (in that order) and stirred for 24 hours at room temperature under argon. Ethylenediaminetetraacetic acid disodium salt dihydrate (EDTA, 50 mg) was added to the reaction and allowed to stir for one hour. The product of the reaction was dialyzed twice against deionized water (10K MWCO membrane) and freeze-dried. GRGDS-functionalized PEG8K-b-Poly(Asp₁₀)-b-Poly(Glu(Bzl)₂₀) was recovered as a fluffy white powder.

### Example 4

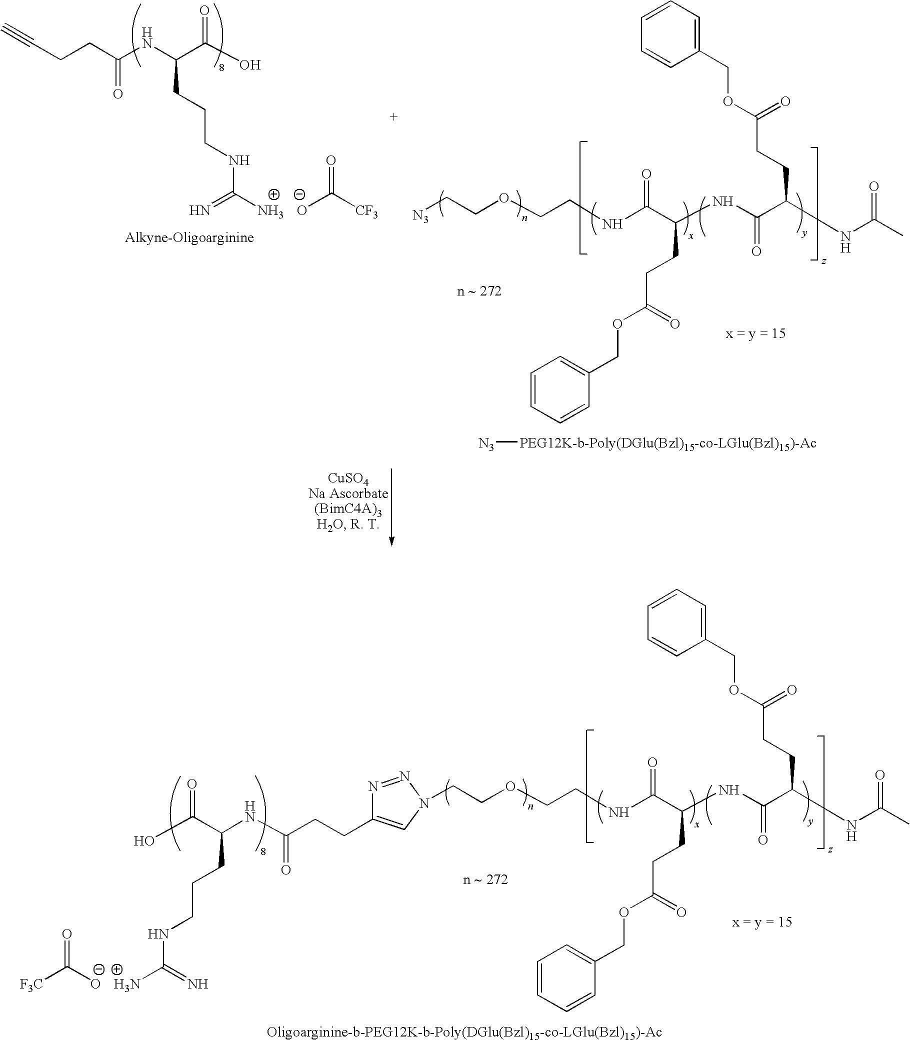

Conjugation of oligoarginine to N₃—PEG12k-b-Poly(DGlu(Bzl)₁₅-co-LGlu(Bzl)₁₅) via “Click” chemistry —N₃—PEG12k-b-Poly(DGlu(Bzl)₁₅-co-LGlu(Bzl)₁₅) (33.0 mg, 1.8 μmol), alkyne-oligoarginine (0.5 mL of a 8.3 mg/mL stock solution in deionized water, 1.8 μmol), CuSO₄ (0.5 mL of a 94.6 mg/L stock solution in deionized water, 0.19 μmol), sodium ascorbate (16.2 mg, 82 μmol), and an ionic benzimidazole ligand (BimC4A)₃ (0.25 mL of a 1 mg/mL aqueous stock solution in deionized water, 0.35 μmol) and 0.5 mL of deionized water were combined (in that order) and stirred for 24 hours at room temperature. Ethylenediaminetetraacetic acid disodium salt dihydrate (EDTA, 6.8 mg, 18.3 μmol) was added to the reaction and allowed to stir for one hour. The product of the reaction was dialyzed twice against deionized water (10K MWCO membrane) and freeze-dried. Oligoarginine-functionalzed PEG12k-b-Poly(DGlu(Bzl)₁₅-co-LGlu(Bzl)₁₅) was recovered as a fluffy white powder (23 mg, Yield=62%). For more details on (BimC4A)₃, see Rodionov, et. al., J. Am. Chem. Soc. 2007, 129, 12696.

### Example 5

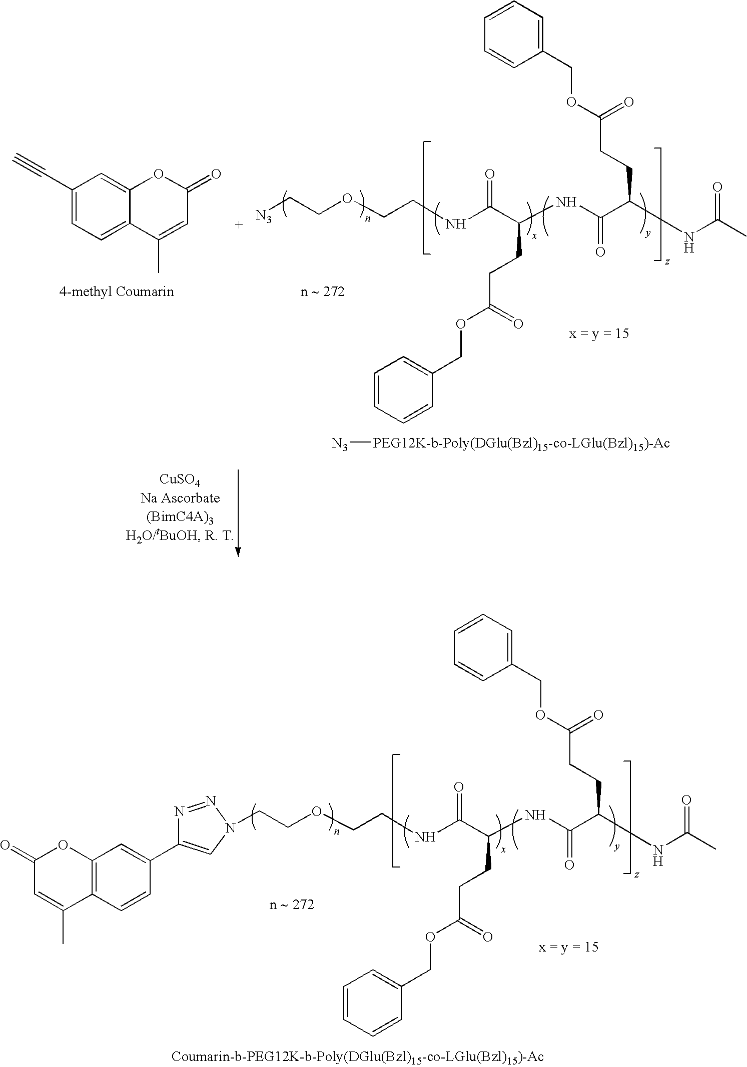

Conjugation of 4-methyl coumarin to N₃—PEG12k-b-Poly(DGlu(Bzl)₁₅-co-LGlu(Bzl)₁₅) via “Click” chemistry —N₃—PEG12k-b-Poly(DGlu(Bzl)₁₅-co-LGlu(Bzl)₁₅) (33.0 mg, 1.8 μmol), acetylene-functionalized, 4-methyl coumarin (0.5 mL of a 0.7 mg/mL stock solution in ^{t}BuOH, 1.9 μmol)) CuSO₄ (0.5 mL of a 94.6 mg/L stock solution in deionized water, 0.19 μmol), sodium ascorbate (16.2 mg, 82 μmol), (BimC4A)₃ (0.25 mL of a 1 mg/mL aqueous stock solution in deionized water, 0.35 μmol) and 0.5 mL of deionized water were combined (in that order) and allowed to stir for 24 hours at room temperature. Ethylenediaminetetraacetic acid disodium salt dihydrate (EDTA, 6.8 mg, 18.3 μmol) was added to the reaction and allowed to stir for one hour. The product of the reaction was dialyzed twice against deionized water (10K MWCO membrane) and freeze-dried. Coumarin-functionalized PEG12k-b-Poly(DGlu(Bzl)₁₅-CO-LGlu(Bzl)₁₅) was recovered as a fluffy white powder (23 mg, Yield=62%).

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

What is claimed is:

1. A click-functionalized targeting group, provided that the click-functionalized targeting group is not: 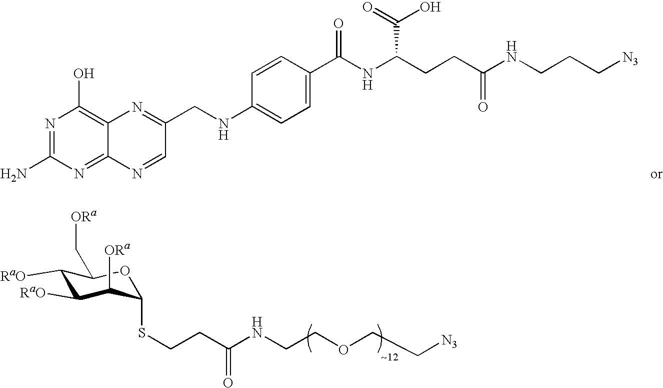
wherein each R^{a} is independently hydrogen or acetyl.

2. The click-functionalized targeting group of claim 1, wherein the targeting group is selected from the group consisting of Her-2 binding peptides, uPAR antagonists, CXCR4 antagonists, GRP78 antagonist peptides, RGD peptides, LHRH antagonists peptides, aminopeptidase N(CD 13) targeting peptides, and cell-permeating peptides.

3. The click-functionalized targeting group of claim 1, wherein the targeting group is selected from the group consisting of brain homing peptides, kidney homing peptides, heart homing peptides, gut homing peptides, integrin homing peptides, RGD-binding determinants, angiogenic tumor endothelium homing peptides, ovary homing peptides, uterus homing peptides, sperm homing peptides, microglia homing peptides, synovium homing peptides, urothelium homing peptides, prostate homing peptides, lung homing peptides, skin homing peptides, retina homing peptides, pancreas homing peptides, liver homing peptides, lymph node homing peptides, adrenal gland homing peptides, thyroid homing peptides, bladder homing peptides, breast homing peptides, neuroblastoma homing peptides, lymphoma homing peptides, muscle homing peptides, wound vasculature homing peptides, adipose tissue homing peptides, anti-viral peptides, fusogenic peptides, tumor homing peptides, prostate specific membrane antigen (PSMA) homing peptides, aminopeptidase N homing peptides, HER-2 homing peptides, colon cancer homing peptides, VEGFR1 homing peptides, and CXCR4 homing peptides.

4. The click-functionalized targeting group of claim 3, wherein the targeting group is selected from the group consisting of SEQ ID Nos. 1-825.

5. The click-functionalized targeting group of claim 2, wherein said click-functionalized targeting group is of any formula I-a, I-b, or I-c: 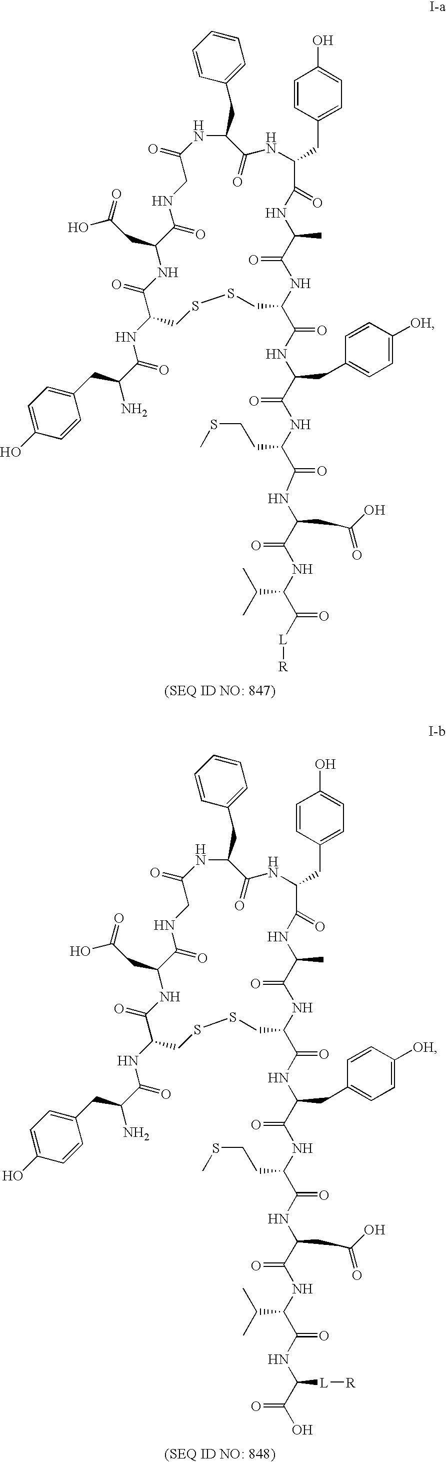
or 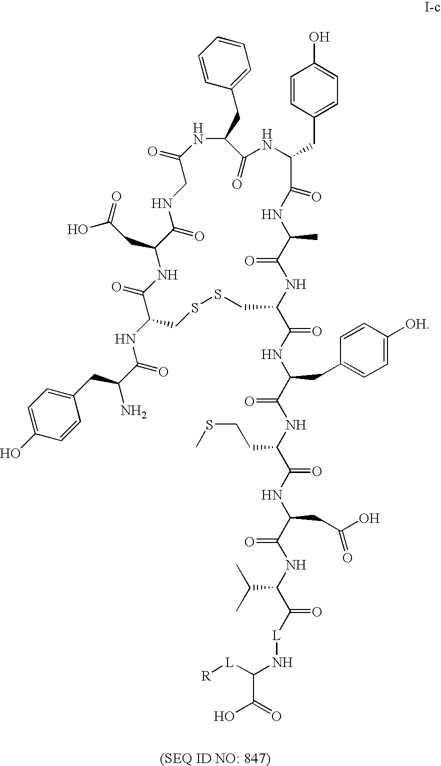
or a salt thereof, wherein each L is independently a valence bond or a bivalent, saturated or unsaturated, straight or branched C₁₋₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
each R is independently alkyne or azide.

6. The click-functionalized targeting group of claim 5, wherein said click-functionalized targeting group is selected from:

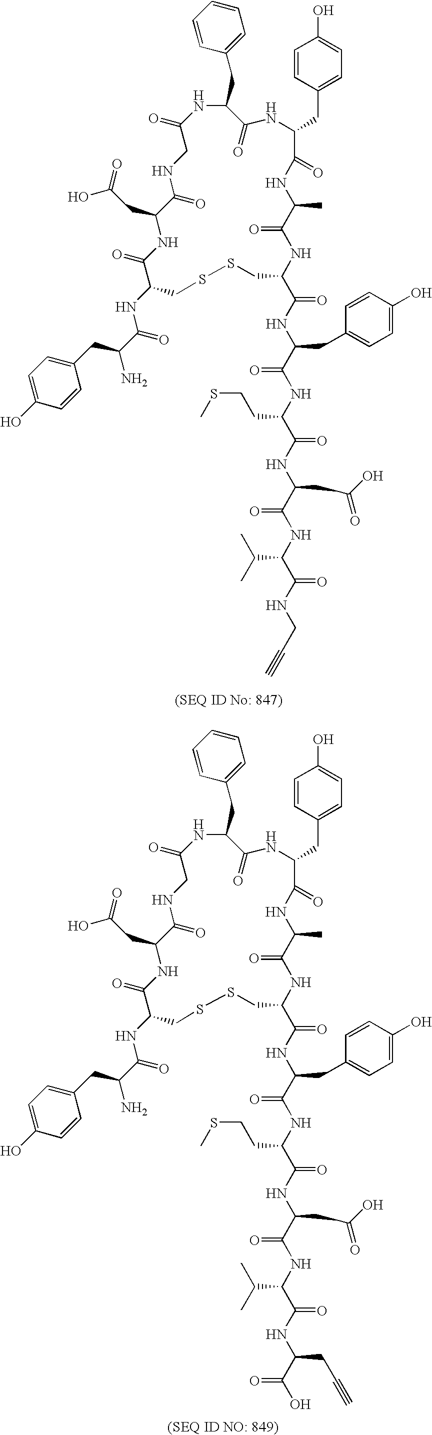

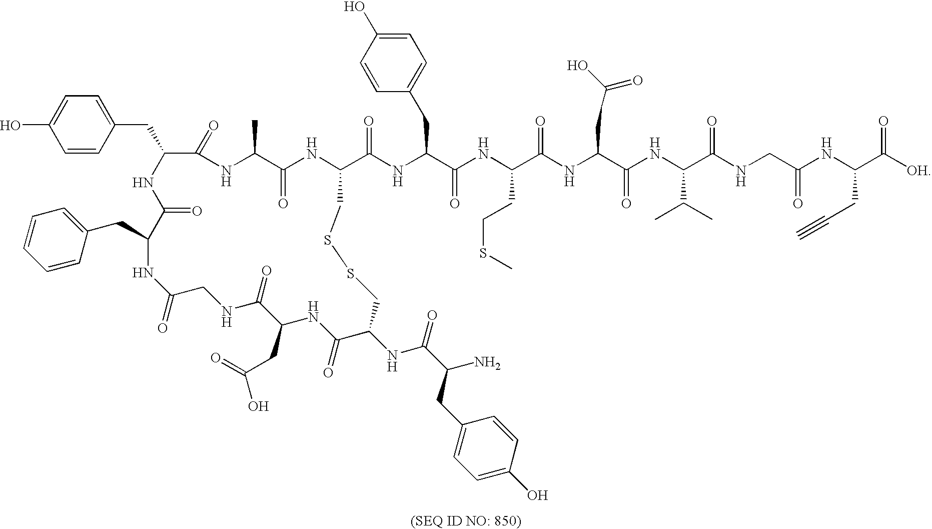

7. The click-functionalized targeting group of claim 2, wherein said click-functionalized targeting group is of any formula II-a, I-b, II-c, II-d, II-e, II-f, II-g, II-h, II-i, II-j, II-k, II-l, II-m, II-n, or II-o: 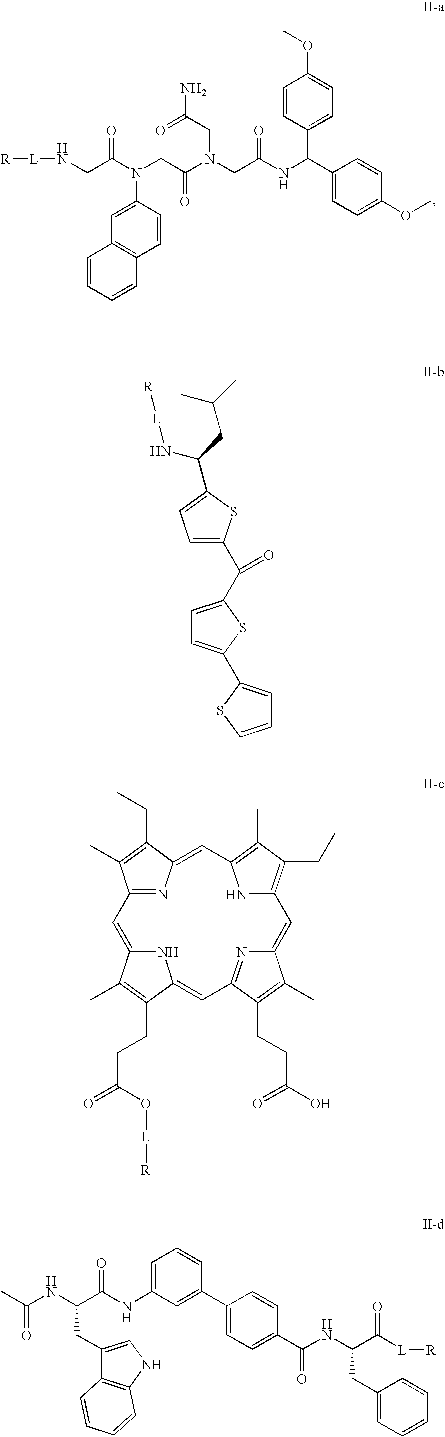 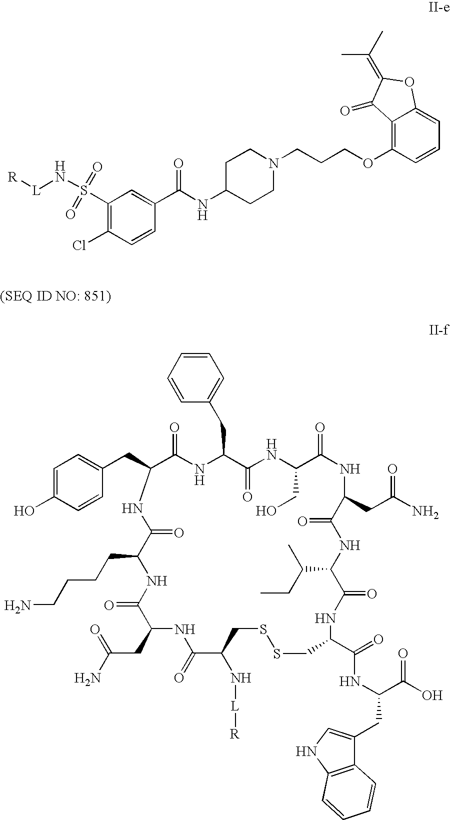 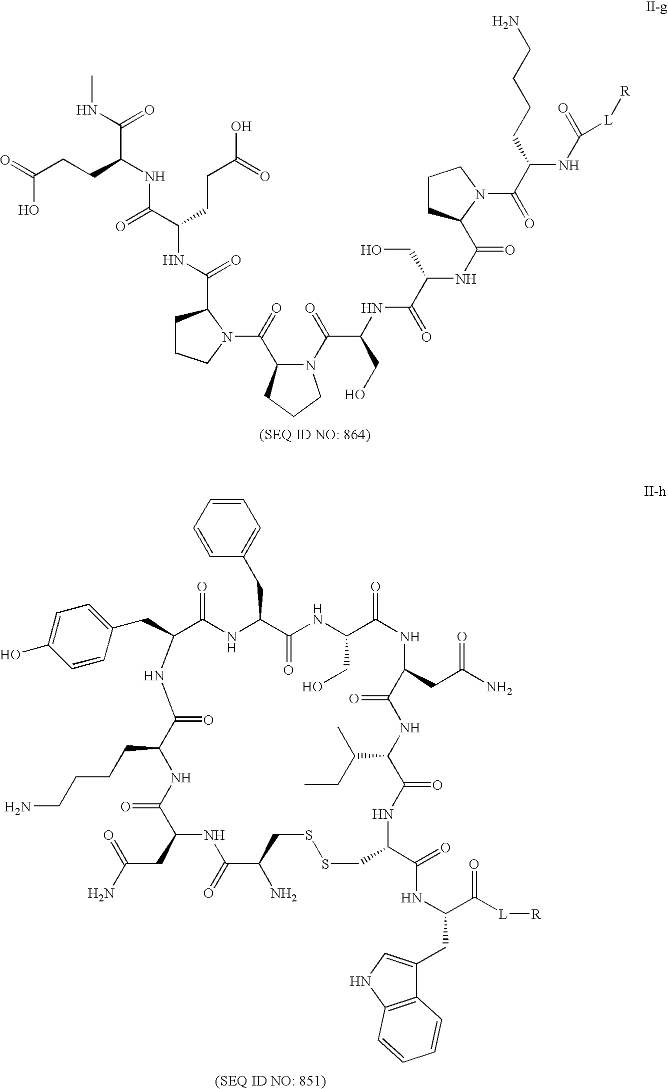 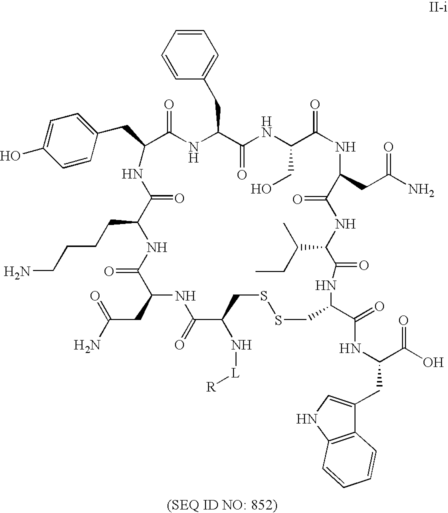 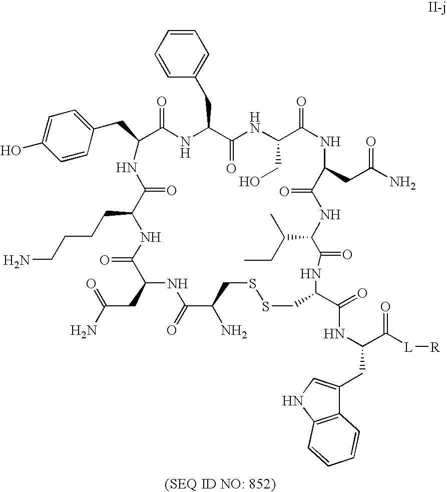 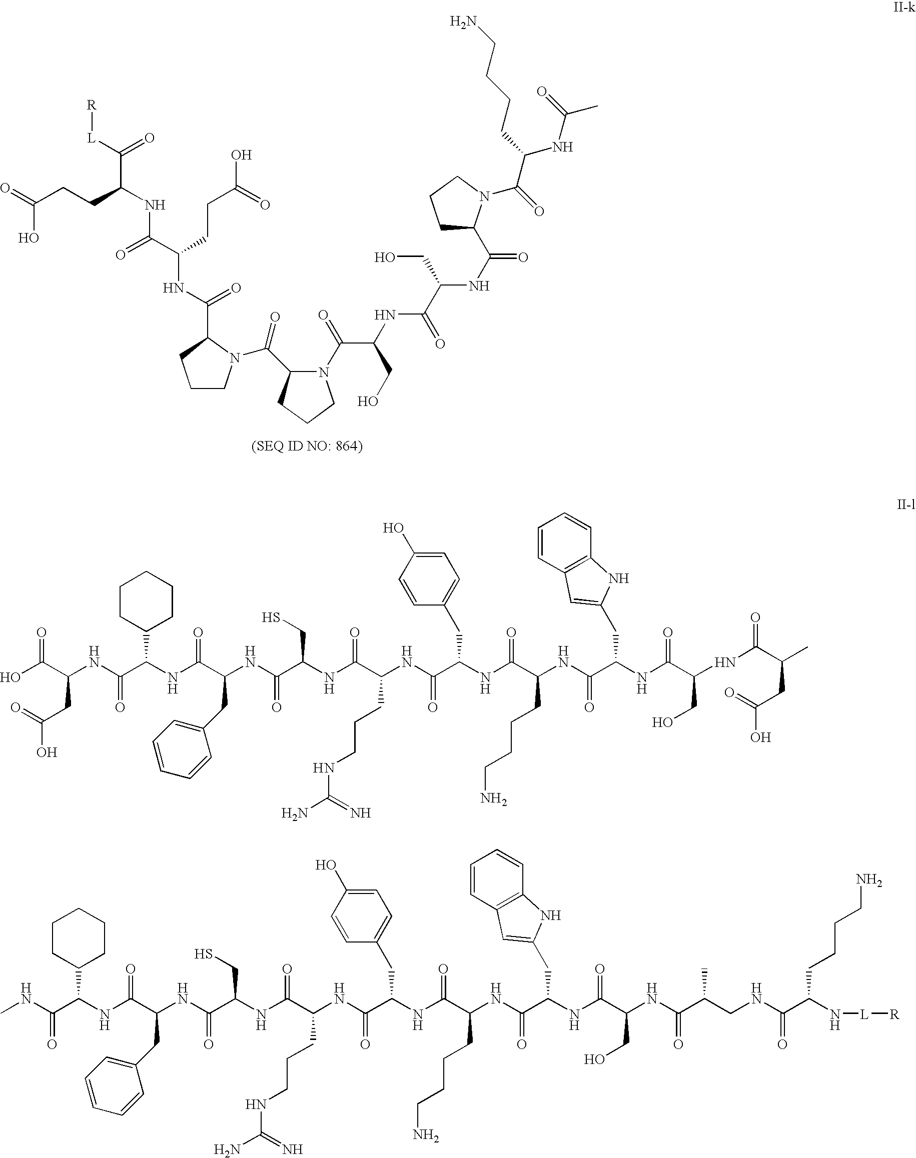 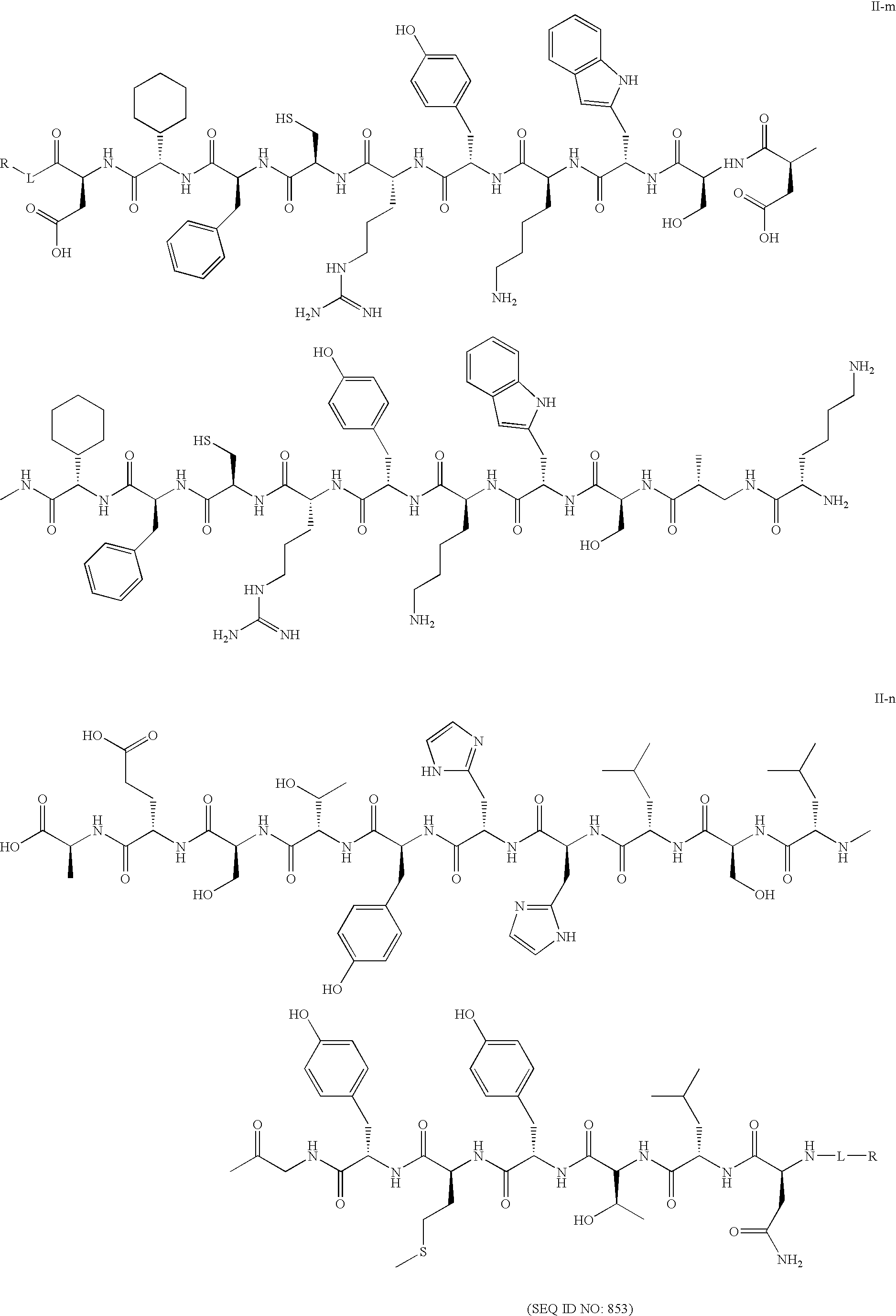 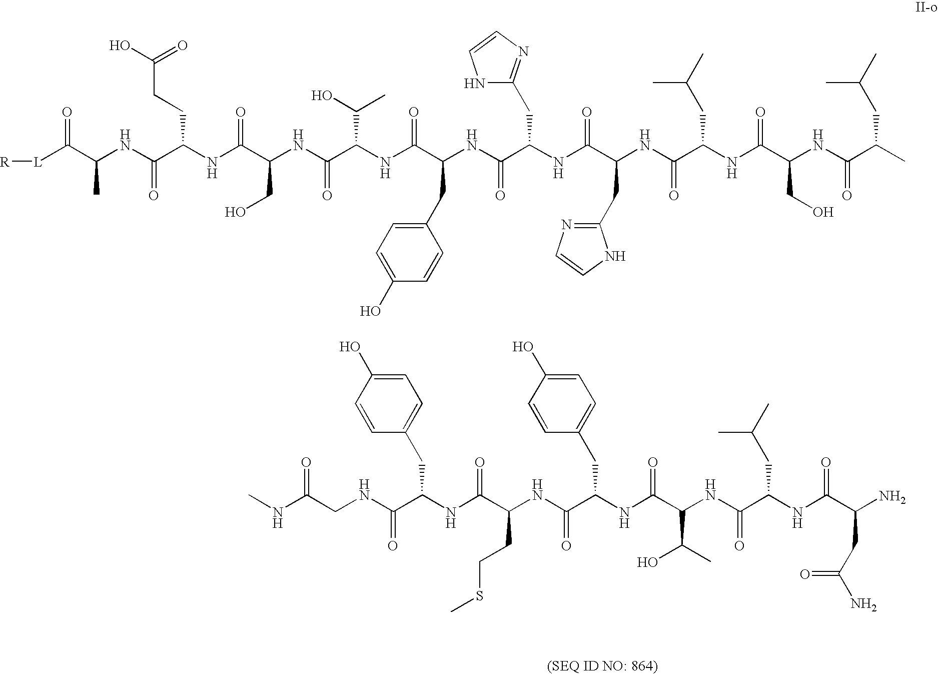
or a salt thereof, wherein each L is independently a valence bond or a bivalent, saturated or unsaturated, straight or branched C₁₋₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
each R is independently alkyne or azide.

8. The click-functionalized targeting group of claim 2, wherein said click-functionalized targeting group is of formula III: 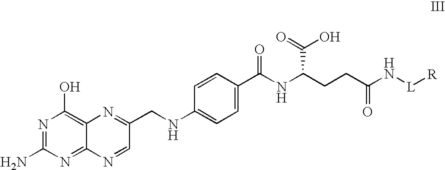
or a salt thereof, wherein each L is independently a valence bond or a bivalent, saturated or unsaturated, straight or branched C₁₋₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
each R is independently alkyne or azide, provided that L is not —(CH₂CH₂CH₂)— when R is N₃.

9. The click-functionalized targeting group of claim 2, wherein said click-functionalized targeting group is of any formula IV-a, IV-b, IV-c, IV-d, IV-e, or IV-f: 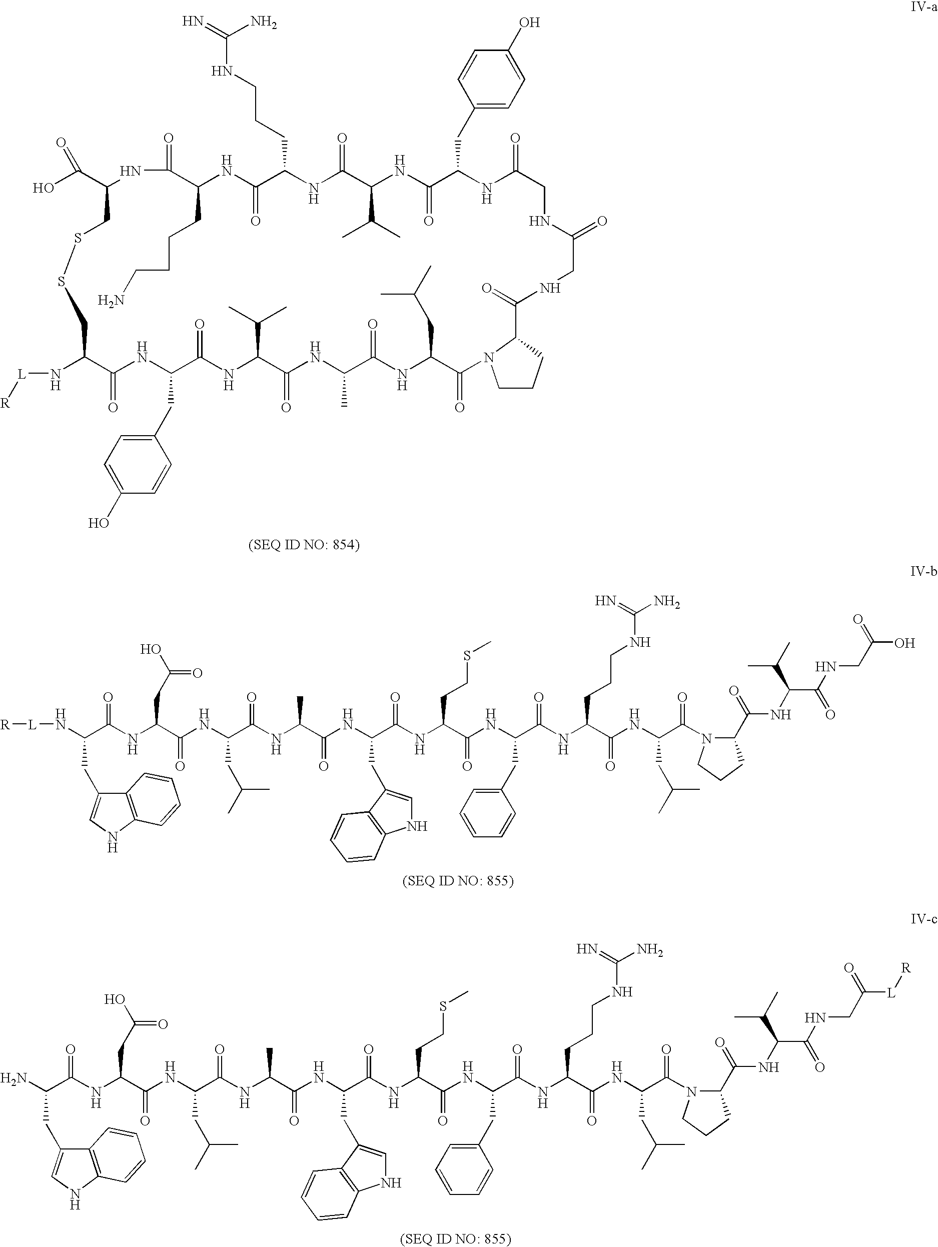 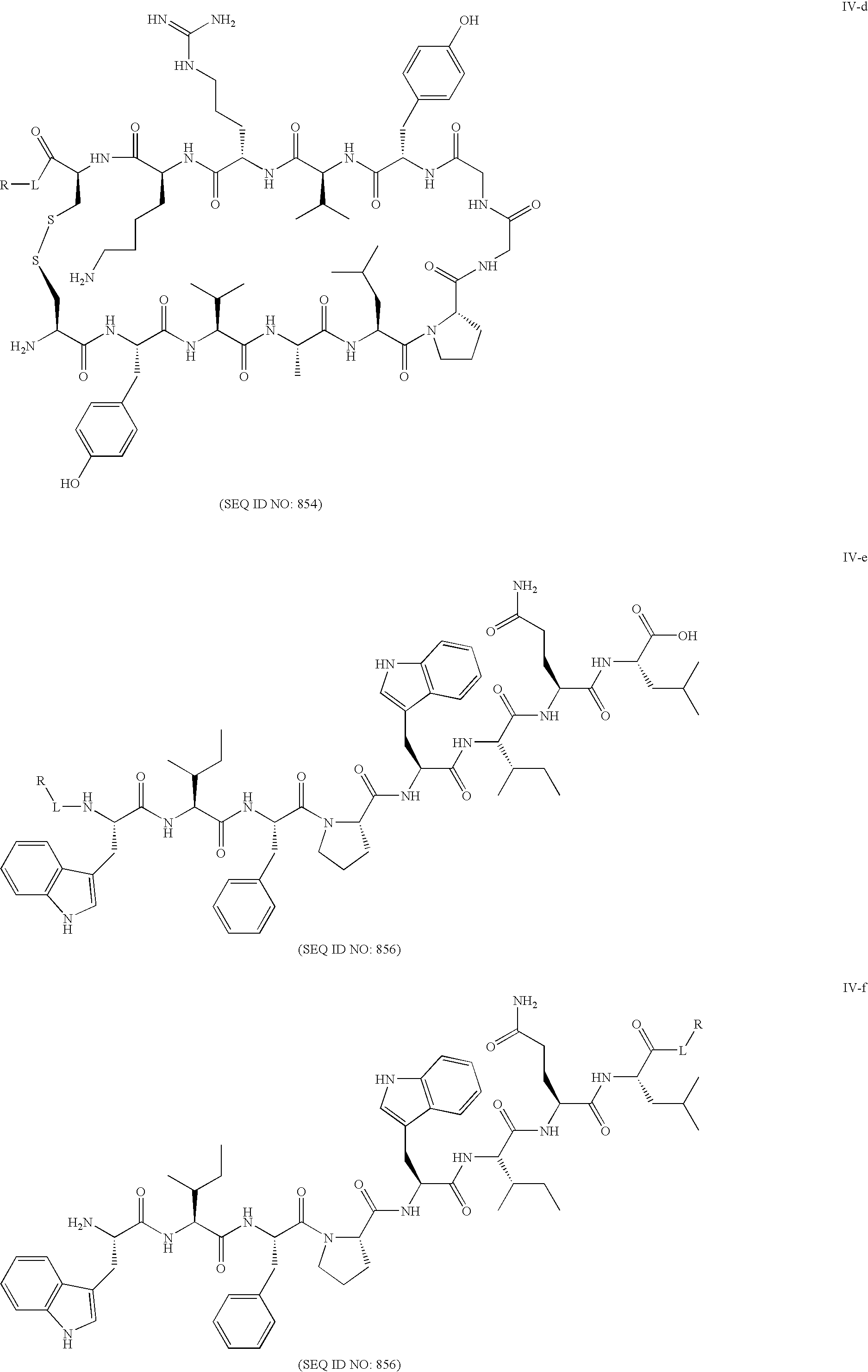
or a salt thereof, wherein each L is independently a valence bond or a bivalent, saturated or unsaturated, straight or branched C₁₋₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
each R is independently alkyne or azide.

10. The click-functionalized targeting group of claim 9, wherein said click-functionalized targeting group is selected from:

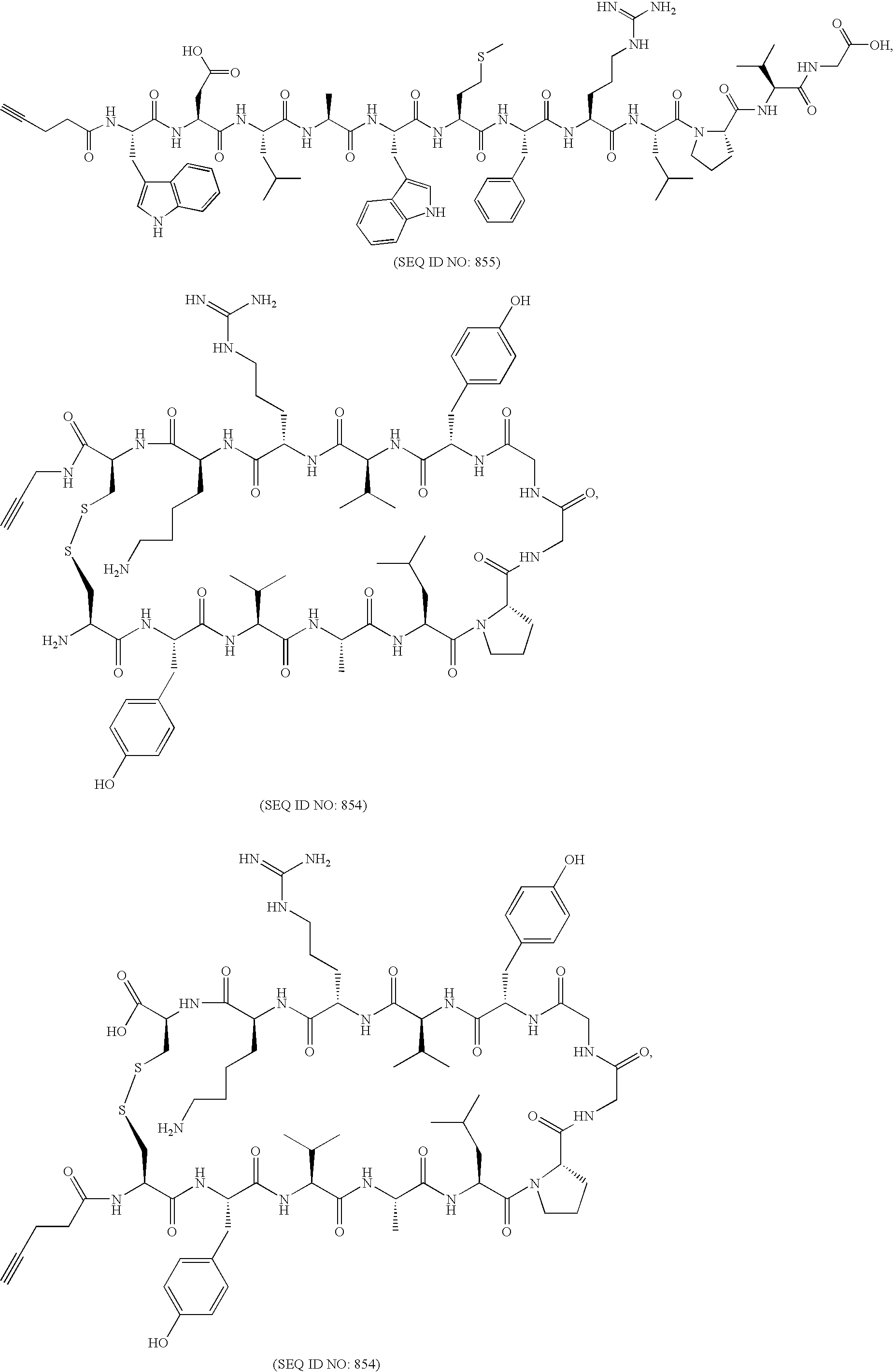

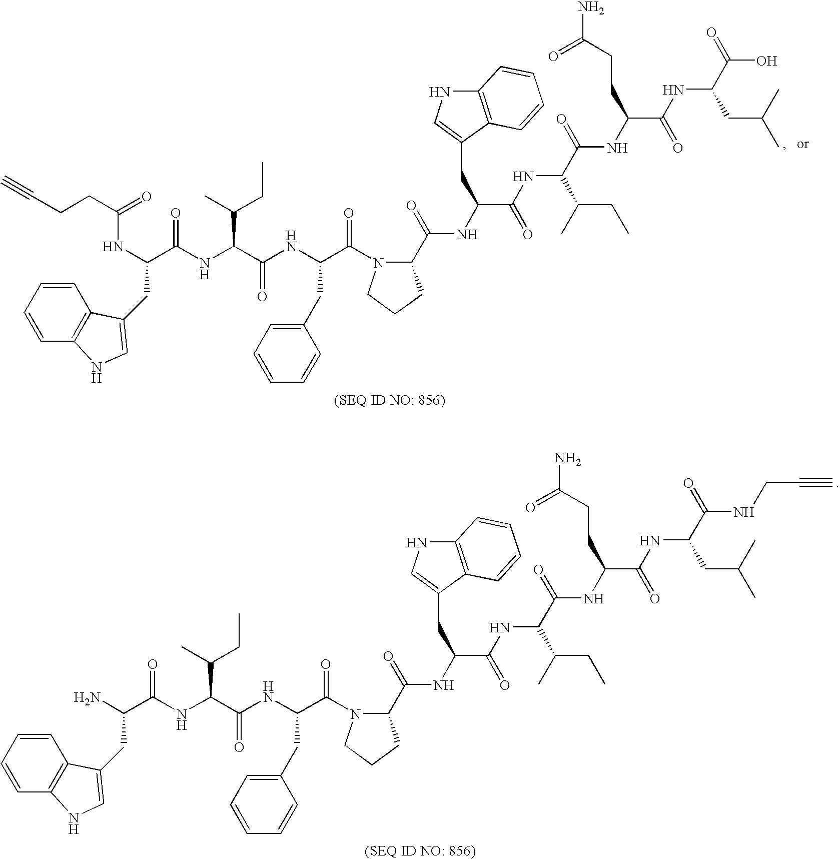

11. The click-functionalized targeting group of claim 2, wherein said click-functionalized targeting group is of any formula V-a, V-b, V-c, V-d, V-e, or V-f: 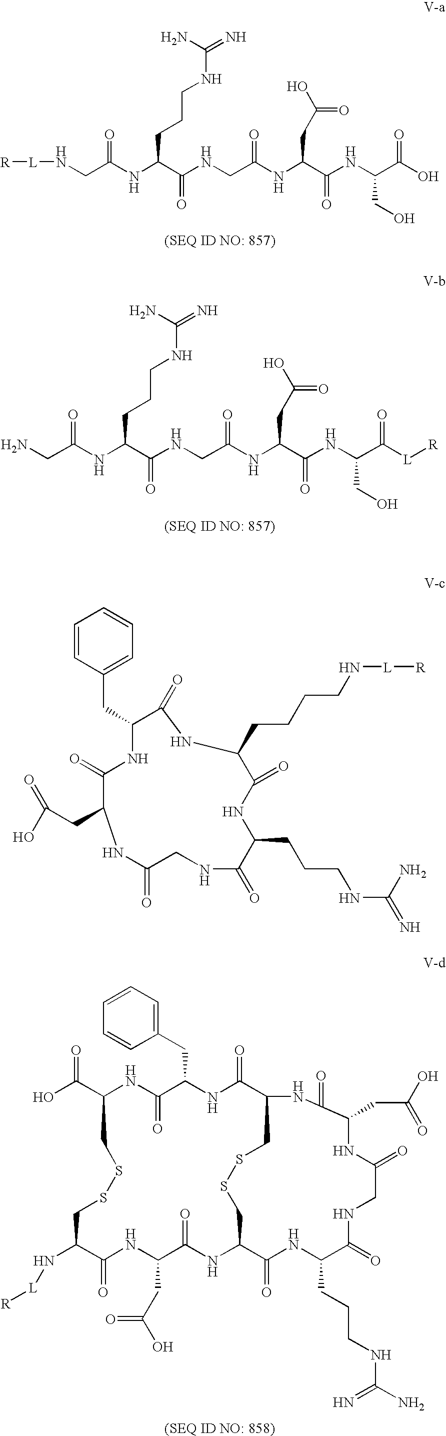 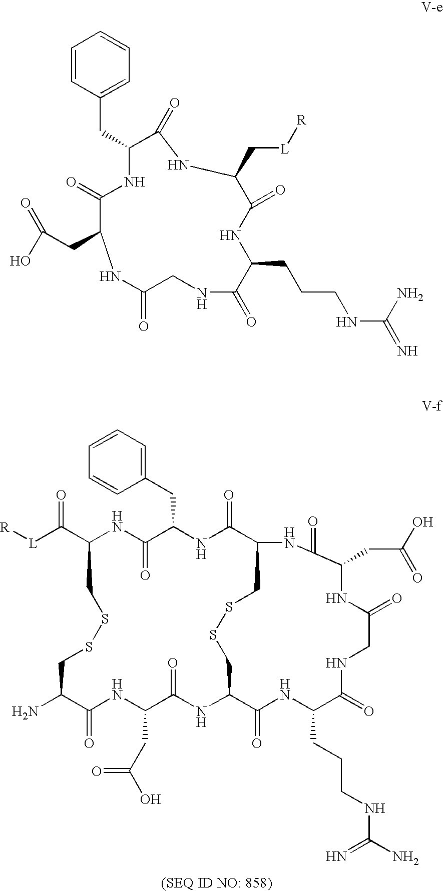
or a salt thereof, wherein each L is independently a valence bond or a bivalent, saturated or unsaturated, straight or branched C₁₋₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
each R is independently alkyne or azide.

12. The click-functionalized targeting group of claim 11, wherein said click-functionalized targeting group is selected from:

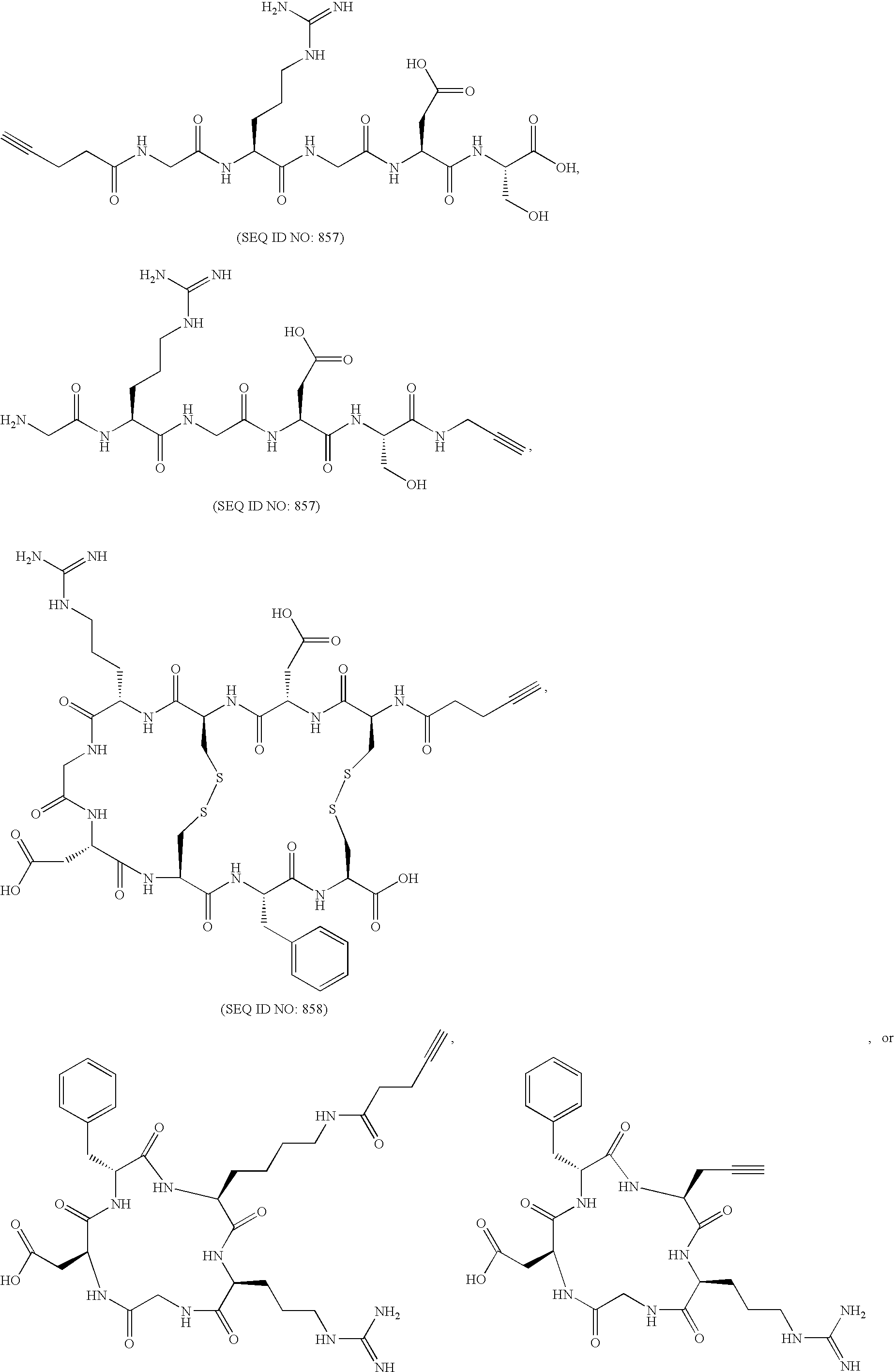

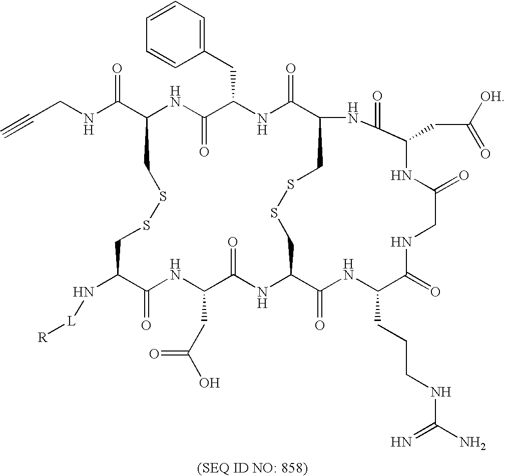

13. The click-functionalized targeting group of claim 2, wherein said click-functionalized targeting group is of any formula VI-a, VI-b, VI-c, VI-d, or VI-e: 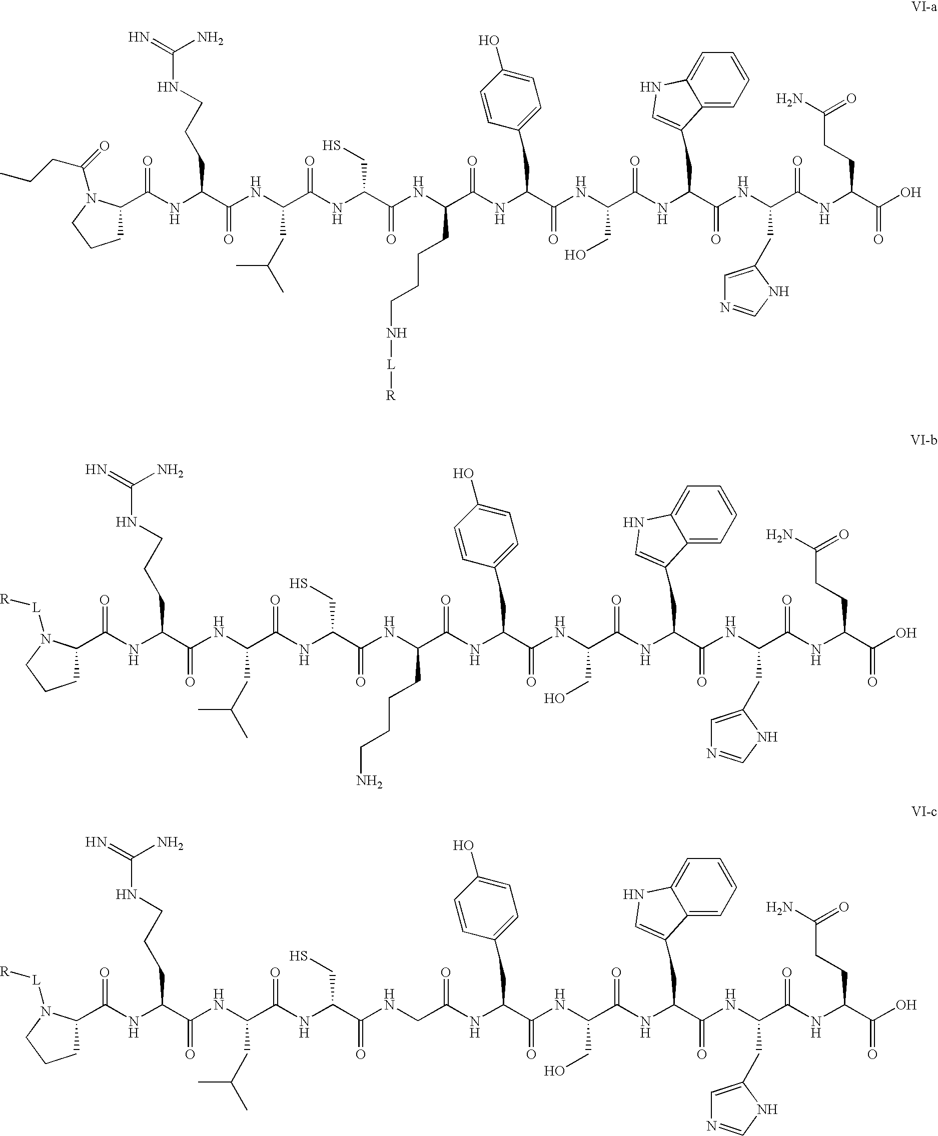 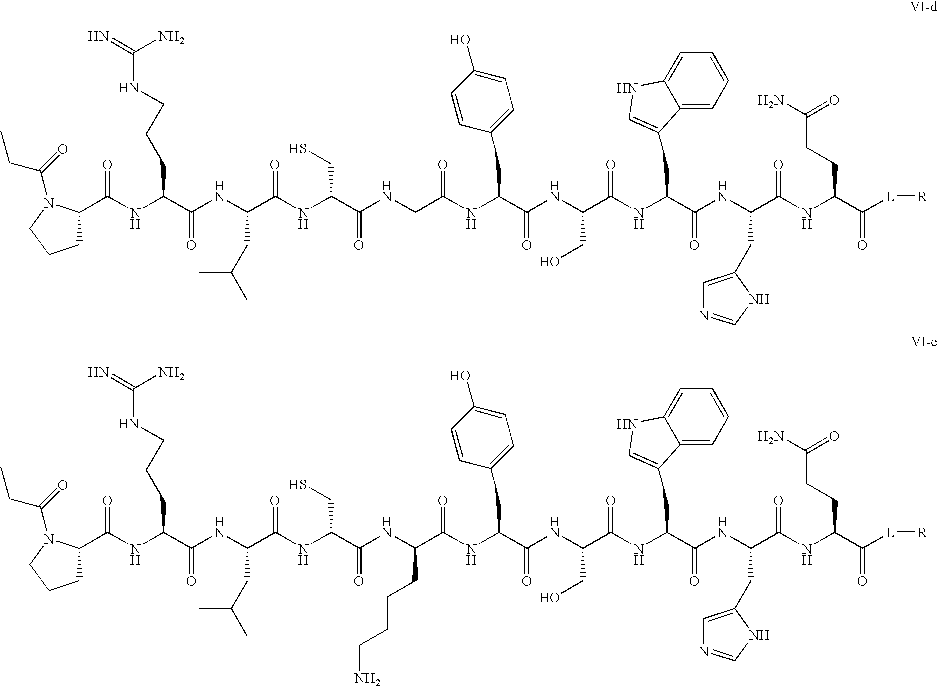
or a salt thereof, wherein each L is independently a valence bond or a bivalent, saturated or unsaturated, straight or branched C₁₋₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
each R is independently alkyne or azide.

14. The click-functionalized targeting group of claim 13, wherein said click-functionalized targeting group is selected from:

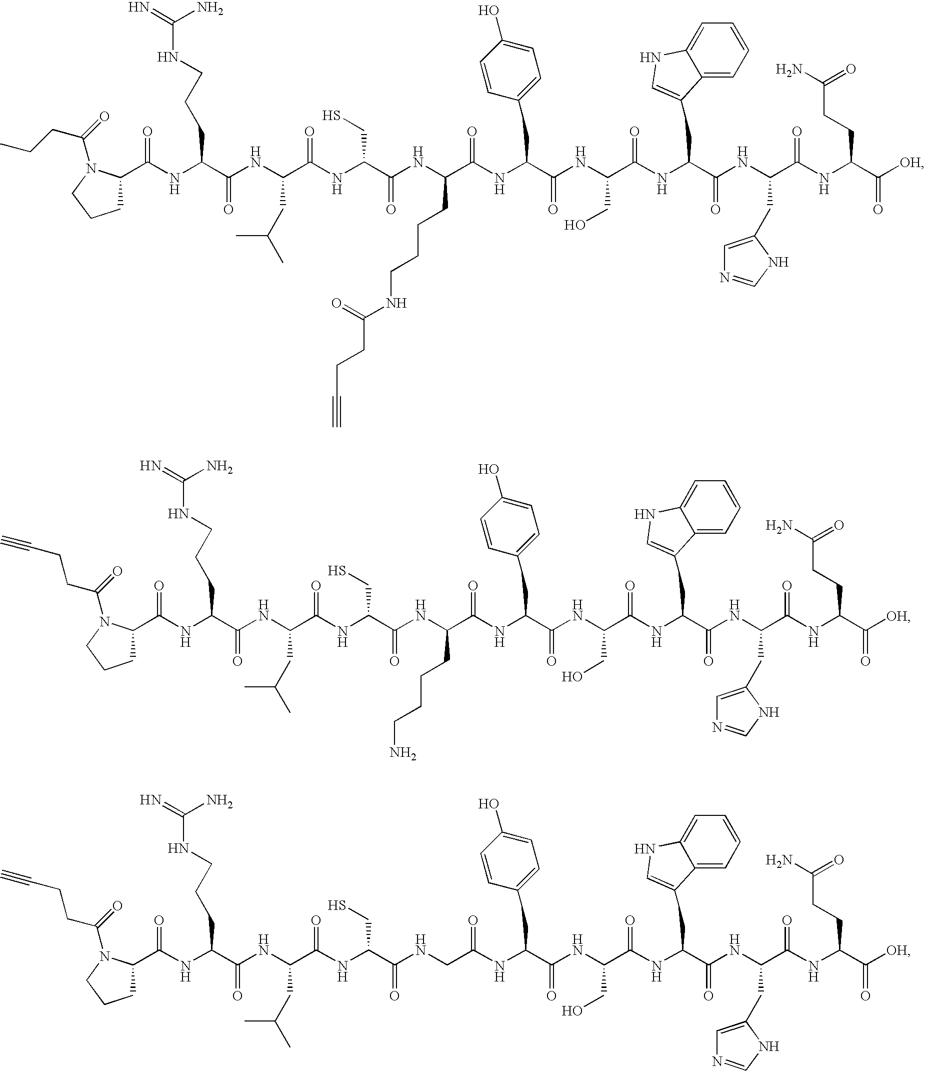

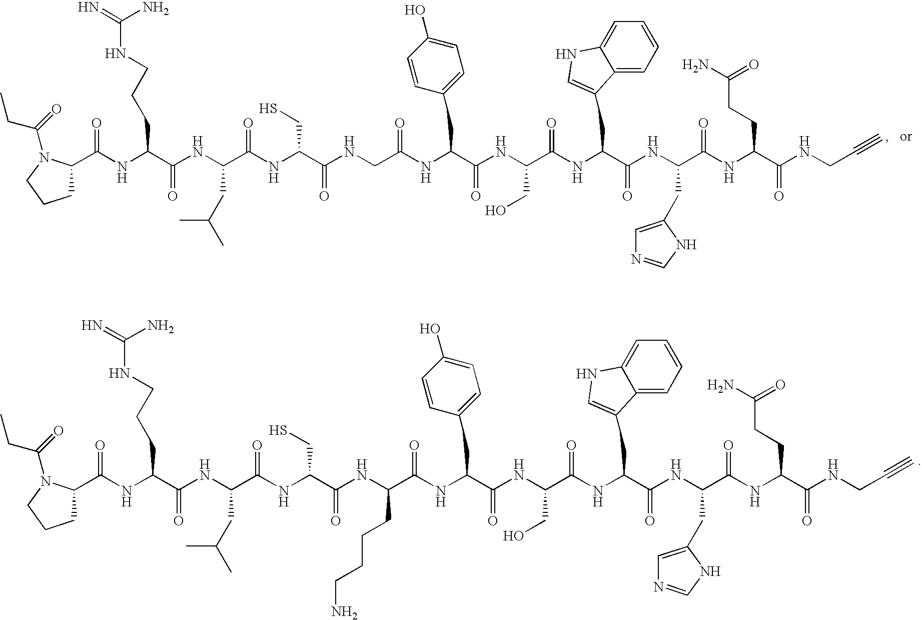

15. The click-functionalized targeting group of claim 2, wherein said click-functionalized targeting group is of any formula VII-a, VII-b, VII-c, or VII-d: 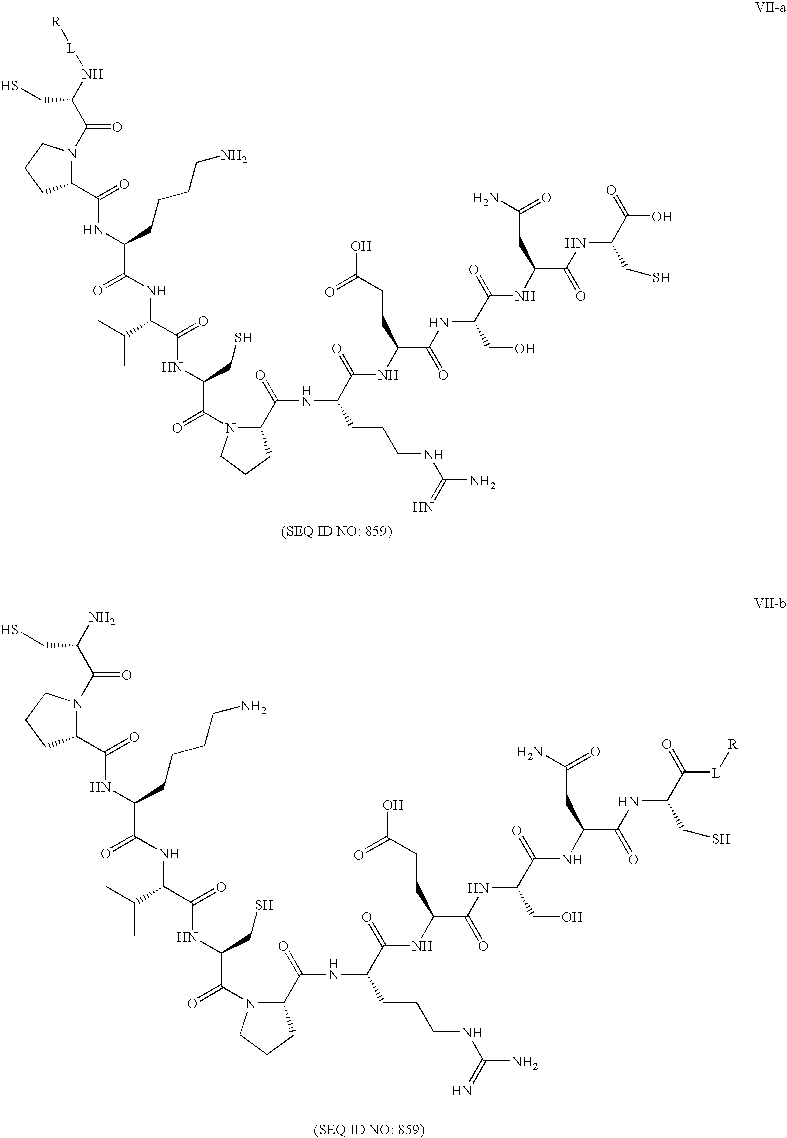 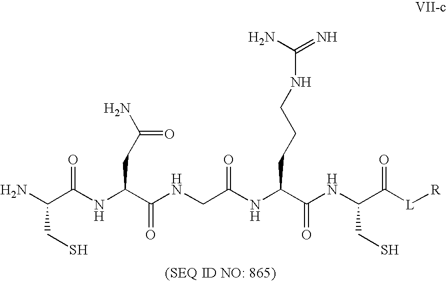 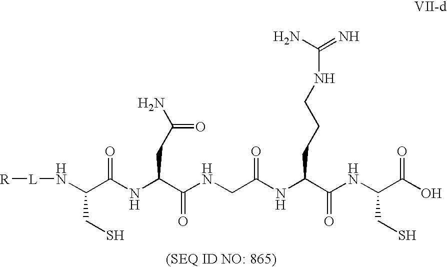
or a salt thereof, wherein each L is independently a valence bond or a bivalent, saturated or unsaturated, straight or branched C₁₋₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
each R is independently alkyne or azide.

16. The click-functionalized targeting group of claim 15, wherein said click-functionalized targeting group is selected from:

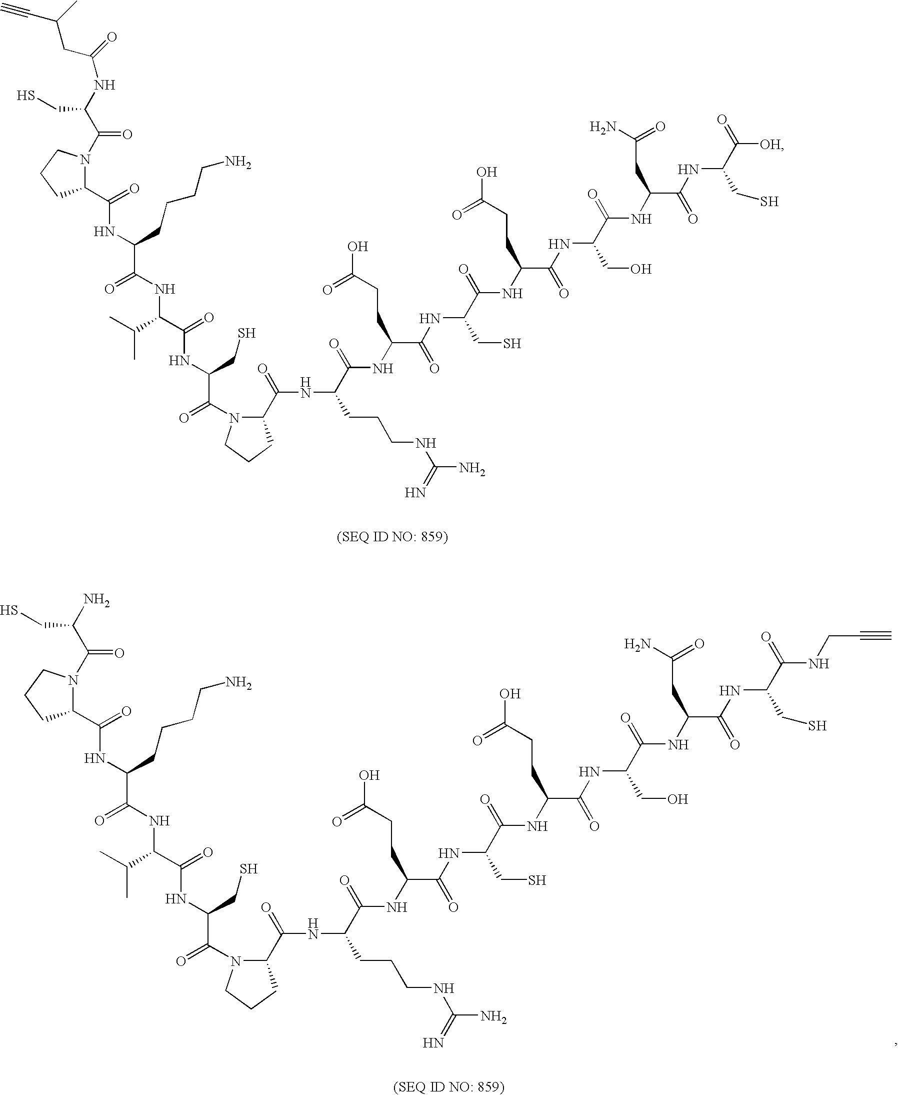

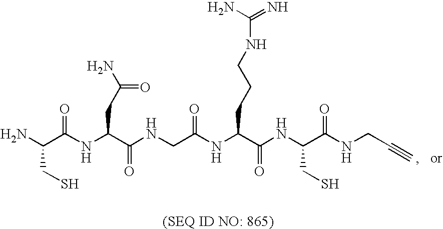

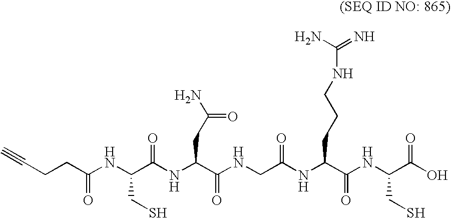

17. The click-functionalized targeting group of claim 2, wherein said click-functionalized targeting group is of any formula VIII-a, VIII-b, VIII-c, VIII-d, VIII-e, or VIII-f: 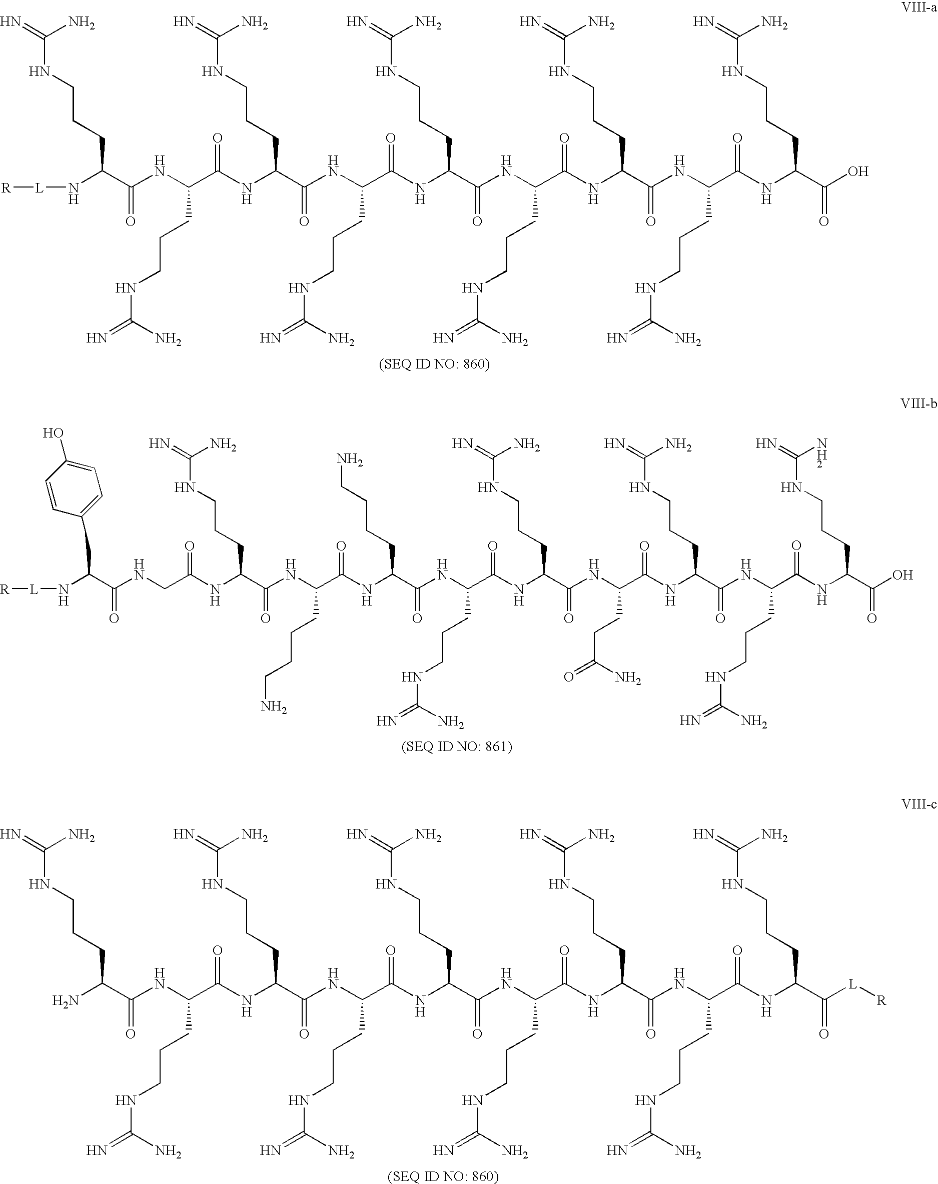 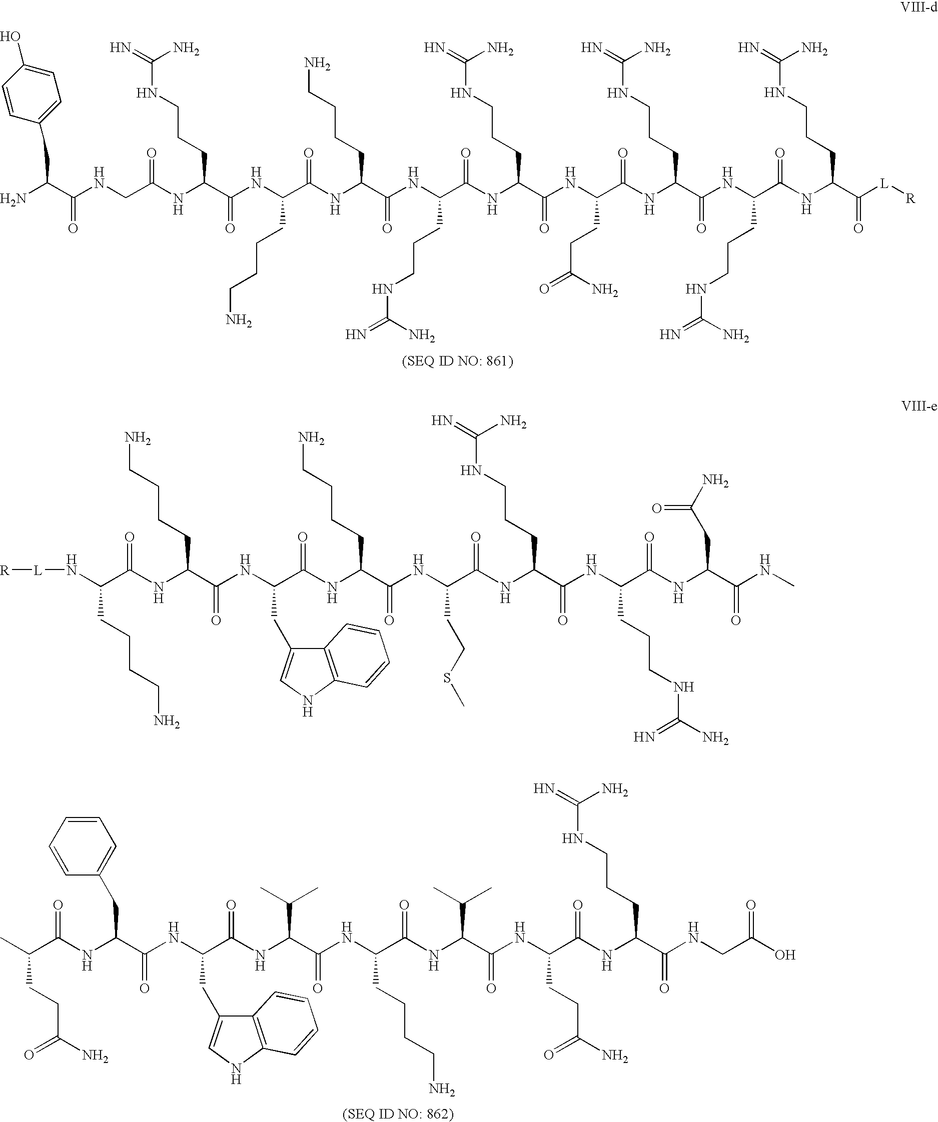 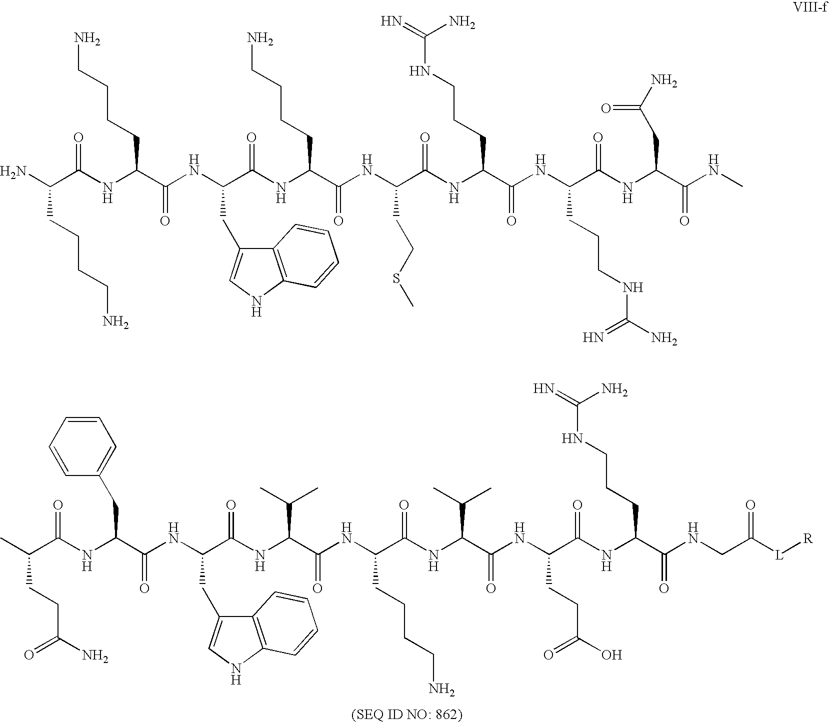
or a salt thereof, wherein each L is independently a valence bond or a bivalent, saturated or unsaturated, straight or branched C₁₋₂ hydrocarbon chain, wherein 0-6 methylene units of L are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
each R is independently alkyne or azide.

18. The click-functionalized targeting group of claim 17, wherein said click-functionalized targeting group is selected from:

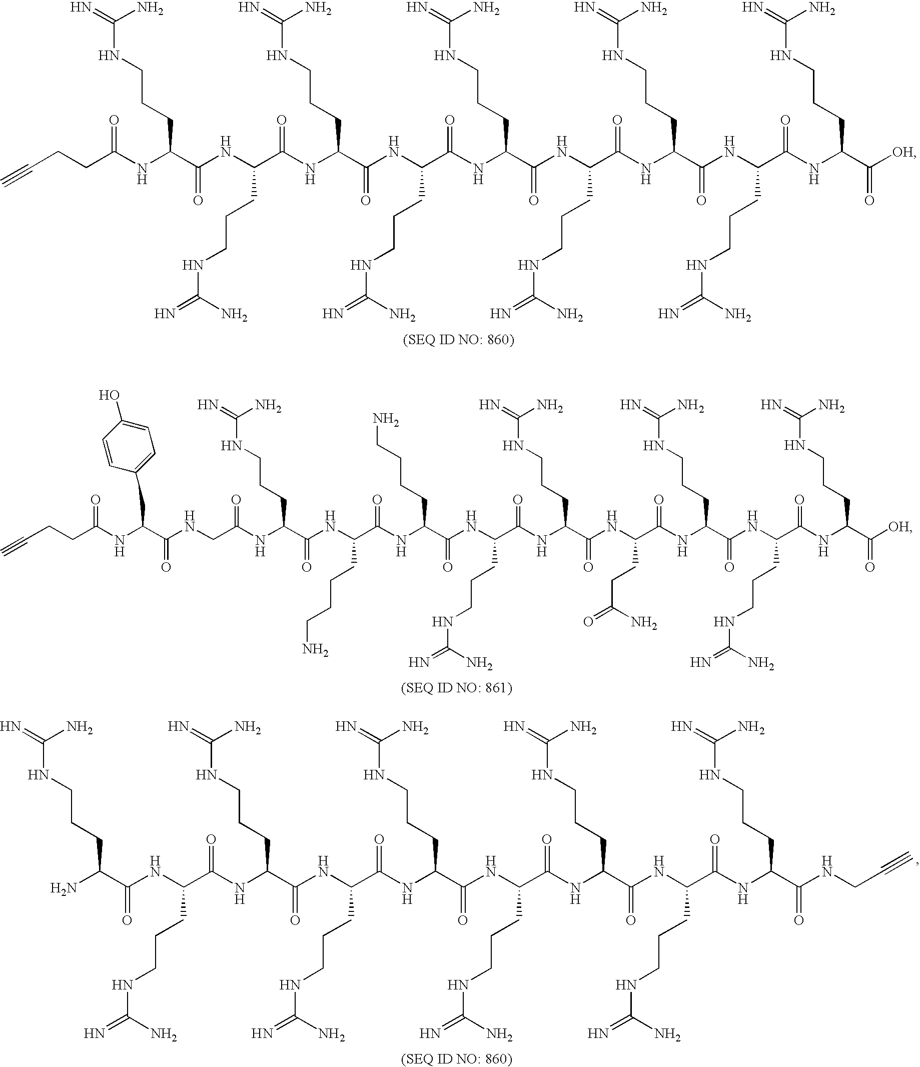

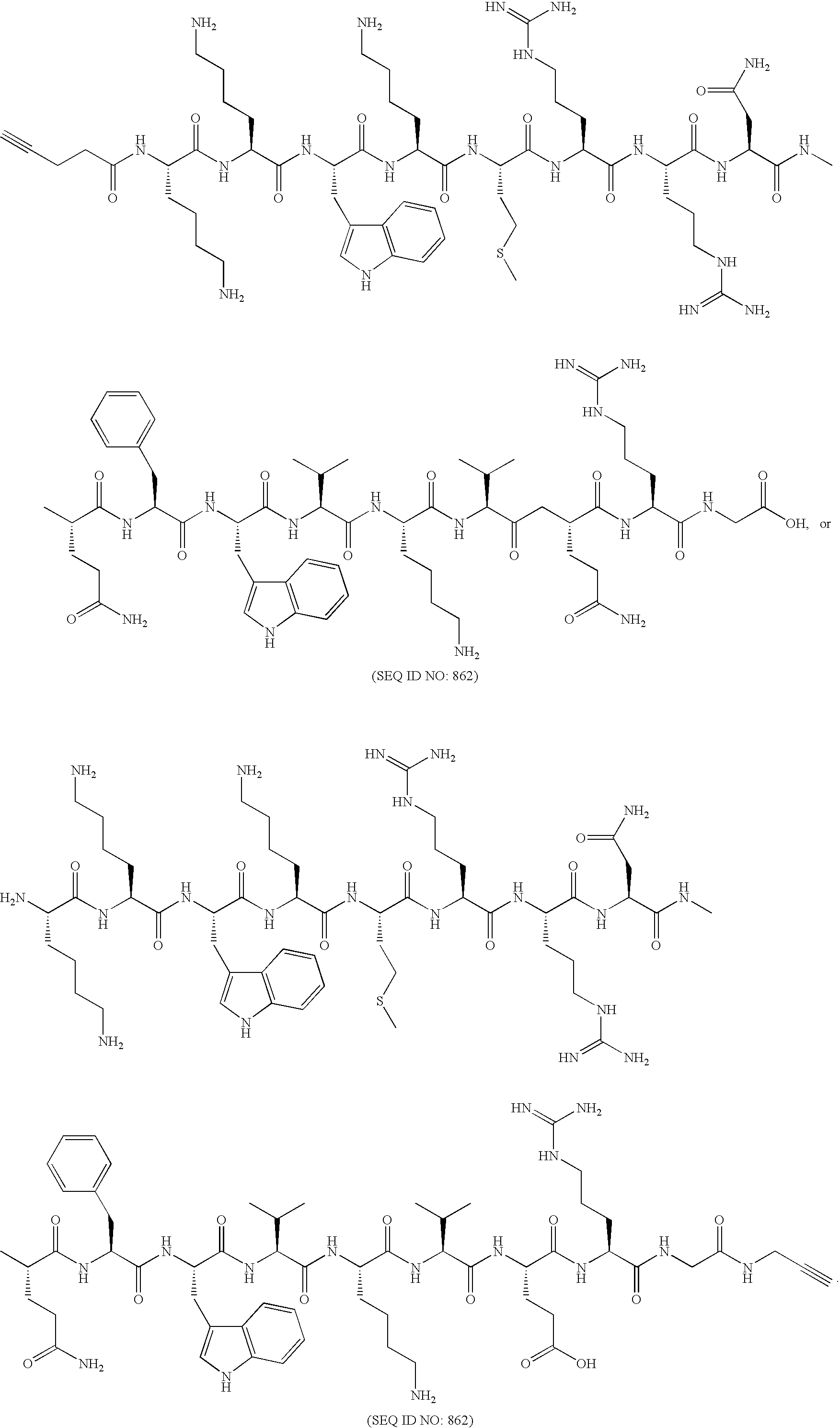

19. The click-functionalized targeting group of claim 1, wherein said click-functionalized targeting group is conjugated to a polymer.

20. The click-functionalized targeting group of claim 19, wherein the polymer is PEG or a functionalized PEG.

21. The click-functionalized targeting group of claim 1, wherein said click-functionalized targeting group is conjugated to a polymer micelle.

22. The click-functionalized targeting group of claim 21, wherein the micelle has a therapeutic agent encapsulated therein, wherein the therapeutic agent is selected from a protein, a virus, a DNA plasmid, a oligonucleotide, a drug, a dye, or a primary or secondary label.

23. The click-functionalized targeting group of claim 22, wherein the drug is a chemotherapeutic agent selected from the group consisting of Abarelix, aldesleukin, Aldesleukin, Alemtuzumab, Alitretinoin, Allopurinol, Altretamine, Amifostine, Anastrozole, Arsenic trioxide, Asparaginase, Azacitidine, BCG Live, Bevacuzimab, Avastin, Fluorouracil, Bexarotene, Bleomycin, Bortezomib, Busulfan, Calusterone, Capecitabine, Camptothecin, Carboplatin, Carmustine, Celecoxib, Cetuximab, Chlorambucil, Cisplatin, Cladribine, Clofarabine, Cyclophosphamide, Cytarabine, Dactinomycin, Darbepoetin alfa, Daunorubicin, Denileukin, Dexrazoxane, Docetaxel, Doxorubicin (neutral), Doxorubicin hydrochloride, Dromostanolone Propionate, Epirubicin, Epoetin alfa, Erlotinib, Estramustine, Etoposide Phosphate, Etoposide, Exemestane, Filgrastim, floxuridine fludarabine, Fulvestrant, Gefitinib, Gemcitabine, Gemtuzumab, Goserelin Acetate, Histrelin Acetate, Hydroxyurea, Ibritumomab, Idarubicin, Ifosfamide, Imatinib Mesylate, Interferon Alfa-2a, Interferon Alfa-2b, Irinotecan, Lenalidomide, Letrozole, Leucovorin, Leuprolide Acetate, Levamisole, Lomustine, Megestrol Acetate, Melphalan, Mercaptopurine, 6-MP, Mesna, Methotrexate, Methoxsalen, Mitomycin C, Mitotane, Mitoxantrone, Nandrolone, Nelarabine, Nofetumomab, Oprelvekin, Oxaliplatin, Paclitaxel, Palifermin, Pamidronate, Pegademase, Pegaspargase, Pegfilgrastim, Pemetrexed Disodium, Pentostatin, Pipobroman, Plicamycin, Porfimer Sodium, Procarbazine, Quinacrine, Rasburicase, Rituximab, Sargramostim, Sorafenib, Streptozocin, Sunitinib Maleate, Talc, Tamoxifen, Temozolomide, Teniposide, VM-26, Testolactone, Thioguanine, 6-TG, Thiotepa, Topotecan, Toremifene, Tositumomab, Trastuzumab, Tretinoin, ATRA, Uracil Mustard, Valrubicin, Vinblastine, Vincristine, Vinorelbine, Zoledronate, and Zoledronic acid, and combinations thereof.

24. The click-functionalized targeting group of claim 22, wherein the drug is a hydrophobic chemotherapeutic agent selected from the group consisting of Exemestance (aromasin), Camptosar (irinotecan), Ellence (epirubicin), Femara (Letrozole), Gleevac (imatinib mesylate), Lentaron (formestane), Cytadren/Orimeten (aminoglutethimide), Temodar, Proscar (finasteride), Viadur (leuprolide), Nexavar (Sorafenib), Kytril (Granisetron), Taxotere (Docetaxel), Taxol (paclitaxel), Kytril (Granisetron), Vesanoid (tretinoin) (retin A), XELODA (Capecitabine), Arimidex (Anastrozole), Casodex/Cosudex (Bicalutamide), Faslodex (Fulvestrant), Iressa (Gefitinib), Nolvadex, Istubal, Valodex (tamoxifen citrate), Tomudex (Raltitrexed), Zoladex (goserelin acetate), Leustatin (Cladribine), Velcade (bortezomib), Mylotarg (gemtuzumab ozogamicin), Alimta (pemetrexed), Gemzar (gemcitabine hydrochloride), Rituxan (rituximab), Revlimid (lenalidomide), Thalomid (thalidomide), Alkeran (melphalan), derivatives thereof, and combinations thereof.

25. A method for conjugating a click-functionalized targeting group with a compound of formula A: 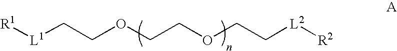
or a salt thereof, wherein:
n is 10-2500;
R¹ and R² are each independently hydrogen, halogen, NO₂, CN, N₃, —N═C═O, —C(R)═NN(R)₂, —P(O)(OR)₂, —P(O)(X)₂, a 9-30 membered crown ether, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, provided that one of R¹ and R² is a moiety suitable for click chemistry;
each X is independently halogen;
each R is independently hydrogen or an optionally substituted selected from aliphatic or a 3-8 membered, saturated, partially unsaturated, or aryl ring having 0-4 heteroatoms independently selected from nitrogen, oxygen, or sulfur; and
L¹ and L² are each independently a valence bond or a bivalent, saturated or unsaturated, straight or branched C₁₋₂ hydrocarbon chain, wherein 0-6 methylene units of L¹ and L² are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur, comprising the steps of:
(a) providing a compound of formula A,
(b) providing a click-functionalized targeting compound, and
(c) conjugating the compound of formula A to the targeting compound via click chemistry to form a conjugate thereof.

26. The method according to claim 25, wherein the conjugate is of formula A-1, A-2, A-3, or A-4:

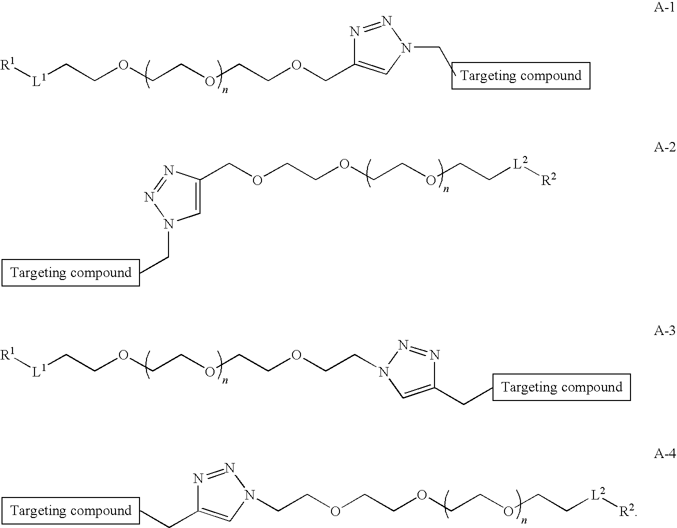

27. A method for conjugating a click-functionalized targeting group with a compound of formula B: 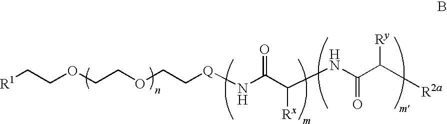
wherein:
n is 10-2500;
m is 0 to 1000;
m′ is 1 to 1000;
R^{x} is a natural or unnatural amino acid side-chain group that is capable of crosslinking;
R^{y} is a hydrophobic or ionic, natural or unnatural amino acid side-chain group;
R¹ is -Z(CH₂CH₂Y)ₚ(CH₂)ₜR³, wherein:
Z is —O—, —S—, —C≡C—, or —CH₂—;
each Y is independently —O— or —S—;
p is 0-10;
t is 0-10; and
R³ is —N₃ or alkyne;
Q is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
R^{2a} is a mono-protected amine, a di-protected amine, —N(R⁴)₂, —NR⁴C(O)R⁴, —NR⁴C(O)N(R⁴)₂, —NR⁴C(O)OR⁴, or —NR⁴SO₂R⁴, provided that one of R¹ and R^{2a} is a moiety suitable for click chemistry; and
each R⁴ is independently an optionally substituted group selected from hydrogen, aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or:
two R⁴ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur,
comprising the steps of:
(a) providing a compound of formula B,
(b) providing a click-functionalized targeting compound, and
(c) conjugating the compound of formula B to the targeting compound via click chemistry to form a conjugate thereof.

28. The method according to claim 27, wherein the conjugate is of formula B-1 or B-2: [chemical structure]


SEQUENCE LISTING (865 sequences)

SEQ ID NO: 1 - protein, 9 aa, Artificial Sequence
  Discovered from phage display experiment
CLSSRLDAC

SEQ ID NO: 2 - protein, 9 aa, Artificial Sequence
  Discovered from phage display experiment
CNSRLQLRC

SEQ ID NO: 3 - protein, 8 aa, Artificial Sequence
  Discovered from phage display experiment
CKDWGRIC

SEQ ID NO: 4 - protein, 8 aa, Artificial Sequence
  Discovered from phage display experiment
CTRITESC

SEQ ID NO: 5 - protein, 8 aa, Artificial Sequence
  Discovered from phage display experiment
CRTGTLFC

SEQ ID NO: 6 - protein, 9 aa, Artificial Sequence
  Discovered from phage display experiment
CRHWFDVVC

SEQ ID NO: 7 - protein, 8 aa, Artificial Sequence
  Discovered from phage display experiment
CGNPSYRC

SEQ ID NO: 8 - protein, 20 aa, Artificial Sequence
  Discovered from phage display experiment
YPCGGEAVAGVSSVRTMCSE

SEQ ID NO: 9 - protein, 18 aa, Artificial Sequence
  Discovered from phage display experiment
CNSRLHLRCCENWWGDVC

SEQ ID NO: 10 - protein, 7 aa, Artificial Sequence
  Discovered from phage display experiment
CVLRGGC

SEQ ID NO: 11 - protein, 8 aa, Artificial Sequence
  Discovered from phage display experiment
CLDWGRIC

SEQ ID NO: 12 - protein, 7 aa, Artificial Sequence
  Discovered from phage display experiment
CETLPAC

SEQ ID NO: 13 - protein, 8 aa, Artificial Sequence
  Discovered from phage display experiment
CGRSLDAC

SEQ ID NO: 14 - protein, 8 aa, Artificial Sequence
  Discovered from phage display experiment
CANAQSHC

SEQ ID NO: 15 - protein, 21 aa, Artificial Sequence
  Discovered from phage display experiment
WRCVLREGPAGGCAWFNRHRL

SEQ ID NO: 16 - protein, 20 aa, Artificial Sequence
  Discovered from phage display experiment
LNCDYQGTNPATSVSVPCTV

SEQ ID NO: 17 - protein, 21 aa, Artificial Sequence
  Discovered from phage display experiment
WRCVLREGPAGGGAWFNRHRL

SEQ ID NO: 18 - protein, 7 aa, Artificial Sequence
  Discovered from phage display experiment
CLPVASC

SEQ ID NO: 19 - protein, 8 aa, Artificial Sequence
  Discovered from phage display experiment
CKGRSSAC

SEQ ID NO: 20 - protein, 8 aa, Artificial Sequence
  Discovered from phage display experiment
CLGRSSVC

SEQ ID NO: 21 - protein, 8 aa, Artificial Sequence
  Discovered from phage display experiment
CMGRWRLC

SEQ ID NO: 22 - protein, 7 aa, Artificial Sequence
  Discovered from phage display experiment
CVAWLNC

SEQ ID NO: 23 - protein, 7 aa, Artificial Sequence
  Discovered from phage display experiment
CLMGVHC

SEQ ID NO: 24 - protein, 8 aa, Artificial Sequence
  Discovered from phage display experiment
CFVGHDLC

SEQ ID NO: 25 - protein, 7 aa, Artificial Sequence
  Discovered from phage display experiment
CKLMGEC

SEQ ID NO: 26 - protein, 7 aa, Artificial Sequence
  Discovered from phage display experiment
CGAREMC

SEQ ID NO: 27 - protein, 8 aa, Artificial Sequence
  Discovered from phage display experiment
CWARAQGC

SEQ ID NO: 28 - protein, 8 aa, Artificial Sequence
  Discovered from phage display experiment
CTSPGGSC

SEQ ID NO: 29 - protein, 8 aa, Artificial Sequence
  Discovered from phage display experiment
CVGECGGC

SEQ ID NO: 30 - protein, 7 aa, Artificial Sequence
  Discovered from phage display experiment
CRRFQDC

SEQ ID NO: 31 - protein, 8 aa, Artificial Sequence
  Discovered from phage display experiment
CKLLSGVC

SEQ ID NO: 32 - protein, 7 aa, Artificial Sequence
  Discovered from phage display experiment
CRCLNVC

SEQ ID NO: 33 - protein, 7 aa, Artificial Sequence
  Discovered from phage display experiment
GGGVFWQ

SEQ ID NO: 34 - protein, 7 aa, Artificial Sequence
  Discovered from phage display experiment
HGRVRPH

SEQ ID NO: 35 - protein, 7 aa, Artificial Sequence
  Discovered from phage display experiment
VVLVTSS

SEQ ID NO: 36 - protein, 5 aa, Artificial Sequence
  Discovered from phage display experiment
CRPPR

SEQ ID NO: 37 - protein, 8 aa, Artificial Sequence
  Discovered from phage display experiment
CLHRGNSC

SEQ ID NO: 38 - protein, 12 aa, Artificial Sequence
  Discovered from phage display experiment
CRSWNKADNRSC

SEQ ID NO: 39 - protein, 20 aa, Artificial Sequence
  Discovered from phage display experiment
WLSEAGPVVTVRALRGTGSW

SEQ ID NO: 40 - protein, 7 aa, Artificial Sequence
  Discovered from phage display experiment
YSGKWGW

SEQ ID NO: 41 - protein, 7 aa, Artificial Sequence
  Discovered from phage display experiment
VRRGSPQ

SEQ ID NO: 42 - protein, 7 aa, Artificial Sequence
  Discovered from phage display experiment
MRRDEQR

SEQ ID NO: 43 - protein, 7 aa, Artificial Sequence
  Discovered from phage display experiment
WELVARS

SEQ ID NO: 44 - protein, 7 aa, Artificial Sequence
  Discovered from phage display experiment
YAGFFLV

SEQ ID NO: 45 - protein, 7 aa, Artificial Sequence
  Discovered from phage display experiment
LRAVGRA

SEQ ID NO: 46 - protein, 7 aa, Artificial Sequence
  Discovered from phage display experiment
GISAVLS

SEQ ID NO: 47 - protein, 7 aa, Artificial Sequence
  Discovered from phage display experiment
LSPPYMW

SEQ ID NO: 48 - protein, 6 aa, Artificial Sequence
  Discovered from phage display experiment
GCRCWA

SEQ ID NO: 49 - protein, 7 aa, Artificial Sequence
  Discovered from phage display experiment
CVESTVA

SEQ ID NO: 50 - protein, 7 aa, Artificial Sequence
  Discovered from phage display experiment
GAVLPGE

SEQ ID NO: 51 - protein, 7 aa, Artificial Sequence
  Discovered from phage display experiment
RGDRPPY

SEQ ID NO: 52 - protein, 7 aa, Artificial Sequence
  Discovered from phage display experiment
RVRGPER

SEQ ID NO: 53 - protein, 7 aa, Artificial Sequence
  Discovered from phage display experiment
GVSASDW

SEQ ID NO: 54 - protein, 7 aa, Artificial Sequence
  Discovered from phage display experiment
RSGARSS

SEQ ID NO: 55 - protein, 7 aa, Artificial Sequence
  Discovered from phage display experiment
LCTAMTE

SEQ ID NO: 56 - protein, 7 aa, Artificial Sequence
  Discovered from phage display experiment
SKVWLLL

SEQ ID NO: 57 - protein, 7 aa, Artificial Sequence
  Discovered from phage display experiment
LVSEQLR

SEQ ID NO: 58 - protein, 7 aa, Artificial Sequence
  Discovered from phage display experiment
SRLSGGT

SEQ ID NO: 59 - protein, 7 aa, Artificial Sequence
  Discovered from phage display experiment
SRRQPLS

SEQ ID NO: 60 - protein, 7 aa, Artificial Sequence
  Discovered from phage display experiment
QVRRVPE

SEQ ID NO: 61 - protein, 6 aa, Artificial Sequence
  Discovered from phage display experiment
MVQSVG

SEQ ID NO: 62 - protein, 7 aa, Artificial Sequence
  Discovered from phage display experiment
MSPQLAT

SEQ ID NO: 63 - protein, 7 aa, Artificial Sequence
  Discovered from phage display experiment
WIEEAER

SEQ ID NO: 64 - protein, 7 aa, Artificial Sequence
  Discovered from phage display experiment
GGRGSWE

SEQ ID NO: 65 - protein, 7 aa, Artificial Sequence
  Discovered from phage display experiment
FRVRGSP

SEQ ID NO: 66 - protein, 6 aa, Artificial Sequence
  Discovered from phage display experiment
SLIDIP

SEQ ID NO: 67 - protein, 6 aa, Artificial Sequence
  Discovered from phage display experiment
NGRAHA

SEQ ID NO: 68 - protein, 7 aa, Artificial Sequence
  Discovered from phage display experiment
VVLVTSS

SEQ ID NO: 69 - protein, 5 aa, Artificial Sequence
  Discovered from phage display experiment
CRGDC

SEQ ID NO: 70 - protein, 4 aa, Artificial Sequence
  Discovered from phage display experiment
KRGD

SEQ ID NO: 71 - protein, 6 aa, Artificial Sequence
  Discovered from phage display experiment
RCDVVV

SEQ ID NO: 72 - protein, 10 aa, Artificial Sequence
  Discovered from phage display experiment
GACRGDCLGA

SEQ ID NO: 73 - protein, 15 aa, Artificial Sequence
  Discovered from phage display experiment
HRWMPHVFAVRQGAS

SEQ ID NO: 74 - protein, 6 aa, Artificial Sequence
  Discovered from phage display experiment
CRGDCA

SEQ ID NO: 75 - protein, 4 aa, Artificial Sequence
  Discovered from phage display experiment
RGDL

SEQ ID NO: 76 - protein, 6 aa, Artificial Sequence
  Discovered from phage display experiment
TIRSVD

SEQ ID NO: 77 - protein, 6 aa, Artificial Sequence
  Discovered from phage display experiment
DGRAHA

SEQ ID NO: 78 - protein, 6 aa, Artificial Sequence
  Discovered from phage display experiment
CRGDCL

SEQ ID NO: 79 - protein, 4 aa, Artificial Sequence
  Discovered from phage display experiment
RRGD

SEQ ID NO: 80 - protein, 15 aa, Artificial Sequence
  Discovered from phage display experiment
FGRIPSPLAYTYSFR

SEQ ID NO: 81 - protein, 15 aa, Artificial Sequence
  Discovered from phage display experiment
VSWFSRHRYSPFAVS

SEQ ID NO: 82 - protein, 11 aa, Artificial Sequence
  Discovered from phage display experiment
CSFGRGDIRNC

SEQ ID NO: 83 - protein, 11 aa, Artificial Sequence
  Discovered from phage display experiment
CSFGKGDNRIC

SEQ ID NO: 84 - protein, 11 aa, Artificial Sequence
  Discovered from phage display experiment
CSFGRVDDRNC

SEQ ID NO: 85 - protein, 11 aa, Artificial Sequence
  Discovered from phage display experiment
CSFGRSVDRNC

SEQ ID NO: 86 - protein, 11 aa, Artificial Sequence
  Discovered from phage display experiment
CSFGRWDARNC

SEQ ID NO: 87 - protein, 11 aa, Artificial Sequence
  Discovered from phage display experiment
CSFGRDDGRNC

SEQ ID NO: 88 - protein, 11 aa, Artificial Sequence
  Discovered from phage display experiment
CSFGRTDQRIC

SEQ ID NO: 89 - protein, 11 aa, Artificial Sequence
  Discovered from phage display experiment
CSFGRNDSRNC

SEQ ID NO: 90 - protein, 11 aa, Artificial Sequence
  Discovered from phage display experiment
CSFGRADRRNC

SEQ ID NO: 91 - protein, 11 aa, Artificial Sequence
  Discovered from phage display experiment
CSFGKRDMRNC

SEQ ID NO: 92 - protein, 11 aa, Artificial Sequence
  Discovered from phage display experiment
CSFGRQDVRNC

SEQ ID NO: 93 - protein, 9 aa, Artificial Sequence
  Discovered from phage display experiment
CDCRGDCFC

SEQ ID NO: 94 - protein, 13 aa, Artificial Sequence
  Discovered from phage display experiment
CNGRCVSGCAGRC

SEQ ID NO: 95 - protein, 7 aa, Artificial Sequence
  Discovered from phage display experiment
GVRTSIW

SEQ ID NO: 96 - protein, 7 aa, Artificial Sequence
  Discovered from phage display experiment
KLVNSSW

SEQ ID NO: 97 - protein, 7 aa, Artificial Sequence
  Discovered from phage display experiment
EVRSRLS

SEQ ID NO: 98 - protein, 7 aa, Artificial Sequence
  Discovered from phage display experiment
RPVGMRK

SEQ ID NO: 99 - protein, 7 aa, Artificial Sequence
  Discovered from phage display experiment
LCERVWR

SEQ ID NO: 100 - protein, 7 aa, Artificial Sequence
  Discovered from phage display experiment
RVGLVAR

SEQ ID NO: 101 - protein, 7 aa, Artificial Sequence
  Discovered from phage display experiment
FGSQAFV

SEQ ID NO: 102 - protein, 7 aa, Artificial Sequence
  Discovered from phage display experiment
AVKDYFR

SEQ ID NO: 103 - protein, 7 aa, Artificial Sequence
  Discovered from phage display experiment
FFAAVRS

SEQ ID NO: 104 - protein, 7 aa, Artificial Sequence
  Discovered from phage display experiment
WLERPEY

SEQ ID NO: 105 - protein, 7 aa, Artificial Sequence
  Discovered from phage display experiment
GGDVMWR

SEQ ID NO: 106 - protein, 7 aa, Artificial Sequence
  Discovered from phage display experiment
VRARLMS

SEQ ID NO: 107 - protein, 7 aa, Artificial Sequence
  Discovered from phage display experiment
RVRLVNL

SEQ ID NO: 108 - protein, 7 aa, Artificial Sequence
  Discovered from phage display experiment
TLRESGP

SEQ ID NO: 109 - protein, 7 aa, Artificial Sequence
  Discovered from phage display experiment
GLSGGRS

SEQ ID NO: 110 - protein, 9 aa, Artificial Sequence
  Discovered from phage display experiment
SWCEPGWCR

SEQ ID NO: 111 - protein, 8 aa, Artificial Sequence
  Discovered from phage display experiment
XLWLLXXG

SEQ ID NO: 112 - protein, 7 aa, Artificial Sequence
  Discovered from phage display experiment
SFTYWTN

SEQ ID NO: 113 - protein, 9 aa, Artificial Sequence
  Discovered from phage display experiment
CKSTHDRLC

SEQ ID NO: 114 - protein, 5 aa, Artificial Sequence
  Discovered from phage display experiment
XGSGL

SEQ ID NO: 115 - protein, 7 aa, Artificial Sequence
  Discovered from phage display experiment
EVQSAKW

SEQ ID NO: 116 - protein, 7 aa, Artificial Sequence
  Discovered from phage display experiment
GRLSVQV

SEQ ID NO: 117 - protein, 7 aa, Artificial Sequence
  Discovered from phage display experiment
FAVRVVG

SEQ ID NO: 118 - protein, 7 aa, Artificial Sequence
  Discovered from phage display experiment
GFYRMLG

SEQ ID NO: 119 - protein, 7 aa, Artificial Sequence
  Discovered from phage display experiment
GSRSLGA

SEQ ID NO: 120 - protein, 6 aa, Artificial Sequence
  Discovered from phage display experiment
GDELLA

SEQ ID NO: 121 - protein, 7 aa, Artificial Sequence
  Discovered from phage display experiment
GSEPMFR

SEQ ID NO: 122 - protein, 5 aa, Artificial Sequence
  Discovered from phage display experiment
WHQPL

SEQ ID NO: 123 - protein, 7 aa, Artificial Sequence
  Discovered from phage display experiment
RGRWLAL

SEQ ID NO: 124 - protein, 7 aa, Artificial Sequence
  Discovered from phage display experiment
LWLSGNW

SEQ ID NO: 125 - protein, 7 aa, Artificial Sequence
  Discovered from phage display experiment
WTFLERL

SEQ ID NO: 126 - protein, 6 aa, Artificial Sequence
  Discovered from phage display experiment
REVKES

SEQ ID NO: 127 - protein, 7 aa, Artificial Sequence
  Discovered from phage display experiment
GEWLGEC

SEQ ID NO: 128 - protein, 7 aa, Artificial Sequence
  Discovered from phage display experiment
PNPLMPL

SEQ ID NO: 129 - protein, 8 aa, Artificial Sequence
  Discovered from phage display experiment
DPRATPGS

SEQ ID NO: 130 - protein, 12 aa, Artificial Sequence
  Discovered from phage display experiment
CXFXXXYXYLMC

SEQ ID NO: 131 - protein, 13 aa, Artificial Sequence
  Discovered from phage display experiment
CVXYCXXXXCWXC

SEQ ID NO: 132 - protein, 7 aa, Artificial Sequence
  Discovered from phage display experiment
SLWYLGA

SEQ ID NO: 133 - protein, 7 aa, Artificial Sequence
  Discovered from phage display experiment
KRVYVLG

SEQ ID NO: 134 - protein, 7 aa, Artificial Sequence
  Discovered from phage display experiment
WKPASLS

SEQ ID NO: 135 - protein, 7 aa, Artificial Sequence
  Discovered from phage display experiment
LVRPLEG

SEQ ID NO: 136 - protein, 7 aa, Artificial Sequence
  Discovered from phage display experiment
EGRPMVY

SEQ ID NO: 137 - protein, 7 aa, Artificial Sequence
  Discovered from phage display experiment
RVWQGDV

SEQ ID NO: 138 - protein, 7 aa, Artificial Sequence
  Discovered from phage display experiment
FVWLVGS

SEQ ID NO: 139 - protein, 7 aa, Artificial Sequence
  Discovered from phage display experiment
VSFLEYR

SEQ ID NO: 140 - protein, 7 aa, Artificial Sequence
  Discovered from phage display experiment
SMSIARL

SEQ ID NO: 141 - protein, 7 aa, Artificial Sequence
  Discovered from phage display experiment
QVEEFPC

SEQ ID NO: 142 - protein, 7 aa, Artificial Sequence
  Discovered from phage display experiment
GPMLSVM

SEQ ID NO: 143 - protein, 7 aa, Artificial Sequence
  Discovered from phage display experiment
VLPGGQW

SEQ ID NO: 144 - protein, 7 aa, Artificial Sequence
  Discovered from phage display experiment
RTPAAVM

SEQ ID NO: 145 - protein, 7 aa, Artificial Sequence
  Discovered from phage display experiment
YVGGWEL

SEQ ID NO: 146 - protein, 10 aa, Artificial Sequence
  Discovered from phage display experiment
CVFXXXYXXC

SEQ ID NO: 147 - protein, 13 aa, Artificial Sequence
  Discovered from phage display experiment
CVXYCXXXXCYVC

SEQ ID NO: 148 - protein, 13 aa, Artificial Sequence
  Discovered from phage display experiment
CGFECVRQCPERC

SEQ ID NO: 149 - protein, 8 aa, Artificial Sequence
  Discovered from phage display experiment
CIKGNVNC

SEQ ID NO: 150 - protein, 8 aa, Artificial Sequence
  Discovered from phage display experiment
CLYIDRRC

SEQ ID NO: 151 - protein, 8 aa, Artificial Sequence
  Discovered from phage display experiment
CSKLMMTC

SEQ ID NO: 152 - protein, 8 aa, Artificial Sequence
  Discovered from phage display experiment
CNSDVDLC

SEQ ID NO: 153 - protein, 8 aa, Artificial Sequence
  Discovered from phage display experiment
CEKKLLYC

SEQ ID NO: 154 - protein, 12 aa, Artificial Sequence
  Discovered from phage display experiment
CVDSQSMKGLVC

SEQ ID NO: 155 - protein, 12 aa, Artificial Sequence
  Discovered from phage display experiment
CRPAQRDAGTSC

SEQ ID NO: 156 - protein, 12 aa, Artificial Sequence
  Discovered from phage display experiment
GGEVASNERIQC

SEQ ID NO: 157 - protein, 8 aa, Artificial Sequence
  Discovered from phage display experiment
CTLRDRNC

SEQ ID NO: 158 - protein, 8 aa, Artificial Sequence
  Discovered from phage display experiment
CRHESSSC

SEQ ID NO: 159 - protein, 8 aa, Artificial Sequence
  Discovered from phage display experiment
CYSLGADC

SEQ ID NO: 160 - protein, 8 aa, Artificial Sequence
  Discovered from phage display experiment
CTFRNASC

SEQ ID NO: 161 - protein, 9 aa, Artificial Sequence
  Discovered from phage display experiment
CRTHGYQGC

SEQ ID NO: 162 - protein, 9 aa, Artificial Sequence
  Discovered from phage display experiment
CKTNHMESC

SEQ ID NO: 163 - protein, 9 aa, Artificial Sequence
  Discovered from phage display experiment
CKDSAMTIC

SEQ ID NO: 164 - protein, 9 aa, Artificial Sequence
  Discovered from phage display experiment
CMSWDAVSC

SEQ ID NO: 165 - protein, 9 aa, Artificial Sequence
  Discovered from phage display experiment
CMSPQRSDC

SEQ ID NO: 166 - protein, 9 aa, Artificial Sequence
  Discovered from phage display experiment
CPQDIRRNC

SEQ ID NO: 167 - protein, 9 aa, Artificial Sequence
  Discovered from phage display experiment
CQTRNFAQC

SEQ ID NO: 168 - protein, 9 aa, Artificial Sequence
  Discovered from phage display experiment
CQDLNIMQC

SEQ ID NO: 169 - protein, 12 aa, Artificial Sequence
  Discovered from phage display experiment
CGYIDPNRISQC

SEQ ID NO: 170 - protein, 12 aa, Artificial Sequence
  Discovered from phage display experiment
CRLRSYGTLSLC

SEQ ID NO: 171 - protein, 9 aa, Artificial Sequence
  Discovered from phage display experiment
TRRTNNPLT

SEQ ID NO: 172 - protein, 12 aa, Artificial Sequence
  Discovered from phage display experiment
CTVNEAYKTRMC

SEQ ID NO: 173 - protein, 13 aa, Artificial Sequence
  Discovered from phage display experiment
CAGTCATGCNGVC

SEQ ID NO: 174 - protein, 12 aa, Artificial Sequence
  Discovered from phage display experiment
CPKARPAPQYKC

SEQ ID NO: 175 - protein, 12 aa, Artificial Sequence
  Discovered from phage display experiment
CQETRTEGRKKC

SEQ ID NO: 176 - protein, 8 aa, Artificial Sequence
  Discovered from phage display experiment
CHEGYLTC

SEQ ID NO: 177 - protein, 8 aa, Artificial Sequence
  Discovered from phage display experiment
CIGEVEVC

SEQ ID NO: 178 - protein, 8 aa, Artificial Sequence
  Discovered from phage display experiment
CLRPYLNC

SEQ ID NO: 179 - protein, 8 aa, Artificial Sequence
  Discovered from phage display experiment
CMELSKQG

SEQ ID NO: 180 - protein, 8 aa, Artificial Sequence
  Discovered from phage display experiment
CGNETLRC

SEQ ID NO: 181 - protein, 8 aa, Artificial Sequence
  Discovered from phage display experiment
CMGSEYWC

SEQ ID NO: 182 - protein, 8 aa, Artificial Sequence
  Discovered from phage display experiment
CAHQHIQC

SEQ ID NO: 183 - protein, 8 aa, Artificial Sequence
  Discovered from phage display experiment
CAQNMLCC

SEQ ID NO: 184 - protein, 12 aa, Artificial Sequence
  Discovered from phage display experiment
CADYDLALGLMC

SEQ ID NO: 185 - protein, 12 aa, Artificial Sequence
  Discovered from phage display experiment
CSSHQGGFQHGC

SEQ ID NO: 186 - protein, 12 aa, Artificial Sequence
  Discovered from phage display experiment
CRPWHNQAHTEC

SEQ ID NO: 187 - protein, 12 aa, Artificial Sequence
  Discovered from phage display experiment
CSEAASRMIGVC

SEQ ID NO: 188 - protein, 12 aa, Artificial Sequence
  Discovered from phage display experiment
CWDADQIEGIKC

SEQ ID NO: 189 - protein, 12 aa, Artificial Sequence
  Discovered from phage display experiment
CRLQTMGQGQSC

SEQ ID NO: 190 - protein, 12 aa, Artificial Sequence
  Discovered from phage display experiment
CGGRDRGTYGPC

SEQ ID NO: 191 - protein, 12 aa, Artificial Sequence
  Discovered from phage display experiment
CNSKSSAELEKC

SEQ ID NO: 192 - protein, 12 aa, Artificial Sequence
  Discovered from phage display experiment
CRGKPLANFEDC

SEQ ID NO: 193 - protein, 12 aa, Artificial Sequence
  Discovered from phage display experiment
CRDRGDRMKSLC

SEQ ID NO: 194 - protein, 12 aa, Artificial Sequence
  Discovered from phage display experiment
CSFGTHDTEPHC

SEQ ID NO: 195 - protein, 12 aa, Artificial Sequence
  Discovered from phage display experiment
CWEEHPSIKWWC

SEQ ID NO: 196 - protein, 9 aa, Artificial Sequence
  Discovered from phage display experiment
CIFREANVC

SEQ ID NO: 197 - protein, 9 aa, Artificial Sequence
  Discovered from phage display experiment
CTRSTNTGC

SEQ ID NO: 198 - protein, 13 aa, Artificial Sequence
  Discovered from phage display experiment
CLVGCEVGCSPAC

SEQ ID NO: 199 - protein, 13 aa, Artificial Sequence
  Discovered from phage display experiment
CDTSCENNCQGPC

SEQ ID NO: 200 - protein, 13 aa, Artificial Sequence
  Discovered from phage display experiment
CRGDCGIGCRRLC

SEQ ID NO: 201 - protein, 13 aa, Artificial Sequence
  Discovered from phage display experiment
CSEGCGPVCWPEC

SEQ ID NO: 202 - protein, 16 aa, Artificial Sequence
  Discovered from phage display experiment
RNVPPIFNDVYYWIAF

SEQ ID NO: 203 - protein, 20 aa, Artificial Sequence
  Discovered from phage display experiment
VSQTMRQTAVPLLWFWTGSL

SEQ ID NO: 204 - protein, 20 aa, Artificial Sequence
  Discovered from phage display experiment
RGDLATLRQLAQEDGVVGVR

SEQ ID NO: 205 - protein, 8 aa, Artificial Sequence
  Discovered from phage display experiment
CGFELETC

SEQ ID NO: 206 - protein, 8 aa, Artificial Sequence
  Discovered from phage display experiment
CVGNLSMC

SEQ ID NO: 207 - protein, 8 aa, Artificial Sequence
  Discovered from phage display experiment
CKGQRDFC

SEQ ID NO: 208 - protein, 8 aa, Artificial Sequence
  Discovered from phage display experiment
CNMGLTRC

SEQ ID NO: 209 - protein, 8 aa, Artificial Sequence
  Discovered from phage display experiment
CGTFGARC

SEQ ID NO: 210 - protein, 12 aa, Artificial Sequence
  Discovered from phage display experiment
CSAHSQEMNVNC

SEQ ID NO: 211 - protein, 13 aa, Artificial Sequence
  Discovered from phage display experiment
CGFECVRQCPERC

SEQ ID NO: 212 - protein, 13 aa, Artificial Sequence
  Discovered from phage display experiment
CRSGCVEGCGGRC

SEQ ID NO: 213 - protein, 13 aa, Artificial Sequence
  Discovered from phage display experiment
CGGECGWECEVSC

SEQ ID NO: 214 - protein, 13 aa, Artificial Sequence
  Discovered from phage display experiment
CKWLCLLLCAVAC

SEQ ID NO: 215 - protein, 13 aa, Artificial Sequence
  Discovered from phage display experiment
CGAACGVGCGGRC

SEQ ID NO: 216 - protein, 13 aa, Artificial Sequence
  Discovered from phage display experiment
CGASCALGCRAYC

SEQ ID NO: 217 - protein, 13 aa, Artificial Sequence
  Discovered from phage display experiment
CSRQCRGACGQPC

SEQ ID NO: 218 - protein, 13 aa, Artificial Sequence
  Discovered from phage display experiment
CAGGGAVRCGGTC

SEQ ID NO: 219 - protein, 13 aa, Artificial Sequence
  Discovered from phage display experiment
CGRPCVGECRMGC

SEQ ID NO: 220 - protein, 12 aa, Artificial Sequence
  Discovered from phage display experiment
CVLNFKNQARDC

SEQ ID NO: 221 - protein, 12 aa, Artificial Sequence
  Discovered from phage display experiment
CEGHSMRGYGLC

SEQ ID NO: 222 - protein, 12 aa, Artificial Sequence
  Discovered from phage display experiment
CDNTCTYGVDDC

SEQ ID NO: 223 - protein, 13 aa, Artificial Sequence
  Discovered from phage display experiment
CGAACGVGCRGRC

SEQ ID NO: 224 - protein, 13 aa, Artificial Sequence
  Discovered from phage display experiment
CLVGCRLSCGGEC

SEQ ID NO: 225 - protein, 13 aa, Artificial Sequence
  Discovered from phage display experiment
CYWWCDGVCALQC

SEQ ID NO: 226 - protein, 8 aa, Artificial Sequence
  Discovered from phage display experiment
CRISAHPC

SEQ ID NO: 227 - protein, 8 aa, Artificial Sequence
  Discovered from phage display experiment
CSYPKILC

SEQ ID NO: 228 - protein, 8 aa, Artificial Sequence
  Discovered from phage display experiment
CSEPSGTC

SEQ ID NO: 229 - protein, 8 aa, Artificial Sequence
  Discovered from phage display experiment
CTLSNRFC

SEQ ID NO: 230 - protein, 8 aa, Artificial Sequence
  Discovered from phage display experiment
CLFSDENC

SEQ ID NO: 231 - protein, 9 aa, Artificial Sequence
  Discovered from phage display experiment
CWRGDRKIC

SEQ ID NO: 232 - protein, 12 aa, Artificial Sequence
  Discovered from phage display experiment
CCFTNFDCYLGC

SEQ ID NO: 233 - protein, 9 aa, Artificial Sequence
  Discovered from phage display experiment
CYEEKSQSC

SEQ ID NO: 234 - protein, 13 aa, Artificial Sequence
  Discovered from phage display experiment
CGGACGGVCTGGC

SEQ ID NO: 235 - protein, 9 aa, Artificial Sequence
  Discovered from phage display experiment
CLHSPRSKC

SEQ ID NO: 236 - protein, 9 aa, Artificial Sequence
  Discovered from phage display experiment
CLYTKEQRC

SEQ ID NO: 237 - protein, 9 aa, Artificial Sequence
  Discovered from phage display experiment
CTGHLSTDC

SEQ ID NO: 238 - protein, 13 aa, Artificial Sequence
  Discovered from phage display experiment
CIARCGGACGRHC

SEQ ID NO: 239 - protein, 13 aa, Artificial Sequence
  Discovered from phage display experiment
CGVGCPGLCGGAC

SEQ ID NO: 240 - protein, 9 aa, Artificial Sequence
  Discovered from phage display experiment
CLAKENVVC

SEQ ID NO: 241 - protein, 13 aa, Artificial Sequence
  Discovered from phage display experiment
CSGSCRRGCGIDC

SEQ ID NO: 242 - protein, 8 aa, Artificial Sequence
  Discovered from phage display experiment
CKGQGDWC

SEQ ID NO: 243 - protein, 13 aa, Artificial Sequence
  Discovered from phage display experiment
CPRTCGAACASPC

SEQ ID NO: 244 - protein, 9 aa, Artificial Sequence
  Discovered from phage display experiment
CERVVGSSC

SEQ ID NO: 245 - protein, 9 aa, Artificial Sequence
  Discovered from phage display experiment
CKWSRLHSC

SEQ ID NO: 246 - protein, 14 aa, Artificial Sequence
  Discovered from phage display experiment
QPFMQCLCIYDASC

SEQ ID NO: 247 - protein, 14 aa, Artificial Sequence
  Discovered from phage display experiment
VFRVRPWYQSTSQS

SEQ ID NO: 248 - protein, 20 aa, Artificial Sequence
  Discovered from phage display experiment
MTVCNASQRQAHAQATAVSL

SEQ ID NO: 249 - protein, 13 aa, Artificial Sequence
  Discovered from phage display experiment
CVGACDLKCTGGC

SEQ ID NO: 250 - protein, 11 aa, Artificial Sequence
  Discovered from phage display experiment
CSTLCGLRCMG

SEQ ID NO: 251 - protein, 13 aa, Artificial Sequence
  Discovered from phage display experiment
CSSGCSKNCLEMC

SEQ ID NO: 252 - protein, 13 aa, Artificial Sequence
  Discovered from phage display experiment
CQGGCGVSCPIFC

SEQ ID NO: 253 - protein, 13 aa, Artificial Sequence
  Discovered from phage display experiment
CGFGCSGSCQMQC

SEQ ID NO: 254 - protein, 13 aa, Artificial Sequence
  Discovered from phage display experiment
CTMGCTAGCAFAC

SEQ ID NO: 255 - protein, 13 aa, Artificial Sequence
  Discovered from phage display experiment
CNQGCSGSCDVMC

SEQ ID NO: 256 - protein, 13 aa, Artificial Sequence
  Discovered from phage display experiment
CVEGCSSGCGPGC

SEQ ID NO: 257 - protein, 13 aa, Artificial Sequence
  Discovered from phage display experiment
CYADCEGTCGMVC

SEQ ID NO: 258 - protein, 13 aa, Artificial Sequence
  Discovered from phage display experiment
CWNICPGGCRALC

SEQ ID NO: 259 - protein, 13 aa, Artificial Sequence
  Discovered from phage display experiment
CMPRCGVNCKWAC

SEQ ID NO: 260 - protein, 13 aa, Artificial Sequence
  Discovered from phage display experiment
CGGGCQWGCAGEC

SEQ ID NO: 261 - protein, 13 aa, Artificial Sequence
  Discovered from phage display experiment
CPSNCVALCTSGC

SEQ ID NO: 262 - protein, 5 aa, Artificial Sequence
  Discovered from phage display experiment
CGKRK

SEQ ID NO: 263 - protein, 12 aa, Artificial Sequence
  Discovered from phage display experiment
TSPLNIHNGQKL

SEQ ID NO: 264 - protein, 13 aa, Artificial Sequence
  Discovered from phage display experiment
CRVVCADGCRLTC

SEQ ID NO: 265 - protein, 13 aa, Artificial Sequence
  Discovered from phage display experiment
CFTFCEYHCQLTC

SEQ ID NO: 266 - protein, 13 aa, Artificial Sequence
  Discovered from phage display experiment
CGRPCRGGCAASC

SEQ ID NO: 267 - protein, 13 aa, Artificial Sequence
  Discovered from phage display experiment
CSTLCGLRCMGTC

SEQ ID NO: 268 - protein, 12 aa, Artificial Sequence
  Discovered from phage display experiment
GPGCEEECQPAC

SEQ ID NO: 269 - protein, 13 aa, Artificial Sequence
  Discovered from phage display experiment
CKGTCVLGCSEEC

SEQ ID NO: 270 - protein, 13 aa, Artificial Sequence
  Discovered from phage display experiment
CVALCREACGEGC

SEQ ID NO: 271 - protein, 13 aa, Artificial Sequence
  Discovered from phage display experiment
CAVRCDGSCVPEC

SEQ ID NO: 272 - protein, 13 aa, Artificial Sequence
  Discovered from phage display experiment
CRVVCADGCRFIC

SEQ ID NO: 273 - protein, 13 aa, Artificial Sequence
  Discovered from phage display experiment
CEGKCGLTCECTC

SEQ ID NO: 274 - protein, 13 aa, Artificial Sequence
  Discovered from phage display experiment
CASGCSESCYVGC

SEQ ID NO: 275 - protein, 13 aa, Artificial Sequence
  Discovered from phage display experiment
CSVRCKSVCIGLC

SEQ ID NO: 276 - protein, 9 aa, Artificial Sequence
  Discovered from phage display experiment
CSRPRRSEC

SEQ ID NO: 277 - protein, 5 aa, Artificial Sequence
  Discovered from phage display experiment
CDTRL

SEQ ID NO: 278 - protein, 9 aa, Artificial Sequence
  Discovered from phage display experiment
CRRIWYAVC

SEQ ID NO: 279 - protein, 9 aa, Artificial Sequence
  Discovered from phage display experiment
CSCFRDVCC

SEQ ID NO: 280 - protein, 8 aa, Artificial Sequence
  Discovered from phage display experiment
CTDNRVGS

SEQ ID NO: 281 - protein, 12 aa, Artificial Sequence
  Discovered from phage display experiment
CTSDISWWDYKC

SEQ ID NO: 282 - protein, 13 aa, Artificial Sequence
  Discovered from phage display experiment
CVGDCIGSCWMFC

SEQ ID NO: 283 - protein, 8 aa, Artificial Sequence
  Discovered from phage display experiment
CVSGHLNC

SEQ ID NO: 284 - protein, 9 aa, Artificial Sequence
  Discovered from phage display experiment
CYTGETWTC

SEQ ID NO: 285 - protein, 9 aa, Artificial Sequence
  Discovered from phage display experiment
CDCRGDCFC

SEQ ID NO: 286 - protein, 12 aa, Artificial Sequence
  Discovered from phage display experiment
CERSQSKGVHHC

SEQ ID NO: 287 - protein, 8 aa, Artificial Sequence
  Discovered from phage display experiment
CFWHNRAC

SEQ ID NO: 288 - protein, 8 aa, Artificial Sequence
  Discovered from phage display experiment
CGEFKVGC

SEQ ID NO: 289 - protein, 13 aa, Artificial Sequence
  Discovered from phage display experiment
CGPGYQAQCSLRC

SEQ ID NO: 290 - protein, 13 aa, Artificial Sequence
  Discovered from phage display experiment
CHMGCVSPCAYVC

SEQ ID NO: 291 - protein, 8 aa, Artificial Sequence
  Discovered from phage display experiment
CISRPYFC

SEQ ID NO: 292 - protein, 12 aa, Artificial Sequence
  Discovered from phage display experiment
CKERPSNGLSAC

SEQ ID NO: 293 - protein, 13 aa, Artificial Sequence
  Discovered from phage display experiment
CKSGCGVACRHMC

SEQ ID NO: 294 - protein, 8 aa, Artificial Sequence
  Discovered from phage display experiment
CMDSQSSC

SEQ ID NO: 295 - protein, 12 aa, Artificial Sequence
  Discovered from phage display experiment
CNIPVTTPIFGC

SEQ ID NO: 296 - protein, 12 aa, Artificial Sequence
  Discovered from phage display experiment
CNRKNSNEQRAC

SEQ ID NO: 297 - protein, 9 aa, Artificial Sequence
  Discovered from phage display experiment
CQIRPIDKC

SEQ ID NO: 298 - protein, 12 aa, Artificial Sequence
  Discovered from phage display experiment
CGRFDTAPQRGC

SEQ ID NO: 299 - protein, 8 aa, Artificial Sequence
  Discovered from phage display experiment
CLLNYTYC

SEQ ID NO: 300 - protein, 8 aa, Artificial Sequence
  Discovered from phage display experiment
CMSLGNNC

SEQ ID NO: 301 - protein, 9 aa, Artificial Sequence
  Discovered from phage display experiment
CQASASDHC

SEQ ID NO: 302 - protein, 12 aa, Artificial Sequence
  Discovered from phage display experiment
CQRVNSVENASC

SEQ ID NO: 303 - protein, 8 aa, Artificial Sequence
  Discovered from phage display experiment
CRRHMERC

SEQ ID NO: 304 - protein, 8 aa, Artificial Sequence
  Discovered from phage display experiment
CTHLVTLC

SEQ ID NO: 305 - protein, 9 aa, Artificial Sequence
  Discovered from phage display experiment
CVTSNLRVC

SEQ ID NO: 306 - protein, 9 aa, Artificial Sequence
  Discovered from phage display experiment
CSAYTTSPC

SEQ ID NO: 307 - protein, 8 aa, Artificial Sequence
  Discovered from phage display experiment
CTDKSWPC

SEQ ID NO: 308 - protein, 8 aa, Artificial Sequence
  Discovered from phage display experiment
CTIADFPC

SEQ ID NO: 309 - protein, 8 aa, Artificial Sequence
  Discovered from phage display experiment
CTVDNELC

SEQ ID NO: 310 - protein, 8 aa, Artificial Sequence
  Discovered from phage display experiment
CVKFTYDC

SEQ ID NO: 311 - protein, 9 aa, Artificial Sequence
  Discovered from phage display experiment
CYGESQQMC

SEQ ID NO: 312 - protein, 8 aa, Artificial Sequence
  Discovered from phage display experiment
CAVSIPRC

SEQ ID NO: 313 - protein, 13 aa, Artificial Sequence
  Discovered from phage display experiment
CGDVCPSECPGWC

SEQ ID NO: 314 - protein, 13 aa, Artificial Sequence
  Discovered from phage display experiment
CGLDCLGDCSGAC

SEQ ID NO: 315 - protein, 13 aa, Artificial Sequence
  Discovered from phage display experiment
CGSHCGQLCKSLC

SEQ ID NO: 316 - protein, 9 aa, Artificial Sequence
  Discovered from phage display experiment
CILSYDNPC

SEQ ID NO: 317 - protein, 12 aa, Artificial Sequence
  Discovered from phage display experiment
CKERLEYTRGVC

SEQ ID NO: 318 - protein, 8 aa, Artificial Sequence
  Discovered from phage display experiment
CKPFRTEC

SEQ ID NO: 319 - protein, 9 aa, Artificial Sequence
  Discovered from phage display experiment
CLKPGGQEC

SEQ ID NO: 320 - protein, 8 aa, Artificial Sequence
  Discovered from phage display experiment
CMNILSGC

SEQ ID NO: 321 - protein, 12 aa, Artificial Sequence
  Discovered from phage display experiment
CNQRTNRESGNC

SEQ ID NO: 322 - protein, 8 aa, Artificial Sequence
  Discovered from phage display experiment
CNRMEMPC

SEQ ID NO: 323 - protein, 9 aa, Artificial Sequence
  Discovered from phage display experiment
CAIDIGGAC

SEQ ID NO: 324 - protein, 9 aa, Artificial Sequence
  Discovered from phage display experiment
CKRANRLSC

SEQ ID NO: 325 - protein, 9 aa, Artificial Sequence
  Discovered from phage display experiment
CLNGLVSMC

SEQ ID NO: 326 - protein, 9 aa, Artificial Sequence
  Discovered from phage display experiment
CNRNRMTPC

SEQ ID NO: 327 - protein, 9 aa, Artificial Sequence
  Discovered from phage display experiment
CQLINSSPC

SEQ ID NO: 328 - protein, 8 aa, Artificial Sequence
  Discovered from phage display experiment
CRKEHYPC

SEQ ID NO: 329 - protein, 9 aa, Artificial Sequence
  Discovered from phage display experiment
CSGRPFKYC

SEQ ID NO: 330 - protein, 9 aa, Artificial Sequence
  Discovered from phage display experiment
CTSSPAYNC

SEQ ID NO: 331 - protein, 9 aa, Artificial Sequence
  Discovered from phage display experiment
CWDSGSHIC

SEQ ID NO: 332 - protein, 9 aa, Artificial Sequence
  Discovered from phage display experiment
CERSHGRLC

SEQ ID NO: 333 - protein, 8 aa, Artificial Sequence
  Discovered from phage display experiment
CINCLSQC

SEQ ID NO: 334 - protein, 8 aa, Artificial Sequence
  Discovered from phage display experiment
CNSRSENC

SEQ ID NO: 335 - protein, 8 aa, Artificial Sequence
  Discovered from phage display experiment
CSHHDTNC

SEQ ID NO: 336 - protein, 8 aa, Artificial Sequence
  Discovered from phage display experiment
CYAGSPLC

SEQ ID NO: 337 - protein, 8 aa, Artificial Sequence
  Discovered from phage display experiment
CQWSMNVC

SEQ ID NO: 338 - protein, 9 aa, Artificial Sequence
  Discovered from phage display experiment
CRDVVSVIC

SEQ ID NO: 339 - protein, 9 aa, Artificial Sequence
  Discovered from phage display experiment
CGNLLTRRC

SEQ ID NO: 340 - protein, 9 aa, Artificial Sequence
  Discovered from phage display experiment
CLRHDFYVC

SEQ ID NO: 341 - protein, 8 aa, Artificial Sequence
  Discovered from phage display experiment
CRYKGPSC

SEQ ID NO: 342 - protein, 8 aa, Artificial Sequence
  Discovered from phage display experiment
CSRWYTTC

SEQ ID NO: 343 - protein, 8 aa, Artificial Sequence
  Discovered from phage display experiment
CQTTSWNC

SEQ ID NO: 344 - protein, 12 aa, Artificial Sequence
  Discovered from phage display experiment
CRARIRAEDISC

SEQ ID NO: 345 - protein, 8 aa, Artificial Sequence
  Discovered from phage display experiment
CRREYSAC

SEQ ID NO: 346 - protein, 13 aa, Artificial Sequence
  Discovered from phage display experiment
CDSLCGGACAARC

SEQ ID NO: 347 - protein, 8 aa, Artificial Sequence
  Discovered from phage display experiment
CFKSTLLC

SEQ ID NO: 348 - protein, 9 aa, Artificial Sequence
  Discovered from phage display experiment
EICQLGSCT

SEQ ID NO: 349 - protein, 9 aa, Artificial Sequence
  Discovered from phage display experiment
RKCLRPDCG

SEQ ID NO: 350 - protein, 9 aa, Artificial Sequence
  Discovered from phage display experiment
LACFVTGCL

SEQ ID NO: 351 - protein, 9 aa, Artificial Sequence
  Discovered from phage display experiment
DMCWLIGCG

SEQ ID NO: 352 - protein, 9 aa, Artificial Sequence
  Discovered from phage display experiment
QRCPRSFCL

SEQ ID NO: 353 - protein, 9 aa, Artificial Sequence
  Discovered from phage display experiment
RECTNEICY

SEQ ID NO: 354 - protein, 9 aa, Artificial Sequence
  Discovered from phage display experiment
SCVFCDWLS

SEQ ID NO: 355 - protein, 9 aa, Artificial Sequence
  Discovered from phage display experiment
QNCPVTRCV

SEQ ID NO: 356 - protein, 8 aa, Artificial Sequence
  Discovered from phage display experiment
CDNREMSC

SEQ ID NO: 357 - protein, 8 aa, Artificial Sequence
  Discovered from phage display experiment
CGEYGREC

SEQ ID NO: 358 - protein, 9 aa, Artificial Sequence
  Discovered from phage display experiment
CKKRLLNVC

SEQ ID NO: 359 - protein, 8 aa, Artificial Sequence
  Discovered from phage display experiment
CMTGRVTC

SEQ ID NO: 360 - protein, 8 aa, Artificial Sequence
  Discovered from phage display experiment
CPDLLVAC

SEQ ID NO: 361 - protein, 9 aa, Artificial Sequence
  Discovered from phage display experiment
CSKAYDLAC

SEQ ID NO: 362 - protein, 9 aa, Artificial Sequence
  Discovered from phage display experiment
CTLKHTAMC

SEQ ID NO: 363 - protein, 8 aa, Artificial Sequence
  Discovered from phage display experiment
CTTEIDYC

SEQ ID NO: 364 - protein, 7 aa, Artificial Sequence
  Discovered from phage display experiment
CRGRRST

SEQ ID NO: 365 - protein, 19 aa, Artificial Sequence
  Discovered from phage display experiment
BCDDDGQRLGNQWAVGHLM

SEQ ID NO: 366 - protein, 9 aa, Artificial Sequence
  Discovered from phage display experiment
CHVLWSTRC

SEQ ID NO: 367 - protein, 20 aa, Artificial Sequence
  Discovered from phage display experiment
GAWEAVRDRIAEWGSWGIPS

SEQ ID NO: 368 - protein, 9 aa, Artificial Sequence
  Discovered from phage display experiment
QSCLWRICI

SEQ ID NO: 369 - protein, 9 aa, Artificial Sequence
  Discovered from phage display experiment
WRCEGFNCQ

SEQ ID NO: 370 - protein, 9 aa, Artificial Sequence
  Discovered from phage display experiment
SWCEPGWCR

SEQ ID NO: 371 - protein, 9 aa, Artificial Sequence
  Discovered from phage display experiment
GLCNGATCM

SEQ ID NO: 372 - protein, 9 aa, Artificial Sequence
  Discovered from phage display experiment
SGCRTMVCV

SEQ ID NO: 373 - protein, 9 aa, Artificial Sequence
  Discovered from phage display experiment
LSCAPVICG

SEQ ID NO: 374 - protein, 9 aa, Artificial Sequence
  Discovered from phage display experiment
NECLMISCR

SEQ ID NO: 375 - protein, 9 aa, Artificial Sequence
  Discovered from phage display experiment
WACEELSCF

SEQ ID NO: 376 - protein, 9 aa, Artificial Sequence
  Discovered from phage display experiment
CATLTNDEC

SEQ ID NO: 377 - protein, 8 aa, Artificial Sequence
  Discovered from phage display experiment
CFMDHSNC

SEQ ID NO: 378 - protein, 9 aa, Artificial Sequence
  Discovered from phage display experiment
CHMKRDRTC

SEQ ID NO: 379 - protein, 8 aa, Artificial Sequence
  Discovered from phage display experiment
CLDYHPKC

SEQ ID NO: 380 - protein, 8 aa, Artificial Sequence
  Discovered from phage display experiment
CNKIVRRC

SEQ ID NO: 381 - protein, 9 aa, Artificial Sequence
  Discovered from phage display experiment
CSDTQSIGC

SEQ ID NO: 382 - protein, 9 aa, Artificial Sequence
  Discovered from phage display experiment
CSKKGPSYC

SEQ ID NO: 383 - protein, 8 aa, Artificial Sequence
  Discovered from phage display experiment
CTQHIANC

SEQ ID NO: 384 - protein, 9 aa, Artificial Sequence
  Discovered from phage display experiment
CVGRSGELC

SEQ ID NO: 385 - protein, 7 aa, Artificial Sequence
  Discovered from phage display experiment
CKAAKNK

SEQ ID NO: 386 - protein, 9 aa, Artificial Sequence
  Discovered from phage display experiment
CVSNPRWKC

SEQ ID NO: 387 - protein, 20 aa, Artificial Sequence
  Discovered from phage display experiment
LSGTPERSGQAVKVKLKAIP

SEQ ID NO: 388 - protein, 9 aa, Artificial Sequence
  Discovered from phage display experiment
LGCFPSWCG

SEQ ID NO: 389 - protein, 9 aa, Artificial Sequence
  Discovered from phage display experiment
RLCSWGGCA

SEQ ID NO: 390 - protein, 7 aa, Artificial Sequence
  Discovered from phage display experiment
ARRGWTL

SEQ ID NO: 391 - protein, 7 aa, Artificial Sequence
  Discovered from phage display experiment
QLTGGCL

SEQ ID NO: 392 - protein, 7 aa, Artificial Sequence
  Discovered from phage display experiment
KAYFRWR

SEQ ID NO: 393 - protein, 7 aa, Artificial Sequence
  Discovered from phage display experiment
VGSFIYS

SEQ ID NO: 394 - protein, 7 aa, Artificial Sequence
  Discovered from phage display experiment
LSTVLWF

SEQ ID NO: 395 - protein, 7 aa, Artificial Sequence
  Discovered from phage display experiment
GRSSLAC

SEQ ID NO: 396 - protein, 7 aa, Artificial Sequence
  Discovered from phage display experiment
CGGAGAR

SEQ ID NO: 397 - protein, 7 aa, Artificial Sequence
  Discovered from phage display experiment
DFLRCRV

SEQ ID NO: 398 - protein, 7 aa, Artificial Sequence
  Discovered from phage display experiment
RALYDAL

SEQ ID NO: 399 - protein, 7 aa, Artificial Sequence
  Discovered from phage display experiment
GMAVSSW

SEQ ID NO: 400 - protein, 7 aa, Artificial Sequence
  Discovered from phage display experiment
WQSVVRV

SEQ ID NO: 401 - protein, 7 aa, Artificial Sequence
  Discovered from phage display experiment
CGNGHSC

SEQ ID NO: 402 - protein, 7 aa, Artificial Sequence
  Discovered from phage display experiment
SLRPDNG

SEQ ID NO: 403 - protein, 12 aa, Artificial Sequence
  Discovered from phage display experiment
TACHQHVRMVRP

SEQ ID NO: 404 - protein, 7 aa, Artificial Sequence
  Discovered from phage display experiment
SRRFVGG

SEQ ID NO: 405 - protein, 7 aa, Artificial Sequence
  Discovered from phage display experiment
ALERRSL

SEQ ID NO: 406 - protein, 7 aa, Artificial Sequence
  Discovered from phage display experiment
RWLAWTV

SEQ ID NO: 407 - protein, 7 aa, Artificial Sequence
  Discovered from phage display experiment
LSLLGIA

SEQ ID NO: 408 - protein, 7 aa, Artificial Sequence
  Discovered from phage display experiment
SLAMRDS

SEQ ID NO: 409 - protein, 7 aa, Artificial Sequence
  Discovered from phage display experiment
SELLGDA

SEQ ID NO: 410 - protein, 7 aa, Artificial Sequence
  Discovered from phage display experiment
WRQNMPL

SEQ ID NO: 411 - protein, 7 aa, Artificial Sequence
  Discovered from phage display experiment
QAGLRCH

SEQ ID NO: 412 - protein, 7 aa, Artificial Sequence
  Discovered from phage display experiment
WVSVLGF

SEQ ID NO: 413 - protein, 7 aa, Artificial Sequence
  Discovered from phage display experiment
SWFFLVA

SEQ ID NO: 414 - protein, 7 aa, Artificial Sequence
  Discovered from phage display experiment
VKSVCRT

SEQ ID NO: 415 - protein, 7 aa, Artificial Sequence
  Discovered from phage display experiment
AEMEGRD

SEQ ID NO: 416 - protein, 7 aa, Artificial Sequence
  Discovered from phage display experiment
PAMGLIR

SEQ ID NO: 417 - protein, 9 aa, Artificial Sequence
  Discovered from phage display experiment
WGCKLRFCS

SEQ ID NO: 418 - protein, 9 aa, Artificial Sequence
  Discovered from phage display experiment
GICATVKCS

SEQ ID NO: 419 - protein, 9 aa, Artificial Sequence
  Discovered from phage display experiment
TTCMSQLCL

SEQ ID NO: 420 - protein, 9 aa, Artificial Sequence
  Discovered from phage display experiment
GCVRRLLCN

SEQ ID NO: 421 - protein, 9 aa, Artificial Sequence
  Discovered from phage display experiment
KYCTPVECL

SEQ ID NO: 422 - protein, 8 aa, Artificial Sequence
  Discovered from phage display experiment
MCPQRNCL

SEQ ID NO: 423 - protein, 9 aa, Artificial Sequence
  Discovered from phage display experiment
AGCSVTVCG

SEQ ID NO: 424 - protein, 9 aa, Artificial Sequence
  Discovered from phage display experiment
GSCSMFPCS

SEQ ID NO: 425 - protein, 9 aa, Artificial Sequence
  Discovered from phage display experiment
SECAYRACS

SEQ ID NO: 426 - protein, 9 aa, Artificial Sequence
  Discovered from phage display experiment
SLCGSDGCR

SEQ ID NO: 427 - protein, 9 aa, Artificial Sequence
  Discovered from phage display experiment
MRCQFSGCT

SEQ ID NO: 428 - protein, 9 aa, Artificial Sequence
  Discovered from phage display experiment
STCGNWTCR

SEQ ID NO: 429 - protein, 9 aa, Artificial Sequence
  Discovered from phage display experiment
CSCTGQLCR

SEQ ID NO: 430 - protein, 9 aa, Artificial Sequence
  Discovered from phage display experiment
GLCQIDECR

SEQ ID NO: 431 - protein, 9 aa, Artificial Sequence
  Discovered from phage display experiment
DRCLDIWCL

SEQ ID NO: 432 - protein, 9 aa, Artificial Sequence
  Discovered from phage display experiment
PLCMATRCA

SEQ ID NO: 433 - protein, 9 aa, Artificial Sequence
  Discovered from phage display experiment
NPCLRAACI

SEQ ID NO: 434 - protein, 9 aa, Artificial Sequence
  Discovered from phage display experiment
LECVANLCT

SEQ ID NO: 435 - protein, 9 aa, Artificial Sequence
  Discovered from phage display experiment
EPCTWNACL

SEQ ID NO: 436 - protein, 9 aa, Artificial Sequence
  Discovered from phage display experiment
LYCLDASCL

SEQ ID NO: 437 - protein, 9 aa, Artificial Sequence
  Discovered from phage display experiment
LVCQGSPCL

SEQ ID NO: 438 - protein, 9 aa, Artificial Sequence
  Discovered from phage display experiment
DXCXDIWCL

SEQ ID NO: 439 - protein, 8 aa, Artificial Sequence
  Discovered from phage display experiment
KTCVGVRV

SEQ ID NO: 440 - protein, 9 aa, Artificial Sequence
  Discovered from phage display experiment
LTCWDWSCR

SEQ ID NO: 441 - protein, 9 aa, Artificial Sequence
  Discovered from phage display experiment
KTCAGSSCI

SEQ ID NO: 442 - protein, 8 aa, Artificial Sequence
  Discovered from phage display experiment
NPCFGLLV

SEQ ID NO: 443 - protein, 9 aa, Artificial Sequence
  Discovered from phage display experiment
RTCTPSRCM

SEQ ID NO: 444 - protein, 9 aa, Artificial Sequence
  Discovered from phage display experiment
QYCWSKGCR

SEQ ID NO: 445 - protein, 9 aa, Artificial Sequence
  Discovered from phage display experiment
VTCSSEWCL

SEQ ID NO: 446 - protein, 9 aa, Artificial Sequence
  Discovered from phage display experiment
STCISVHCS

SEQ ID NO: 447 - protein, 9 aa, Artificial Sequence
  Discovered from phage display experiment
IACDGYLCG

SEQ ID NO: 448 - protein, 9 aa, Artificial Sequence
  Discovered from phage display experiment
XGCYQKRCT

SEQ ID NO: 449 - protein, 9 aa, Artificial Sequence
  Discovered from phage display experiment
IRCWGGRCS

SEQ ID NO: 450 - protein, 9 aa, Artificial Sequence
  Discovered from phage display experiment
AGCVQSQCY

SEQ ID NO: 451 - protein, 9 aa, Artificial Sequence
  Discovered from phage display experiment
KACFGADCX

SEQ ID NO: 452 - protein, 9 aa, Artificial Sequence
  Discovered from phage display experiment
SACWLSNCA

SEQ ID NO: 453 - protein, 9 aa, Artificial Sequence
  Discovered from phage display experiment
GLCQEHRCW

SEQ ID NO: 454 - protein, 9 aa, Artificial Sequence
  Discovered from phage display experiment
EDCREWGCR

SEQ ID NO: 455 - protein, 9 aa, Artificial Sequence
  Discovered from phage display experiment
CGNKRTRGC

SEQ ID NO: 456 - protein, 9 aa, Artificial Sequence
  Discovered from phage display experiment
CLSDGKRKC

SEQ ID NO: 457 - protein, 9 aa, Artificial Sequence
  Discovered from phage display experiment
CREAGRKAC

SEQ ID NO: 458 - protein, 9 aa, Artificial Sequence
  Discovered from phage display experiment
MECIKYSCL

SEQ ID NO: 459 - protein, 9 aa, Artificial Sequence
  Discovered from phage display experiment
PRCQLWACT

SEQ ID NO: 460 - protein, 9 aa, Artificial Sequence
  Discovered from phage display experiment
SHCPMASLC

SEQ ID NO: 461 - protein, 9 aa, Artificial Sequence
  Discovered from phage display experiment
TSCRLFSCA

SEQ ID NO: 462 - protein, 9 aa, Artificial Sequence
  Discovered from phage display experiment
RGCNGSRCS

SEQ ID NO: 463 - protein, 9 aa, Artificial Sequence
  Discovered from phage display experiment
PECEGVSCI

SEQ ID NO: 464 - protein, 9 aa, Artificial Sequence
  Discovered from phage display experiment
IPCYWESCR

SEQ ID NO: 465 - protein, 9 aa, Artificial Sequence
  Discovered from phage display experiment
QDCVKRPCV

SEQ ID NO: 466 - protein, 9 aa, Artificial Sequence
  Discovered from phage display experiment
WSCARPLCG

SEQ ID NO: 467 - protein, 9 aa, Artificial Sequence
  Discovered from phage display experiment
RLCPSSPCT

SEQ ID NO: 468 - protein, 9 aa, Artificial Sequence
  Discovered from phage display experiment
RYCYPDGCL

SEQ ID NO: 469 - protein, 9 aa, Artificial Sequence
  Discovered from phage display experiment
LPCTGASCP

SEQ ID NO: 470 - protein, 9 aa, Artificial Sequence
  Discovered from phage display experiment
LECRRWRCD

SEQ ID NO: 471 - protein, 9 aa, Artificial Sequence
  Discovered from phage display experiment
TACKVAACH

SEQ ID NO: 472 - protein, 9 aa, Artificial Sequence
  Discovered from phage display experiment
XXXQGSPCL

SEQ ID NO: 473 - protein, 9 aa, Artificial Sequence
  Discovered from phage display experiment
RDCSHRSCE

SEQ ID NO: 474 - protein, 9 aa, Artificial Sequence
  Discovered from phage display experiment
PTCAYGWCA

SEQ ID NO: 475 - protein, 9 aa, Artificial Sequence
  Discovered from phage display experiment
RKCGEEVCT

SEQ ID NO: 476 - protein, 9 aa, Artificial Sequence
  Discovered from phage display experiment
LVCPGTACV

SEQ ID NO: 477 - protein, 9 aa, Artificial Sequence
  Discovered from phage display experiment
ERCPMAKCY

SEQ ID NO: 478 - protein, 9 aa, Artificial Sequence
  Discovered from phage display experiment
QQCQDPYCL

SEQ ID NO: 479 - protein, 9 aa, Artificial Sequence
  Discovered from phage display experiment
QPCRSMVCA

SEQ ID NO: 480 - protein, 9 aa, Artificial Sequence
  Discovered from phage display experiment
WSCHEFNCR

SEQ ID NO: 481 - protein, 9 aa, Artificial Sequence
  Discovered from phage display experiment
SLCRLSTCS

SEQ ID NO: 482 - protein, 9 aa, Artificial Sequence
  Discovered from phage display experiment
VICTGRQCG

SEQ ID NO: 483 - protein, 9 aa, Artificial Sequence
  Discovered from phage display experiment
SLCTAFNCH

SEQ ID NO: 484 - protein, 9 aa, Artificial Sequence
  Discovered from phage display experiment
QSCLWRICI

SEQ ID NO: 485 - protein, 9 aa, Artificial Sequence
  Discovered from phage display experiment
LGCFPSWCG

SEQ ID NO: 486 - protein, 9 aa, Artificial Sequence
  Discovered from phage display experiment
RLCSWGGCA

SEQ ID NO: 487 - protein, 9 aa, Artificial Sequence
  Discovered from phage display experiment
EVCLVLSCQ

SEQ ID NO: 488 - protein, 9 aa, Artificial Sequence
  Discovered from phage display experiment
RDCVKNLCR

SEQ ID NO: 489 - protein, 9 aa, Artificial Sequence
  Discovered from phage display experiment
LGCFXSWCG

SEQ ID NO: 490 - protein, 9 aa, Artificial Sequence
  Discovered from phage display experiment
IPCSLLGCA

SEQ ID NO: 491 - protein, 9 aa, Artificial Sequence
  Discovered from phage display experiment
PRCWERVCS

SEQ ID NO: 492 - protein, 9 aa, Artificial Sequence
  Discovered from phage display experiment
TLCPLVACE

SEQ ID NO: 493 - protein, 9 aa, Artificial Sequence
  Discovered from phage display experiment
SECYTGSCP

SEQ ID NO: 494 - protein, 9 aa, Artificial Sequence
  Discovered from phage display experiment
VECGFSAVF

SEQ ID NO: 495 - protein, 9 aa, Artificial Sequence
  Discovered from phage display experiment
HWCRLLACR

SEQ ID NO: 496 - protein, 9 aa, Artificial Sequence
  Discovered from phage display experiment
CAGRRSAYC

SEQ ID NO: 497 - protein, 9 aa, Artificial Sequence
  Discovered from phage display experiment
CNRRTKAGC

SEQ ID NO: 498 - protein, 9 aa, Artificial Sequence
  Discovered from phage display experiment
WGCKLRFCS

SEQ ID NO: 499 - protein, 9 aa, Artificial Sequence
  Discovered from phage display experiment
GICATVKCS

SEQ ID NO: 500 - protein, 9 aa, Artificial Sequence
  Discovered from phage display experiment
TTCMSQLCL

SEQ ID NO: 501 - protein, 9 aa, Artificial Sequence
  Discovered from phage display experiment
GCVRRLLCN

SEQ ID NO: 502 - protein, 9 aa, Artificial Sequence
  Discovered from phage display experiment
KYCTPVECL

SEQ ID NO: 503 - protein, 8 aa, Artificial Sequence
  Discovered from phage display experiment
MCPQRNCL

SEQ ID NO: 504 - protein, 9 aa, Artificial Sequence
  Discovered from phage display experiment
AGCSVTVCG

SEQ ID NO: 505 - protein, 9 aa, Artificial Sequence
  Discovered from phage display experiment
GSCSMFPCS

SEQ ID NO: 506 - protein, 9 aa, Artificial Sequence
  Discovered from phage display experiment
SECAYRACS

SEQ ID NO: 507 - protein, 9 aa, Artificial Sequence
  Discovered from phage display experiment
SLCGSDGCR

SEQ ID NO: 508 - protein, 9 aa, Artificial Sequence
  Discovered from phage display experiment
MRCQFSGCT

SEQ ID NO: 509 - protein, 9 aa, Artificial Sequence
  Discovered from phage display experiment
STCGNWTCR

SEQ ID NO: 510 - protein, 9 aa, Artificial Sequence
  Discovered from phage display experiment
CSCTGQLCR

SEQ ID NO: 511 - protein, 9 aa, Artificial Sequence
  Discovered from phage display experiment
GLCQIDECR

SEQ ID NO: 512 - protein, 9 aa, Artificial Sequence
  Discovered from phage display experiment
DRCLDIWCL

SEQ ID NO: 513 - protein, 9 aa, Artificial Sequence
  Discovered from phage display experiment
PLCMATRCA

SEQ ID NO: 514 - protein, 9 aa, Artificial Sequence
  Discovered from phage display experiment
NPCLRAACI

SEQ ID NO: 515 - protein, 9 aa, Artificial Sequence
  Discovered from phage display experiment
LECVANLCT

SEQ ID NO: 516 - protein, 9 aa, Artificial Sequence
  Discovered from phage display experiment
EPCTWNACL

SEQ ID NO: 517 - protein, 9 aa, Artificial Sequence
  Discovered from phage display experiment
LYCLDASCL

SEQ ID NO: 518 - protein, 9 aa, Artificial Sequence
  Discovered from phage display experiment
LVCQGSPCL

SEQ ID NO: 519 - protein, 9 aa, Artificial Sequence
  Discovered from phage display experiment
DXCXDIWCL

SEQ ID NO: 520 - protein, 8 aa, Artificial Sequence
  Discovered from phage display experiment
KTCVGVRV

SEQ ID NO: 521 - protein, 9 aa, Artificial Sequence
  Discovered from phage display experiment
LTCWDWSCR

SEQ ID NO: 522 - protein, 9 aa, Artificial Sequence
  Discovered from phage display experiment
KTCAGSSCI

SEQ ID NO: 523 - protein, 8 aa, Artificial Sequence
  Discovered from phage display experiment
NPCFGLLV

SEQ ID NO: 524 - protein, 9 aa, Artificial Sequence
  Discovered from phage display experiment
RTCTPSRCM

SEQ ID NO: 525 - protein, 9 aa, Artificial Sequence
  Discovered from phage display experiment
QYCWSKGCR

SEQ ID NO: 526 - protein, 9 aa, Artificial Sequence
  Discovered from phage display experiment
VTCSSEWCL

SEQ ID NO: 527 - protein, 9 aa, Artificial Sequence
  Discovered from phage display experiment
STCISVHCS

SEQ ID NO: 528 - protein, 9 aa, Artificial Sequence
  Discovered from phage display experiment
IACDGYLCG

SEQ ID NO: 529 - protein, 9 aa, Artificial Sequence
  Discovered from phage display experiment
XGCYQKRCT

SEQ ID NO: 530 - protein, 9 aa, Artificial Sequence
  Discovered from phage display experiment
IRCWGGRCS

SEQ ID NO: 531 - protein, 9 aa, Artificial Sequence
  Discovered from phage display experiment
AGCVQSQCY

SEQ ID NO: 532 - protein, 9 aa, Artificial Sequence
  Discovered from phage display experiment
KACGGADCX

SEQ ID NO: 533 - protein, 9 aa, Artificial Sequence
  Discovered from phage display experiment
SACWLSNCA

SEQ ID NO: 534 - protein, 9 aa, Artificial Sequence
  Discovered from phage display experiment
GLCQEHRCW

SEQ ID NO: 535 - protein, 9 aa, Artificial Sequence
  Discovered from phage display experiment
EDCREWGCR

SEQ ID NO: 536 - protein, 7 aa, Artificial Sequence
  Discovered from phage display experiment
LMLPRAD

SEQ ID NO: 537 - protein, 9 aa, Artificial Sequence
  Discovered from phage display experiment
MECIKYSCL

SEQ ID NO: 538 - protein, 9 aa, Artificial Sequence
  Discovered from phage display experiment
PRCQLWACT

SEQ ID NO: 539 - protein, 9 aa, Artificial Sequence
  Discovered from phage display experiment
SHCPMASLC

SEQ ID NO: 540 - protein, 9 aa, Artificial Sequence
  Discovered from phage display experiment
TSCRLFSCA

SEQ ID NO: 541 - protein, 9 aa, Artificial Sequence
  Discovered from phage display experiment
RGCNGSRCS

SEQ ID NO: 542 - protein, 9 aa, Artificial Sequence
  Discovered from phage display experiment
PECEGVSCI

SEQ ID NO: 543 - protein, 9 aa, Artificial Sequence
  Discovered from phage display experiment
IPCYWESCR

SEQ ID NO: 544 - protein, 9 aa, Artificial Sequence
  Discovered from phage display experiment
QDCVKRPCV

SEQ ID NO: 545 - protein, 9 aa, Artificial Sequence
  Discovered from phage display experiment
WSCARPLCG

SEQ ID NO: 546 - protein, 9 aa, Artificial Sequence
  Discovered from phage display experiment
RLCPSSPCT

SEQ ID NO: 547 - protein, 9 aa, Artificial Sequence
  Discovered from phage display experiment
RYCYPDGCL

SEQ ID NO: 548 - protein, 9 aa, Artificial Sequence
  Discovered from phage display experiment
LPCTGASCP

SEQ ID NO: 549 - protein, 9 aa, Artificial Sequence
  Discovered from phage display experiment
LECRRWRCD

SEQ ID NO: 550 - protein, 9 aa, Artificial Sequence
  Discovered from phage display experiment
TACKVAACH

SEQ ID NO: 551 - protein, 9 aa, Artificial Sequence
  Discovered from phage display experiment
XXXQGSPCL

SEQ ID NO: 552 - protein, 9 aa, Artificial Sequence
  Discovered from phage display experiment
RDCSHRSCE

SEQ ID NO: 553 - protein, 9 aa, Artificial Sequence
  Discovered from phage display experiment
PTCAYGWCA

SEQ ID NO: 554 - protein, 9 aa, Artificial Sequence
  Discovered from phage display experiment
RKCGEEVCT

SEQ ID NO: 555 - protein, 9 aa, Artificial Sequence
  Discovered from phage display experiment
LVCPGTACV

SEQ ID NO: 556 - protein, 9 aa, Artificial Sequence
  Discovered from phage display experiment
ERCPMAKCY

SEQ ID NO: 557 - protein, 9 aa, Artificial Sequence
  Discovered from phage display experiment
QQCQDPYCL

SEQ ID NO: 558 - protein, 9 aa, Artificial Sequence
  Discovered from phage display experiment
QPCRSMVCA

SEQ ID NO: 559 - protein, 9 aa, Artificial Sequence
  Discovered from phage display experiment
WSCHEFNCR

SEQ ID NO: 560 - protein, 9 aa, Artificial Sequence
  Discovered from phage display experiment
SLCRLSTCS

SEQ ID NO: 561 - protein, 9 aa, Artificial Sequence
  Discovered from phage display experiment
VICTGRQCG

SEQ ID NO: 562 - protein, 9 aa, Artificial Sequence
  Discovered from phage display experiment
SLCTAFNCH

SEQ ID NO: 563 - protein, 12 aa, Artificial Sequence
  Discovered from phage display experiment
VPWMEPAYQRFL

SEQ ID NO: 564 - protein, 12 aa, Artificial Sequence
  Discovered from phage display experiment
HLQLQPWYPQIS

SEQ ID NO: 565 - protein, 9 aa, Artificial Sequence
  Discovered from phage display experiment
LVRSTGQFV

SEQ ID NO: 566 - protein, 9 aa, Artificial Sequence
  Discovered from phage display experiment
ALRPSGEWL

SEQ ID NO: 567 - protein, 9 aa, Artificial Sequence
  Discovered from phage display experiment
QILASGRWL

SEQ ID NO: 568 - protein, 9 aa, Artificial Sequence
  Discovered from phage display experiment
DNNRPANSM

SEQ ID NO: 569 - protein, 9 aa, Artificial Sequence
  Discovered from phage display experiment
PLSGDKSST

SEQ ID NO: 570 - protein, 15 aa, Artificial Sequence
  Discovered from phage display experiment
RMWPSSTVNLSAGRR

SEQ ID NO: 571 - protein, 15 aa, Artificial Sequence
  Discovered from phage display experiment
GRVPSMFGGHFFFSR

SEQ ID NO: 572 - protein, 9 aa, Artificial Sequence
  Discovered from phage display experiment
LVSPSGSWT

SEQ ID NO: 573 - protein, 9 aa, Artificial Sequence
  Discovered from phage display experiment
AIMASGQWL

SEQ ID NO: 574 - protein, 9 aa, Artificial Sequence
  Discovered from phage display experiment
RRPSHAMAR

SEQ ID NO: 575 - protein, 9 aa, Artificial Sequence
  Discovered from phage display experiment
LQDRLRFAT

SEQ ID NO: 576 - protein, 7 aa, Artificial Sequence
  Discovered from phage display experiment
IELLQAR

SEQ ID NO: 577 - protein, 15 aa, Artificial Sequence
  Discovered from phage display experiment
PNLDFSPTCSFRFGC

SEQ ID NO: 578 - protein, 12 aa, Artificial Sequence
  Discovered from phage display experiment
TARGEHKEEELI

SEQ ID NO: 579 - protein, 20 aa, Artificial Sequence
  Discovered from phage display experiment
TGGETSGIKKAPYASTTRNR

SEQ ID NO: 580 - protein, 20 aa, Artificial Sequence
  Discovered from phage display experiment
SHHGVAGVDLGGGADFKSIA

SEQ ID NO: 581 - protein, 7 aa, Artificial Sequence
  Discovered from phage display experiment
ASSLNIA

SEQ ID NO: 582 - protein, 10 aa, Artificial Sequence
  Discovered from phage display experiment
CGLIIQKNEC

SEQ ID NO: 583 - protein, 10 aa, Artificial Sequence
  Discovered from phage display experiment
CNAGESSKNC

SEQ ID NO: 584 - protein, 9 aa, Artificial Sequence
  Discovered from phage display experiment
CKGGRAKDC

SEQ ID NO: 585 - protein, 20 aa, Artificial Sequence
  Discovered from phage display experiment
RRKKAAVALLPAVLLALLAP

SEQ ID NO: 586 - protein, 19 aa, Artificial Sequence
  Discovered from phage display experiment
TDVILMCFSIDSPDSLENI

SEQ ID NO: 587 - protein, 4 aa, Artificial Sequence
  Discovered from phage display experiment
KALA

SEQ ID NO: 588 - protein, 16 aa, Artificial Sequence
  Discovered from phage display experiment
RQIKIWFQNRRMKWKK

SEQ ID NO: 589 - protein, 13 aa, Artificial Sequence
  Discovered from phage display experiment
CGRECPRLCQSSC

SEQ ID NO: 590 - protein, 9 aa, Artificial Sequence
  Discovered from phage display experiment
SKVLYYNWE

SEQ ID NO: 591 - protein, 10 aa, Artificial Sequence
  Discovered from phage display experiment
CPTCNGRCVR

SEQ ID NO: 592 - protein, 10 aa, Artificial Sequence
  Discovered from phage display experiment
CAVCNGRCGF

SEQ ID NO: 593 - protein, 10 aa, Artificial Sequence
  Discovered from phage display experiment
CVQCNGRCAL

SEQ ID NO: 594 - protein, 10 aa, Artificial Sequence
  Discovered from phage display experiment
CEGVNGRRLR

SEQ ID NO: 595 - protein, 7 aa, Artificial Sequence
  Discovered from phage display experiment
KMGPKVW

SEQ ID NO: 596 - protein, 7 aa, Artificial Sequence
  Discovered from phage display experiment
CWSGVDC

SEQ ID NO: 597 - protein, 9 aa, Artificial Sequence
  Discovered from phage display experiment
CVMVRDGDC

SEQ ID NO: 598 - protein, 9 aa, Artificial Sequence
  Discovered from phage display experiment
CPEHRSLVC

SEQ ID NO: 599 - protein, 9 aa, Artificial Sequence
  Discovered from phage display experiment
CAQLLQVSC

SEQ ID NO: 600 - protein, 9 aa, Artificial Sequence
  Discovered from phage display experiment
CTAMRNTDC

SEQ ID NO: 601 - protein, 8 aa, Artificial Sequence
  Discovered from phage display experiment
CYLVNVDC

SEQ ID NO: 602 - protein, 9 aa, Artificial Sequence
  Discovered from phage display experiment
QWCSRRWCT

SEQ ID NO: 603 - protein, 9 aa, Artificial Sequence
  Discovered from phage display experiment
AGCINGLCG

SEQ ID NO: 604 - protein, 9 aa, Artificial Sequence
  Discovered from phage display experiment
LDCLSELCS

SEQ ID NO: 605 - protein, 9 aa, Artificial Sequence
  Discovered from phage display experiment
RWCREKSCW

SEQ ID NO: 606 - protein, 10 aa, Artificial Sequence
  Discovered from phage display experiment
CEQCNGRCGQ

SEQ ID NO: 607 - protein, 10 aa, Artificial Sequence
  Discovered from phage display experiment
CSCCNGRCGD

SEQ ID NO: 608 - protein, 10 aa, Artificial Sequence
  Discovered from phage display experiment
CASNNGRVVL

SEQ ID NO: 609 - protein, 10 aa, Artificial Sequence
  Discovered from phage display experiment
CEVCNGRCAL

SEQ ID NO: 610 - protein, 7 aa, Artificial Sequence
  Discovered from phage display experiment
SPGSWTW

SEQ ID NO: 611 - protein, 7 aa, Artificial Sequence
  Discovered from phage display experiment
SKSSGVS

SEQ ID NO: 612 - protein, 7 aa, Artificial Sequence
  Discovered from phage display experiment
CQLAAVC

SEQ ID NO: 613 - protein, 7 aa, Artificial Sequence
  Discovered from phage display experiment
CYVELHC

SEQ ID NO: 614 - protein, 8 aa, Artificial Sequence
  Discovered from phage display experiment
CKALSQAC

SEQ ID NO: 615 - protein, 8 aa, Artificial Sequence
  Discovered from phage display experiment
CGTRVDHC

SEQ ID NO: 616 - protein, 9 aa, Artificial Sequence
  Discovered from phage display experiment
ISCAVDACL

SEQ ID NO: 617 - protein, 9 aa, Artificial Sequence
  Discovered from phage display experiment
NRCRGVSCT

SEQ ID NO: 618 - protein, 13 aa, Artificial Sequence
  Discovered from phage display experiment
CGEACGGQCALPC

SEQ ID NO: 619 - protein, 13 aa, Artificial Sequence
  Discovered from phage display experiment
CERACRNLCREGC

SEQ ID NO: 620 - protein, 10 aa, Artificial Sequence
  Discovered from phage display experiment
CRNCNGRCEG

SEQ ID NO: 621 - protein, 10 aa, Artificial Sequence
  Discovered from phage display experiment
CWGCNGRCRM

SEQ ID NO: 622 - protein, 10 aa, Artificial Sequence
  Discovered from phage display experiment
CGRCNGRCLL

SEQ ID NO: 623 - protein, 7 aa, Artificial Sequence
  Discovered from phage display experiment
CGSLVRC

SEQ ID NO: 624 - protein, 7 aa, Artificial Sequence
  Discovered from phage display experiment
NPRWFWD

SEQ ID NO: 625 - protein, 7 aa, Artificial Sequence
  Discovered from phage display experiment
IVADYQR

SEQ ID NO: 626 - protein, 7 aa, Artificial Sequence
  Discovered from phage display experiment
CGVGSSC

SEQ ID NO: 627 - protein, 6 aa, Artificial Sequence
  Discovered from phage display experiment
CWRKYC

SEQ ID NO: 628 - protein, 7 aa, Artificial Sequence
  Discovered from phage display experiment
CTDYVRC

SEQ ID NO: 629 - protein, 9 aa, Artificial Sequence
  Discovered from phage display experiment
VTCRSLMCQ

SEQ ID NO: 630 - protein, 9 aa, Artificial Sequence
  Discovered from phage display experiment
RHCFSQWCS

SEQ ID NO: 631 - protein, 9 aa, Artificial Sequence
  Discovered from phage display experiment
NACESAICG

SEQ ID NO: 632 - protein, 9 aa, Artificial Sequence
  Discovered from phage display experiment
KGCGTRQCW

SEQ ID NO: 633 - protein, 9 aa, Artificial Sequence
  Discovered from phage display experiment
IYCPGQECE

SEQ ID NO: 634 - protein, 12 aa, Artificial Sequence
  Discovered from phage display experiment
CNKTDGDEGVTC

SEQ ID NO: 635 - protein, 10 aa, Artificial Sequence
  Discovered from phage display experiment
CVTCNGRCRV

SEQ ID NO: 636 - protein, 10 aa, Artificial Sequence
  Discovered from phage display experiment
CKSCNGRCLA

SEQ ID NO: 637 - protein, 10 aa, Artificial Sequence
  Discovered from phage display experiment
CSKCNGRCGH

SEQ ID NO: 638 - protein, 7 aa, Artificial Sequence
  Discovered from phage display experiment
HHTRFVS

SEQ ID NO: 639 - protein, 7 aa, Artificial Sequence
  Discovered from phage display experiment
IKARASP

SEQ ID NO: 640 - protein, 7 aa, Artificial Sequence
  Discovered from phage display experiment
VVDRFPD

SEQ ID NO: 641 - protein, 7 aa, Artificial Sequence
  Discovered from phage display experiment
CGLSDSC

SEQ ID NO: 642 - protein, 8 aa, Artificial Sequence
  Discovered from phage display experiment
CYSYFLAC

SEQ ID NO: 643 - protein, 9 aa, Artificial Sequence
  Discovered from phage display experiment
VPCRFKQCW

SEQ ID NO: 644 - protein, 8 aa, Artificial Sequence
  Discovered from phage display experiment
CYLGVSNC

SEQ ID NO: 645 - protein, 9 aa, Artificial Sequence
  Discovered from phage display experiment
RSCIKHQCP

SEQ ID NO: 646 - protein, 9 aa, Artificial Sequence
  Discovered from phage display experiment
FGCVMASCR

SEQ ID NO: 647 - protein, 9 aa, Artificial Sequence
  Discovered from phage display experiment
PSCAYMCIT

SEQ ID NO: 648 - protein, 10 aa, Artificial Sequence
  Discovered from phage display experiment
CKVCNGRCCG

SEQ ID NO: 649 - protein, 10 aa, Artificial Sequence
  Discovered from phage display experiment
CTECNGRCQL

SEQ ID NO: 650 - protein, 10 aa, Artificial Sequence
  Discovered from phage display experiment
CVPCNGRCHE

SEQ ID NO: 651 - protein, 10 aa, Artificial Sequence
  Discovered from phage display experiment
CVWCNGRCGL

SEQ ID NO: 652 - protein, 7 aa, Artificial Sequence
  Discovered from phage display experiment
SKGLRHR

SEQ ID NO: 653 - protein, 7 aa, Artificial Sequence
  Discovered from phage display experiment
SGWCYRC

SEQ ID NO: 654 - protein, 7 aa, Artificial Sequence
  Discovered from phage display experiment
LSMFTRP

SEQ ID NO: 655 - protein, 7 aa, Artificial Sequence
  Discovered from phage display experiment
CGEGHPC

SEQ ID NO: 656 - protein, 7 aa, Artificial Sequence
  Discovered from phage display experiment
CPRGSRC

SEQ ID NO: 657 - protein, 9 aa, Artificial Sequence
  Discovered from phage display experiment
TDCTPSRCT

SEQ ID NO: 658 - protein, 8 aa, Artificial Sequence
  Discovered from phage display experiment
CISLDRSC

SEQ ID NO: 659 - protein, 9 aa, Artificial Sequence
  Discovered from phage display experiment
EACEMAGCL

SEQ ID NO: 660 - protein, 9 aa, Artificial Sequence
  Discovered from phage display experiment
EPCEGKKCL

SEQ ID NO: 661 - protein, 9 aa, Artificial Sequence
  Discovered from phage display experiment
KRCSSSLCA

SEQ ID NO: 662 - protein, 9 aa, Artificial Sequence
  Discovered from phage display experiment
EDCTSRFCS

SEQ ID NO: 663 - protein, 10 aa, Artificial Sequence
  Discovered from phage display experiment
CPLCNGRCAL

SEQ ID NO: 664 - protein, 10 aa, Artificial Sequence
  Discovered from phage display experiment
CETCNGRCAL

SEQ ID NO: 665 - protein, 10 aa, Artificial Sequence
  Discovered from phage display experiment
CRTCNGRCQV

SEQ ID NO: 666 - protein, 10 aa, Artificial Sequence
  Discovered from phage display experiment
CGECNGRCVE

SEQ ID NO: 667 - protein, 7 aa, Artificial Sequence
  Discovered from phage display experiment
WRVLAAF

SEQ ID NO: 668 - protein, 7 aa, Artificial Sequence
  Discovered from phage display experiment
LWAEMTG

SEQ ID NO: 669 - protein, 7 aa, Artificial Sequence
  Discovered from phage display experiment
IMYPGWL

SEQ ID NO: 670 - protein, 9 aa, Artificial Sequence
  Discovered from phage display experiment
CELSLISKC

SEQ ID NO: 671 - protein, 7 aa, Artificial Sequence
  Discovered from phage display experiment
CDDSWKC

SEQ ID NO: 672 - protein, 8 aa, Artificial Sequence
  Discovered from phage display experiment
CMEMGVKC

SEQ ID NO: 673 - protein, 9 aa, Artificial Sequence
  Discovered from phage display experiment
LVCLPPSCE

SEQ ID NO: 674 - protein, 9 aa, Artificial Sequence
  Discovered from phage display experiment
GICKDLWCQ

SEQ ID NO: 675 - protein, 9 aa, Artificial Sequence
  Discovered from phage display experiment
DTCRALRCN

SEQ ID NO: 676 - protein, 9 aa, Artificial Sequence
  Discovered from phage display experiment
YRCIARECE

SEQ ID NO: 677 - protein, 9 aa, Artificial Sequence
  Discovered from phage display experiment
RKCEVPGCQ

SEQ ID NO: 678 - protein, 10 aa, Artificial Sequence
  Discovered from phage display experiment
CEMCNGRCMG

SEQ ID NO: 679 - protein, 10 aa, Artificial Sequence
  Discovered from phage display experiment
CRTCNGRCLE

SEQ ID NO: 680 - protein, 10 aa, Artificial Sequence
  Discovered from phage display experiment
CQSCNGRCVR

SEQ ID NO: 681 - protein, 10 aa, Artificial Sequence
  Discovered from phage display experiment
CIRCNGRCSV

SEQ ID NO: 682 - protein, 7 aa, Artificial Sequence
  Discovered from phage display experiment
VASVSVA

SEQ ID NO: 683 - protein, 7 aa, Artificial Sequence
  Discovered from phage display experiment
ALVGLMR

SEQ ID NO: 684 - protein, 7 aa, Artificial Sequence
  Discovered from phage display experiment
GLPVKWS

SEQ ID NO: 685 - protein, 7 aa, Artificial Sequence
  Discovered from phage display experiment
CYTADPC

SEQ ID NO: 686 - protein, 7 aa, Artificial Sequence
  Discovered from phage display experiment
CRLGIAC

SEQ ID NO: 687 - protein, 9 aa, Artificial Sequence
  Discovered from phage display experiment
SWCQFEKCL

SEQ ID NO: 688 - protein, 8 aa, Artificial Sequence
  Discovered from phage display experiment
CAMVSMED

SEQ ID NO: 689 - protein, 9 aa, Artificial Sequence
  Discovered from phage display experiment
PRCESQLCP

SEQ ID NO: 690 - protein, 9 aa, Artificial Sequence
  Discovered from phage display experiment
ADCRQKPCL

SEQ ID NO: 691 - protein, 8 aa, Artificial Sequence
  Discovered from phage display experiment
ICLLAHCA

SEQ ID NO: 692 - protein, 9 aa, Artificial Sequence
  Discovered from phage display experiment
LECVVDSCR

SEQ ID NO: 693 - protein, 9 aa, Artificial Sequence
  Discovered from phage display experiment
IWSGYGVYW

SEQ ID NO: 694 - protein, 13 aa, Artificial Sequence
  Discovered from phage display experiment
CPRGCLAVCVSQC

SEQ ID NO: 695 - protein, 9 aa, Artificial Sequence
  Discovered from phage display experiment
QACPMLLCM

SEQ ID NO: 696 - protein, 9 aa, Artificial Sequence
  Discovered from phage display experiment
EICVDGLCV

SEQ ID NO: 697 - protein, 10 aa, Artificial Sequence
  Discovered from phage display experiment
CGVCNGRCGL

SEQ ID NO: 698 - protein, 10 aa, Artificial Sequence
  Discovered from phage display experiment
CRDLNGRKVM

SEQ ID NO: 699 - protein, 10 aa, Artificial Sequence
  Discovered from phage display experiment
CRCCNGRCSP

SEQ ID NO: 700 - protein, 10 aa, Artificial Sequence
  Discovered from phage display experiment
CLSCNGRCPS

SEQ ID NO: 701 - protein, 7 aa, Artificial Sequence
  Discovered from phage display experiment
IFSGSRE

SEQ ID NO: 702 - protein, 7 aa, Artificial Sequence
  Discovered from phage display experiment
DTLRLRI

SEQ ID NO: 703 - protein, 7 aa, Artificial Sequence
  Discovered from phage display experiment
CVRIRPC

SEQ ID NO: 704 - protein, 9 aa, Artificial Sequence
  Discovered from phage display experiment
CLVVHEAAC

SEQ ID NO: 705 - protein, 7 aa, Artificial Sequence
  Discovered from phage display experiment
CYPADPC

SEQ ID NO: 706 - protein, 8 aa, Artificial Sequence
  Discovered from phage display experiment
CRESLKNC

SEQ ID NO: 707 - protein, 8 aa, Artificial Sequence
  Discovered from phage display experiment
CIRSAVSC

SEQ ID NO: 708 - protein, 9 aa, Artificial Sequence
  Discovered from phage display experiment
MFCRMRSCD

SEQ ID NO: 709 - protein, 9 aa, Artificial Sequence
  Discovered from phage display experiment
RSCAEPWCY

SEQ ID NO: 710 - protein, 8 aa, Artificial Sequence
  Discovered from phage display experiment
AGCRVESC

SEQ ID NO: 711 - protein, 9 aa, Artificial Sequence
  Discovered from phage display experiment
FRCLERVCT

SEQ ID NO: 712 - protein, 9 aa, Artificial Sequence
  Discovered from phage display experiment
WESLYFPRE

SEQ ID NO: 713 - protein, 12 aa, Artificial Sequence
  Discovered from phage display experiment
RLCRIVVIRVCR

SEQ ID NO: 714 - protein, 9 aa, Artificial Sequence
  Discovered from phage display experiment
HTCLVALCA

SEQ ID NO: 715 - protein, 9 aa, Artificial Sequence
  Discovered from phage display experiment
RPCGDQACE

SEQ ID NO: 716 - protein, 10 aa, Artificial Sequence
  Discovered from phage display experiment
CVLCNGRCWS

SEQ ID NO: 717 - protein, 10 aa, Artificial Sequence
  Discovered from phage display experiment
CPLCNGRCAR

SEQ ID NO: 718 - protein, 10 aa, Artificial Sequence
  Discovered from phage display experiment
CWLCNGRCGR

SEQ ID NO: 719 - protein, 7 aa, Artificial Sequence
  Discovered from phage display experiment
GRSQMQI

SEQ ID NO: 720 - protein, 7 aa, Artificial Sequence
  Discovered from phage display experiment
GRWYKWA

SEQ ID NO: 721 - protein, 7 aa, Artificial Sequence
  Discovered from phage display experiment
VWRTGHL

SEQ ID NO: 722 - protein, 7 aa, Artificial Sequence
  Discovered from phage display experiment
CVSGPRC

SEQ ID NO: 723 - protein, 7 aa, Artificial Sequence
  Discovered from phage display experiment
CFWPNRC

SEQ ID NO: 724 - protein, 7 aa, Artificial Sequence
  Discovered from phage display experiment
CGETMRC

SEQ ID NO: 725 - protein, 8 aa, Artificial Sequence
  Discovered from phage display experiment
CNNVGSYC

SEQ ID NO: 726 - protein, 9 aa, Artificial Sequence
  Discovered from phage display experiment
FYCPGVGCR

SEQ ID NO: 727 - protein, 9 aa, Artificial Sequence
  Discovered from phage display experiment
APCGLLACI

SEQ ID NO: 728 - protein, 9 aa, Artificial Sequence
  Discovered from phage display experiment
GRCVDGGCT

SEQ ID NO: 729 - protein, 9 aa, Artificial Sequence
  Discovered from phage display experiment
RLCSLYGCV

SEQ ID NO: 730 - protein, 13 aa, Artificial Sequence
  Discovered from phage display experiment
CNGRCVSGCAGRC

SEQ ID NO: 731 - protein, 13 aa, Artificial Sequence
  Discovered from phage display experiment
CGLMCQGACFDVC

SEQ ID NO: 732 - protein, 34 aa, Artificial Sequence
  Discovered from phage display experiment
YVPLPNVPQPGRRPFPTFPGQGPFNPKIKWPQGY

SEQ ID NO: 733 - protein, 35 aa, Artificial Sequence
  Discovered from phage display experiment
VFIDILDKVENAIHNAAQVGIGFAKPFEKHLINPK

SEQ ID NO: 734 - protein, 18 aa, Artificial Sequence
  Discovered from phage display experiment
GNNRPVYIPQPRPPHPRI

SEQ ID NO: 735 - protein, 18 aa, Artificial Sequence
  Discovered from phage display experiment
GNNRPVYIPQPRPPHPRL

SEQ ID NO: 736 - protein, 18 aa, Artificial Sequence
  Discovered from phage display experiment
GNNRPIYIPQPRPPHPRL

SEQ ID NO: 737 - protein, 42 aa, Artificial Sequence
  Discovered from phage display experiment
RFRPPIRRPPIRPPFYPPFRPPIRPPIFPPIRPPFRPPLRFP

SEQ ID NO: 738 - protein, 60 aa, Artificial Sequence
  Discovered from phage display experiment
RRIRPRPPRLPRPRPRPLPFPRPGPRPIPRPLPFPRPGPRPIPRLPLPFFRPGPRPIPRP

SEQ ID NO: 739 - protein, 20 aa, Artificial Sequence
  Discovered from phage display experiment
PRPIPRPLPFFRPGPRPIPR

SEQ ID NO: 740 - protein, 36 aa, Artificial Sequence
  Discovered from phage display experiment
WNPFKELERAGQRVRDAVISAAPAVATVGQAALARG

SEQ ID NO: 741 - protein, 35 aa, Artificial Sequence
  Discovered from phage display experiment
WNPFKELERAGQRVRDAIISAGPAVATVGQAAAIA

SEQ ID NO: 742 - protein, 37 aa, Artificial Sequence
  Discovered from phage display experiment
WNPFKELERAGQRVRDAIISAAPAVATVGQAAAIARG

SEQ ID NO: 743 - protein, 38 aa, Artificial Sequence
  Discovered from phage display experiment
WNPFKELERAGQRVRDAVISAAPAVATVGQAAAIARGG

SEQ ID NO: 744 - protein, 24 aa, Artificial Sequence
  Discovered from phage display experiment
GIGALSAKGALKGLAKGLAZHFAN

SEQ ID NO: 745 - protein, 27 aa, Artificial Sequence
  Discovered from phage display experiment
GIGASILSAGKSALKGLAKGLAEHFAN

SEQ ID NO: 746 - protein, 27 aa, Artificial Sequence
  Discovered from phage display experiment
GIGSAILSAGKSALKGLAKGLAEHFAN

SEQ ID NO: 747 - protein, 17 aa, Artificial Sequence
  Discovered from phage display experiment
IKITTMLAKLGKVLAHV

SEQ ID NO: 748 - protein, 18 aa, Artificial Sequence
  Discovered from phage display experiment
SKITDILAKLGKVLAIIV

SEQ ID NO: 749 - protein, 19 aa, Artificial Sequence
  Discovered from phage display experiment
RPDFCLEPPYTGPCKARII

SEQ ID NO: 750 - protein, 17 aa, Artificial Sequence
  Discovered from phage display experiment
RYFYNAKAGLCQTFVYG

SEQ ID NO: 751 - protein, 23 aa, Artificial Sequence
  Discovered from phage display experiment
GCRAKRINNFKSAEDCMRTCGGA

SEQ ID NO: 752 - protein, 24 aa, Artificial Sequence
  Discovered from phage display experiment
FLPLLAGLAANFLPKIFCKITRKC

SEQ ID NO: 753 - protein, 33 aa, Artificial Sequence
  Discovered from phage display experiment
GIMDTLKNLAKTAGKGALQSLLNKASCKLSGQC

SEQ ID NO: 754 - protein, 37 aa, Artificial Sequence
  Discovered from phage display experiment
KWKLFKKIEKVGQNIRDGIIKAGPAVAVVGQATQIAK

SEQ ID NO: 755 - protein, 36 aa, Artificial Sequence
  Discovered from phage display experiment
KWKVFKIKIEKMGRNIRNGIVKAGPAIAVLGEAKAL

SEQ ID NO: 756 - protein, 41 aa, Artificial Sequence
  Discovered from phage display experiment
GWILKKLGKRIERIGQHTRDATIQGLGIAQQAANVAATARG

SEQ ID NO: 757 - protein, 37 aa, Artificial Sequence
  Discovered from phage display experiment
WNPFKELEKVGQRVRDAVISAGPAVATVAAQATALAK

SEQ ID NO: 758 - protein, 31 aa, Artificial Sequence
  Discovered from phage display experiment
SWLSKTAKKLENSAKKRISEGIAIAIQGGPR

SEQ ID NO: 759 - protein, 37 aa, Artificial Sequence
  Discovered from phage display experiment
ZFTNVSCTTSKECWSVCQRLHNTSRGKCMNKKCRCYS

SEQ ID NO: 760 - protein, 13 aa, Artificial Sequence
  Discovered from phage display experiment
FLPLILRKIVTAL

SEQ ID NO: 761 - protein, 35 aa, Artificial Sequence
  Discovered from phage display experiment
LRDLVCYCRSRGCKGRERMNGTCRKGHLLYTLCCR

SEQ ID NO: 762 - protein, 35 aa, Artificial Sequence
  Discovered from phage display experiment
LRDLVCYCRTRGCKRRERMNGTCRKGHLMYTLCCR

SEQ ID NO: 763 - protein, 34 aa, Artificial Sequence
  Discovered from phage display experiment
VVCACRRALCLPRERRAGFCRIRGRIHTPLCCRR

SEQ ID NO: 764 - protein, 33 aa, Artificial Sequence
  Discovered from phage display experiment
VVCACRRALCLPLERRAGFCRIRGRIHPLCCRR

SEQ ID NO: 765 - protein, 31 aa, Artificial Sequence
  Discovered from phage display experiment
RRCICTTRTCRFPYRRLGTCIFQNRVYTFCC

SEQ ID NO: 766 - protein, 31 aa, Artificial Sequence
  Discovered from phage display experiment
RRCICTTRTCRFPYRRLGTCLFQNRVYTFCC

SEQ ID NO: 767 - protein, 30 aa, Artificial Sequence
  Discovered from phage display experiment
ACYCRIPACIAGERRYGTCIYQGRLWAFCC

SEQ ID NO: 768 - protein, 29 aa, Artificial Sequence
  Discovered from phage display experiment
CYCRIPACIAGERRYGTCIYQGRLWAFCC

SEQ ID NO: 769 - protein, 33 aa, Artificial Sequence
  Discovered from phage display experiment
VVCACRRALCLPRERRAGFCRIRGRIHPLCCRR

SEQ ID NO: 770 - protein, 33 aa, Artificial Sequence; identical to SEQ ID NO: 764
  Discovered from phage display experiment

SEQ ID NO: 771 - protein, 32 aa, Artificial Sequence
  Discovered from phage display experiment
VTCYCRRTRCGFRERLSGACGYRGRIYRLCCR

SEQ ID NO: 772 - protein, 32 aa, Artificial Sequence
  Discovered from phage display experiment
VTCYCRSTRCGFRERLSGACGYRGRIYRLCCR

SEQ ID NO: 773 - protein, 38 aa, Artificial Sequence
  Discovered from phage display experiment
DFASCHTNGGICLPNRCPGHMIQIGICFRPRVKCCRSW

SEQ ID NO: 774 - protein, 40 aa, Artificial Sequence
  Discovered from phage display experiment
VRNHVTCRINRGFCVPIRCPGRTRQIGTCFGPRIKCCRSW

SEQ ID NO: 775 - protein, 38 aa, Artificial Sequence
  Discovered from phage display experiment
NPVSCVRNKGICVPIRCPGSMKQIGTCVGRAVKCCRKK

SEQ ID NO: 776 - protein, 40 aa, Artificial Sequence
  Discovered from phage display experiment
ATCDLLSGTGINHSACAAHCLLRGNRGGYCNGKAVCVCRN

SEQ ID NO: 777 - protein, 38 aa, Artificial Sequence
  Discovered from phage display experiment
GFGCPLDQMQCHRHCQTITGRSGGYCSGPLKLTCTCYR

SEQ ID NO: 778 - protein, 38 aa, Artificial Sequence
  Discovered from phage display experiment
GFGCPLNQGACHRHCRSIRRRGGYCAGFFKQTCTCYRN

SEQ ID NO: 779 - protein, 32 aa, Artificial Sequence
  Discovered from phage display experiment
ALWKTMLKKLGTMALHAGKAALGAADTISQTQ

SEQ ID NO: 780 - protein, 19 aa, Artificial Sequence
  Discovered from phage display experiment
GKPRPYSPRPTSHPRPIRV

SEQ ID NO: 781 - protein, 46 aa, Artificial Sequence
  Discovered from phage display experiment
GIFSKLGRKKIKNLLISGLKNVGKEVGMDVVRTGIDIAGCKIKGEC

SEQ ID NO: 782 - protein, 13 aa, Artificial Sequence
  Discovered from phage display experiment
ILPWKWPWWPWRR

SEQ ID NO: 783 - protein, 25 aa, Artificial Sequence
  Discovered from phage display experiment
FKCRRWQWRMKKLGAPSITCVRRAP

SEQ ID NO: 784 - protein, 34 aa, Artificial Sequence
  Discovered from phage display experiment
ITSISLCTPGCKTGALMGCNMKTATCHCSIHVSK

SEQ ID NO: 785 - protein, 34 aa, Artificial Sequence
  Discovered from phage display experiment
TAGPAIRASVKQCQKTLKATRLFTVSCKGKNGCK

SEQ ID NO: 786 - protein, 56 aa, Artificial Sequence
  Discovered from phage display experiment
MSKFDDFDLDVVKVSKQDSKITPQWKSESLCTPGCVTGALQTCFLQTLTCNCKISK

SEQ ID NO: 787 - protein, 17 aa, Artificial Sequence
  Discovered from phage display experiment
KYYGNGVHCTKSGCSVN

SEQ ID NO: 788 - protein, 20 aa, Artificial Sequence
  Discovered from phage display experiment
WGEAFSAGVHRLANGGNGFW

SEQ ID NO: 789 - protein, 23 aa, Artificial Sequence
  Discovered from phage display experiment
GIGKFLHSAGKFGKAFVGEIMKS

SEQ ID NO: 790 - protein, 23 aa, Artificial Sequence
  Discovered from phage display experiment
GIGKFLHSAKKFGKAFVGEIMNS

SEQ ID NO: 791 - protein, 21 aa, Artificial Sequence
  Discovered from phage display experiment
GMASKAGAIAGKIAKVALKAL

SEQ ID NO: 792 - protein, 24 aa, Artificial Sequence
  Discovered from phage display experiment
GVLSNVIGYLKKLGTGALNAVLKG

SEQ ID NO: 793 - protein, 25 aa, Artificial Sequence
  Discovered from phage display experiment
GWASKIGQTLGKIAKVGLKELIQPK

SEQ ID NO: 794 - protein, 14 aa, Artificial Sequence
  Discovered from phage display experiment
INLKALAALAKKIL

SEQ ID NO: 795 - protein, 26 aa, Artificial Sequence
  Discovered from phage display experiment
GIGAVLKVLTTGLPALISWIKRKRQQ

SEQ ID NO: 796 - protein, 40 aa, Artificial Sequence
  Discovered from phage display experiment
ATCDLLSGTGINHSACAAHCLLRGNRGGYCNGKGVCVCRN

SEQ ID NO: 797 - protein, 39 aa, Artificial Sequence
  Discovered from phage display experiment
ATCDLLSGTGINHSACAAHCLLRGNRGGYCNRKGVCVRN

SEQ ID NO: 798 - protein, 18 aa, Artificial Sequence
  Discovered from phage display experiment
RRWCFRVCYRGFCYRKCR

SEQ ID NO: 799 - protein, 18 aa, Artificial Sequence
  Discovered from phage display experiment
RRWCFRVCYKGFCYRKCR

SEQ ID NO: 800 - protein, 18 aa, Artificial Sequence
  Discovered from phage display experiment
RGGRLCYCRRRFCVCVGR

SEQ ID NO: 801 - protein, 18 aa, Artificial Sequence
  Discovered from phage display experiment
RGGGLCYCRRRFCVCVGR

SEQ ID NO: 802 - protein, 51 aa, Artificial Sequence
  Discovered from phage display experiment
VTCDLLSFKGQVNDSACAANCLSLGKAGGHCEKGVCICRKTSFKDLWDKYF

SEQ ID NO: 803 - protein, 39 aa, Artificial Sequence
  Discovered from phage display experiment
GWLKKIGKKIERVGQHTRDATIQGLGIAQQAANVAATAR

SEQ ID NO: 804 - protein, 47 aa, Artificial Sequence
  Discovered from phage display experiment
SDEKASPDKHHRFSLSRYAKLANRLANPKLLETFLSKWIGDRGNRSV

SEQ ID NO: 805 - protein, 17 aa, Artificial Sequence
  Discovered from phage display experiment
KWCFRVCYRGICYRRCR

SEQ ID NO: 806 - protein, 17 aa, Artificial Sequence
  Discovered from phage display experiment
RWCFRVCYRGICYRKCR

SEQ ID NO: 807 - protein, 45 aa, Artificial Sequence
  Discovered from phage display experiment
KSCCKDTLARNCYNTCRFAGGSRPVCAGACRCKIIGPKCPSDYPK

SEQ ID NO: 808 - protein, 23 aa, Artificial Sequence
  Discovered from phage display experiment
GGKPDLRPCIIPPCHYIPRPKPR

SEQ ID NO: 809 - protein, 63 aa, Artificial Sequence
  Discovered from phage display experiment
VKDGYIVDDVNCTYFCGRNAYCNEECTKLKGESGYCQWASPYGNACYCKLPDHVRTKGPGRCH

SEQ ID NO: 810 - protein, 31 aa, Artificial Sequence
  Discovered from phage display experiment
KDEPQRRSARLSAKPAPPKPEPKPKKAPAKK

SEQ ID NO: 811 - protein, 28 aa, Artificial Sequence
  Discovered from phage display experiment
AESGDDYCVLVFTDSAWTKICDWSHFRN

SEQ ID NO: 812 - protein, 12 aa, Artificial Sequence
  Discovered from phage display experiment
WQPDTAHHWATL

SEQ ID NO: 813 - protein, 9 aa, Artificial Sequence
  Discovered from phage display experiment
CTITSKRTC

SEQ ID NO: 814 - protein, 9 aa, Artificial Sequence
  Discovered from phage display experiment
CQKHHNYLC

SEQ ID NO: 815 - protein, 9 aa, Artificial Sequence
  Discovered from phage display experiment
CTLVPHTRC

SEQ ID NO: 816 - protein, 13 aa, Artificial Sequence
  Discovered from phage display experiment
CNGRCVSGCAGRC

SEQ ID NO: 817 - protein, 9 aa, Artificial Sequence
  Discovered from phage display experiment
CVCNGRMEC

SEQ ID NO: 818 - protein, 6 aa, Artificial Sequence
  Discovered from phage display experiment
KCCYSL

SEQ ID NO: 819 - protein, 7 aa, Artificial Sequence
  Discovered from phage display experiment
VHLGYAT

SEQ ID NO: 820 - protein, 9 aa, Artificial Sequence
  Discovered from phage display experiment
CPIEDRPMC

SEQ ID NO: 821 - protein, 16 aa, Artificial Sequence
  Discovered from phage display experiment
NGYEIEWYSWVTHGMY

SEQ ID NO: 822 - protein, 6 aa, Artificial Sequence
  Discovered from phage display experiment
RRKRRR

SEQ ID NO: 823 - protein, 7 aa, Artificial Sequence
  Discovered from phage display experiment
ATWLPPR

SEQ ID NO: 824 - protein, 15 aa, Artificial Sequence
  Discovered from phage display experiment
ASSSYPLIHWRPWAR

SEQ ID NO: 825 - protein, 17 aa, Artificial Sequence
  Discovered from phage display experiment
KGVSLSYRKRYSLSVGK

SEQ ID NO: 826 - protein, 9 aa, Artificial Sequence
  Discovered from phage display experiment
EVCLVLSCQ

SEQ ID NO: 827 - protein, 9 aa, Artificial Sequence
  Discovered from phage display experiment
RDCVKNLCR

SEQ ID NO: 828 - protein, 9 aa, Artificial Sequence
  Discovered from phage display experiment
LGCFXSWCG

SEQ ID NO: 829 - protein, 9 aa, Artificial Sequence
  Discovered from phage display experiment
IPCSLLGCA

SEQ ID NO: 830 - protein, 9 aa, Artificial Sequence
  Discovered from phage display experiment
PRCWERVCS

SEQ ID NO: 831 - protein, 9 aa, Artificial Sequence
  Discovered from phage display experiment
TLCPLVACE

SEQ ID NO: 832 - protein, 9 aa, Artificial Sequence
  Discovered from phage display experiment
SECYTGSCP

SEQ ID NO: 833 - protein, 9 aa, Artificial Sequence
  Discovered from phage display experment
VECGFSAVF

SEQ ID NO: 834 - protein, 9 aa, Artificial Sequence
  Discovered from phage display experiment
HWCRLLACR

SEQ ID NO: 835 - protein, 7 aa, Artificial Sequence
  Discovered from phage display experiment
SRESPHP

SEQ ID NO: 836 - protein, 7 aa, Artificial Sequence
  Discovered from phage display experiment
HTFEPGV

SEQ ID NO: 837 - protein, 9 aa, Artificial Sequence
  Discovered from phage display experiment
CSNRDARRC

SEQ ID NO: 838 - protein, 9 aa, Artificial Sequence
  Discovered from phage display experiment
CXNXDXRXC

SEQ ID NO: 839 - protein, 5 aa, Artificial Sequence
  Discovered from phage display experiment
SSSPL

SEQ ID NO: 840 - protein, 4 aa, Artificial Sequence
  Discovered from phage display experiment
PHSK

SEQ ID NO: 841 - protein, 4 aa, Artificial Sequence
  Discovered from phage display experiment
LSAN

SEQ ID NO: 842 - protein, 4 aa, Artificial Sequence
  Discovered from phage display experiment
KHST

SEQ ID NO: 843 - protein, 4 aa, Artificial Sequence
  Discovered from phage display experiment
TLLS

SEQ ID NO: 844 - protein, 4 aa, Artificial Sequence
  Discovered from phage display experiment
SSTA

SEQ ID NO: 845 - protein, 4 aa, Artificial Sequence
  Discovered from phage display experiment
TSAH

SEQ ID NO: 846 - protein, 9 aa, Artificial Sequence
  Discovered from phage display experiment
CPGPEGAGC

SEQ ID NO: 847 - protein, 12 aa, Artificial Sequence
  Discovered from phage display experiment
YCDGFYACYMDV

SEQ ID NO: 848 - protein, 13 aa, Artificial Sequence
  Discovered from phage display experiment
YCDGFYACYMDVX

SEQ ID NO: 849 - protein, 13 aa, Artificial Sequence
  Discovered from phage display experiment
YCDGFYACYMDVX

SEQ ID NO: 850 - protein, 14 aa, Artificial Sequence
  Discovered from phage display experiment
YCDGFYACYMDVGX

SEQ ID NO: 851 - protein, 10 aa, Artificial Sequence
  Discovered from phage display experiment
CNKYFSNICW

SEQ ID NO: 852 - protein, 10 aa, Artificial Sequence
  Discovered from phage display experiment
CNKYFSNICW

SEQ ID NO: 853 - protein, 17 aa, Artificial Sequence
  Discovered from phage display experiment
NLTYMYGLSLHHYTSEA

SEQ ID NO: 854 - protein, 13 aa, Artificial Sequence
  Discovered from phage display experiment
CYVALPGGYVRKC

SEQ ID NO: 855 - protein, 12 aa, Artificial Sequence
  Discovered from phage display experiment
WDLAWMFRLPVG

SEQ ID NO: 856 - protein, 8 aa, Artificial Sequence
  Discovered from phage display experiment
WIFPWIQL

SEQ ID NO: 857 - protein, 5 aa, Artificial Sequence
  Discovered from phage display experiment
GRGDS

SEQ ID NO: 858 - protein, 9 aa, Artificial Sequence
  Discovered from phage display experiment
CDCRGDCFC

SEQ ID NO: 859 - protein, 13 aa, Artificial Sequence
  Discovered from phage display experiment
CPKVCPRECESNC

SEQ ID NO: 860 - protein, 9 aa, Artificial Sequence
  Discovered from phage display experiment
RRRRRRRRR

SEQ ID NO: 861 - protein, 11 aa, Artificial Sequence
  Discovered from phage display experiment
YGRKKRRQRRR

SEQ ID NO: 862 - protein, 17 aa, Artificial Sequence
  Discovered from phage display experiment
KKWKMRRNQFWVKVERG

SEQ ID NO: 863 - protein, 8 aa, Artificial Sequence
  Discovered from phage display experiment
RRRRRRRR

SEQ ID NO: 864 - protein, 8 aa, Artificial Sequence
  Discovered from phage display experiment
EEPPSSPK

SEQ ID NO: 865 - protein, 5 aa, Artificial Sequence
  Discovered from phage display experiment
CNGRC